(19) United States
(12) Patent Application Publication
(10) Pub. No.: US 2015/0299797 A1
(43) Pub. Date: Oct. 22, 2015

(54) COMPOSITIONS AND METHODS RELATING TO BLOOD-BASED BIOMARKERS OF BREAST CANCER

(71) Applicant: UNIVERSITY OF UTAH RESEARCH FOUNDATION, Salt Lake City, UT (US)

(72) Inventors: Andrea BILD, Salt Lake City, UT (US); Saundra BUYS, Salt Lake City, UT (US); William Evan JOHNSON, Wakefield, MA (US)

(21) Appl. No.: 14/423,390

(22) PCT Filed: Aug. 22, 2013
(86) PCT No.: PCT/US2013/056201

§ 371 Date: Feb. 23, 2015

Related U.S. Application Data
(60) Provisional application No. 61/693,014, filed on Aug. 24, 2012.
(60) Provisional application No. 61/787,790, filed on Mar. 15, 2013.

(51) Int. Cl. C12Q 1/68

(52) U.S. Cl. CPC C12Q 1/6886; C12Q 2600/16; C12Q 2600/112; C12Q 2600/156; C12Q 2600/158

(57) ABSTRACT

The present invention relates generally to the field of breast cancer. More specifically, the invention concerns methods and compositions useful for diagnosing treating breast cancer.

### CROSS-REFERENCE TO RELATED APPLICATIONS

This application claims benefit of U.S. Provisional Application No. 61/787,790, filed Mar. 15, 2013 and U.S. Provisional Application No. 61/693,014, filed Aug. 24, 2012, each of which are hereby incorporated herein by reference in their entirety.

### BACKGROUND

Family history is an important indicator of a woman's risk of developing breast cancer (Amir 2010), but women with a family history of breast cancer and their physicians face uncertainty regarding how to manage disease risk. Various prevention strategies exist, including chemoprevention via selective estrogen receptor modulators (SERMs) (Poweles 2002), prophylactic mastectomy or oophorectomy, and increased surveillance. However, tradeoffs accompany each of these strategies (Vogelstein 2004). What is needed in the art is a way to accurately predict breast-cancer development in individuals from high-risk families, which enables more effective prioritization of such prevention strategies.

In recent years, various breast-cancer risk models have been proposed. Several models have attempted to account for the combinatory effects of clinically observable factors such as age, hormonal and reproductive history, breast-disease history, and family history. (Amir 2010) And because breast tumors arise from molecular-level aberrations, (Vogelstein 2004) some models have also incorporated information about germline variants. (Tyrer 2004; Wacholder 2012). In evaluation analyses, the models have demonstrated some ability to predict who will develop breast cancer, attaining area under the receiver operating characteristic curve (AUC) values ranging from 0.540 to 0.762. (Wacholder 2012; Rockhill 2001; Amir 2003; Boughey 2010) However, discrete genetic events may not portray an adequate picture of the underlying molecular mechanisms influencing breast-cancer susceptibility.

Highly penetrant mutations in breast-cancer susceptibility genes including BRCA1 (Miki 1994), BRCA2 (Easton 1997), TP53 (Sidranksky 1992), PTEN (Liaw 1997), and ATM (Swift 1987) are relatively rare in the population. Mutations in these genes account for only about 20% of overall familial breast-cancer risk. (Stratton 2008) Additionally, low- and moderate-penetrance variants explain only about 13% of familial risk, even though they are relatively common. (Maraddat 2012). Variants of any penetrance or population frequency may lack utility in prospective models if they affect risk only subtly or only in conjunction with other risk factors. Complicating matters further is that predisposing genetic events are not limited to single-nucleotide variants. Structural variants including insertions, deletions, block substitutions, inversions, and copy-number variations can also be inherited and are believed to contribute to many phenotypes (Frazer 2004). In humans, it is believed that structural variants account for at least 20% of genetic variation (Frazer 2004).

An alternative to estimating risk with genetic variants is to model the downstream effects of such events. Multiple variants within a given gene or pathway may have similar downstream effects if they drive coordinate changes in mRNA transcription levels. Thus even though two individuals may differ in the germline variant(s) they carry, changes in breast-cancer susceptibility, mediated through mRNA transcription, may be similar for both. Variants within a gene's regulatory elements can drive mRNA-expression changes within that gene (cis-acting effects); however, genetic variants can also influence expression of genes on other chromosomes via trans-acting effects (Schadt 2003; Morley 2004). In fact, recent studies have demonstrated that genetic variation can be associated consistently with global mRNA-expression patterns and that these patterns can reflect heritable disease susceptibility (Cookson 2009; Stranger 2007). Such recognizable expression patterns may result from aberrant pathway activity driven by germline genetic variation; they may also be influenced by molecular-level factors not encoded in DNA, including epigenetic modifications and splice variation.

Specific to breast cancer, tumor expression has been shown to correlate with germline mutation status. In a multivariate analysis, tumors of patients carrying BRCA1 or BRCA2 mutations could be distinguished from each other with high accuracy, and these tumors could be distinguished from sporadic tumors (Hedenfalk 2001). In a later study, lymphoblastoid cell lines derived from BRCA1/2 carriers expressed mRNA signatures that were distinct from those of “BRCAX” carriers (patients with a family history but no known BRCA1/2 mutation) (Waddell 2008). Therefore, what is needed is global mRNA-expression biomarkers that can serve as an intermediate phenotype representing heritable breast-cancer susceptibility.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE FIGURES

The accompanying figures, which are incorporated in and constitute a part of this specification, illustrate several aspects and together with the description serve to explain the principles of the invention.

FIG. 1 shows an overview of criteria used to filter exome-sequencing variants. Variants were filtered based on frequency, location within protein-coding regions, conservation, and effect on protein.

FIG. 2 shows the variants identified via exome-capture sequencing in known breast-cancer susceptibility genes. Patients are grouped according to BRCA1/2 mutation status (determined via commercial genetic testing) and diagnosis. Exome-sequencing variant calls for BRCA1 and BRCA2 agreed closely with commercial test results (exceptions are noted).

FIG. 3 is a schematic showing the risk groups for gene-expression analysis. Peripheral blood mononuclear cells for a cohort of 124 Utah women of northern European descentwere profiled using gene-expression microarrays. Each woman fell into one of six groups, divided along three axes: 1) family history of breast cancer, 2) presence of a pathogenic germline variant in BRCA1 or BRCA2, and 3) previous diagnosis with early-onset breast cancer.

FIG. 4 shows ROC curve representing the ability to differentiate high-risk cancer patients who develop breast cancer from controls for the Utah cohort. Prior to constructing SVM classification models, the SVM-RFE selection method was applied to all genes.

FIG. 5 shows ROC curve representing the ability to differentiate high-risk cancer patients who develop breast cancer from controls for the Ontario cohorts. Prior to constructing SVM classification models, the SVM-RFE selection method was applied to all genes. Data for both Ontario cohorts are combined in the figures.

FIG. 6 shows the distribution of AUC values cross-validation assignments were randomly varied and the experiment was repeated 1,000 times.

FIG. 7 shows AUC performance versus the number of top-ranked genes that were included in SVM classification models across 1,000 permutations of cross-validation assignments. Patients from the Utah cohort were used, and SVM-RFE was used for gene selection. The vertical red line indicates the number of genes that attained the highest average AUC.

FIG. 8 shows the distribution of AUC values obtained when the class labels were permuted and the experiment was repeated 1,000 times. The dotted, red vertical line indicates the non-permuted performance. The percentage of times that the actual result was higher than the permuted results is also shown.

FIG. 9 is a schematic diagram showing the risk groups for exome-capture sequencing. Peripheral-blood DNA for a cohort of 35 women from Utah were sequenced. Each woman fell into one of four groups, divided along two axes: 1) prior identification (via commercial genetic testing) of a pathogenic germline variant in BRCA1 or BRCA2, and 2) prior diagnosis with early-onset breast cancer. “BRCAX” individuals carry a genetic predisposition to breast cancer, but no known causal variant had not been identified.

FIG. 10 shows the selection of threshold used to filter frequently mutated genes. Genes that were mutated in a relatively high number of TCGA germline samples were excluded from the pathway level mutation analyses. The number of genes that would be excluded for thresholds ranging between 0.2% and 10% were calculated. A threshold of 1.8% was selected based on the maximal difference in number of excluded genes.

FIGS. 11A and 11B show flowcharts that show the high-level design of the experiments. A) Gene-expression and exome-sequencing data were acquired for women who developed hereditary breast cancer and for controls. Expression values and mutation counts were aggregated at the pathway level and compared between the two groups. Pathways for which the greatest level of discrimination between the groups could be attained were considered candidates for further investigation via cell-based and microscopy analyses. B) A gene-expression biomarker was derived from genome-wide expression values; the biomarker's ability to generate accurate predictions was validated in retrospective and prospective cohorts from Ontario, Canada.

FIGS. 12A, 12B and 12C show a summary of pathways that performed best in both gene-expression and exomesequencing analyses. A) These pathways performed well both in gene-expression (p<0.05 for Utah/Ontario) and exome-sequencing analyses (p<0.10) that compared hereditary breast cancer (HBC) profiles against controls. The values listed for each cohort represent empirical pvalues attained through a permutation approach. “Differentially expressed” genes are those for which expression was consistently higher/lower in HBC compared to controls in the Utah and Ontario cohorts. B) Heatmaps show average expression levels for HBC individuals and controls in the Utah and Ontario cohorts. They include top-ranked genes (as determined by SVM-RFE) that exhibit a consistent fold change across the cohorts. Warmer colors indicate higher relative expression; cooler colors indicate the opposite. These visualizations highlight that for many genes in these pathways, average expression levels are considerably different for HBC relative to controls. C) Summary of mutations observed in key pathways. The number of mutations represents how many samples in each cohort had at least one mutation. Genes for which at least one mutation was observed across the HBC samples are listed.

FIG. 13 is an overview of criteria used to filter exome-sequencing variants. Variants were filtered based on frequency, location within protein-coding regions, conservation, and effect on protein coding. Variants were also collapsed to gene-level values before pathway-level comparisons were performed. All statistics listed on this diagram are per sample.

FIG. 14 shows a gene-expression heatmap for KEGGcell-adhesion molecules pathway. This heatmap shows average expression levels for HBC individuals and controls in the Utah and Ontario cohorts. It includes top-ranked genes (as determined by SVM-RFE) only for this pathway.

FIG. 15 shows a gene-expression heatmap for Reactome integrin cell-surface interactions pathway. This heatmap shows average expression levels for HBC individuals and controls in the Utah and Ontario cohorts. It includes top-ranked genes (as determined by SVM-RFE) only for this pathway.

FIG. 16 shows the relationship between mutation status and gene expression for 34 samples profiled using both technologies. Data for 357 genes that exhibited a strong association (see Methods) between the presence of a suspected pathogenic variant and expression of the gene containing the variant(s) are displayed. Dark dots indicate mutations within a given sample. Expression values for each gene were standardized to a consistent scale for illustration purposes.

FIG. 17 shows Gefitinib assay results. Primary breast cells were treated with the EGFR inhibitor Gefitinib, and similar responses were observed for HBC women and controls.

FIG. 18 shows Afatinib assay results. Primary breast cells were treated with the tyrosine kinase inhibitor Afatinib, and similar responses were observed for HBC women and controls.

FIG. 19 shows TRAIL assay results for individual cell lines. A TRAIL assay was used to compare responsiveness to apoptotic signals in primary breast cells for HBC women and controls. Each line represents the fitted response for an individual patient.

FIGS. 20A and B show cell-based assay results. A) B) A cell-adhesion assay was used to compare adhesiveness to the extracellular matrix of primary breast cells in HBC women compared to controls. HBC cells were significantly less adherent than control cells. C) For primary breast cells, a TRAIL assay was used to compare responsiveness to apoptotic signals for HBC women and controls. HBC cells were significantly more responsive to TNF signaling.

FIG. 21 shows biomarker prediction results when randomly permuted class labels were used for Ontario data. This validation approach enabled us to estimate the probability of observing the prediction accuracy observed for the actual predictions.

FIG. 22 shows gene-expression levels for ITGA6 across subgroups.

FIGS. 23A, B, C, and D show gene-expression biomarker prediction results. A) The support vector machines algorithm was used to produce a probability that each patient belongs to the hereditary breast cancer (HBC) group. This plot shows probabilities for the Utah cohort, estimated via crossvalidation. Probabilities were considerably higher for HBC individuals than for controls, which indicates the algorithm's ability to discriminate the groups, regardless of BRCA mutation status. B) A receiver operating characteristic (ROC) curve illustrates the tradeoff between sensitivity and specificity when various probability thresholds are used to discriminate the groups in the Utah cohort. C) HBC probabilities for the Ontario cohort, estimated via a training-testing design. D) ROC curve for the Ontario cohort.

FIG. 24 shows gene-expression levels for PTK2 across subgroups.

FIG. 25 shows gene-expression levels for NEO1 across subgroups.

FIG. 26 shows a summary of variant effects on protein function. Most suspected pathogenic variants were non-synonymous substitutions. However, many other types of variant effect were also observed.

FIG. 27 shows a summary of DNA alterations that resulted from suspected pathogenic variants. Single-nucleotide substitutions exhibited a non-uniform distribution; G-to-A and C-to-T transitions were most common. Deletions were more common than insertions.

FIG. 28 shows a net gain/loss in nucleotide bases resulting from suspected pathogenic variants. Most variants resulted in no gain or loss (predominantly single-nucleotide substitutions). However, as many as 39/36 nucleotides were gained/lost.

FIG. 29 shows the variants identified via exome-capture sequencing in BRCA1 and BRCA2. Patients are grouped according to BRCA1/2 mutation status (determined via commercial genetic testing) and diagnosis. Exome-sequencing variant calls for BRCA1 and BRCA2 agreed closely with commercial test results. Exceptions are noted. “False positive” variants were indicated by exome-sequencing analysis to be pathogenic, whereas commercial testing suggested otherwise. “False negative” variants were identified as pathogenic in commercial testing but did not pass filtering criteria in the analysis.

FIG. 30 shows the BRCA1 variants identified via exome sequencing. Various BRCA1 variants were identified in the hereditary breast-cancer cohort from Utah. The horizontal axis depicts genomic position along the gene. The right panel indicates relative gene expression for each sample.

FIG. 31 shows the BRCA2 variants identified via exome sequencing. Various BRCA2 variants were identified in the hereditary breast-cancer cohort from Utah. The horizontal axis depicts genomic position along the gene. The right panel indicates relative gene expression for each sample.

### DETAILED DESCRIPTION OF THE INVENTION

Women with a strong family history of breast cancer face crucial decisions regarding disease risk. Preventative medicines, surgeries, and surveillance may be instrumental in averting tumor development and prolonging life; however, considerable adverse effects may accompany these strategies. Thus techniques that target prevention strategies at individuals with the highest risk can help strike a balance between benefits and risks. Risk-stratification tools that focus on clinically observable factors alone may lack accuracy by not accounting for molecular-level factors that drive tumor development. Such tools can also be subject to recall errors and may not generalize to populations with multiple ethnicities.

Genetic screening for mutations in well-recognized breast-cancer genes like BRCA1 and BRCA2 can provide valuable information in addressing this problem, but mutations in these genes account for only a portion of high-risk breast-cancer cases. Mutations in other genes are believed to influence breast-cancer risk but likely only moderately and perhaps only in combination with other mutations. Further complicating the matter, various types of genomic aberrations beyond single-nucleotide variants can be inherited and can influence an individual's susceptibility to breast cancer.

Disclosed herein are methods and compositions that account for the downstream effects of a variety of genetic aberrations by profiling mRNA expression levels in peripheral blood samples. It was also observed that such expression patterns can accurately identify high-risk patients who develop breast tumors. This finding applied not only to multiple patient sets, an indication that such patterns are predictive of risk status before tumors develop and that such patterns are recognizable in multiple ethnicities.

This approach aims to account for gene-expression changes that reflect both initiating events and downstream pathway consequences.

The gene signatures disclosed herein characterize pathway-level activity that differs long before a tumor develops between high-risk individuals who develop breast tumors and other individuals.

The gene signatures disclosed herein can be combined with results from genetic screening for mutations in well-recognized breast-cancer genes like BRCA1 and BRCA2. Thus, disclosed are methods of assessing a subject's susceptibility to develop cancer by obtaining the expression profile of the genes from Tables 1-4 in combination with screening for BRCA1 or BRCA2 mutations. The combination can also be narrowed to only looking at the expression profile of genes in the pathways identified in Tables 8 and 9 (such as the genes in Tables 11, 12, and 13) in combination with BRCA1/2 mutations.

All patents, patent applications and publications cited herein, whether supra or infra, are hereby incorporated by reference in their entireties into this application in order to more fully describe the state of the art as known to those skilled therein as of the date of the invention described and claimed herein.

It is to be understood that this invention is not limited to specific synthetic methods, or to specific recombinant biotechnology methods unless otherwise specified, or to particular reagents unless otherwise specified, to specific pharmaceutical carriers, or to particular pharmaceutical formulations or administration regimens, as such may, of course, vary. It is also to be understood that the terminology used herein is for the purpose of describing particular embodiments only and is not intended to be limiting.

### DEFINITIONS AND NOMENCLATURE

The terminology used herein is for the purpose of describing particular embodiments only and is not intended to be limiting.

As used in the specification and the appended claims, the singular forms “a,” “an” and “the” can include plural referents unless the context clearly dictates otherwise. Thus, for example, reference to “a compound” includes mixtures of compounds, reference to “a pharmaceutical carrier” includes mixtures of two or more such carriers, and the like.

Ranges may be expressed herein as from “about” one particular value, and/or to “about” another particular value. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

The word “or” as used herein means any one member of a particular list and also includes any combination of members of that list.

By “sample” is meant an animal; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

By “modulate” is meant to alter, by increasing or decreasing.

By “normal subject” is meant an individual who does not have breast cancer.

The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

“Peptide” as used herein refers to any peptide, oligopeptide, polypeptide, gene product, expression product, or protein. For example, a peptide can be a receptor. A polypeptide is comprised of consecutive amino acids. The term “polypeptide” encompasses naturally occurring or synthetic molecules.

In addition, as used herein, the term “peptide” or “polypeptide” refers to amino acids joined to each other by peptide bonds or modified peptide bonds, e.g., peptide isosteres, etc. and may contain modified amino acids other than the 20 gene-encoded amino acids. The polypeptides can be modified by either natural processes, such as posttranslational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given polypeptide. Also, a given polypeptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pergylation, proteolytic processing, phosphorylation, prenylation, racemization, selenoylation, sulfation, and transfer-RNA mediated addition of amino acids to protein such as arginylation. (See Proteins—Structure and Molecular Properties 2nd Ed., T. E. Creighton, W.H. Freeman and Company, New York (1993); Posttranslational Covalent Modification of Proteins, B. C. Johnson, Ed., Academic Press, New York, pp. 1-12 (1983)).

As used herein, the term “amino acid sequence” refers to a list of abbreviations, letters, characters or words representing amino acid residues.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof

By an “effective amount” of a compound as provided herein is meant a sufficient amount of the compound to provide the desired effect. The exact amount required will vary from subject to subject, depending on the species, age, and general condition of the subject, the severity of disease (or underlying genetic defect) that is being treated, the particular compound used, its mode of administration, and the like. Thus, it is not possible to specify an exact “effective amount.” However, an appropriate “effective amount” may be determined by one of ordinary skill in the art using only routine experimentation.

By “treat” is meant to administer a compound or molecule to a subject, such as a human or other mammal (for example, an animal model), that has a condition or disease, such as b, an increased susceptibility for developing such a disease, in order to prevent or delay a worsening of the effects of the disease or condition, or to partially or fully reverse the effects of the disease. To “treat” can also refer to non-pharmacological methods of preventing or delaying a worsening of the effects of the disease or condition, or to partially or fully reversing the effects of the disease. For example, “treat” is meant to mean a course of action to prevent or delay a worsening of the effects of the disease or condition, or to partially or fully reverse the effects of the disease other than by administering a compound.

By “prevent” is meant to minimize the chance that a subject who has a susceptibility for developing breast cancer will develop a such a disease, or one or more symptoms associated with the disease.

By “probe,” “primer,” or oligonucleotide is meant a single-stranded DNA or RNA molecule of defined sequence that can base-pair to a second DNA or RNA molecule that contains a complementary sequence (the “target”). The stability of the resulting hybrid depends upon the extent of the base-pairing that occurs. The extent of base-pairing is affected by parameters such as the degree of complementarity between the probe and target molecules and the degree of stringency of the hybridization conditions. The degree of hybridization stringency is affected by parameters such as temperature, salt concentration, and the concentration of organic molecules such as formamide, and is determined by methods known to one skilled in the art. Probes or primers specific for c-Met nucleic acids (for example, genes and/or mRNAs) have at least 80%-90% sequence complementarity, preferably at least 91%-95% sequence complementarity, more preferably at least 96%-99% sequence complementarity, and most preferably 100% sequence complementarity to the region of the nucleic acid to which they hybridize. Probes, primers, and oligonucleotides may be detectably-labeled, either radioactively, or non-radioactively, by methods well-known to those skilled in the art. Probes, primers, and oligonucleotides are used for methods involving nucleic acid hybridization, such as: nucleic acid sequencing, reverse transcription and/or nucleic acid amplification by the polymerase chain reaction, single stranded conformational polymorphism (SSCP) analysis, restriction fragment polymorphism (RFLP) analysis, Southern hybridization, Northern hybridization, in situ hybridization, electrophoretic mobility shift assay (EMSA).

By “specifically hybridizes” is meant that a probe, primer, or oligonucleotide recognizes and physically interacts (that is, base-pairs) with a substantially complementary nucleic acid (for example, a c-met nucleic acid) under high stringency conditions, and does not substantially base pair with other nucleic acids.

By “high stringency conditions” is meant conditions that allow hybridization comparable with that resulting from the use of a DNA probe of at least 40 nucleotides in length, in a buffer containing 0.5 M NaHPO₄, pH 7.2, 7% SDS, 1 mM EDTA, and 1% BSA (Fraction V), at a temperature of 65° C., or a buffer containing 48% formamide, 4.8×SSC, 0.2 M Tris-Cl, pH 7.6, 1×Denhardt's solution, 10% dextran sulfate, and 0.1% SDS, at a temperature of 42° C. Other conditions for high stringency hybridization, such as for PCR, Northern, Southern, or in situ hybridization, DNA sequencing, etc., are well-known by those skilled in the art of molecular biology. (See, for example, F. Ausubel et al., Current Protocols in Molecular Biology, John Wiley & Sons, New York, N.Y., 1998).

The nucleic acids, such as, the polynucleotides described herein, can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 3rd Edition (Cold Spring Harbor Laboratory Press, Cold Spring Harbor, N.Y., 2001) Chapters 5, 6) to purely synthetic methods, for example, by the cyanoethyl phosphoramidite method using a Milligen or Beckman System 1Plus DNA synthesizer. Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

### Compositions

Although family history can increase breast-cancer risk, women from high-risk families face substantial uncertainty regarding their personal risk. In an attempt to guide prevention efforts, various risk models have been proposed. However, existing models do not account for the various types of molecular-level aberrations including single-nucleotide variants, structural variants, and epigenetic modifications that may modulate heritable risk. The potential to personalize risk estimates by modeling the downstream effects of such aberrations on transcription were studied. Having profiled global genomic variation in peripheral blood mononuclear cells, the support vector machines classification algorithm was used to discriminate high-risk individuals who develop breast cancer from control individuals. When tested on a geographically and ethnically distinct cohort that was recruited both retrospectively and prospectively, the models attained an area under the receiver operating characteristic curve of 0.733. Thus, even though individual patients vary in the risk-conferring aberrations they carry in the germline, genomic patterns that characterize familial breast-cancer risk are detectable in peripheral blood. Upon examining genes and signaling pathways that were prominent in the models, biochemical approaches were used to test the hypothesis that deregulation of TGF-β signaling, cell adhesion, and tumor necrosis factors can lead to an increased propensity for breast cells to transition into a mesenchymal state.

Disclosed herein are a gene expression panels and arrays indicative of the risk of developing breast cancer, said panel or array consisting of primers or probes capable of detecting at least 1 gene selected from: (i) the genes of Table 1; (ii) the genes of Table 2; and (iii) genes of Table 3; or (iv) the genes of Table 4.

The disclosed gene expression panels or arrays can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, or 604 genes, or any amount in between, of the genes found in Table 1, Table 2, Table 3, Table 4, or any combination of these tables, as disclosed herein.

The genes can all be selected from Table 1, all from Table 2, all from Table 3, all from Table 4, or from any combination or permutation of these tables. For instance, one could select 20 genes from Table 1, and 20 genes from Table 3 to use in the analysis. Alternatively, one could select 5 genes from Table 1, 5 genes from Table 2, 5 genes from Table 3, and 5 genes from Table 4.

The disclosed gene expression panels or arrays can consist of primers or probes capable of detecting or amplifying any number of the genes found in Tables 1-4, and in particular, can detect anywhere from 1-604 genes. For example, the primers or probes can detect or amplify between 5 and 600 genes, 10 and 500 genes, 20 and 300 genes, 30 and 200 genes, or any variation between

In one embodiment, the disclosed arrays or panels can be specific to hereditary breast cancer. Hereditary breast cancer is suspected when there is a strong family history of breast cancer, such as occurrences of the disease in at least three first or second-degree relatives (sisters, mothers, aunts). The risk of breast cancer determined by the methods disclosed herein does not have to be specific to hereditary breast cancer. Other factors can contribute to being high risk, such as lifestyle (smoking, exposure to various toxins) or mutations found in other genes which are known to be associated with cancer development.

Also disclosed herein is are gene expression panels or arrays indicative of the risk of developing breast cancer, said panel or array consisting of primers or probes capable of detecting genes selected from: (i) the genes of Table 5 or; (ii) the genes of Table 6. These panels can be used alone or in combination with one or more of the other gene expression panels or arrays disclosed herein. For example, disclosed herein is are gene expression panels or arrays indicative of the risk of developing breast cancer, said panel or array consisting of primers or probes capable of detecting one or more genes selected from: (i) the genes of Table 5; (ii) the genes of Table 6; (iiii) the genes of Table 1; (iv) the genes of Table 2; (v) genes of Table 3; or (vi) the genes of Table 4.

Table 5 comprises 79 genes. As such, disclosed herein are gene expression panels or arrays that comprise anywhere from 1-79 of these genes, including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79 of these genes, in any order, combination, or permutation.

Table 6 comprises 121 genes. As such, disclosed herein are gene expression panels or arrays that comprise anywhere from 1-121 of these genes, including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, or 121 of these genes, in any order, combination, or permutation.

The disclosed gene expression panels and arrays can consist of primers or probes capable of detecting or amplifying any number of the genes found in Table 5 or Table 6, and in particular, can detect anywhere from 1-200 genes. For example, the primers or probes can detect or amplify between 20 and 200 genes.

The disclosed gene expression panels and arrays can be used in methods that generate a specific profile. The profile can be provided in the form of a heatmap or boxplot.

The profile of the expression levels of the genes can be used to compute a statistically significant value based on differential expression of the group of genes, wherein the computed value correlates to a diagnosis for a subgroup of breast cancer. The variance in the obtained profile of expression levels of the said selected genes or gene expression products can be either upregulated or downregulated in high risk individuals as compared to controls. This is described in more detail herein. For example, when the genes of Tables 1 and 3 are downregulated, these are indicative of a higher risk of breast cancer. When the genes of Tables 2 and 4 are upregulated, these are indicative of a higher risk of breast cancer as well. As described herein, one of skill in the art can use a combination of the genes from any of these Tables to form a profile, which can then be used to analyze the risk of developing breast cancer, or to determine if a subject has breast cancer.

The gene expression panel or array can consist of primers or probes capable of detecting, amplifying or otherwise measuring the presence or expression of one or more genes disclosed herein. For example, specific primers that can be used in the methods or with the compositions disclosed herein include, but are not limited to the primers suitable for use in the standard exon array from the Affymetrix website listed at: http://www.affymetrix.com.

Also disclosed are diagnostics kit containing one or more probes or primers capable of detecting, amplifying or otherwise measuring the presence or expression of one or more genes of Table 1, Table 2, Table 3, Table 4, Table 5, and/or Table 6.

Disclosed herein are solid supports comprising one or more primers, probes, polypeptides, or antibodies capable of hybridizing or binding to one or more of the genes found in any of the Tables 1-6 found herein. Solid supports are solid-state substrates or supports with which molecules, such as analytes and analyte binding molecules, can be associated. Analytes, such as calcifying nano-particles and proteins, can be associated with solid supports directly or indirectly. For example, analytes can be directly immobilized on solid supports. Analyte capture agents, such a capture compounds, can also be immobilized on solid supports.

The term “differentially expressed” or “differential expression,” as well as the term “variant,” as used herein refers to a difference in the level of expression of the biomarkers that can be assayed by measuring the level of expression of the products of the biomarkers, such as the difference in level of messenger RNA transcript or a portion thereof expressed or of proteins expressed of the biomarkers. In a preferred embodiment, the difference is statistically significant. The term “difference in the level of expression” refers to an increase or decrease in the measurable expression level of a given biomarker, for example as measured by the amount of messenger RNA transcript and/or the amount of protein in a sample as compared with the measurable expression level of a given biomarker in a control. In one embodiment, the differential expression can be compared using the ratio of the level of expression of a given biomarker or biomarkers (such as the genes found in Table 1) as compared with the expression level of the given biomarker or biomarkers of a control, wherein the ratio is not equal to 1.0. For example, an RNA or protein is differentially expressed if the ratio of the level of expression in a first sample as compared with a second sample is greater than or less than 1.0. For example, a ratio of greater than 1, 1.2, 1.5, 1.7, 2, 3, 3, 5, 10, 15, 20 or more, or a ratio less than 1, 0.8, 0.6, 0.4, 0.2, 0.1, 0.05, 0.001 or less. In another embodiment the differential expression is measured using p-value. For instance, when using p-value, a biomarker is identified as being differentially expressed as between a first sample and a second sample when the p-value is less than 0.1, preferably less than 0.05, more preferably less than 0.01, even more preferably less than 0.005, the most preferably less than 0.001.

The term “similarity in expression” as used herein means that there is no or little difference in the level of expression of the biomarkers between the test sample and the control or reference profile. For example, similarity can refer to a fold difference compared to a control. In one example, there is no statistically significant difference in the level of expression of the biomarkers.

The term “most similar” in the context of a reference profile refers to a reference profile that is associated with a clinical outcome that shows the greatest number of identities and/or degree of changes with the subject profile.

The phrase “determining the expression of biomarkers” as used herein refers to determining or quantifying RNA or proteins or protein activities or protein-related metabolites expressed by the genes disclosed herein. The term “RNA” includes mRNA transcripts, and/or specific spliced or other alternative variants of mRNA, including antisense products. The term “RNA product of the biomarker” as used herein refers to RNA transcripts transcribed from the biomarkers and/or specific spliced or alternative variants. In the case of “protein”, it refers to proteins translated from the RNA transcripts transcribed from the biomarkers. The term “protein product of the biomarker” refers to proteins translated from RNA products of the biomarkers.

A person skilled in the art will appreciate that a number of methods can be used to detect or quantify the level of RNA products of the biomarkers within a sample, including arrays, such as microarrays, RT-PCR (including quantitative RT-PCR), nuclease protection assays and Northern blot analyses.

Accordingly, in one example, the biomarker expression levels are determined using arrays, optionally microarrays, RT-PCR, optionally quantitative RT-PCR, nuclease protection assays or Northern blot analyses.

A form of solid support is an array. Another form of solid support is an array detector. An array detector is a solid support to which multiple different capture compounds or detection compounds have been coupled in an array, grid, or other organized pattern.

Solid-state substrates for use in solid supports can include any solid material to which molecules can be coupled. This includes materials such as acrylamide, agarose, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A form for a solid-state substrate is a microtiter dish, such as a standard 96-well type. In preferred embodiments, a multiwell glass slide can be employed that normally contain one array per well. This feature allows for greater control of assay reproducibility, increased throughput and sample handling, and ease of automation.

Different compounds can be used together as a set. The set can be used as a mixture of all or subsets of the compounds used separately in separate reactions, or immobilized in an array. Compounds used separately or as mixtures can be physically separable through, for example, association with or immobilization on a solid support. An array can include a plurality of compounds immobilized at identified or predefined locations on the array. Each predefined location on the array generally can have one type of component (that is, all the components at that location are the same). Each location will have multiple copies of the component. The spatial separation of different components in the array allows separate detection and identification of the polynucleotides or polypeptides disclosed herein.

It is not required that a given array be a single unit or structure. The set of compounds may be distributed over any number of solid supports. For example, at one extreme, each compound may be immobilized in a separate reaction tube or container, or on separate beads or microparticles. Different modes of the disclosed method can be performed with different components (for example, different compounds specific for different proteins) immobilized on a solid support.

Some solid supports can have capture compounds, such as antibodies, attached to a solid-state substrate. Such capture compounds can be specific for calcifying nano-particles or a protein on calcifying nano-particles. Captured calcifying nano-particles or proteins can then be detected by binding of a second, detection compound, such as an antibody. The detection compound can be specific for the same or a different protein on the calcifying nano-particle.

Methods for immobilizing nucleic acids, peptides or antibodies (and other proteins) to solid-state substrates are well established. Immobilization can be accomplished by attachment, for example, to aminated surfaces, carboxylated surfaces or hydroxylated surfaces using standard immobilization chemistries. Examples of attachment agents are cyanogen bromide, succinimide, aldehydes, tosyl chloride, avidin-biotin, photocrosslinkable agents, epoxides and maleimides. A preferred attachment agent is the heterobifunctional cross-linker N-[γ-Maleimidobutyryloxy] succinimide ester (GMBS). These and other attachment agents, as well as methods for their use in attachment, are described in Protein immobilization: fundamentals and applications, Richard F. Taylor, ed. (M. Dekker, New York, 1991); Johnstone and Thorpe, Immunochemistry In Practice (Blackwell Scientific Publications, Oxford, England, 1987) pages 209-216 and 241-242, and Immobilized Affinity Ligands; Craig T. Hermanson et al., eds. (Academic Press, New York, 1992) which are incorporated by reference in their entirety for methods of attaching antibodies to a solid-state substrate. Antibodies can be attached to a substrate by chemically cross-linking a free amino group on the antibody to reactive side groups present within the solid-state substrate. For example, antibodies may be chemically cross-linked to a substrate that contains free amino, carboxyl, or sulfur groups using glutaraldehyde, carbodiimides, or GMBS, respectively, as cross-linker agents. In this method, aqueous solutions containing free antibodies are incubated with the solid-state substrate in the presence of glutaraldehyde or carbodiimide.

A method for attaching antibodies or other proteins to a solid-state substrate is to functionalize the substrate with an amino- or thiol-silane, and then to activate the functionalized substrate with a homobifunctional cross-linker agent such as (Bis-sulfo-succinimidyl suberate (BS3) or a heterobifunctional cross-linker agent such as GMBS. For cross-linking with GMBS, glass substrates are chemically functionalized by immersing in a solution of mercaptopropyltrimethoxysilane (1% vol/vol in 95% ethanol pH 5.5) for 1 hour, rinsing in 95% ethanol and heating at 120° C. for 4 hrs. Thiol-derivatized slides are activated by immersing in a 0.5 mg/ml solution of GMBS in 1% dimethylformamide, 99% ethanol for 1 hour at room temperature. Antibodies or proteins are added directly to the activated substrate, which are then blocked with solutions containing agents such as 2% bovine serum albumin, and air-dried. Other standard immobilization chemistries are known by those of skill in the art.

Each of the components (compounds, for example) immobilized on the solid support preferably is located in a different predefined region of the solid support. Each of the different predefined regions can be physically separated from each other of the different regions. The distance between the different predefined regions of the solid support can be either fixed or variable. For example, in an array, each of the components can be arranged at fixed distances from each other, while components associated with beads will not be in a fixed spatial relationship. In particular, the use of multiple solid support units (for example, multiple beads) will result in variable distances.

Components can be associated or immobilized on a solid support at any density. Components preferably are immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

Optionally, at least one address on the solid support can be a probe specific for one or more of the genes disclosed in Table 1. Disclosed are solid supports where at least one address is the sequences or portion of sequences set forth in any of the peptide sequences disclosed herein. Solid supports can also contain at least one address is a variant of the sequences or part of the sequences set forth in any of the nucleic acid sequences disclosed herein. Solid supports can also contain at least one address is a variant of the sequences or portion of sequences set forth in any of the peptide sequences disclosed herein.

In addition, the genes described herein may be used as markers for presence or progression of breast cancer. The methods and assays described elsewhere herein may be performed over time, and the change in the level of reactive polypeptide(s) or polynucleotide(s) evaluated. For example, the assays may be performed every 24-72 hours for a period of 6 months to 1 year, and thereafter performed as needed. Assays can be performed prior to, during, or after a treatment protocol. Together, the genes can be used to profile an individual's risk of hereditary breast cancer, and, in some aspects, can give a measure of risk.

As noted herein, to improve sensitivity, multiple genes may be assayed within a given sample. Binding agents specific for different proteins, antibodies, nucleic acids thereto provided herein may be combined within a single assay. Further, multiple primers or probes may be used concurrently. The selection of receptors may be based on routine experiments to determine combinations that results in optimal sensitivity. To assist with such assays, specific biomarkers can assist in the specificity of such tests. As such, disclosed herein is a biomarker, wherein the biomarker is capable of binding to or hybridizing with a metabolite detecting, a gene or peptide as disclosed herein.

According to a further aspect, there is provided a computer implemented product for predicting a prognosis or classifying a subject with breast cancer comprising (a) a means for receiving values corresponding to a subject expression profile in a subject sample; and (b) a database comprising a reference expression profile associated with a prognosis, wherein the subject biomarker expression profile and the biomarker reference profile each have at least three values representing the expression level of at least one biomarker selected from Table 1, Table 2, Table 3, and/or Table 4, wherein the computer implemented product selects the biomarker reference expression profile most similar to the subject biomarker expression profile, to thereby predict a prognosis or classify the subject.

Preferably, a computer implemented product described herein is for use with a method described herein.

According to a further aspect, there is provided a computer implemented product for determining therapy for a subject with breast cancer comprising: (a) a means for receiving values corresponding to a subject expression profile in a subject sample; and (b) a database comprising a reference expression profile associated with a therapy, wherein the subject biomarker expression profile and the biomarker reference profile each have at least one value, the at least one value representing the expression level of at least one biomarker selected from Table 1, Table 2, Table 3, and/or Table 4, wherein the computer implemented product selects the biomarker reference expression profile most similar to the subject biomarker expression profile, to thereby predict the therapy.

According to a further aspect, there is provided a computer readable medium having stored thereon a data structure for storing a computer implemented product described herein.

Preferably, the data structure is capable of configuring a computer to respond to queries based on records belonging to the data structure, each of the records comprising: (a) a value that identifies a biomarker reference expression profile of at least one gene selected from Table 1, Table 2, Table 3, and/or Table 4; (b) a value that identifies the probability of a prognosis associated with the biomarker reference expression profile.

According to a further aspect, there is provided a computer system comprising (a) a database including records comprising a biomarker reference expression profile of at least one gene selected from Table 1, Table 2, Table 3, and/or Table 4; associated with a prognosis or therapy; (b) a user interface capable of receiving a selection of gene expression levels of the at least one gene for use in comparing to the biomarker reference expression profile in the database; (c) an output that displays a prediction of prognosis or therapy according to the biomarker reference expression profile most similar to the expression levels of the at least one gene.

In a further aspect, the application provides computer programs and computer implemented products for carrying out the methods described herein. Accordingly, in one embodiment, the application provides a computer program product for use in conjunction with a computer having a processor and a memory connected to the processor, the computer program product comprising a computer readable storage medium having a computer mechanism encoded thereon, wherein the computer program mechanism may be loaded into the memory of the computer and cause the computer to carry out the methods described herein.

### Methods

The disclosed gene and peptides, including those found in Tables 1-6, can be used in a variety of different methods, for example in prognostic, predictive, diagnostic, and therapeutic methods and as a variety of different compositions.

Disclosed herein are methods of assessing a subject's susceptibility to develop cancer, wherein said cancer is a breast cancer comprising the steps of: (a) obtaining a nucleic acid for variant detection and/or deregulation (ie. up- or down-regulation) of genes or gene expression products, wherein said genes or gene expression products are selected from: (i) the genes of Table 1; (ii) the genes of Table 2; and (iii) genes of Table 3; or (iv) the genes of Table 4; and wherein 1-300 genes or gene expression products are selected; (b) obtaining a profile of the expression levels of the selected genes or gene expression products in said sample; and (c) assessing a subject's susceptibility to develop breast cancer based upon genetic variants and/or a variance in the obtained profile of expression levels of the said selected genes or gene expression products in said subject's sample from the same selected genes or gene expression products of a control gene expression profile from a similar biological sample of a healthy subject, or assessing a subject's susceptibility to develop breast cancer based upon a similarity in the obtained profile of expression levels of said selected genes or gene expression products in said subject's sample to the same selected genes or gene expression products in a gene expression profile characteristic of a subject with breast cancer or a subject who has a proven susceptibility to develop breast cancer.

The disclosed method of assessing a subject's susceptibility to develop cancer can further comprise performing a BRCA1/2 mutation analysis or at least analyzing the results from a BRCA1/2 mutation analysis and using the results from the BRCA1/2 screen in combination with the results from the gene expression profile obtained with steps (a) and (b). Thus step (c) would further comprise assessing the subject's susceptibility to develop breast cancer by not only comparing the expression profile of the sample to a healthy, control sample but also considering the results of the BRCA1/2 analysis.

The gene profiles disclosed herein can be formed by a decreased level of one or more of the genes of Table 1 and Table 3, and/or increased levels of one or more of the genes of Table 2 and Table 4, as compared to said healthy profile is indicative of breast cancer or a subject's susceptibility to develop breast cancer.

Also disclosed herein are methods for diagnosing breast cancer in a mammalian subject comprising: (a) obtaining a nucleic acid for analysis of genes or gene expression products, wherein said genes or gene expression products are selected from: (i) the genes of Table 1; (ii) the genes of Table 2; and (iii) genes of Table 3; or (iv) the genes of Table 4; and wherein at least 5 genes or gene expression products are selected; (b) obtaining a profile of the expression levels of the selected genes or gene expression products in said sample; and (c) diagnosing breast cancer based upon a pattern of obtained expression levels of the said genes or gene expression products that form a gene expression profile characteristic of breast cancer in said subject's sample.

The pattern of obtained expression levels of the genes or gene expression products can be compared to a control gene expression profile. For example, the control gene expression profile can be from a similar biological sample of a healthy subject, or from another subject that has been diagnosed with breast cancer, or can be compared to both a healthy subject and a subject with breast cancer.

The expression levels in the methods disclosed herein can be normalized using protocols known in the art. For example, disclosed is a method of assessing a subject's susceptibility to develop cancer, wherein said cancer is a breast cancer comprising: (a) obtaining a nucleic acid for analysis of genes or gene expression products, wherein said genes or gene expression products are selected from: (i) the genes of Table 1; (ii) the genes of Table 2; and (iii) genes of Table 3; or (iv) the genes of Table 4; and wherein at least 20 genes or gene expression products are selected; (b) obtaining a profile of the expression levels of the selected genes or gene expression products in said sample; and (c) normalizing said expression level to obtain a normalized expression level of the genes of (b); and (d) assessing a subject's susceptibility to develop breast cancer based upon a variance in the obtained profile of expression levels of the said selected genes or gene expression products in said subject's sample from the same selected genes or gene expression products of a control gene expression profile from a similar biological sample of a healthy subject, or assessing a subject's susceptibility to develop breast cancer based upon a similarity in the obtained profile of expression levels of said selected genes or gene expression products in said subject's sample to the same selected genes or gene expression products in a gene expression profile characteristic of a subject with breast cancer or a subject who has a proven susceptibility to develop breast cancer.

The term “normalization” means to convert a numerical value, such as fluorescence intensity, which has been obtained by a gene expression analysis or the like, into a numerical value that permits a comparison with all measurement values obtained by other gene expression analyses. Expression data may be normalized with respect to one or more genes with invariant expression, such as “housekeeping” genes. When normalization is used with the methods disclosed herein, one can use one of the many methods of normalization known in the art. Here, as the expression level, one obtained by normalizing the determined raw data of the expression level, for example, by RMA algorithm, MASS algorithm, DWD algorithm, SCAN algorithm, PLIER algorithm, or the like can be used. The RMA algorithm is available, for example, on the analysis software (manufactured by Affymetrix, Inc., trade name: Affymetrix Expression Console software).

Further disclosed herein are methods for diagnosing breast cancer in a mammalian subject comprising: (a) obtaining a nucleic acid for analysis of genes or gene expression products, wherein said genes or gene expression products are selected from: (i) the genes of Table 1; (ii) the genes of Table 2; and (iii) genes of Table 3; or (iv) the genes of Table 4; and wherein at least 20 genes or gene expression products are selected; (b) obtaining a profile of the expression levels of the selected genes or gene expression products in said sample; (c) normalizing said expression level to obtain a normalized expression level of the genes of (a); and (d) diagnosing breast cancer based upon a pattern of obtained expression levels of the said genes or gene expression products that form a gene expression profile characteristic of breast cancer in said subject's sample.

Disclosed herein are methods of preparing a personalized genomics profile for a breast cancer subject, comprising: (a) obtaining a nucleic acid for analysis of genes or gene expression products, wherein said genes or gene expression products are selected from: (i) the genes of Table 1; (ii) the genes of Table 2; and (iii) genes of Table 3; or (iv) the genes of Table 4; and wherein at least 20 genes or gene expression products are selected (b) obtaining the expression levels of said genes or gene expression products in said sample; wherein the expression level is normalized against the expression level of at least one reference gene to obtain normalized data or the expression levels in a breast cancer reference tissue set; and (c) creating a report summarizing the normalized data obtained by said gene expression analysis, wherein said report includes a prediction of a subject's increased likelihood to develop breast cancer. The methods of preparing a personalized genomics profile can further comprise including the results of a BRCA1/2 mutation analysis in the report.

Also disclosed are methods of assessing a subject's susceptibility to develop cancer, wherein said cancer is a breast cancer comprising: (a) obtaining a nucleic acid for amplification of genes or gene expression products, wherein said genes or gene expression products are selected from: (i) the genes of Table 5; or (ii) the genes of Table 6; and wherein at least 1 gene or gene expression products are selected; (b) obtaining a profile of the expression levels of the selected genes or gene expression products in said sample; and (c) assessing a subject's susceptibility to develop breast cancer based upon a variance in the obtained profile of expression levels of the said selected genes or gene expression products in said subject's sample from the same selected genes or gene expression products of a control gene expression profile from a similar biological sample of a healthy subject, or assessing a subject's susceptibility to develop breast cancer based upon a similarity in the obtained profile of expression levels of said selected genes or gene expression products in said subject's sample to the same selected genes or gene expression products in a gene expression profile characteristic of a subject with breast cancer or a subject who has a proven susceptibility to develop breast cancer.

In combination with analyzing the expression profile of genes from Tables 5 or 6, the disclosed method of assessing a subject's susceptibility to develop cancer can further comprise performing a BRCA1/2 mutation analysis or at least analyzing the results from a BRCA1/2 mutation analysis and using the results from the BRCA1/2 screen in combination with the results from the gene expression profile obtained with steps (a) and (b). Thus step (c) would further comprise assessing the subject's susceptibility to develop breast cancer by not only comparing the expression profile of the sample to a healthy, control sample but also considering the results of the BRCA1/2 analysis. As described herein, disclosed are methods of determining the risk of developing breast cancer in a subject comprising determining the expression level of one or more genes in a sample and comparing those expression levels to the expression levels of a normal sample, wherein the expression level of one or more said genes or peptides is increased or decreased by 10%, 15%, 20%, 25%, 30%, 35%, 40%, 45%, 50%, 55%, 60%, 65%, 70%, 75%, 80%, 85%, 90%, 95%, or 100% when compared to the expression level of a “normal” subject is indicative of breast cancer formation. In addition, the expression level of one or more genes or peptides as found in any of Tables 1-4 can be increased or decreased by 10%, 15%, 20%, 25%, 30%, 35%, 40%, 45%, 50%, 55%, 60%, 65%, 70%, 75%, 80%, 85%, 90%, 95%, or 100% when compared to the expression level of a “normal” subject is indicative of a breast cancer formation.

By “susceptibility” is meant the likelihood of an individual being clinically diagnosed with breast cancer.

An increase or decrease in the expression level of the genes or peptides disclosed herein is not always required to indicate breast cancer risk. There can be signature patterns of increased or decreased expression levels of one or more of the genes or peptides.

“Survival time” or “survival rate” indicates the likelihood for survival of the disease for a specific period of time after the diagnosis of a subject. For example, this can refer to a five year breast cancer survival rate, meaning the chance that a given individual will survive 5 years from the time of their initial diagnosis, or from another given point. Along with the analysis described herein, other factors that can affect the survival rate, which can also be considered when calculating the rate, include the stage of breast cancer when diagnosed, whether a given immunoglobulin (antibody) is present, and the subject's age and general health.

“Prognosis” refers to a clinical outcome group such as a poor survival group or a good survival group associated with breast cancer which is reflected by a reference profile, or reflected by an expression level of the methods disclosed herein. The prognosis provides an indication of disease progression and includes an indication of likelihood of death due to breast cancer. In one embodiment the clinical outcome class includes a good survival group and a poor survival group.

The term “prognosing” or “classifying” as used herein means predicting or identifying the clinical outcome group that a subject belongs to according to the subject's similarity to a reference profile associated with the prognosis. For example, prognosing or classifying comprises a method or process of determining whether an individual with breast cancer has a good or poor survival outcome, or grouping an individual with breast cancer into a good survival group or a poor survival group, or predicting whether or not an individual with breast cancer will respond to therapy. Also included is determining the risk level of developing breast cancer, in a subject that has not been diagnosed with the disease.

The term “good survival” as used herein refers to an increased chance of survival as compared to patients in the “poor survival” group. For example, the genes in Tables 1-4 can be used to prognose or classify subjects into a “good survival group”. These patients are at a lower risk of death. Good survival, as used herein, is defined as being expected to survive for five years or more.

The term “poor survival” as used herein refers to an increased risk of death as compared to subjects in the “good survival” group. For example, the genes in Tables 1-4 can be used to prognose or classify subjects into a “poor survival group”. These patients are at greater risk of death from breast cancer. Poor survival, as used herein, is defined as being expected to survive for less than one year.

In one example, the variance in the obtained profile of expression levels of the said selected genes or gene expression products in said subject's sample can be used to determine whether a subject is at a low, intermediate, or high-risk of developing breast cancer. The terms “low, intermediate, and high” are relative terms, which can mean, for example, that the subject is at low risk (25% or less chance of developing breast cancer), intermediate (50% or less chance of developing breast cancer) or high risk (75% chance or greater of developing breast cancer).

In the methods disclosed above, the nucleic acid can be obtained by extracting nucleic acid from a biological sample of the subject containing immune cells, peripheral blood, epithelial cells or cancer cells. If the sample is peripheral blood, immune cells can be obtained which are peripheral blood mononuclear cells. Furthermore, the nucleic acid can be RNA and/or DNA. In one example, cDNA can be generated from the RNA.

The variance in the obtained profile of expression levels of the said selected genes or gene expression products in said subject's sample can be used to determine the type of treatment, or combination of treatments, that the subject should receive. The standard methods of treatment for a subject diagnosed with breast cancer include, but are not limited to, surgery, including breast-conserving surgery, lumpectomy, partial masectomy, full masectomy; modified radical masectomy, and sentinel lymph node biopsy followed by surgery. Any of these may also include neoadjuvant surgery. Treatment can also include radiation therapy, chemotherapy, hormone therapy, targeted therapy, and monoclonal antibody therapy. Examples of these include, but are not limited to, Trastuzumab (Herceptin), which is a monoclonal antibody that blocks the effects of the growth factor protein HER2, which sends growth signals to breast cancer cells. Tyrosine kinase inhibitors are targeted therapy drugs that block signals needed for tumors to grow. Tyrosine kinase inhibitors may be used in combination with other anticancer drugs as adjuvant therapy. Lapatinib is a tyrosine kinase inhibitor that blocks the effects of the HER2 protein and other proteins inside tumor cells. It may be used with other drugs to treat patients with HER2-positive breast cancer that has progressed following treatment with trastuzumab. PARP inhibitors are a type of targeted therapy that block DNA repair and may cause cancer cells to die. PARP inhibitor therapy is being studied for the treatment of triple-negative breast cancer. Another option is high-dose chemotherapy with stem cell transplant.

Also disclosed are methods of assessing a subject's susceptibility to develop cancer, comprising performing exome-capture sequencing to determine the presence of mutations that differ from a subject with hereditary breast cancer and a subject without hereditary breast cancer. The information obtained from determining the presence of mutations can be used to identify molecular pathways affected by the mutations. A pathway can be considered to be affected if any suspected mutation had been observed in any gene in that pathway. The identification of affected pathways can then be used to assess a subject's susceptibility to develop cancer. For example, if a pathway has a p-value similar to the p-values shown in Table 9 or the AUC values of Table 16 can indicate a subject's higher susceptibility to develop cancer. Alternatively, or in addition to the pathway information, mutations in one or more of the genes identified in Tables 11, 12, 13 can be used to assess a subject's susceptibility to develop cancer. A combination of the pathway data, the gene mutations identified with exome-capture sequencing, and BRCA1/2 mutation data can be used to assess a subject's susceptibility to develop cancer. Thus one or more assays can be combined to assess a subject's susceptibility to develop cancer.

Also disclosed are methods for treating breast cancer in an individual, comprising the step of: modulating expression of one or more genes identified in one or more of the Tables disclosed herein; thereby altering differential expression of the breast cancer-related genes to treat the individual. Also disclosed herein are methods that can be used to evaluate the efficacy of various clinical interventions.

The term “modulate”, as used herein, refers to a change or an alteration in the biological activity of a gene or a gene product, such as a polypeptide. Modulation may be an increase or a decrease in expression level or peptide activity, a change in binding characteristics, or any other change in the biological, functional or immunological properties of the nucleic acid or polypeptide. In one example, some genes can be upregulated, and others downregulated, simulataneously.

Disclosed herein are functional nucleic acids that can interact with the disclosed receptor nucleic acids. Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of polynucleotide sequences disclosed herein or the genomic DNA of the polynucleotide sequences disclosed herein or they can interact with the polypeptide encoded by the polynucleotide sequences disclosed herein. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation.

Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (kd) less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437 each of which is herein incorporated by reference in its entirety for their teaching of modifications and methods related to the same.

Disclosed are aptamers that interact that interact with the disclosed nucleic acids and could thus inhibit the expression of such Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with kds from the target molecule of less than 10-12 M. It is preferred that the aptamers bind the target molecule with a kd less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a kd with the target molecule at least 10, 100, 1000, 10,000, or 100,000 fold lower than the kd with a background binding molecule. It is preferred when doing the comparison for a polypeptide for example, that the background molecule be a different polypeptide. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Disclosed are ribozymes that interact with the disclosed nucleic acids and could thus inhibit the expression of such. Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Disclosed are triplex forming functional nucleic acid molecules that interact with the disclosed nucleic acids and could thus inhibit the expression of such. Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a kd less than 10⁻⁶, 10⁻⁸, 10⁻¹⁰, or 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

Disclosed are external guide sequences that form a complex with the disclosed nucleic acids and could thus inhibit the expression of such. External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules can be found in the following non-limiting list of U.S. Pat. Nos. 5,168,053, 5,624,824, 5,683,873, 5,728,521, 5,869,248, and 5,877,162.

Disclosed are polynucleotides that contain peptide nucleic acids (PNAs) compositions that interact with the disclosed nucleic acids and could thus inhibit the expression of such. PNA is a DNA mimic in which the nucleobases are attached to a pseudopeptide backbone (Good and Nielsen, Antisense Nucleic Acid Drug Dev. 1997; 7(4) 431-37). PNA is able to be utilized in a number of methods that traditionally have used RNA or DNA. Often PNA sequences perform better in techniques than the corresponding RNA or DNA sequences and have utilities that are not inherent to RNA or DNA. A review of PNA including methods of making, characteristics of, and methods of using, is provided by Corey (Trends Biotechnol 1997 June; 15(6):224-9). As such, in certain embodiments, one may prepare PNA sequences that are complementary to one or more portions of an mRNA sequence based on the disclosed polynucleotides, and such PNA compositions may be used to regulate, alter, decrease, or reduce the translation of the disclosed polynucleotides transcribed mRNA, and thereby alter the level of the disclosed polynucleotide's activity in a host cell to which such PNA compositions have been administered.

PNAs have 2-aminoethyl-glycine linkages replacing the normal phosphodiester backbone of DNA (Nielsen et al., Science Dec. 6, 1991; 254(5037):1497-500; Hanvey et al., Science. Nov. 27, 1992; 258(5087):1481-5; Hyrup and Nielsen, Bioorg Med Chem. 1996 January; 4(1):5-23). This chemistry has three important consequences: firstly, in contrast to DNA or phosphorothioate oligonucleotides, PNAs are neutral molecules; secondly, PNAs are achirial, which avoids the need to develop a stereoselective synthesis; and thirdly, PNA synthesis uses standard Boc or Fmoc protocols for solid-phase peptide synthesis, although other methods, including a modified Merrifield method, have been used.

PNA monomers or ready-made oligomers are commercially available from PerSeptive Biosystems (Framingham, Mass.). PNA syntheses by either Boc or Fmoc protocols are straightforward using manual or automated protocols (Norton et al., Bioorg Med Chem. 1995 April; 3(4):437-45). The manual protocol lends itself to the production of chemically modified PNAs or the simultaneous synthesis of families of closely related PNAs.

As with peptide synthesis, the success of a particular PNA synthesis will depend on the properties of the chosen sequence. For example, while in theory PNAs can incorporate any combination of nucleotide bases, the presence of adjacent purines can lead to deletions of one or more residues in the product. In expectation of this difficulty, it is suggested that, in producing PNAs with adjacent purines, one should repeat the coupling of residues likely to be added inefficiently. This should be followed by the purification of PNAs by reverse-phase high-pressure liquid chromatography, providing yields and purity of product similar to those observed during the synthesis of peptides.

Modifications of PNAs for a given application may be accomplished by coupling amino acids during solid-phase synthesis or by attaching compounds that contain a carboxylic acid group to the exposed N-terminal amine. Alternatively, PNAs can be modified after synthesis by coupling to an introduced lysine or cysteine. The ease with which PNAs can be modified facilitates optimization for better solubility or for specific functional requirements. Once synthesized, the identity of PNAs and their derivatives can be confirmed by mass spectrometry. Several studies have made and utilized modifications of PNAs (for example, Norton et al., Bioorg Med Chem. 1995 April; 3(4):437-45; Petersen et al., J Pept Sci. 1995 May-June; 1(3):175-83; Orum et al., Biotechniques. 1995 September; 19(3):472-80; Footer et al., Biochemistry. Aug. 20, 1996; 35(33): 10673-9; Griffith et al., Nucleic Acids Res. Aug. 11, 1995; 23(15):3003-8; Pardridge et al., Proc Natl Acad Sci USA. Jun. 6, 1995; 92(12):5592-6; Boffa et al., Proc Natl Acad Sci USA. Mar. 14, 1995; 92(6):1901-5; Gambacorti-Passerini et al., Blood. Aug. 15, 1996; 88(4):1411-7; Armitage et al., Proc Natl Acad Sci USA. Nov. 11, 1997; 94(23):12320-5; Seeger et al., Biotechniques. 1997 September; 23(3):512-7). U.S. Pat. No. 5,700,922 discusses PNA-DNA-PNA chimeric molecules and their uses in diagnostics, modulating protein in organisms, and treatment of conditions susceptible to therapeutics.

Methods of characterizing the antisense binding properties of PNAs are discussed in Rose (Anal Chem. Dec. 15, 1993; 65(24):3545-9) and Jensen et al. (Biochemistry. Apr. 22, 1997; 36(16):5072-7). Rose uses capillary gel electrophoresis to determine binding of PNAs to their complementary oligonucleotide, measuring the relative binding kinetics and stoichiometry. Similar types of measurements were made by Jensen et al. using BIAcore™ technology.

Other applications of PNAs that have been described and will be apparent to the skilled artisan include use in DNA strand invasion, antisense inhibition, mutational analysis, enhancers of transcription, nucleic acid purification, isolation of transcriptionally active genes, blocking of transcription factor binding, genome cleavage, biosensors, in situ hybridization, and the like.

In addition, antibodies to the proteins disclosed herein can be used to inhibit the function of the receptors. For example, isolated antibodies, antibody fragments and antigen-binding fragments thereof. Optionally, the isolated antibodies, antibody fragments, or antigen-binding fragment thereof can be neutralizing antibodies. The antibodies, antibody fragments and antigen-binding fragments thereof disclosed herein can be identified using the methods disclosed herein.

The term “antibodies” is used herein in a broad sense and includes both polyclonal and monoclonal antibodies. In addition to intact immunoglobulin molecules, disclosed are antibody fragments or polymers of those immunoglobulin molecules, and human or humanized versions of immunoglobulin molecules or fragments thereof, as long as they are chosen for their ability to interact with the polypeptides disclosed herein. As used herein, the term “antibody” or “antibodies” can also refer to a human antibody or a humanized antibody.

“Antibody fragments” are portions of a complete antibody. A complete antibody refers to an antibody having two complete light chains and two complete heavy chains. An antibody fragment lacks all or a portion of one or more of the chains. Examples of antibody fragments include, but are not limited to, half antibodies and fragments of half antibodies. A half antibody is composed of a single light chain and a single heavy chain. Half antibodies and half antibody fragments can be produced by reducing an antibody or antibody fragment having two light chains and two heavy chains. Such antibody fragments are referred to as reduced antibodies. Reduced antibodies have exposed and reactive sulfhydryl groups. These sulfhydryl groups can be used as reactive chemical groups or coupling of biomolecules to the antibody fragment. A preferred half antibody fragment is a F(ab). The hinge region of an antibody or antibody fragment is the region where the light chain ends and the heavy chain goes on.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules.

The invention will be further described with reference to the following examples; however, it is to be understood that the invention is not limited to such examples. Rather, in view of the present disclosure that describes the current best mode for practicing the invention, many modifications and variations would present themselves to those of skill in the art without departing from the scope and spirit of this invention. All changes, modifications, and variations coming within the meaning and range of equivalency of the claims are to be considered within their scope.

### EXAMPLES

### Example 1

### Genomic Variation in Peripheral Blood can Predict Hereditary Breast-Cancer Development

### Methods

### Patient Cohorts

The initial patient cohort was identified in the High Risk Breast Cancer Clinic at the Huntsman Cancer Institute, University of Utah. Women were identified who fell into one of three risk groups: 1) women at high risk for breast cancer due to family history and a putative germline mutation in BRCA1 or BRCA2, 2) women at high risk for breast cancer due to family history and no known mutation in BRCA1/2 (“BRCAX”), and 3) women considered not to have high risk for breast cancer due to a lack of family history and no known mutations in BRCA1/2. The risk groups were then subdivided according to whether they had previously been diagnosed with breast cancer. Participants were considered to have a family history if two or more first-degree relatives had been diagnosed with breast cancer before 45 years of age. Participants with a family history who had not been diagnosed with breast cancer were at least 54 years old at the time data were collected. Participants were matched for age as well as for other characteristics, including SERM use and number of pregnancies. To control for disease-specific noise, all subjects with disease occurrence had no evidence of disease at sample collection time or for at least 6 months prior to sample collection. Across all risk groups, 124 subjects were recruited: 83 with a family history (39 of which had developed breast cancer) and 41 with no family history (22 of which had developed sporadic breast cancer). Table 7 summarizes the various patient risk groups and cohorts.

Women with no family history include those that had sporadic breast cancer to control for signal due to any variation in gene expression due to past cancer occurrence and normal healthy controls. These controls also allowed to account for non-heritable factors that may impact disease risk.

Data from two additional patient cohorts were obtained as a means of testing external validity. Two series of samples were obtained. The first cohort included 13 samples from women who had developed hereditary breast cancer, 15 samples from high-risk women who had not developed breast cancer, and 8 sporadic-cancer controls. Tamoxifen use among those that did and did not develop cancer was assessed, and genes that correlated to tamoxifen use were filtered out of the subsequent analysis. As with the Utah cohort, these samples were acquired retrospectively (for example, after cancer had developed and been treated). Conversely, the second validation cohort consisted of samples that had been obtained, isolated, and stored prospectively when the women first were enrolled in the BFCR, prior to disease occurrence, prior to any prophylactic treatment, and before menopause onset. These participants were followed over time and monitored for cancer development. Among these participants, 15 samples were from high-risk women who eventually developed breast cancer, 17 from high-risk women who had not developed breast cancer by the time of the study, and 5 from controls who had no family history and had not developed breast cancer. This cohort was particularly important for validation because it represents the desired target population.

TABLE 7
Summary of patient groups within each cohort.

| | | Ontario | Ontario |
|---|---|---|---|
| Category | Utah | Retrospective | Prospective |
| Family History, BRCA1/2, | 17 | 3 | 8 |
| Developed Cancer | | | |
| Family History, BRCAX, | 22 | 10 | 7 |
| Developed Cancer | | | |
| Family History, BRCA1/2, | 20 | 5 | 9 |
| No Cancer | | | |
| Family History, BRCAX, | 24 | 10 | 8 |
| No Cancer | | | |
| No Family History, | 22 | 8 | 0 |
| Sporadic Cancer | | | |
| No Family History, No Cancer | 19 | 0 | 5 |
| Total | 124 | 36 | 37 |

### DNA Mutation Analysis

BRCA mutation analysis was performed by Mryiad Genentics according to their standard protocols.

### Microarray Analysis

After providing informed consent, patients donated a peripheral blood sample, and PBMCs were isolated from whole blood in CPT tubes following manufacturer protocol (BectonDickinson). RNA was then extracted using the RiboPure RNA Isolation Kit and hybridized to the Genechip Human Exon 1.0 ST microarray (Affymetrix, Calif.).

### Covariate Analysis

To identify blood markers that could influence mRNA expression but that are likely not related to hereditary breast cancer development, a total lymphocyte enumeration test for the blood draw was contemplated. This test provides total counts of CD4-positive T cells, CD8-positive T-cells, CD3-positive T-cells, B-cells and NK-cells. These counts were available for 22 samples in the Utah cohort. Further, epidemiological and demographic data were obtained on 61 patients in the Utah cohort; these factors were age, education level, marital status, religious preference, health, age at menarche, contraception use, total number of pregnancies, total number of live births, age at first giving birth, age at last giving birth, breastfeeding status, age at first period, age at menopause, prophylactic drug use (SERMS), alcohol use, tobacco use, employment-status, and-drug use. Finally, genes whose expression patterns correlated with population variances for any of the lymphocyte subtypes or epidemiological/demographic variables at a 0.01 significance level (tested using ANCOVA analyses) were excluded.

The Single-Channel Array Normalization method (Piccolo et al. Genomics. 2012; 100(6):337-44) was applied to correct for array- and probe level background noise; this method is applied to each sample individually, thus averting intersample biases that can arise with multisample normalization techniques (Giorgi F M, et al. BMC Bioinformatics. 2010; 11(1):553). Probes were subsequently filtered using the PLANdbAffy database, which assigns quality labels to probes based on cross-hybridization potential, location of probes within genes, and whether SNVs fall within target regions (Nurtdinov et al. Nucleic Acids Res. 2010; 38(Database issue):D726-30). Any probe that was not classified as “green” or that mapped to an SNV was excluded. For each array, the remaining 2,201,005 probes were summarized into gene-level values using a 10% trimmed mean; genes that contained fewer than five probes were discarded.

Because the microarrays were hybridized and scanned in different facilities and at different times, ComBat (JohnsonWE, et al. Biostatistics. 2007; 8(1):118-2751) was used to adjust for potential confounding effects that might arise due to these differences. This adjustment was performed twice independently: 1) between the Utah cohort and the Ontario retrospective cohort, and 2) between the Utah cohort and the Ontario prospective cohort.

To identify blood markers that could influence mRNA expression but that are likely not related to hereditary breast cancer development, a total lymphocyte enumeration test was compiled for the blood draw used in the study. This test provides total counts of CD4-positive T cells, CD8-positive T-cells, CD3-positive T-cells, B-cells and NK-cells. These counts were available for 22 samples in the Utah cohort. Further, epidemiological and demographic data was obtained on 61 patients in the Utah cohort. Genes whose expression patterns correlated with population variances for any of such variable at a 0.01 significance level were excluded.

### Biomarker Development

A gene-expression biomarker that could identify individuals most likely to develop hereditary breast cancer (HBC) was developed. The biomarker was derived in two successive steps: 1) the most discriminatory genes were identified using the Support VectorMachines-Recursive Feature Elimination (SVM-RFE) algorithm, then 2) the Support Vector Machines (SVM) algorithm (Vapnik V N. Statistical learning theory. New York: Wiley; 1998) was used to generate a probability that each patient belonged to the HBC group; these probabilities were termed HBC Scores. The SVM algorithm uses a kernel function to identify a maximum-margin hyperplane that separates data instances belonging to particular groups (i.e., HBC or control). This algorithm has been shown to perform exceptionally well on high dimensional microarray data (Statnikov et al. Bioinformatics. 2005; 21(5):631-43). The SVM-RFE algorithm (Guyon I, et al. Mach Learn. 2002; 46(1):389-422), which is based on SVM, assigns weights to each variable, estimating their ability to discriminate the groups. Using these weights, a backward search is performed: variables with the lowest assigned weights are removed iteratively; iterations continue until one or a few variables remain. Ranks are assigned according to the order in which variables were removed. Genes that ranked in the top 25, 50, 75, 100, 125 . . . 300 (when that many were available) were used in the SVM models, and the number of genes included in the SVM models was optimized via internal cross validation.

SVM-RFE was performed using the SVMAttributeEval module within the Weka software package (Hall M, et al. ACM SIGKDD Explorations Newsletter. 2009; 11(1):10). It was configured to remove 10% of genes in each iteration; when less than 1% of genes remained, a single gene was removed per iteration. Otherwise, default configuration settings were used. The e1071 package (E1071: Misc functions of the department of statistics (e1071), TU wien [computer program]. Dimitriadou E, Hornik K, Leisch F, Meyer D, Weingessel A. 2011. Available from cran.r-project.org/package=e1071) within the R statistical package (R: A language and environment for statistical computing [computer program]. R Development Core Team. 2011. Available from r-project.org/) was used for SVM predictions. This package provides an interface to the LIBSVM library (Chang C, Lin C. ACM Transactions on Intelligent Systems and Technology. 2011; 2(3):27:1-27:27). In deriving the models, the radial-basis-function kernel was used, and the C parameter was tuned via internal cross validation. Additionally, the ML-Flex software package (Piccolo et al. Journal of Machine Learning Research. 2012; 13:555-559) enabled the analysis to be executed in parallel on a high-performance computing cluster.

The quality of the models was assessed via ten-fold cross validation for the Utah cohort and via a training/testing design for the Ontario cohorts. In training/testing, gene selection and optimization were performed via nested cross validation on the Utah data only. HBC Scores were compared against known classes (HBC or not), and an AUC value was calculated. In this setting, the AUC quantifies the model's ability to discriminate the groups at various HBC Score thresholds; it can be interpreted as the frequency with which the model would assign two randomly selected patients to the correct group.

For additional validation on the Utah data, the same analysis steps were executed using randomly permuted class labels. This process was repeated 1,000 times, and an empirical p-value was calculated by comparing permuted AUC values against non-permuted values.

To identify which pathways may contribute most to hereditary breast-cancer development, an independent biomarker for each pathway was tested. For a given pathway, the gene expression data were filtered initially to include only genes that belonged to that pathway. Then SVMRFE was used for further gene selection, and predictions were made using the SVM algorithm. Pathways were ranked according to average AUC across the cohorts. This approach takes advantage of the SVM algorithm's ability to model subtle patterns across multiple genes.

### Model Development

We aimed to demonstrate that high-risk individuals who develop breast cancer (BRCA-Cancer) can be differentiated from high-risk individuals who do not develop breast cancer and from individuals who have no family predisposition to breast cancer (Controls). Initially, this hypothesis was tested within the Utah data set alone, using a ten-fold cross-validation strategy. Samples were randomly assigned to one of ten partitions; in turn, samples in each partition were held separately (test set) and a classification model was derived from the remaining samples (training set). Models were then used to make predictions for patients in respective test sets. After performing cross-validation, the full Utah cohort was used as a training set, and the Ontario cohorts were used for testing.

Within each training set, classification models were derived in two successive steps: 1) the Support Vector Machines-Recursive Feature Elimination (SVM-RFE) algorithm attempted to identify genes most relevant to hereditary-breast-cancer status, and 2) the Support Vector Machines (SVM) algorithm attempted to account for combinations of mRNA expression levels in those genes that influence hereditary-breast-cancer status. The SVM-RFE algorithm [Guyon 2002] is based on SVM, which separate instances from each class using a multidimensional hyperplane. [Vapnik 1998] In SVM-RFE, weights are assigned to each variable, estimating their ability to discriminate the classes. Using these weights, a backward search is performed: variables that have been assigned the lowest weights are removed in each iteration, and iterations continue until a single variable remains. Ranks are assigned to each variable according to the order in which they were removed.

Only genes ranked in the top 25, 50, 75, 100, 125 . . . 300 (when that many were available) were used in SVM models. The number of top-ranked genes that performed best (according to AUC) in cross validation was considered “optimal” and used for external-validation predictions; however, predictions also were made using other quantities of top-ranked genes to enable evaluation of model robustness.

SVM-RFE was performed using the SVMAttributeEval module within the Weka software package [Hall 2009]. It was configured to remove 10% of genes in each iteration; when less than 1% of genes remained, a single gene was removed per iteration. Otherwise, default configuration settings were used. The e1071 package [Dimitridaiou 2011] within the R statistical package [Developmental Core Team, 2011] was used for SVM predictions. This package provides an interface to the LIBSVM library. [Chang 2011] In deriving the models, the radial-basis-function kernel was used, and the C parameter was tuned via internal cross validation.

### Model Evaluation

In this study, the output of SVM models was a probability value for each patient. This value suggested how likely a given patient belonged in the BRCA-Cancer class. This value is referred to as the Hereditary Breast Cancer Score (HBC Score). The quality of the models was assessed via a comparison of HBC Scores with known statuses (BRCA-Cancer or Control), which resulted in AUC values representing a model's ability to discriminate between patients in the two classes. The ROCR R package was used for the AUC calculations and for producing receiver operating characteristic (ROC) graphs. [ROCR 2009]

For additional validation, the same analysis steps using randomly permuted class labels (BRCA-Cancer or Control) were used. This process was repeated 1,000 times, and an empirical p-value was calculated by comparing results obtained using the actual class labels with those obtained using permuted class labels.

### Experimental Reproducibility

All Python and R scripts that were developed for this study have been aggregated into a software pipeline that can be executed at the command line on UNIX-based systems. This pipeline can be downloaded online.

### Additional Analytic Approaches

Additional classification algorithms were also applied to the data. These included the sequential minimal optimization (SMO) implementation of support vector machines and the Naive Bayes classifier from the Weka software package. Default settings were used. A principal-components based classification algorithm was also attempted. For gene selection, the RELIEF-F algorithm and a Bayesian approach were also attempted. These other algorithms also discrimated BRCA-Cancer individuals from controls. LibSVM was also conducted on the data and in general for high-dimensional microarray data. [Statnikov 2005].

### Results

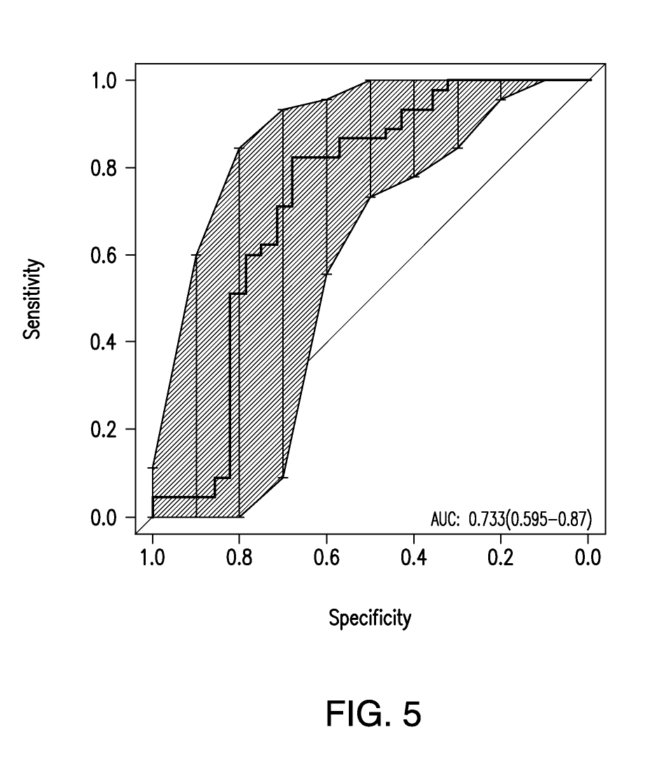

Using the methods described above, a genomic biomarker weas identified that can predict which women at high risk for hereditary breast cancer develop cancer. As shown in the figures, high risk individuals who developed breast cancer had higher HBC Scores than the high risk women who never developed breast cancer. Additionally, the individuals who developed sporadic breast cancer had relatively low HBC Scores. Such information provides evidence that these models are not characterizing general breast-cancer risk but rather risk specific to hereditary breast cancer. The resulting AUC values were 0.763* and 0.733 (see FIGS. 4 and 5). Predictive performance increased considerably compared to the first experiment, and performance tended to increase as the number of selected genes increased, reaching a climax at 250 genes. Overall, HBC Scores for the BRCAX and BRCA1/2 groups who developed cancer were similar to each other.

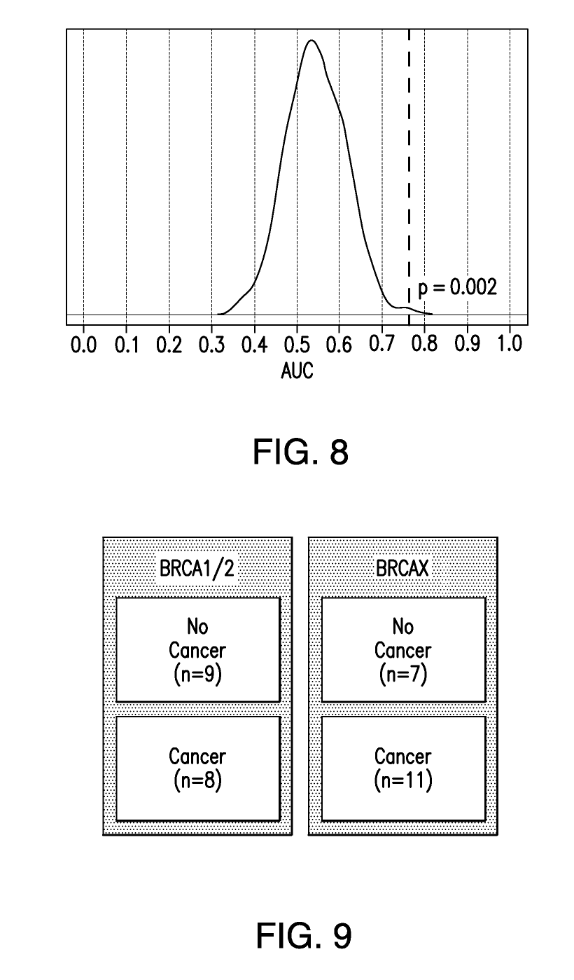

When the class labels were permuted for validation purposes, mean AUC values across 1,000 iterations ranged from 0.495 to 0.545 for the various cohorts. The actual AUC value from the Utah cohort was higher than the respective permuted values in every iteration but two (p=0.002; see FIG. 8). For the Ontario cohorts, the actual AUC values were higher than 98.5% of the permuted values (p=0.015).

In a bid to elucidate which known biological pathways were most affected by germline aberrations, a follow-up experiment was performed in which the data were filtered down to gene sets that are believed to orchestrate specific biological processes. These gene sets were extracted from Kyoto Encyclopedia of Genes and Genomes (KEGG) pathways, which are based on manual literation curation [Kanehisa 2006]. Table 7 lists the results of these experiments. In particular, genes in pathways that include cell adhesion/focal adhesion, phosphatidylinositol signaling, and cancer pathways were significantly predictive.

Having used the data for classification purposes, a “consensus” set of genes was sought that followed consistent expression patterns across the Utah and Ontario data sets. “Consensus” genes were required to have either a positive fold change within each data set or a negative fold change within each. The genes then were ranked by mean fold change across the data sets. The tables herein list the top genes that met these criteria. As described, the consensus gene set, derived from all three datasets, predicted breast cancer risk with highly significant accuracy.

### Example 2

### Pathway-Based Analysis to Discover Signaling Events in Hereditary Breast Cancer

### Background

Individuals who have a family history of breast cancer face greater risk than the general population of developing a breast tumor (Peto J, et al. Nat Genet. 2000; 26(4):411-4; Collaborative Group on Hormonal Factors in Breast Cancer. Lancet. 2001; 358(9291):1389-99). Effective prevention strategies exist—including chemoprevention via selective estrogen receptor modulators (SERMs) or aromatase inhibitors (Umar A, et al. Nature Reviews Cancer. 2012; 12(12):835-848), prophylactic mastectomy or oophorectomy (Hartmann L C, et al. N Engl J Med. 1999; 340(2):77-84; Rebbeck T R, et al. J Clin Oncol. 2004; 22(6):1055-62), and increased surveillance (Brekelmans C T, et al. J Clin Oncol. 2001; 19(4):924-30; Warner E, et al. J Clin Oncol. 2011; 29(13):1664-9). However, because tradeoffs accompany each of these strategies (Gail M H, et al. J Natl Cancer Inst. 1999; 91(21):1829-46), prevention efforts ideally should be focused on individuals most likely to develop a tumor.

Among women who develop breast cancer, 10-15% have at least one first-degree relative who previously developed a breast tumor. Researchers have examined this “high-risk” population for genetic variants that might explain the mechanisms underlying susceptibility. Rare, highly penetrant variants in the BRCA1 and BRCA2 genes are observed in approximately 16% of familial cases (Stratton M R, Nat Genet. 2008; 40(1):17-22) with a higher prevalence in multi-case families (Peto J, J Natl Cancer Inst. 1999; 91(11):943-9). Rare susceptibility variants also have been observed in genes associated with multicancer predisposition syndromes; these genes include TP53 (Sidransky D, et al. Cancer Res. 1992; 52(10):2984-6), PTEN (Liaw D, et al. Nat Genet. 1997; 16(1):64-7), STK11 (Boardman L A, et al. Ann InternMed. 1998; 128(11):896-9), and CDH1 (Brooks-Wilson A R, et al. J Med Genet. 2004; 41(7):508-17). Moderately penetrant variants in various DNA repair genes—including CHEK2, ATM, NBN, RAD50, BRIP1, PALB2, and XRCC2—have also been reported (Walsh T, et al. Cancer Cell. 2007; 11(2):103-5; Park D J, et al. Am J HumGenet. 2012; 90(4):734-739). Finally, genome-wide association studies have identified a series of common variants that appear to modulate breast-cancer risk—although with weak effects (Stratton M R, et al. Nat Genet. 2008; 40(1):17-22). Unfortunately, in total, these three classes of variant explain less than 30% of overall familial risk, and it is unlikely that additional highly or moderately penetrant variants that account for a substantial portion of familial risk will be discovered (Stratton M R, et al. Nat Genet. 2008; 40(1):17-22). Ongoing efforts to genotype ever-larger cohorts may yield additional common variants; however, the clinical implications of common variants can be difficult to decipher. Wacholder, et al. illustrated this point, incorporating ten, common, breast-cancer risk variants into logistic-regression models (Wacholder S, et al. N Engl J Med. 2010; 362(11):986-93); the models distinguished cases from controls with an area under the receiver-operating-characteristic curve (AUC) of 0.589-0.597, only a modest improvement over what was expected by random chance (0.500).

The proteins encoded by BRCA1 and BRCA2 perform complementary functions in maintaining genome integrity (Roy R, et al. Nat Rev Cancer. 2012; 12(1):68-78). Together with various other proteins, BRCA1/2 repair double-stranded breaks that occur during DNA replication as a result of exposure to exogenous or endogenous compounds (Roy R, et al. Nat Rev Cancer. 2012; 12(1):68-78). Particularly during the menstrual cycle, hormonal stimulation sends strong growth signals to breast epithelial cells; amidst these intervals of rapid cell division, reactive oxygen species may accumulate, leading to DNA damage during replication (Hamada J, et al. J Natl Cancer Inst. 2001; 93(3):214-9). When the BRCA1/2 pathway fails to repair such damage, due to inherited (or acquired) mutations, genomic instability ensues (Roy R, et al. Nat Rev Cancer. 2012; 12(1):68-78). In BRCA1/2 mutation carriers, breast tumors exhibit distinctive transcriptional patterns (Hedenfalk I, et al. N Engl J Med. 2001; 344(8):539-48), perhaps a downstream effect of genomic instability. BRCA1/2 mutations also manifest themselves transcriptionally in normal cells. In experiments where breast tissue that has been exposed to DNA-damaging agents, gene-expression analyses have revealed clear transcriptional differences between patients with BRCA1/2 mutations and control patients having no known mutation (nor a family history of breast cancer) (Kote-Jarai Z. Clinical Cancer Research. 2004; 10(3):958-963). A similar experiment on lymphoblastoid cell lines (derived from peripheral blood) revealed a similar pattern (Waddell N, et al. PLoS Genet. 2008; 4(5)). Despite these clear patterns, many BRCA1/2 mutation carriers never develop cancer. And tumors develop in many individuals who have a family history of breast cancer but no known DNA repair mutation. These observations indicate that impaired DNA damage response is often insufficient to support breast tumorigenesis.

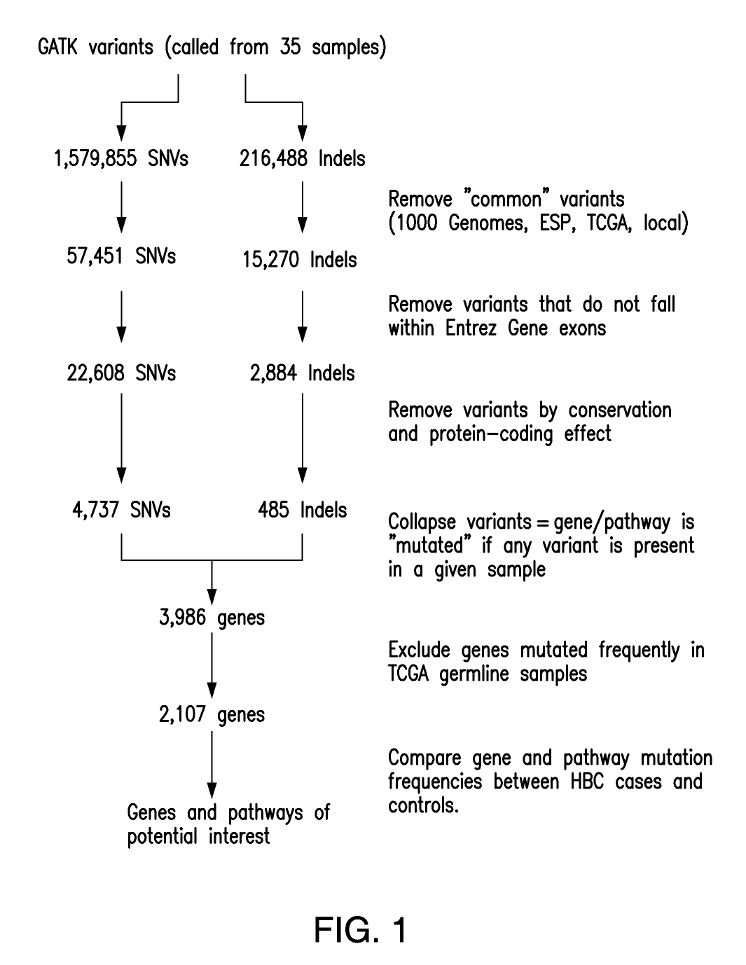

To assess the mutational landscape of hereditary breast cancer, exome-capture sequencing was used to profile a cohort of women carrying BRCA1 or BRCA2 mutations that were deemed to be pathogenic in clinical genetic testing. Women who tested negative for BRCA1/2 mutations but who have a strong family history of breast cancer (“BRCAX” carriers) were also profiled. Approximately half of these participants developed early-onset breast cancer, whereas the remaining participants have not been diagnosed with breast cancer and were at least 54 years old when peripheral blood was acquired for this study (see FIG. 1). Individuals who developed a tumor carried a relative abundance of “suspected pathogenic” germline variants in pathways that mediate cell growth, cell adhesion, and transcription. Thus deregulation of these pathways may play an important role in breast tumorigenesis for many high-risk women and may magnify risk when coupled with deregulated DNA repair processes.

In addition to single-nucleotide variants (SNVs) and short insertions/deletions (indels), various other types of (epi)genomic variation can influence transcriptional levels (Cookson W, et al. Nat Rev Genet. 2009; 10(3):184-94). These variations may reside within or outside protein-code regions (Dunham I, et al. Nature. 2012; 489(7414):57-74). Even though individuals who develop breast cancer may harbor different pathogenic variations, the downstream transcriptional effects of such variations can be similar for individuals who experience the same phenotype. Accordingly, detecting such patterns can enable identification of women who carry the highest risk of tumor development. Gene-expression microarrays can be used to profile peripheral blood of high-risk individuals and controls; the support vector machines algorithm (Vapnik V N. Statistical learning theory. New York: Wiley; 1998; Noble W S. Nat Biotechnol. 2006; 24(12):1565-7) can then be used to derive multigene signatures to differentiate the groups. For two retrospective cohorts from distinct geographic areas, the models predicted breast-cancer development more accurately than existing risk-prediction models (Wacholder S, et al. N Engl J Med. 2010; 362(11):986-93; Gail M H, et al. J Natl Cancer Inst. 1989; 81(24):1879-86; Costantino J P, et al. J Natl Cancer Inst. 1999; 91(18):1541-8). More notably, the approach described herein predicted hereditary breast-cancer development for a cohort of women whose peripheral blood was obtained prospectively—years before anyone in the cohort developed a tumor. Additionally, follow-up pathway-level analyses pointed to pathways known to influence tumorigenesis and some of the same biological processes identified via exome sequencing.

### Methods

### Patient Cohorts

Gene expression data for pathway-based analyses was as described above.

### Additional Data Cohorts

Among the 124 Utah participants, 34 were selected for exome sequencing. One additional individual was subsequently added to the exome-sequencing cohort.

### TCGA Cohort

Germline exome-sequencing data for 611 patient samples was downloaded from The Cancer Genome Atlas (TCGA) via the Cancer Genomics Hub. Most samples were from peripheral blood; the remaining 57 samples profiled were from normal breast tissue. Additionally, RNA-Seq (v2) normalized read counts for the same samples were downloaded from the TCGA data portal.

### Utah Exome Sequencing Cohort

Genomic DNA from peripheral blood was used for exome sequencing at the Huntsman Cancer Institute. Genomic DNA was then hybridized using Agilent SureSelect Human All Exon v4+UTRs kits. Captured libraries were sequenced on an Illumina Hi-Seq 2000 instrument, and barcoding techniques were used for multiplexing (seven lanes, five samples per lane). This process resulted in 101-bp paired-end reads (58,032,900 unique reads per sample).

For the Utah samples, reads were aligned to the hg19 human reference genome using the Burrows-Wheeler Aligner software (BWA, version 0.6.1) (Li H, Durbin R. Bioinformatics. 2009; 25(14):1754-60). Duplicate reads were marked using Picard tools (v. 1.64, http://picard.sourceforge.net/), and reads were sorted using samtools (v. 0.1.18) (Li H, et al. Bioinformatics. 2009; 25(16):2078-9). Using the Genome Analysis Toolkit (GATK, v. 1.5.3), reads were subsequently passed through various processing steps to realign and recalibrate the reads and then to detect SNVs and short indels; the GATK Best Practice Variant Detection guide was followed. For the TCGA samples, the same steps were followed; however, because the samples were processed at a later date, slightly newer software versions (BWA v. 0.6.2, Picard 1.81, and GATK 2.3.4) were used.

Multiple criteria were used to filter the initial variant call set (see FIG. 1). Any variant for which a minor-allele frequency greater than one percent had been reported in any ethnic population in either the 1000 Genomes ((Abecasis G R, et al. Nature. 2012; 491(7422): 56-65) (phase 1, release 3) or Exome Sequencing Project 6500 (NHLBI GO Exome Sequencing Project (ESP). Exome Variant Server Web Site. http://evs.gs.washington.edu/EVS. Accessed Feb. 2, 2013) data was excluded. Additionally, variants for which a minor-allele frequency greater than three percent was observed in the germline TCGA data were excluded; a higher threshold was used for TCGA because this population was likely enriched for disease-causing variants. Next variants that fell outside exons (plus/minus two bases to allow for detection of splice-site mutations) used in each gene's primary transcript were excluded; gene/transcript definitions were extracted from Entrez Gene (Maglott D, et al. Nucleic Acids Res. 2005; 33(Database issue):D54-8).

The remaining variants were annotated for effect on protein coding using the snpEff tool (Cingolani P, et al. Fly (Austin). 2012; 6(2):80-92). With this tool, variants are assigned a severity level based on their likelihood to impact the downstream protein. Variants that were assigned a severity of “MODIFIER” or “LOW” were excluded. Any variant that was assigned a “HIGH” severity was retained; these consisted primarily of truncating, frameshift, and splice-site variants. “MODERATE” indelswere also retained. Nonsynonymous coding SNVs were furthered examined using the Condel tool (González-Pérez A, et al. Am J Hum Genet. 2011; 88(4):440-9). This tool aggregates evidence from multiple algorithms (SIFT (Kumar P, et al. Nat Protoc. 2009; 4(7):1073-81), Polyphen (Adzhubei I A, et al. Nat Methods. 2010; 7(4):248-9), Mutation Assessor (Reva B, et al. Nucleic Acids Res. 2011; 39(17):e11843)) that use evolutionary conservation and/or impact on protein structure to estimate whether variants are pathogenic. Condel generates an aggregate score for each variant and then identifies a threshold that separates “deleterious” variants from “neutral” ones. Any missense SNV called as “neutral” was excluded. The remaining variants constituted the final set of “suspected pathogenic” variants. For simplicity, heterozygous variants and homozygous variants for which both alleles were the minor allele were considered to have an equivalent effect; the great majority of variants were heterozygous.

To enhance biological interpretation, variants were aggregated at the gene and pathway levels. If a given sample carried any suspected pathogenic variant in a given gene, that gene was considered “mutated” (typically, a gene had no more than a single variant in a given sample). Under the assumption that frequently mutated genes are unlikely to play a role in cancer biology due to selective pressure, we excluded any gene that was mutated in more than 1.8% of TCGA germline samples. This threshold was selected based on the maximal difference in number of excluded genes for thresholds that fell between 0.2% and 10%. Then if a given sample carried any variant in any gene from a given pathway, that pathway was considered “mutated”. Comparisons between the number of mutated and non-mutated samples were performed using Fisher's exact test. For a given pathway, the results varied only modestly with the choice of gene-filtering threshold described above.

Gene-pathway relationships were obtained from two literature-curated sources. Kyoto Encyclopedia of Genes and Genomes (KEGG) pathways (Kanehisa M, et al. Nucleic Acids Res. 2006; 34(Database issue):D354-7) were downloaded directly from the KEGG FTP site on Jun. 16, 2011. The remaining gene lists were obtained from the Molecular Signatures Database (v3.0) (Subramanian A, et al. Proc Natl Acad Sci USA. 2005; 102(43):15545-15550), which aggregates pathways from various sources, including REACTOME (Matthews L, et al. Nucleic Acids Res. 2009; 37(Database issue):D619-2246) and Biocarta (biocarta.com website).

The relationship between variants in a given gene and expression of that gene was estimated using Spearman's rank correlation coefficient. A threshold was determined using a local False Discovery Rate approach (Strimmer K. BMC Bioinformatics. 2008; 9:303). Variants with absolute correlation coefficients more extreme than this threshold were considered to be associated with expression of that gene.

### Software

All software scripts that were used for this study have been aggregated into a software pipeline that can be executed at the command line on UNIX-based systems. This pipeline is available upon request.

The pROC R package was used to produce ROC curve graphs (Robin X, et al. BMC Bioinformatics. 2011; 12:77). The fdrtool package as used to calculate FalseDiscovery Rates (Strimmer K. Fdrtool: Bioinformatics. 2008; 24(12):1461-2). The Python programming language (python.org) was used for parsing and summarizing data files. The DendroPy package (Sukumaran J, Holder M T. Bioinformatics. 2010; 26(12):1569-71) was used for performing Fisher's exact test; right-tailed p-values were used, which signified the odds that mutations were enriched in individuals who developed cancer.

### Results

Exome-Sequencing Variant Calls are Highly Concordant with Commercial Genetic Test Results for BRCA1 and BRCA2

Exome-capture sequencing was used to profile 35 women from Utah of northern European descent who had a strong family history of breast cancer (two or more first-degree relatives diagnosed). In prior commercial genetic testing, pathogenic BRCA1 or BRCA2 variants had been observed in 17 of the women; the remaining “BRCAX” women had tested negative for BRCA1/2 variants. Within the entire cohort, 19 women had developed early-onset breast cancer; the remaining women had not been diagnosed with breast cancer and were at least 54 years old (see FIG. 9).

On average per sample, 5,848,610,129 bases aligned to the reference genome, and 88.24% of bases fell within exome-capture target regions, resulting in a mean target coverage of 58.10. Across all samples, variants were observed at 1,796,343 unique loci (1,579,855 SNVs and 216,488 short insertions/deletions). After exome-wide filtering by minor allele frequency, effect on protein coding, and conservation (see Methods), 6,573 “suspected pathogenic” variants were observed at 5,222 loci.

To assess the validity of the variant calls, the results were compared against the PCR-based, commercial genetic test results for BRCA1/2. The variant calls agreed nearly perfectly with those results. One false-negative call (94.1% sensitivity) and two false-positive calls (88.89% specificity) were observed. The false-negative variant (rs80358061) resides in a BRCA1 intronic region outside the splice-site junction points. This variant was identified via exome sequencing; however, it was filtered out due to its intronic location. The false positives (rs28897683 and rs80356935) are nonsynonymous SNVs that lie outside the BRCT and RING domains of the BRCA1 protein and thus are likely not to be pathogenic despite evolutionary conservation; neither individual from the study who carried a false-positive variant developed cancer, and one had a known pathogenic variant in BRCA2.

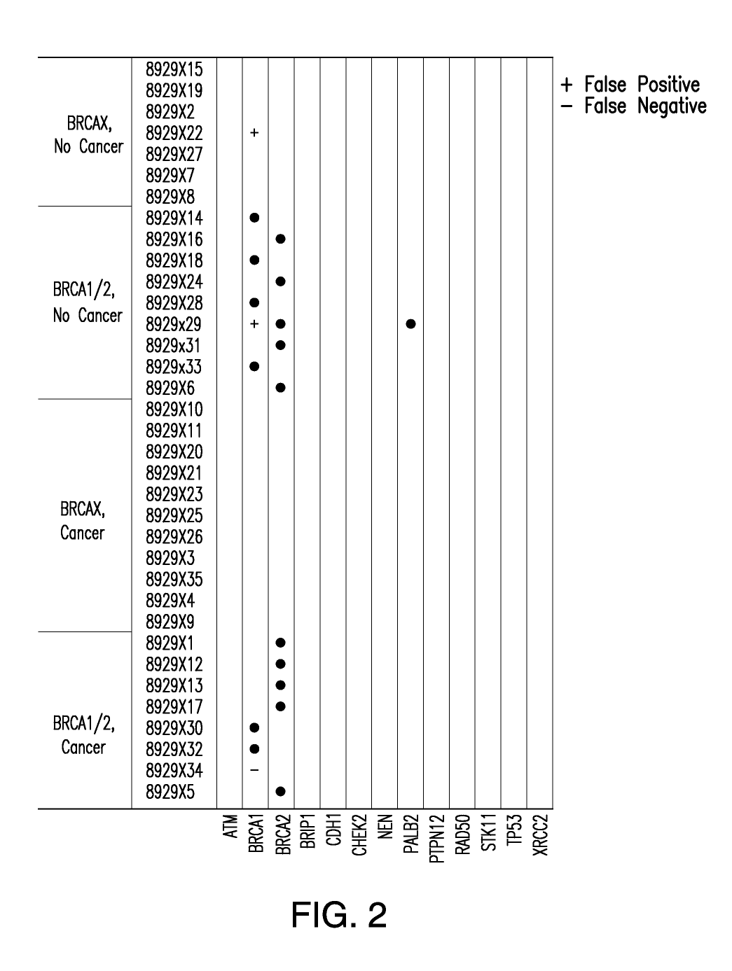

Most suspected pathogenic variants were non-synonymous substitutions. The most frequent substitutions were G-to-A and C-to-T transitions. Most insertions and deletions resulted in a net gain/loss of less than seven nucleotides; however, a few were larger. At least one variant was observed in 4,256 unique genes. A few variants occurred in known breast-cancer susceptibility genes; however, these variants often occurred concomitantly with BRCA1/2 variants or in individuals who did not develop cancer (see FIG. 2).

Hereditary Breast Cancer Enriched for Germline Variants in Genes Associated with Biological Processes Known to Influence Cancer Development

The mutational profiles of individuals who developed hereditary breast cancer (regardless of BRCA1/2 status) were compared against those of individuals who had a strong familial predispositionbut did not develop a tumor. For each sample, a simple rare-variant collapsing method (Li B, Leal S M. The American Journal of Human Genetics. 2008; 83(3):311-21) was used to indicate whether a given gene or biological pathway had been “mutated” by one or more variants. When comparing the number of mutated cancer cases versus non-cancer controls, significant differences were observed for pathways related to growth signaling, cell adhesion, metabolism, immune response, and DNA repair (Tables 8 and 9). Each of these biological processes has been shown to influence tumor development in different settings. These findings indicate that germline genetic variation plays an important role in perturbing biological pathways that can drive cellular transformation; due to genetic heterogeneity, mechanisms likely vary considerably from one tumor to the next.

TABLE 8
Pathway-level result summary. Pathways for which the pathway-based biomarkers attained an AUC of at least 0.63 in
both the Utah and Ontario cohorts and for which the number of samples with a “mutated” pathway differed substantially
(p <= 0.1) between individuals who developed breast cancer or did not. Pathways are sorted by average AUC.

| | Utah AUC | Ontario AUC | # Controls | # Cancer | Fisher p-value |
|---|---|---|---|---|---|
| Pathway | (Expression) | (Expression) | Mutated | Mutated | (empirical) |
| KEGG PHOSPHATIDYLINOSITOL SIGNALING SYSTEM | 0.647 | 0.705 | 4/16 | 10/19 | 0.092 |
| KEGG CELL ADHESION MOLECULES CAMS | 0.635 | 0.729 | 2/16 | 8/19 | 0.076 |
| KEGG FRUCTOSE AND MANNOSE METABOLISM | 0.851 | 0.684 | 1/16 | 7/19 | 0.037 |
| KEGG CITRATE CYCLE TCA CYCLE | 0.695 | 0.660 | 0/16 | 7/19 | 0.01 |
| REACTOME INTEGRIN CELL SURFACE INTERACTIONS | 0.72 | 0.654 | 2/16 | 8/19 | 0.048 |

Table 9: Top pathways for which the number of mutations differed significantly between hereditary breast cancer cases and controls. For a given sample, a pathway was considered to be affected if any suspected pathogenic mutation had been observed in any gene in that pathway. The Fisher's exact test was used to compare the number of mutated samples between those who developed cancer and those who did not.

Taken together, these analyses indicated that—in addition to impaired DNA repair response—mutational deregulation of several other biological processes plays an important role in breast-cancer susceptibility. Although the precise pathways differed among these data sets, pathways involved in cell adhesion, PI3K, ERBB signaling, apoptosis and metabolism consistently rose to the top of the lists and correlated to breast cancer risk phenotypes.

Tables 12 and 13 show the top genes selected from the Utah 1 and Utah 2 cohorts, respectively.

### Gene-Expression Profiles of Peripheral Blood Point to Similar Biological Processes

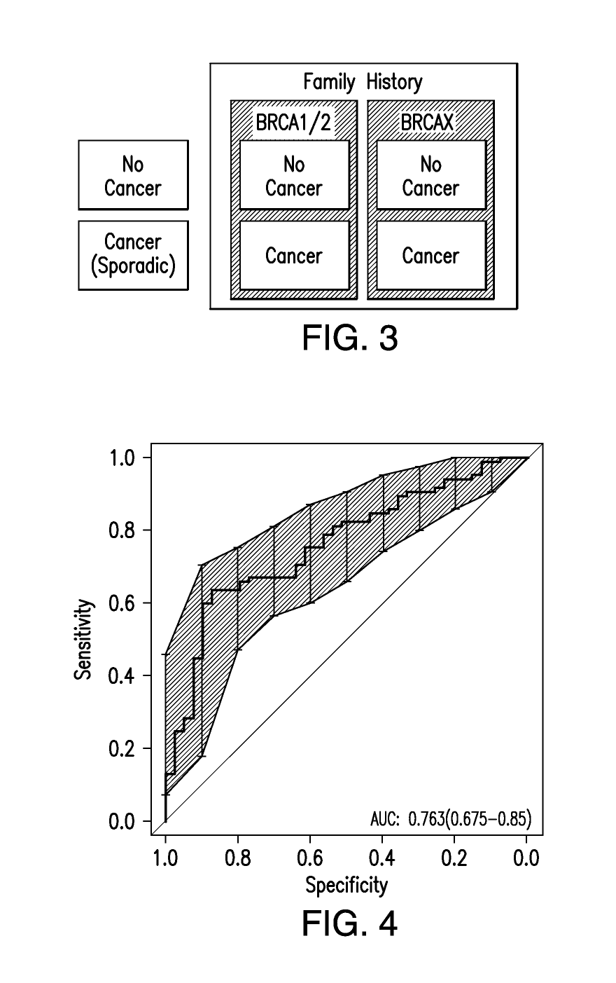

Although genetic variants in coding regions play an important role in disease etiology, they only tell part of the story. Variants that occur in non-coding regions broadly influence transcription (Dunham I, et al. Nature. 2012; 489(7414):57-74) and may deregulate biological processes. Additionally, epigenetic modifications and non-coding RNA can play important roles in human disease (Portela A, Esteller M. Nat Biotechnol. 2010; 28(10):1057-1068; Esteller M. Nat Rev Genet. 2011; 12(12):861-74). Any single variation can manifest itself via changes in gene expression in one or multiple genes (Emilsson V, et al. Nature. 2008; 452(7186):423-8). In aggregate, many such variations leave a complex footprint on the gene-expression landscape. The balance may be tipped toward stronger disease susceptibility when that footprint disturbs biological processes that are designed to maintain homeostasis. On the premise that such patterns will manifest themselves in normal tissue before tumorigenesis, the potential to derive gene-expression biomarkers that identify individuals at the highest risk for developing breast cancer were explored. Gene-expression microarrays were used to profile peripheral blood mononuclear cells for a cohort of 124 women from Utah (34 of whom overlapped with the exome-sequencing cohort). Seventy-three women from Ontario, Canada were also profiled; blood samples for 37 of the Ontario women were acquired prospectively—years before any of the women were diagnosed with breast cancer. Each woman fell into one of six groups, divided along three axes: 1) family history of breast cancer, 2) presence of a pathogenic germline variant in BRCA1 or BRCA2, and 3) previous diagnosis with early-onset breast cancer (see FIG. 3).

Using expression data for all genes, a biomarker that characterizes individuals with a family history of breast cancer who actually developed a tumor was developed. The remaining individuals served as a control population, which included women who developed sporadic tumors. This design allowed for the control of any latent effects due to cancer treatments. Using the support vectormachines (SVM) algorithm (Vapnik V N. Statistical learning theory. New York: Wiley; 1998), how well hereditary breast-cancer development could be predicted in the retrospective Utah population was assessed. In cross validation, individuals with a family history who developed breast cancer were assigned considerably higher “hereditary breast cancer scores” than the controls (see FIG. 7), attaining an AUC value of 0.763 (0.675-0.850; see FIG. 4).

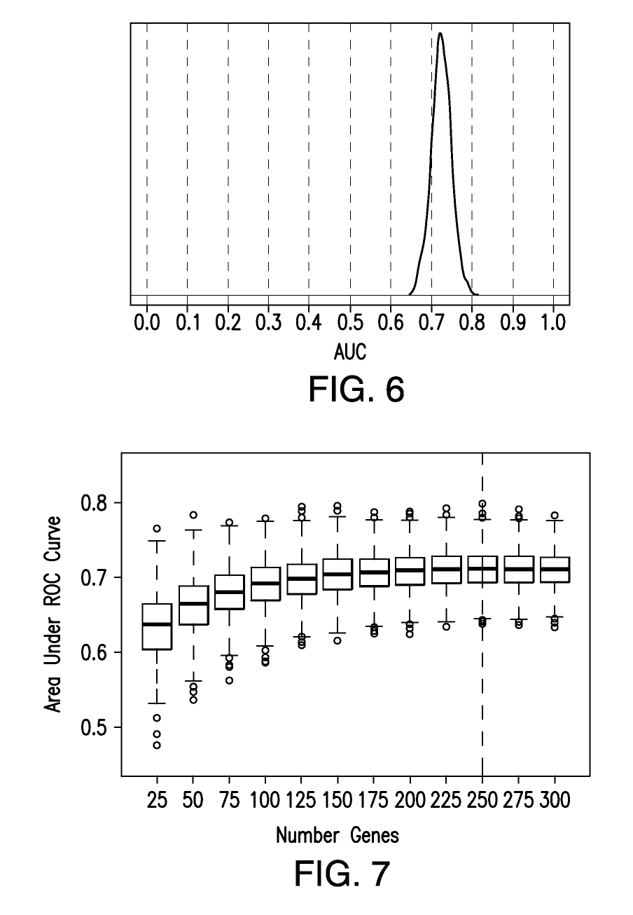

Three approaches were used to further assess the predictive ability of this biomarker. Firstly, the cross-validation analysis was repeated 1,000 times, using a different random seed each time; AUC values were consistently higher than 0.7 (see FIG. 6), an indication that the initial result was not simply due to fortuitous cross-validation fold assignments. These results also were robust to the number of genes included in the biomarker; on average, the best cross-validation results were attained using 250 genes and varied little between 150 and 300 genes (see FIG. 7). Secondly, the analysis was repeated 1,000 times with randomly permuted class labels; as should be expected, AUC values were centered near 0.5, and only two values were higher than 0.763 (p=0.002, see FIG. 8). Finally, whether the biomarker would generalize to a geographically and ethnically distinct population was assessed. The SVM model was trained on the Utah data and predicted hereditary-breast cancer status for the Ontario samples. These predictions attained a combined AUC of 0.733 (see FIG. 5). Importantly, blood samples for many of the Ontario cohort had been acquired years before each patient's outcome was known.

Taken together, these results provide evidence that a gene-expression biomarker based on peripheral blood can be used to prospectively inform women from high-risk breast-cancer families regarding their individual likelihood of developing breast cancer. The biomarker's predictive capability also indicates that even though each individual carries a highly unique genetic/epigenetic profile (and in many cases, different BRCA1/2 variants), common patterns are detectable at the gene-expression level.

To shed additional light on the biology underlying these gene-expression patterns, two follow-up analyses were performed. A set of “consensus” genes that are consistently deregulated across the Utah and Ontario data sets were identified. Among genes that were either upregulated or downregulated in each cohort, the genes were then ranked according to expression difference between HBC samples and controls. The highest-ranked genes were associated (Chang J T, Nevins J R. Bioinformatics. 2006; 22(23):2926-33) with KEGG pathways related to cellular adhesion and metabolism (see Tables 13 and 15).

Table 13: Top genes consistently differentially expressed between individuals who developed hereditary breast cancer and controls.

TABLE 15
KEGG pathways associated with genes from Table 13.

| | | Bayes | |
|---|---|---|---|
| Term ID | Term | Factor | p-value |
| hsa04520 | Adherens junction | 2.89 | 0.000762 |
| hsa04510 | Focal adhesion | 0.59 | 0.00865 |
| hsa04512 | ECM-receptor interaction | 0.56 | 0.00919 |
| hsa00061 | Fatty acid biosynthesis (path 1) | 0.29 | 0.0123 |
| hsa00590 | Prostaglandin and leukotriene | 0.23 | 0.0132 |
| | metabolism | | |

In a follow-up analysis, the gene-expression data was filtered to include only genes that participate in a given biological pathway. Pathway-gene relationships were extracted from various literature-curated resources (see Methods). Table 16 lists the results of these experiments. As in the analyses of germline variants, many of the top-performing pathways play a role in cell adhesion, apoptosis, metabolism, and immune response.

TABLE 16
Pathway-based biomarker prediction results. Hereditary breast cancer
status was predicted based on genes that belong to literature-curated
biological pathways. For a given pathway, the gene-expression data
was preliminarily filtered to include only genes from that pathway.
Pathways are sorted by average AUC across the cohorts.

| Pathway | Utah | Ontario |
|---|---|---|
| KEGG SMALL CELL LUNG CANCER | 0.741 | 0.680 |
| KEGG PEROXISOME | 0.674 | 0.712 |
| BIOCARTA DREAM PATHWAY | 0.656 | 0.701 |
| KEGG PHOSPHATIDYLINOSITOL SIGNALING | 0.647 | 0.705 |
| SYSTEM | | |
| BIOCARTA BCR PATHWAY | 0.698 | 0.705 |
| REACTOME FORMATION OF PLATELET PLUG | 0.704 | 0.668 |
| REACTOME CELL DEATH SIGNALLING VIA | 0.653 | 0.684 |
| NRAGE NRIF AND NADE | | |
| KEGG ARRHYTHMOGENIC RIGHT | 0.619 | 0.733 |
| VENTRICULAR CARDIOMYOPATHY AR . . . | | |
| BIOCARTA AT1R PATHWAY | 0.736 | 0.643 |
| KEGG PATHWAYS IN CANCER | 0.793 | 0.634 |
| BIOCARTA STRESS PATHWAY | 0.683 | 0.691 |
| KEGG CELL ADHESION MOLECULES CAMS | 0.635 | 0.729 |
| BIOCARTA CELL2CELL PATHWAY | 0.628 | 0.703 |
| KEGG OSTEOCLAST DIFFERENTIATION | 0.663 | 0.684 |
| REACTOME PLATELET DEGRANULATION | 0.739 | 0.656 |
| REACTOME RNA POLYMERASE I | 0.701 | 0.635 |
| PROMOTER CLEARANCE | | |
| KEGG CYTOKINE CYTOKINE | 0.673 | 0.677 |
| RECEPTOR INTERACTION | | |
| KEGG FC EPSILON RI SIGNALING PATHWAY | 0.637 | 0.682 |
| BIOCARTA PYK2 PATHWAY | 0.762 | 0.625 |
| REACTOME FANCONI ANEMIA PATHWAY | 0.58 | 0.688 |

### Example 3

### Individualized Multi-Omic Risk Assessment of Hereditary Breast Cancer

### Summary

While prevention strategies such as prophylactic surgery and chemoprevention are available for women with a family history of breast cancer, these treatments cause physical and psychological effects that make them unacceptable to many individuals. In addition, individual cancer risk may vary for women from high-risk families, as not all women in these families develop breast cancer even if they inherit the same highly penetrant predisposition genes or other identifiable genetic modifiers. To predict a woman's individual risk for development of hereditary breast cancer and gain insight into novel susceptibility signaling pathways, the germline transcriptome and genetic variants were profiled from peripheral blood of affected high-risk women as well as three groups of controls: unaffected high-risk women, women with sporadic breast cancer, and normal controls without breast cancer. Across multiple cohorts from distinct geographical regions, multigene expression profiles and mutation spectra for pathways related to cell-cell adhesion and integrin signaling consistently distinguished affected high-risk women from controls better than for hundreds of other pathways. Validation studies using cell-based assays on an independent set of primary breast cells support the concept that cell-cell and cell—extracellular matrix adhesion processes are disrupted in non-malignant mammary epithelial cells of high-risk women. To further optimize clinical application, a blood-based biomarker was empirically derived from genome-wide expression levels. This biomarker predicted a high-risk individual's likelihood of developing hereditary breast cancer with high accuracy and was validated in an independent high-risk population. Together, these studies reflect a new approach to identification of novel susceptibility pathways and prediction of hereditary breast cancer risk.

### Background

High-throughput genomic profiling has been instrumental to recent biomedical advances including defining novel cancer subtypes, refining diagnosis or prognosis estimates, and tailoring therapeutic interventions to individual patients. Whereas DNA sequencing analysis can identify discrete variants that influence cancer predisposition or tumorigenesis, transcriptional profiling can reflect more complex dynamics that occur within a cell, driven by genetic and epigenetic variation, as well as co-regulation of genes within a biological pathway. Complementary insights can be gained when multiple genetic/genomic approaches are used for a given sample. For example, in breast and brain tumors, somatic mutation data have been combined with gene-expression data to improve the understanding of the molecular basis of tumorigenesis and mechanisms of treatment response. In addition, transcription levels in normal cells reflect intermediate effects arising from germline variation and thus can aid in interpreting the clinical significance of such variants.

Researchers have identified various genes that may play a role in breast tumor development. Although much can be gained from examining the roles of such genes or alleles in isolation, a more holistic, pathway-based approach is necessary to elucidate the broad impact of genomic variation. For example, candidate driver mutations vary widely across individuals and are often unique to each tumor. Wood, et al. showed that p110α, the active component of PI3K, was mutated in 11.9% of breast tumors; however, 33.3% of tumors contained mutations in the PI3K pathway if other genes in the network are considered. Pathway-level aggregation can place such observations in biological context and reduce data dimensionality, thus aiding discovery of biological processes that are differentially regulated between conditions. Unfortunately, in many genetic-predisposition studies, pathway-based summarization is complicated by loci falling outside coding regions; additionally, de novo efforts to identify alleles that coordinately influence disease risk are hindered by the extreme statistical burden of testing higher order interactions. Transcriptional profiling can overcome these limitations by capturing downstream functional effects of multiple types of germline aberrations. This association between genetic and transcriptomic variation was initially mapped in yeast, which linked genetic variants in parental yeast to expression traits in progeny. Subsequent studies have identified thousands of expression quantitative trait loci in the human genome. In fact, recent studies have demonstrated that genetic variation can be associated consistently with global mRNA-expression patterns and that these patterns can reflect heritable disease susceptibility. Different variants within a given gene or pathway may have similar downstream effects if they drive coordinated changes in mRNA transcription levels. For both normal and cancer cells, previous studies highlight the ability of transcriptional profiling to reveal pathway-activation status, independent of the underlying mechanisms such as genetic variants. Specific to breast cancer, peripheral blood cells and tumors from women with BRCA1/2 mutations express distinct patterns of gene expression from controls, indicating that genetic variance is reflected in gene expression data, which can be used as a marker for disease risk.

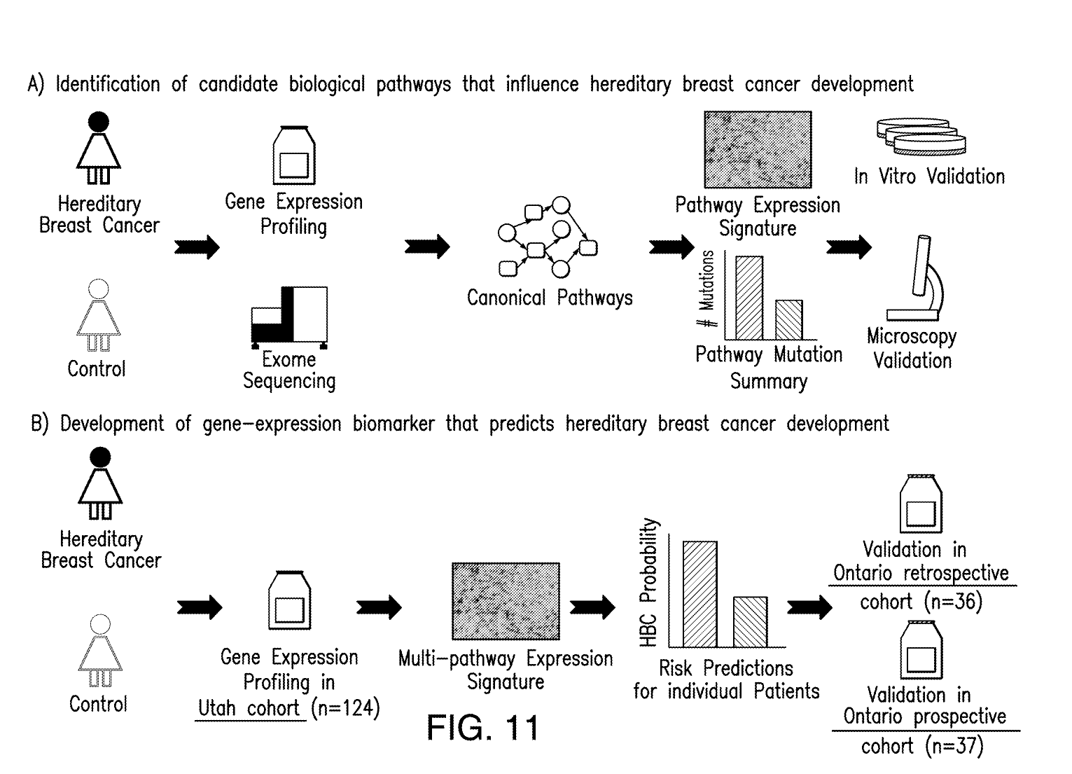

To interrogate signaling pathways dysregulated in women who develop hereditary breast cancer (HBC), both gene expression and DNA-sequencing data was leveraged from peripheral blood mononuclear cells (PBMCs) (FIG. 11); this data was used to discriminate between individuals who develop HBC and those who do not by using genes from each of over 900 canonical signaling pathways. Pathways were identified that discriminate between women with HBC and controls in three independent gene-expression cohorts and a DNA-sequencing cohort. Multigene expression signatures for cell adhesion pathways distinguished women who developed HBC from controls; the women with HBC also carried a relative abundance of suspected pathogenic germline variants in these pathways. Based on these data, cell adhesion properties of normal mammary epithelial cells from either control breast reduction patients or prophylactic mastectomies from high-risk women were compared. Both fluorescence microscopy and in vitro assays provided evidence that cell adhesion mechanisms are disrupted in normal cells of high-risk individuals, supporting the pathway-based biomarker findings. As pathway-summarized transcriptomic data showed discriminatory patterns in affected high-risk women compared to controls, an empirical gene expression-based biomarker of HBC development was tested. The model predicted HBC development at high accuracy levels and was able to predict hereditary breast cancer development for an independent, demographically distinct cohort of high-risk women. Together, transcriptomic, genetic, and phenotypic variation implicate dysregulated cell adhesion pathways in HBC susceptibility. The findings also indicate that transcriptomic profiles of normal cells from high-risk women have potential to predict subsequent development of breast cancer and therefore guide individual patients' decisions regarding medical management.

### Results

### Overview of Patient Cohorts and Genomic Data Acquisition

Exon microarrays were used to quantify gene-expression levels in PBMCs for an original cohort of 124 women from the High Risk Breast Cancer Clinic at the Huntsman Cancer Institute (Utah, USA) and for an independent validation cohort of 73 women from the Ontario, Canada site of the Breast Cancer Family Registry (see Tables 17 and Table 7). Importantly, the validation cohort was from a geographically distinct region and included many women who were premenopausal. Each cohort contained women from HBC families—each of these participants had two or more relatives with breast cancer. Nearly half of these women developed HBC, whereas the others met the family history criteria but had not developed breast cancer by at least 54 years of age. Women from HBC families were equally distributed between those that carried pathogenic mutations in BRCA1/2 and “BRCAX” women did not. Both cohorts also contained women with no family history of breast cancer and who either developed sporadic breast cancer or did not. For the gene-expression analyses, the women were categorized into two main groups: “HBC affected” and “controls” (FIG. 3 and Methods). The former group contained affected women from HBC families; the latter group contained unaffected women from HBC families and women who were not from HBC families and who did or did not develop sporadic breast cancer. To correct for potential confounding effects, expression data were filtered to remove genes whose expression correlated to epidemiological or demographic variance or to lymphocyte subpopulations (see Methods). Finally, a gene expression dataset of normal breast tissue from control or high-risk women was used to focus on signaling events present in breast epithelial cells.

TABLE 17
Data set summary. Biospecimens were acquired from
various sources and profiled using gene-expression
microarrays and/or exome-capture sequencing.
A) Gene expression

| | Tissue | | # Sam- | | # |
|---|---|---|---|---|---|
| Description | Source | Platform | ples | Facility | Genes |
| Utah | PBMCs | Affymetrix | 124 | Boston U. | 25,195 |
| | | Exon array | | | |
| Ontario | PBMCs | Affymetrix | 36 | Duke U. | 25,195 |
| (Retro- | | Exon array | | | |
| spective) | | | | | |
| Ontario | PBMCs | Affymetrix | 37 | Boston U. | 25,195 |
| (Pro- | | Exon array | | | |
| spective) | | | | | |
| Lim, | Pre- | Illumina | 20 | N/A | 25,186 |
| et al. | neoplastic | Human WG-g | | | |
| | breast | beadchip | | | |

B) DNA Sequencing

| Description | Tissue Source | #Sample |
|---|---|---|
| Utah | PBMCs | 124 |
| Cousin pairs . . . | ?? | 40 |
| The Cancer Genome | Blood/normal | 611 |

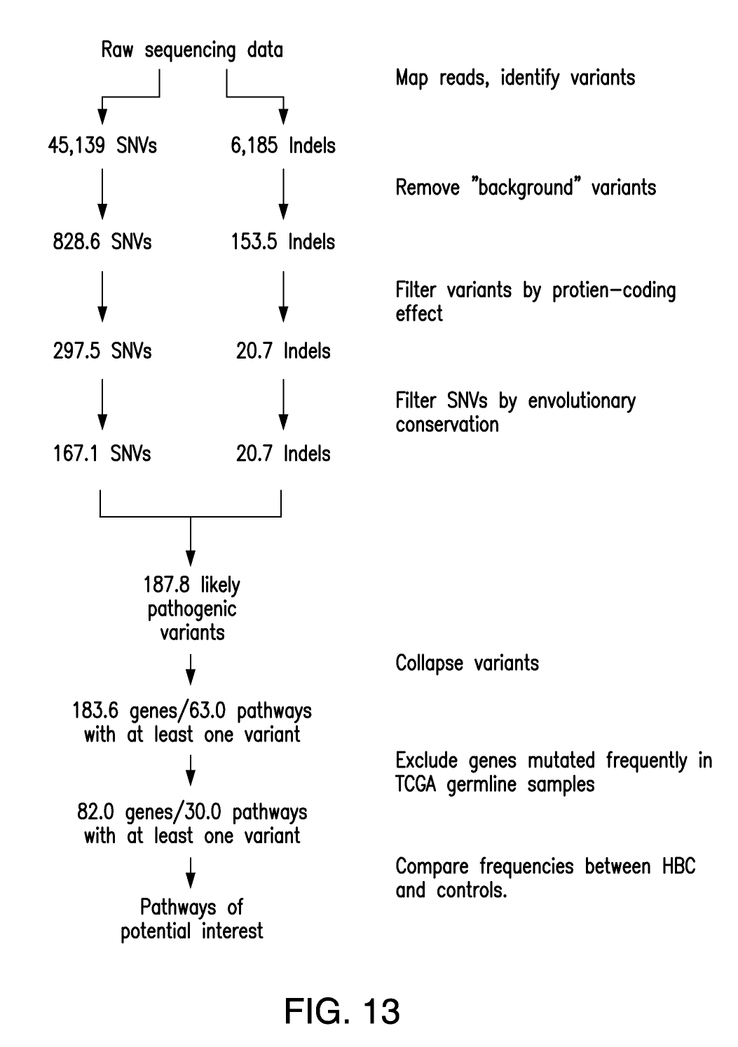

In conjunction with the expression data, the exomes of 35 patients in the Utah cohort for which we had matching DNA were sequenced. Suspected pathogenic variants were identified by developing a computational pipeline that filtered variants by population frequency, effect on protein coding, and evolutionary conservation (Methods and FIG. 13). Variants that have been observed frequently (>1%) in large population-based studies were excluded. In an important additional filtering step, the computational pipeline was used to process 611 germline samples from The Cancer Genome Atlas (TCGA); this step excluded 7.0% of single nucleotide variants (SNVs) and 35.7% of short insertion/deletion variants (InDels) that remained after population-based filtering. Finally, variants that occurred frequently (>15%) in the Utah samples were excluded. This step excluded an additional 34.6% and 34.8% of SNVs and InDels, respectively. For BRCA1/2, these variant calls were highly sensitive (94.1%) and specific (88.9%) compared to observations from commercial genetic testing. To enable pathway-based interpretation, variants at the gene and pathway levels were aggregated and frequently mutated genes were excluded. For a given sample, a pathway was considered to be “mutated” if a variant was observed in any gene within that pathway. On average, 82.0 genes and 30.0 pathways were mutated per sample.

### Multi-Omic Pathway-Specific Classification Hereditary Breast Cancer Risk

To gain a better understanding of biological processes that play a role in HBC development and to test the predictive capability of each individual pathway, the genomic data described above was mapped to 929 literature-curated pathway maps (FIG. 11). For the gene-expression data, a machine-learning classification approach was used, support vector machines (SVM), to evaluate the ability of expression levels from genes in a given pathway to distinguish affected HBC women from controls (unaffected HBC, sporadic cancer, and unaffected non-HBC); the statistical significance was calculated using a permutation approach (see Methods for additional details). For the sequencing data, significance was assessed based on the number of mutations in affected HBC versus controls using Fisher's exact test. Pathways were prioritized based on the combined level of statistical significance across the four cohorts comprised of two types of genomic data using Fisher's combined probability test. After a conservative Bonferroni correction, eight signaling pathways showed significant dysregulation (Bonferroni adjusted p-value<0.05, Table 18). The genes in these pathways are important in cell adhesion, oncogenic signaling networks (including cell cycle, MAPK, PI3K and TNF signaling), and metabolic processes.

TABLE 18
Pathways that fell below 0.05 significance threshold. Pathways that passed filtering
thresholds on Fisher combined probability test after Bonferroni correction.

| | Utah | Ontario | Lim | Utah | Combined | Corrected |
|---|---|---|---|---|---|---|
| Pathway | (RNA | (RNA) | (RNA) | (DNA) | p-value | p-value |
| KEGG SMALL CELL LUNG | 0.001 | 0.006 | 0.016 | 0.132 | 1.5e−05 | 0.0139 |
| REACTOME SIGNALING IN | 0.001 | 0.100 | 0.017 | 0.0144 | 2.62e−05 | 0.0242 |
| IMMUNE SYSTEM | | | | | | |
| KEGG CELL ADHESION | 0.005 | 0.001 | 0.090 | 0.058 | 2.76e−05 | 0.0256 |
| MOLECULES CAMS | | | | | | |
| BIOCARTA BCR PATHWAY | 0.001 | 0.004 | 0.028 | 0.287 | 3.29e−05 | 0.0305 |
| REACTOME INTEGRIN CELL | 0.001 | 0.017 | 0.037 | 0.058 | 3.65e−05 | 0.0339 |
| SURFACE INTE . . . | | | | | | |
| REACTOME RNA POLYMERASE I | 0.001 | 0.020 | 0.002 | 0.944 | 3.76e−05 | 0.0349 |
| PROMOTER- | | | | | | |
| KEGG CITRATE CYCLE TCA | 0.001 | 0.015 | 0.442 | 0.00749 | 4.73e−05 | 0.0438 |
| CYCLE | | | | | | |
| BIOCARTA STRESS | 0.002 | 0.008 | 0.016 | 0.227 | 5.39e−05 | 0.0499 |

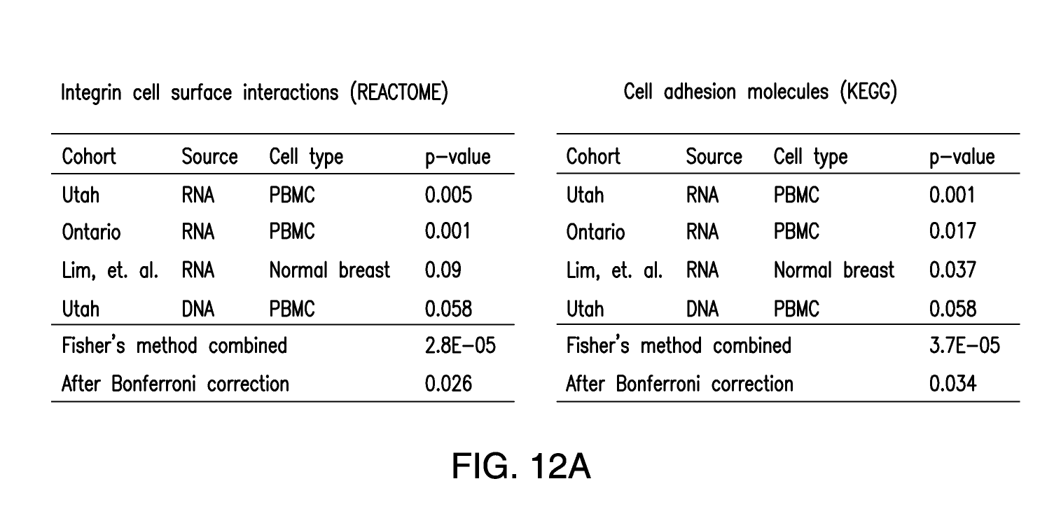

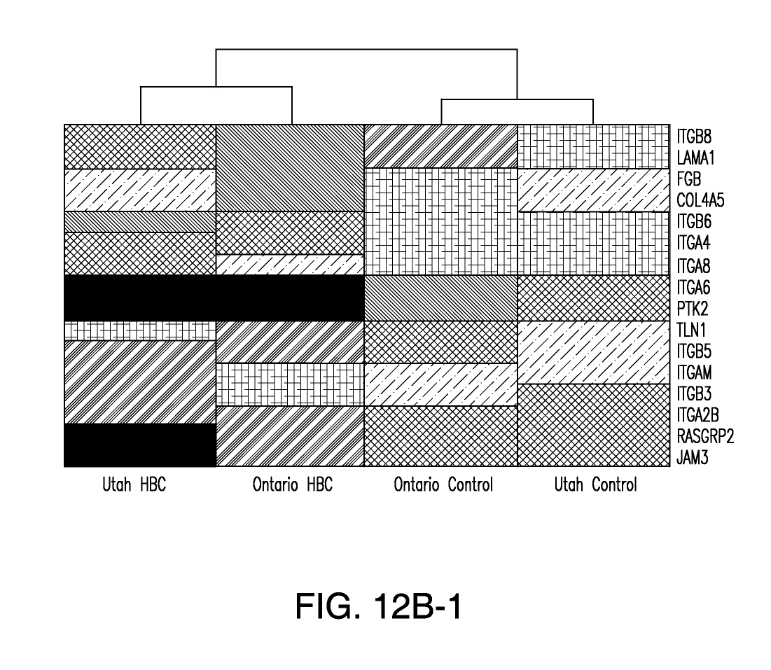

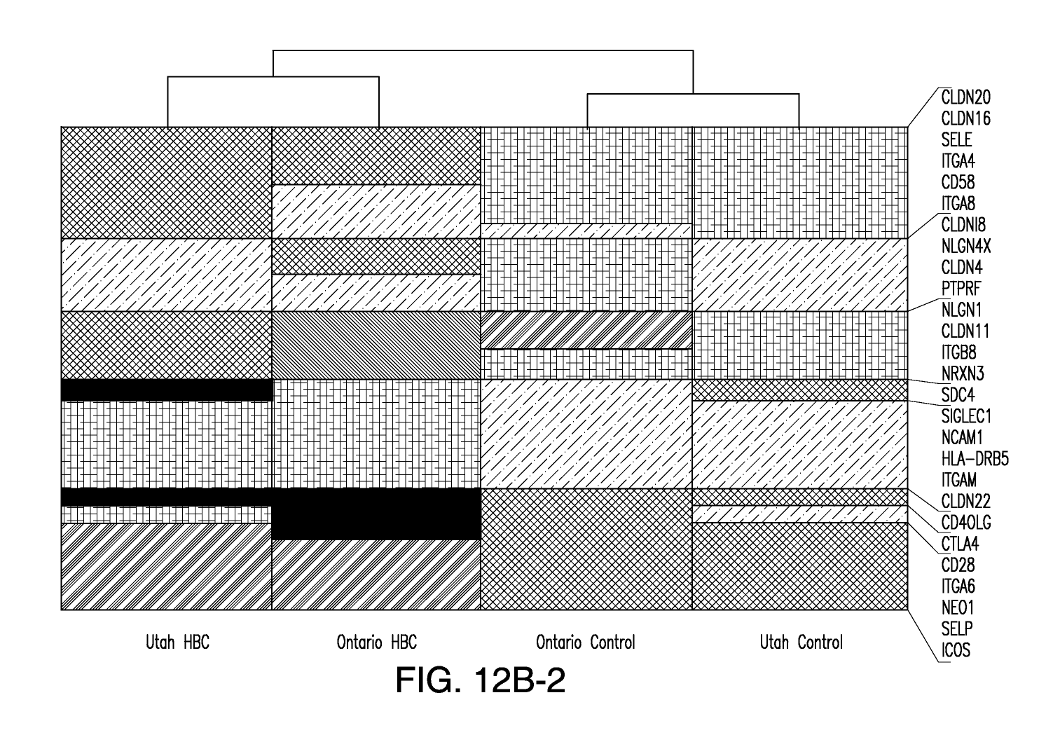

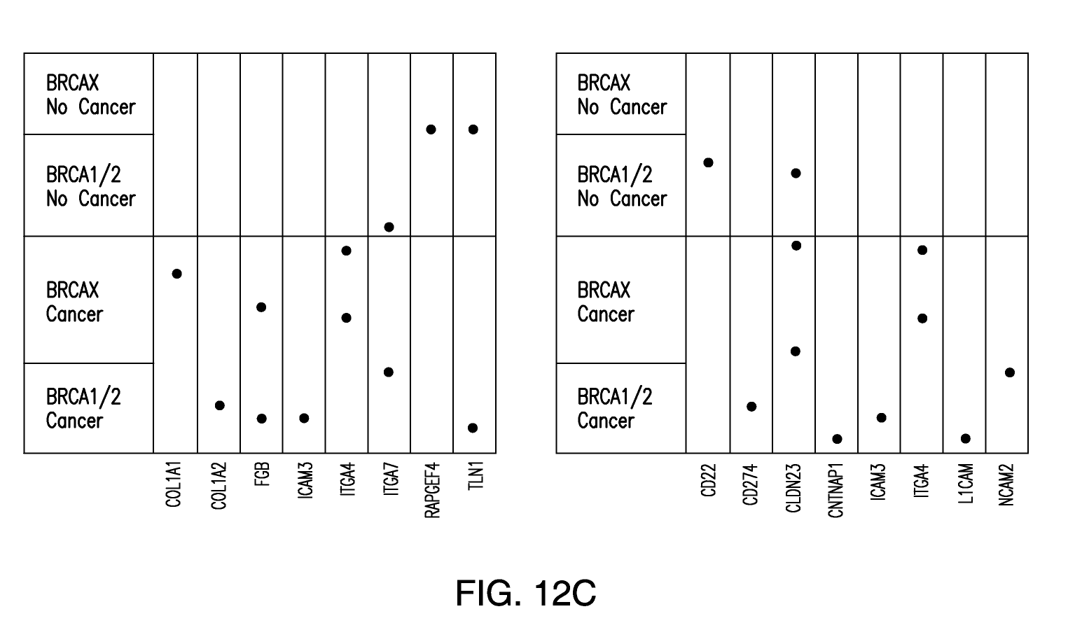

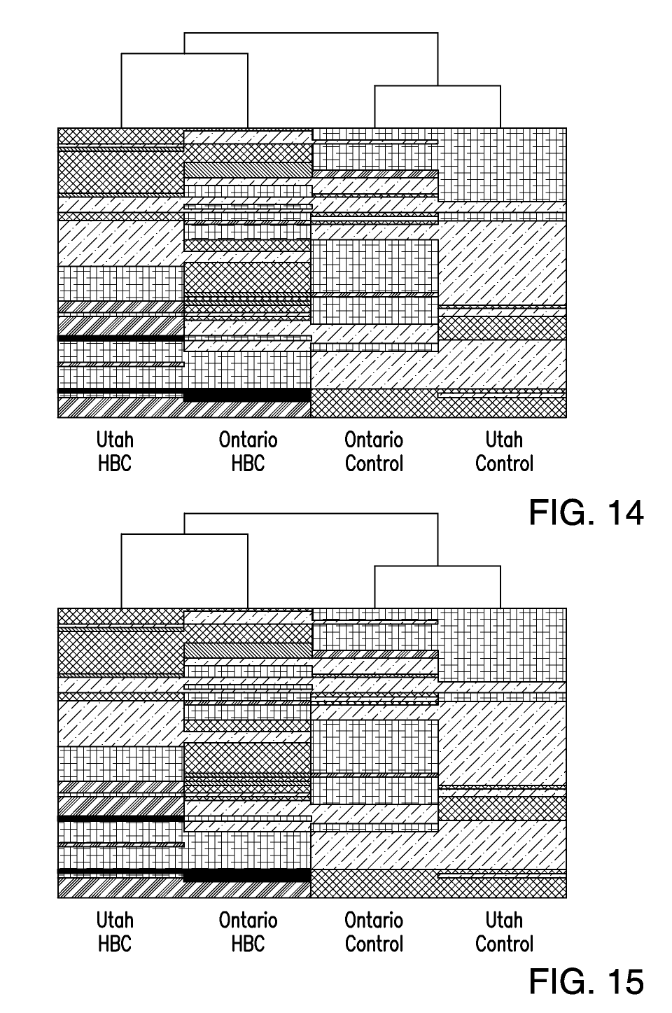

Two of these pathways—Integrin cell surface interactions and Cell adhesion molecules—were focused on as they were the only pathways with both a Bonferonni adjusted combined p-value less than 0.05 and individual p-values less than 0.10 for each-omic dataset examined (FIG. 12A). Specifically, classification models based on these pathways effectively discriminated “HBC affected” women from controls in the initial Utah training dataset. Importantly, these models were also able to accurately distinguish affected HBC individuals from controls in the Ontario validation dataset. Furthermore, in an independent dataset of normal breast tissue (Lim 2009, GSE17072), gene-expression profiles for these cell adhesion pathways were effective at classifying women with strong family histories of breast cancer from controls. FIG. 12B highlights specific genes whose expression was consistently different for these pathways between the two groups in the samples (see also FIGS. 14 and 15). As an independent statistical test, the GATHER algorithm was also applied to the genes that best distinguished affected HBC women from controls; this approach also indicated a significant association between HBC development and Kyoto Encyclopedia or Genes and Genomes (KEGG) pathways related to cell adhesion: Adherens junctions, extracellular matrix (ECM)-receptor interaction (p-values<0.02, Table 19).

TABLE 19
Summary of alternative cell-adhesion pathways. Pathways that did not pass filtering
thresholds but that provide additional evidence that cell-adhesion processes
are deregulated in hereditary breast cancer.

| | Utah | Ontario | Lim | Utah | Combined | Corrected |
|---|---|---|---|---|---|---|
| Pathway | (RNA | (RNA) | (RNA) | (DNA) | p-value | p-value |
| KEGG FOCAL ADHESION | 0.001 | 0.024 | 0.011 | 0.596 | 0.000123 | 0.114 |
| SIG CHEMOTAXIS | 0.002 | 0.059 | 0.024 | 0.072 | 0.000152 | 0.141 |
| BIOCARTA ECM PATHWAY | 0.001 | 0.053 | 0.026 | 0.227 | 0.000215 | 0.199 |
| REACTOME CELL SURFACE | 0.001 | 0.069 | 0.049 | 0.0935 | 0.000217 | 0.201 |
| INTERACTIONS . . . | | | | | | |
| ST INTEGRIN SIGNALING | 0.001 | 0.066 | 0.023 | 0.212 | 0.00022 | 0.204 |
| PATHWAY | | | | | | |

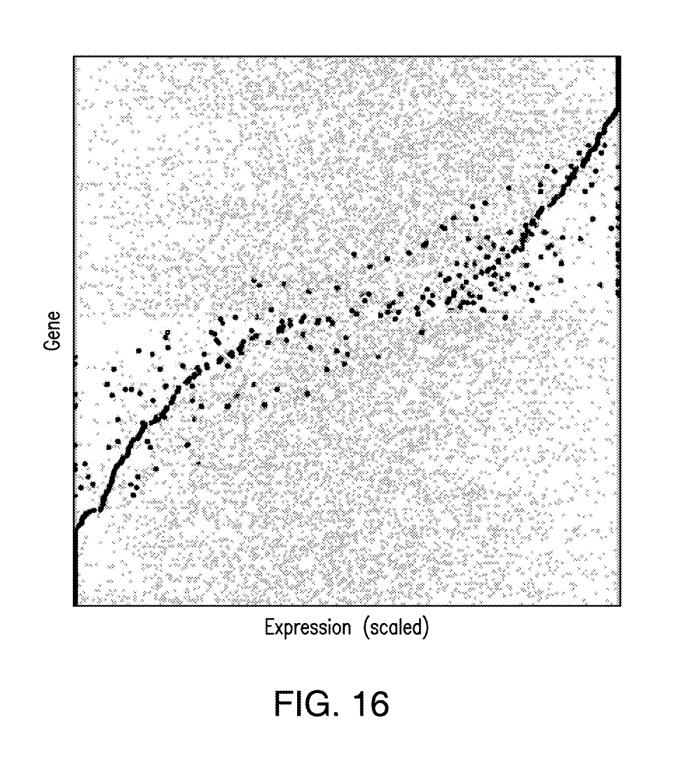

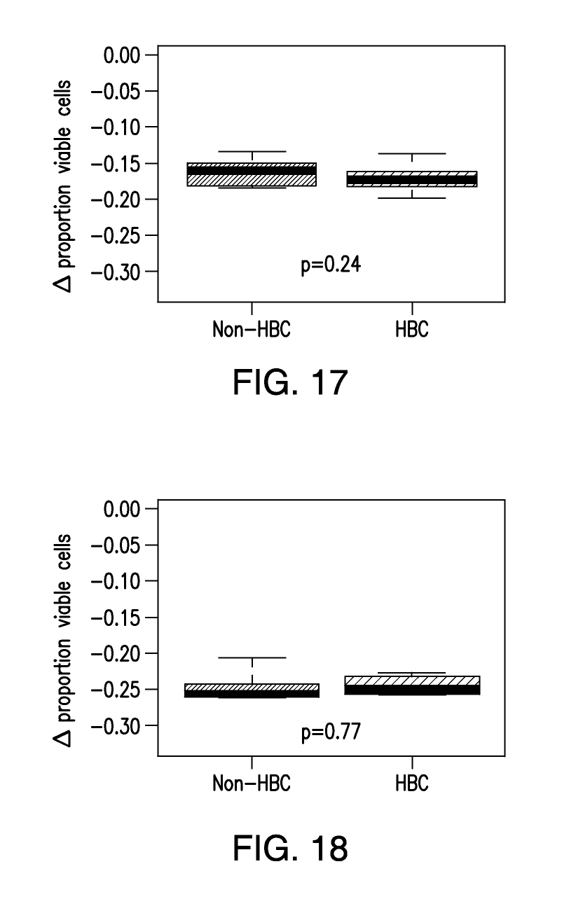

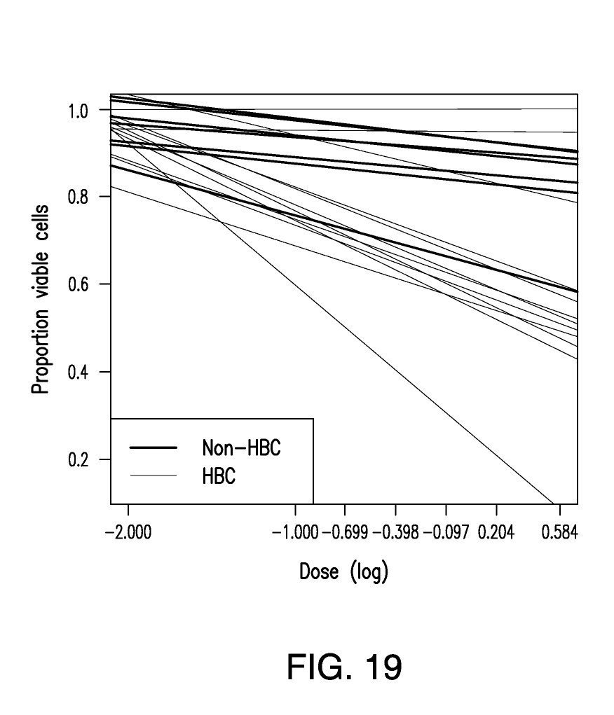

Additionally, affected HBC women carried a relative abundance of suspected pathogenic variants in these pathways in the matched samples from the Utah cohort (FIGS. 12C, 16). A variety of other pathways related to cell adhesion—including Focal Adhesion, Chemotaxis, and ECM—also performed well in the analyses, even though they fell above the significance thresholds in some cases. Lastly, the study highlights the ability of these pathways to predict an individual's risk of HBC development by projection of pathway-specific gene expression signatures generated in the Utah training cohort into the independent and external Ontario cohort. Both Integrin cell surface interactions and Cell adhesion molecules pathways function as relatively accurate gene expression based biomarkers, with area under receiver operating characteristic curve (AUC) values of 0.72 and 0.64, respectively, in the training Utah cohort and values of 0.65 and 0.71 in the independent validation Ontario cohort (FIGS. 16-19).

Together, these findings indicate that genetic variation and aberrant transcriptional activity (in part driven by genetic variation, see FIG. 7) perturb normal cell adhesion activity, potentially leading to an increased risk of breast tumors in HBC women. In cancer, expression of genes that participate in cell-cell and cell-ECM interactions are often downregulated. Although the precise mechanisms by which this perturbation occurs may vary considerably from one individual to the next, the results consistently point to disrupted cell adhesion mechanisms as a predictor of breast cancer risk.

### Aberrant Cell Adhesion in Normal Mammary Epithelial Cells of High-Risk Women

As the gene expression and DNA-mutation genomic studies consistently show dysregulation of cell adhesion pathways in the peripheral blood of high-risk women who develop breast cancer and in normal-appearing mammary tissue from prophylactic surgeries of high-risk women, primary mammary epithelial cells were utilized to investigate cell adhesion phenotypes of breast cancer susceptibility. These studies aimed to determine if normal breast tissue from high-risk women has aberrant cell-cell and cell-ECM interactions when compared to normal breast tissue from control women who underwent breast reduction surgeries for non-cancer related reasons.

To determine if a qualitative cytoskeletal cell-cell adhesion phenotype could be detected between cells isolated from unaffected control patients compared to cells isolated from high-risk patients, fluorescence microscopy was used. Normal primary mammary epithelial cells from each of the 24 patient cultures were seeded onto glass slides, grown for 3-5 days, then fixed, stained, and imaged in a blinded manner. Cells stained for F-actin, focal adhesions, and nuclei are presented from ten patients. Primary breast epithelial cells from control unaffected patients or from high-risk unaffected patients were cultured on glass slides for 3-5 days and subsequently fixed and stained for F-actin, focal adhesions and nuclei. Widefield epifluorescence microscopy of the cells was used for cell imaging. Cell morphology ranged from compact clusters of tightly bunched cells, to dispersed single cells with well-developed actin cytoskeletons and adhesion sites. After the cells were unblinded, the control cell cultures were found more often clustered together and displayed actin filaments and focal adhesions as commonly seen in normal mammary epithelial cell cultures, while over 45% of the high risk cell cultures exhibited well-spread cells with remarkable actin cytoskeletons. Specifically, the high-risk cultures showed decreased cell-cell contacts as evidenced largely by their loosely associated growth patterns and diminished cytoskeletal interactions as seen by the F-actin structures. These observations indicate the actin regulatory pathways in HBC women can be phenotypically distinct from normal controls, leading to decreased cell-cell contact disposition in response to growth in vitro.

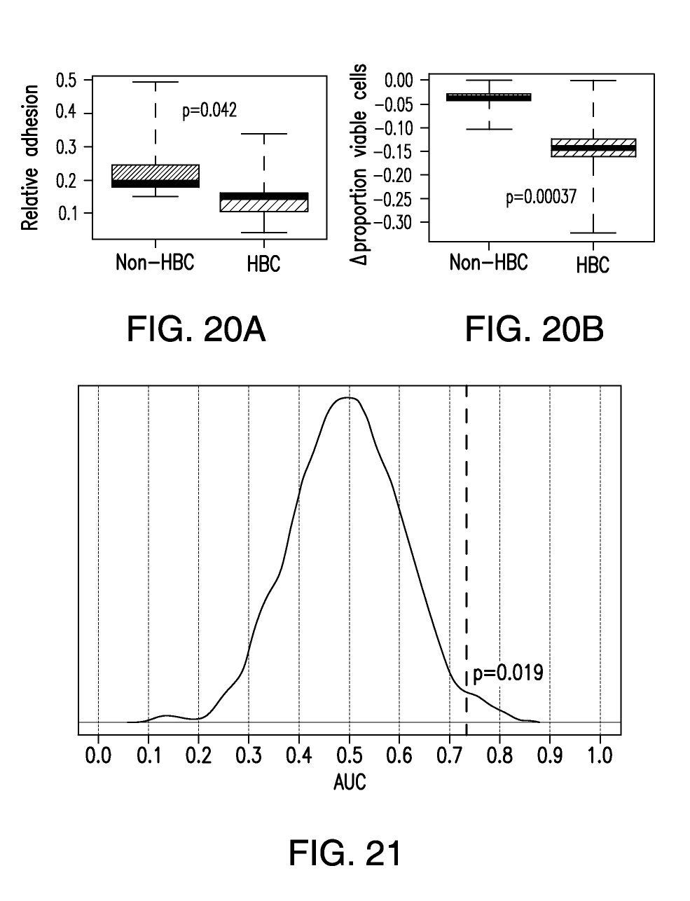

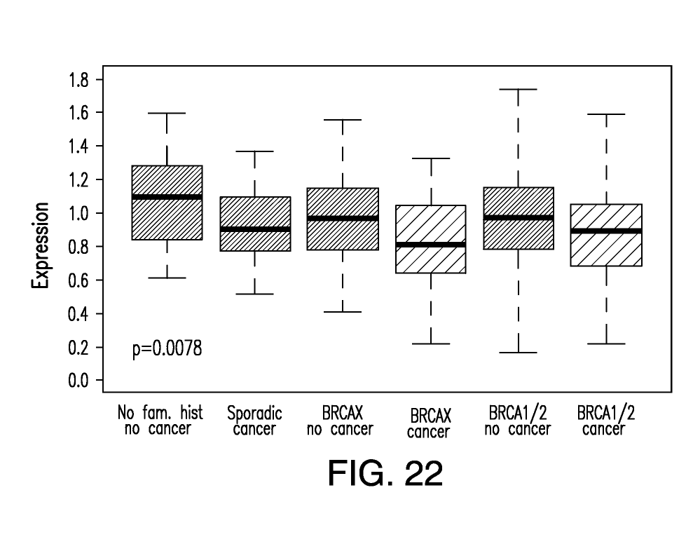

The genomic data also indicate a dysregulation in cell-ECM pathways. To evaluate the ability of cells to adhere to the ECM, an in vitro cell adhesion assay was used. Specifically, a quantitative assay was performed in which single cells from the same primary normal (non-malignant) mammary epithelial cultures described above were allowed to adhere to laminin coated plates for three hours to test for cell-ECM interaction and adherence. The results show a modest but significant decrease in cell adhesion in HBC samples compared to control women (FIG. 20A, p-value=0.04), again supporting the findings of the genomic studies, which indicate significant difference in integrin-based signaling between HBC and control phenotypes. Of note, the adhesion phenotype observed with the primary cultures can be time-in-culture dependent, as a difference is not seen in focal adhesions of HBC cells grown longer-term in culture, such as observed in the fluorescence microscopy. These results indicate subtle variance in these pathways that are uncovered in short-term culture acute settings but compensated for, and lost in longer-term culture experiments. Lastly, as cell adhesion is intricately linked to apoptosis, we performed a dose-response assay for TNF-related apoptosis inducing ligand (TRAIL) and control drugs that target cell growth, such as ErbB antagonists, on these cells. While we saw no difference in response of control and HBC normal epithelial cells to drugs that target growth response pathways not closely related to cell adhesion (FIGS. 8-21), we saw a significantly increased response of HBC cells to TRAIL when compared to control cells (p-value<0.001, FIGS. 20B and 22). As previous studies link a decrease in cell adhesion to increased responsiveness to TRAIL, this result supports the hypothesis that HBC patient cells have dysregulated cell adhesion phenotypes.

### Empirical Gene-Expression Biomarker Robustly Predicts Hereditary Breast Cancer Development

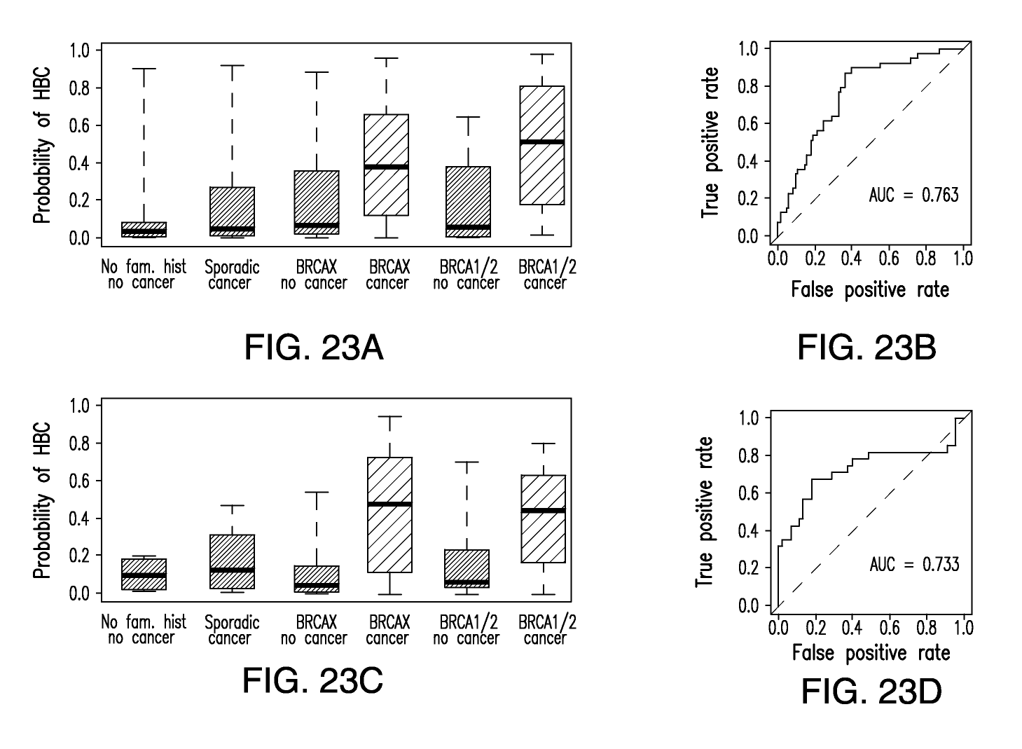

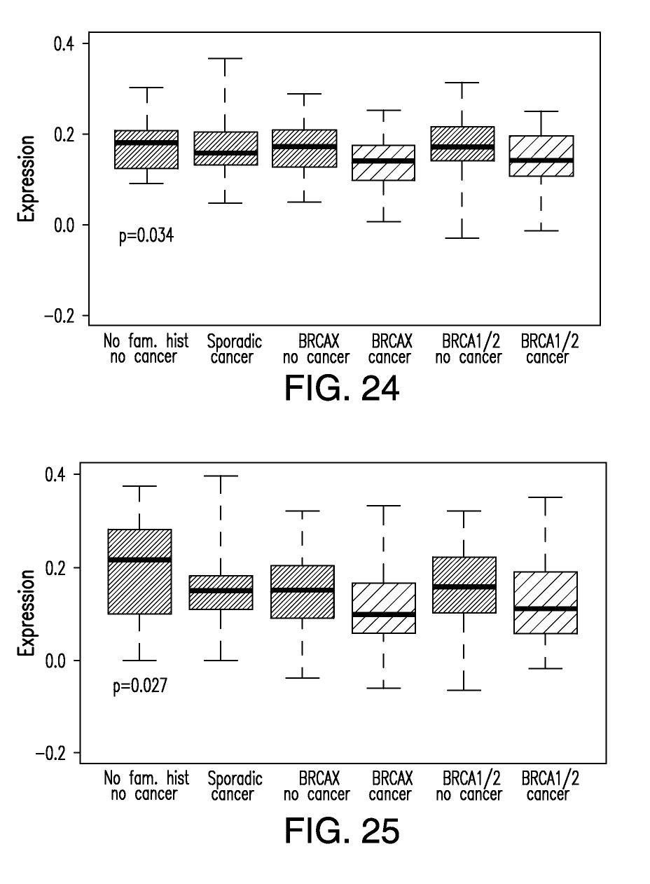

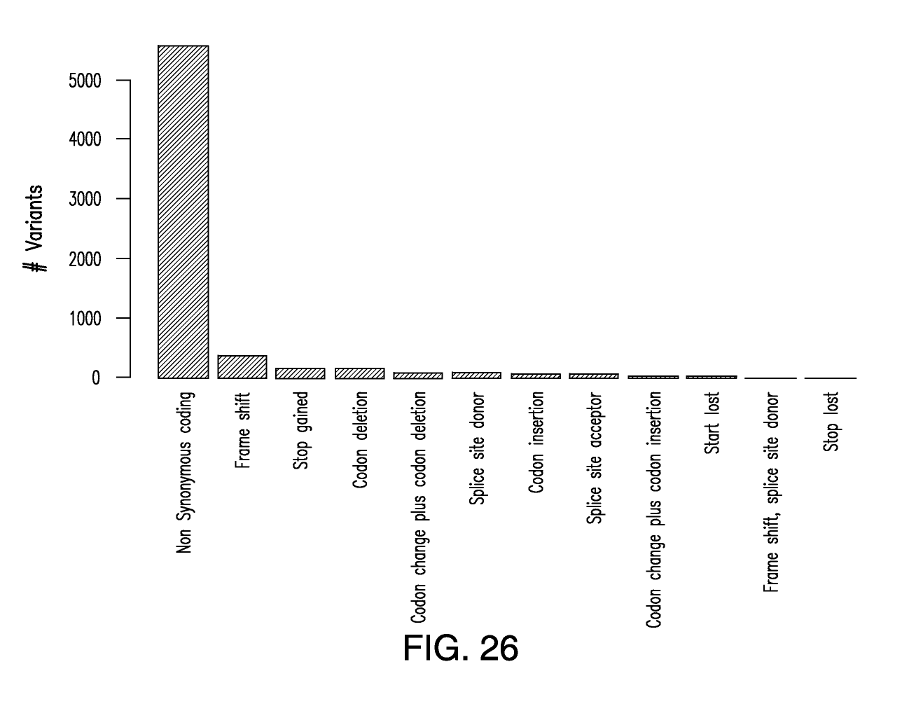

Women from HBC families face greater uncertainty regarding their personal risk than the general population. Various risk-prediction models based on clinical and/or genomic data have been proposed, yet the discriminatory accuracy of these models has been modest (AUC 0.56-0.66). As the data highlight the ability of gene expression profiling to classify affected HBC women from controls using pathway-specific gene sets and to identify novel signaling events potentially involved in HBC development, optimizing HBC risk prediction using a minimally invasive empirical biomarker derived from all human genes measured in the peripheral blood was next. Specifically, the analysis was not limited to genes from any single, literature-based pathway; instead all genes for which expression measurements were available were used. Gene subsets that best differentiated HBC individuals from controls were identified using the Support Vector Machines-Recursive Feature Elimination (SVM-RFE) algorithm, and the SVM algorithm was used to predict HBC status. Initially, this approach was evaluated in the Utah cohort via cross validation, and the data showed consistently higher probabilities of HBC development in women from HBC families who had developed a tumor versus controls, attaining an AUC value (equivalent to discriminatory accuracy) of 0.763 (FIG. 23A-B). In repeated cross-validation (see Methods), it was observed that on average the best results were attained using 250 genes, yet the accuracy was consistently high independent of gene number (FIG. 24). As a negative control, the analysis was repeated using randomly permuted class labels and show the biomarker accuracy was highly significant (p=0.002, FIG. 25).

To test the empirical biomarker derived from the Utah dataset, HBC development was predicted in the independent dataset described above from Ontario. Specifically, the predictive model was trained solely on the Utah data; the fixed model for prediction of HBC development status was then tested on the Ontario samples. Very similar to the Utah results, these predictions attained an AUC of 0.733 (permutation p-value=0.019; FIGS. 23C-D and 26). The high predictive accuracy of these results was consistent even though these cohorts were from distinct geographical regions. Importantly, the data were processed in two different facilities, and many women from the Ontario cohort had not yet experienced menopause. The predictive capability of the empirical biomarker indicates that even though individuals from HBC families carry highly unique genomic profiles, common transcriptional patterns are detectable across families and even populations. Interestingly, many genes in the empirical biomarker are involved in regulation of cell-cell adhesion and cell-ECM interactions, including DSC1, FN1, ST6GALNAC5, TP63, SHB, and WNT3. Together, these results indicate that the germline transcriptome can identify biological processes that influence HBC risk and serve as a biomarker for individualized assessment of HBC risk.

### Discussion

Since the discovery of BRCA1 and BRCA2 as breast cancer susceptibility genes, much focus has been placed on discovering additional DNA repair genes that influence HBC development. Ongoing efforts to genotype ever-larger cohorts also are yielding common susceptibility variants; however, in total, known susceptibility variants explain less than 30% of familial risk. Accordingly, additional studies are needed to identify the remaining drivers of HBC risk. In this study, a new approach for discovery of susceptibility factors using canonical pathways and multiple types of genomic data is addressed. The approach is based on the premise that genomic alterations—including DNA variants, epigenetic modifications, and non-coding RNA—cause gene-expression changes that influence tumor development and are observable in normal (non-malignant) cells. Such gene-expression patterns can help predict disease risk and identify signaling pathways associated with HBC. In particular, the pathway-based analyses revealed that cell adhesion and integrin signaling processes exhibit differential regulation between individuals who develop HBC and controls; these observations were consistent across multiple gene-expression data sets, protein-coding variants, and laboratory-based validations of non-malignant cells. These patterns were consistent for women who did or did not carry germline mutations in BRCA1 or BRCA2. In addition, perturbation of these pathways likely was not due to a prior cancer diagnosis or treatment because women with sporadic cancer did not show the same patterns of pathway dysregulation in the gene-expression analyses.

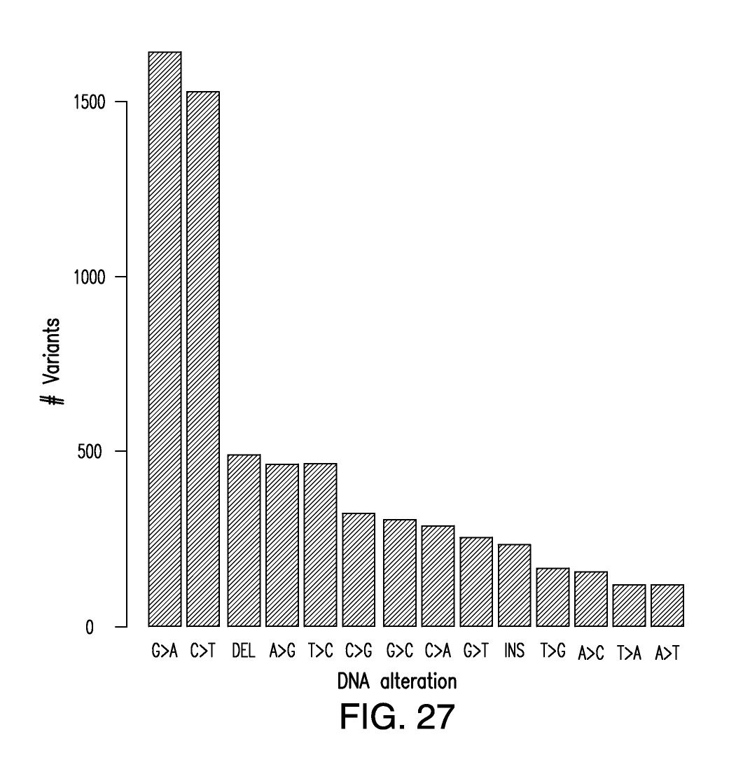

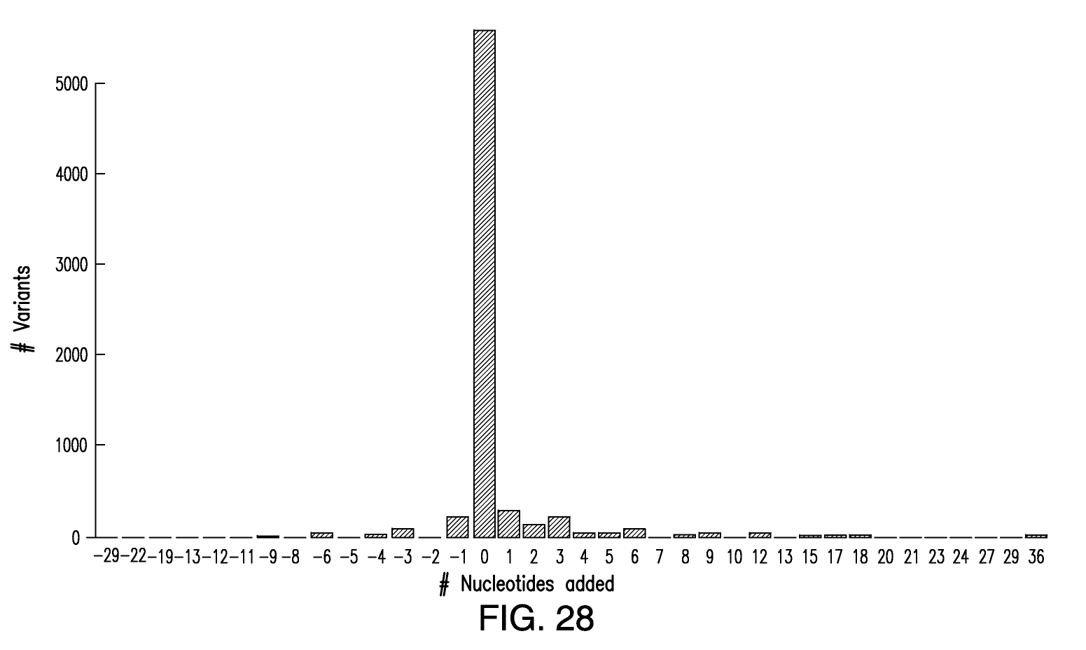

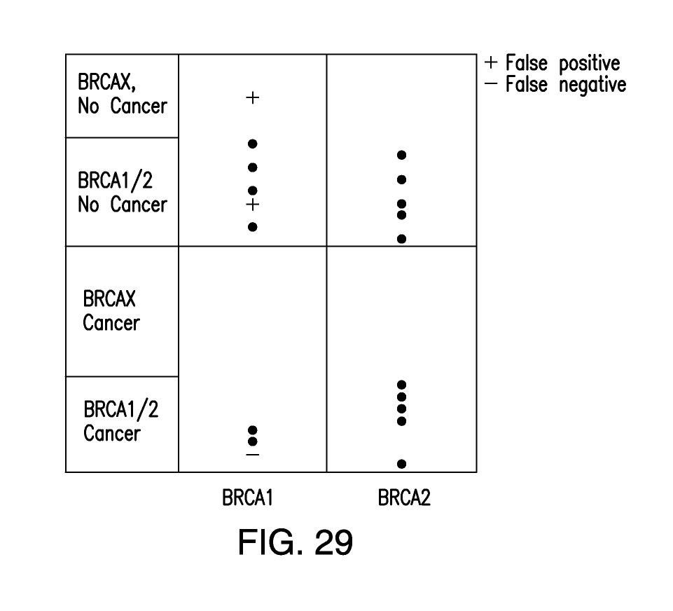

Nguyen-Ngoc, et al. have shown that mammary epithelial cells transition to carcinoma cells when two conditions are met: 1) a cell adhesion gene has been deleted and 2) the basement membrane is disrupted such that the cells are exposed to collagen 1. In these samples, suspected pathogenic variants occurred in various cell adhesion genes (FIG. 12), and consistently lower expression levels in HBC women compared to controls were observed in various cell adhesion genes, including ITGA6, PTK2, and NEO1 (FIGS. 27-29). While the validation studies focused on two key cell adhesion pathways (FIG. 12), various related pathways also performed consistently well and shared a small number of genes with each other. This indicates that the conclusions rely not on a particular set of genes but rather that they follow a consistent biological theme. Additionally, the peripheral blood-based gene expression profiles reflect immune responses that differ from what would be observed in primary breast tissue. To accommodate this, the Lim, et al. data was used in the analyses and performed the microscopy and cell adhesion assays; both experiments confirm dysregulation of cell adhesion pathways, consistent with the genomic analyses. Of interest, the pathway analyses also pointed to other biological processes that have a plausible connection to cancer susceptibility. For example, several KEGG pathways that represent cancer-signaling networks were ranked highly. These findings are consistent with a recent study showing pathway-based enrichment of cancer (and cell adhesion) pathways in DNA methylation profiles of women who developed familial breast cancer. The cancer related pathways identified in the study include genes known to play diverse roles in regulating cellular proliferation, differentiation, and cell adhesion; these pathways include well-known cancer genes such as ERBB2, EGFR, PIK3CA, MYC, and JUN. Accordingly, the results indicate that women who develop HBC can begin life with a higher rate of aberrantly expressed genes and/or mutations in pathways that drive tumorigenesis and that these conditions increase the likelihood that tumors will arise as somatic insults accumulate.

Current clinical standards define a woman's risk based on population averages, and a strict surveillance regimen is recommended to high-risk individuals that typically includes twice-yearly physical exams and yearly mammograms and MRIs. The biomarker presented here has the ability to provide a risk assessment for each individual; therefore, women within the same family could be assigned different risks based on their genomic profile, leading to more personalized treatment decisions. These personalized risk assessments can provide reassurance for women who are not as highly predisposed and who may opt then for monitoring and chemoprevention rather than prophylactic mastectomy or oophorectomy; alternatively, a high predictive risk can provide evidence to support more aggressive prophylactic or preventive intervention.

### Methods

### Description of Patient Cohorts and Data Sets

### Utah

Participants were recruited via the High Risk Breast Cancer Clinic at the Huntsman Cancer Institute (Utah, USA) under IRB approved protocols (#00022886 and #4965). Blood samples were collected after breast cancer occurrence and were obtained when participants were in remission for at least six months. Participants were considered to have a family history of breast cancer if two or more first-degree relatives (mother, sister, daughter) had been diagnosed with breast cancer. Among 83 individuals in the Utah cohort who had a family history of breast cancer, 39 had been diagnosed with breast cancer (“HBC affected”), whereas 44 women were at least 54 years of age (average age=62) and had never been diagnosed with breast cancer. Of the 83 participants, 37 carried a pathogenic variant in BRCA1 or BRCA2. In addition, 41 individuals with no known family history of breast cancer were identified, 22 of whom had developed sporadic breast cancer. There was no significant difference in the age at which blood was drawn (p-value=0.73). RNA from peripheral blood was processed within two hours, and RNA was used for expression-array analyses following confirmation of RNA quality.

Reduction mammoplasty cells (n=9) and normal cells from prophylactic surgeries (n=15) for high-risk women were obtained in cooperation with the Breast Disease Oriented Team and Tissue Resource and Applications Core at the Huntsman Cancer Institute (IRB #10924). Family history status was determined via examination of medical health records.

### Ontario

De-identified samples were also obtained via the Ontario, Canada site of the Breast Cancer Family Registry (BCFR). This cohort included 28 samples from women who had developed HBC, 32 from women with a family history who had not developed breast cancer, 8 from women who developed sporadic breast cancer, and 5 from women who had no known family history and had not developed cancer. Of these 73 women, approximately half were post-menopausal, as with the Utah cohort, whereas the remaining women were premenopausal. Blood samples were obtained, isolated, and stored when the women first enrolled in the BCFR before menopause onset and before cancer diagnosis.

### TCGA

Exome-sequencing data was downloaded for 611 samples from TCGA via the Cancer Genomics Hub (cghub.ucsc.edu). Fifty-seven samples were derived from normal breast tissue, whereas the remaining samples were derived from peripheral blood. This population was used as a control group—containing mostly sporadic breast cancer cases—against which variant frequencies could be compared. Variants were detected in these samples using the same pipeline that was used to detect variants in the Utah samples, thus reducing potential confounding effects due to technology differences.

### Genomic Data Acquisition

PBMCs were isolated from whole blood in cell preparation tubes following manufacturer protocol (Becton-Dickinson). RNA was then extracted using the RiboPure RNA Isolation Kit and hybridized to Affymetrix Genechip Human Exon 1.0 ST microarrays. Raw data files have been deposited in Gene Expression Omnibus (GEO) under accession number for the SuperSeries GSE47862.

Gene-expression data representing normal breast-tissue expression were downloaded from GEO (GSE17072). These data were obtained using the Illumina HumanWG-6 v3.0 expression beadchip. We used these data in preprocessed form as provided by the authors.

Genomic DNA from peripheral blood was also used for exome sequencing. At the Huntsman Cancer Institute, genomic DNA was hybridized using Agilent SureSelect Human All Exon v4+UTRs kits. Captured libraries were sequenced on an Illumina Hi-Seq 2000 instrument, and barcoding techniques were used for multiplexing (seven lanes, five samples per lane). This process resulted in 101-bp paired-end reads (58,032,900 unique reads per sample).

### Microarray Data Normalization and Filtering

To correct for array- and probe-level background noise, the Single-channel Array Normalization (SCAN) algorithm was applied; this method is applied to each sample individually, thus averting intersample biases and computational processing limits that can arise with multisample normalization techniques. Probes were subsequently filtered using the PLANdbAffy database, which assigns quality labels to probes based on cross-hybridization potential, location of probes within genes, and whether SNVs fall within target regions. Any probe that was not classified as “green” or that mapped to an SNV was excluded. For each array, the remaining 2,201,005 probes were summarized into gene-level values using a 10% trimmed mean; genes that contained fewer than five probes were discarded.

Because the microarrays had been hybridized and scanned in different facilities and at different times, ComBat was used to adjust for potential confounding effects that might arise due to these differences. Prior to biomarker derivation (see below), the Ontario and Utah samples were adjusted for batch using cohort and processing facility to define the batches.

To identify blood markers that could influence mRNA expression but that are unlikely related to HBC development, a total lymphocyte enumeration test for the blood draw used in the study could be used. This test provides total counts of CD4-positive T cells, CD8-positive T-cells, CD3-positive T-cells, B-cells and NK-cells. These counts were available for 22 samples in the Utah cohort. Furthermore, epidemiological and demographic data was obtained via a health-assessment survey for 63 patients in the Utah cohort (there was not a health survey for sporadic cancer and normal controls); these factors were age, education level, marital status, religious preference, health status, physical activity, age at menarche, contraceptive use, total number of pregnancies, total number of live births, age at first live birth, age at last live birth, breastfeeding status, time since last menstrual period, age when menstrual periods stopped, chemopreventive drug use (selective estrogen receptor modulators), alcohol use, tobacco use, occupational history, immunological disorder history, hypertension drug use, and anti-inflammatory drug use. Using a multifactor ANCOVA model, genes whose expression patterns correlated with any of these variables at a 0.01 significance level were excluded. For a subset of 34 participants, exome sequencing was also performed. One additional individual that was not profiled for the gene-expression analyses was added to the exome-sequencing cohort. See Table 7. Where gene expression values were compared among groups for a given gene, p-values were calculated using a one-way analysis of variance test.

### Exome Variant Filtering and Aggregation

Exome-capture sequencing was used to profile 35 women from the Utah cohort. In prior commercial genetic testing, pathogenic BRCA1 or BRCA2 variants had been observed in 17 of the women; the remaining “BRCAX” women had tested negative for BRCA1/2 variants. Within the entire cohort, 19 women had developed early-onset breast cancer; the remaining women had not been diagnosed with breast cancer and were at least 54 years old.

Reads were aligned to the hg19 human reference genome using the Burrows-Wheeler Aligner software (BWA, version 0.6.1). Duplicate reads were marked using Picard tools (v. 1.64, picard.sourceforge.net/), and reads were sorted and indexed using samtools (v. 0.1.18). Using the Genome Analysis Toolkit (GATK, v. 1.5.3), reads were subsequently passed through various processing steps to realign and recalibrate the reads and then to detect SNVs and short InDels; the GATK Best Practice Variant Detection guide was followed. The TCGA samples were analyzed at a later date, so newer software versions (Picard 1.82 and GATK 2.3.4) were used.

On average per sample, 5,848,610,129 bases aligned to the reference genome, and 88.24% of bases fell within exome-capture target regions, resulting in a mean target coverage of 58.10. Across all samples, variants were observed at 1,796,343 unique loci (1,579,855 SNVs and 216,488 InDels).

Multiple criteria were used to filter the initial variant call set (FIG. 13). Any variant for which a minor-allele frequency greater than one percent had been reported in any ethnic population in either the 1000 Genomes (phase 1, release 3) or Exome Sequencing Project 6500 data (evs.gs.washington.edu/EVS) were excluded. Additionally, GATK was used to identify variants in the TCGA germline samples that had a minor-allele frequency greater than three percent—a higher threshold was used for TCGA because this population was likely enriched for susceptibility variants. Finally, variants were excluded that occurred in more than 15% of the samples.

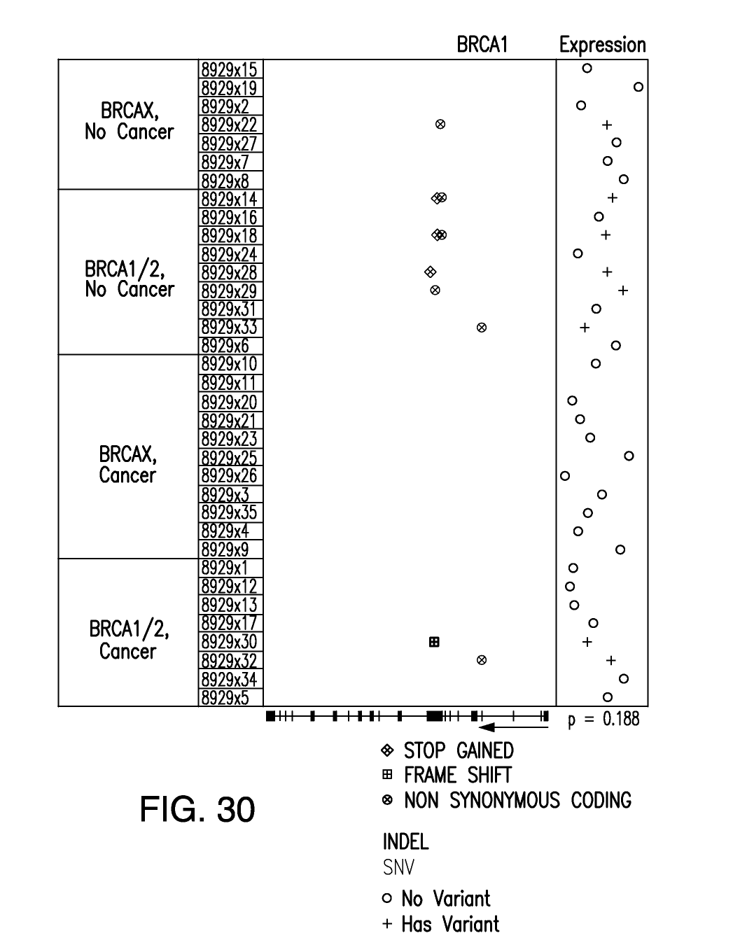

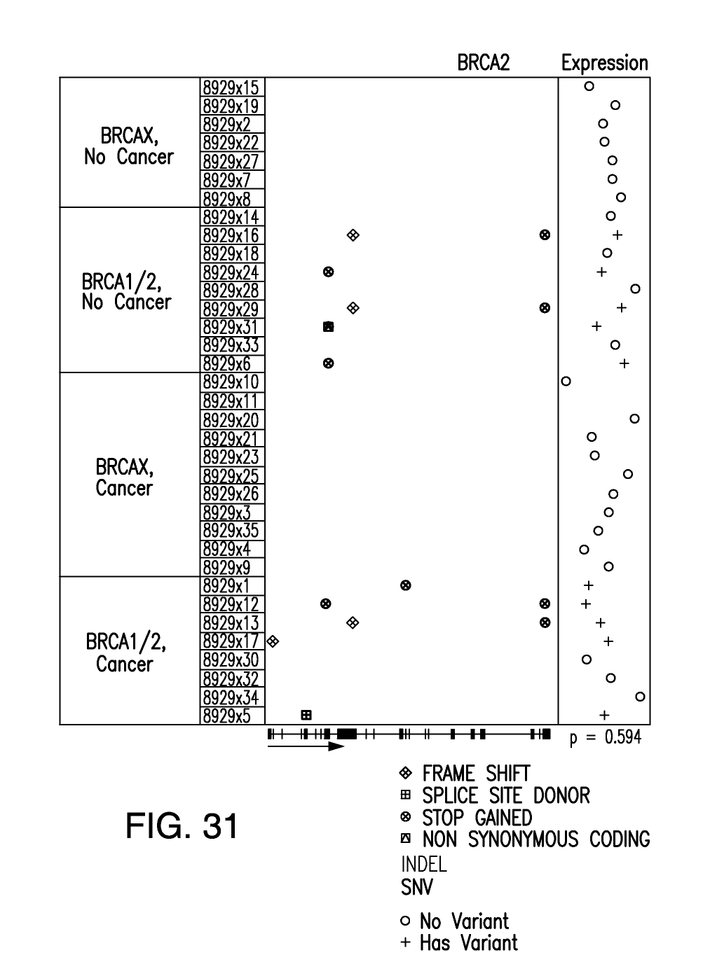

Next variants were excluded that fell outside exons (plus/minus two bases to allow for detection of splice-site mutations) used in each gene's primary transcript; gene/transcript definitions were extracted from Entrez Gene. The remaining variants were annotated for protein-coding effect using snpEff. Variants that were assigned a severity level of “MODIFIER” or “LOW” were excluded. Any variant that was assigned a “HIGH” severity was retained; these consisted primarily of truncating, frameshift, and splice-site variants (FIG. 30). “MODERATE” InDels were also retained. Nonsynonymous coding SNVs were further examined using Condel. Any missense SNV called as neutral was excluded. The remaining variants constituted the final set of “suspected pathogenic” variants. For simplicity, heterozygous variants and homozygous-rare variants were considered to have an equivalent effect; the great majority of variants were heterozygous.

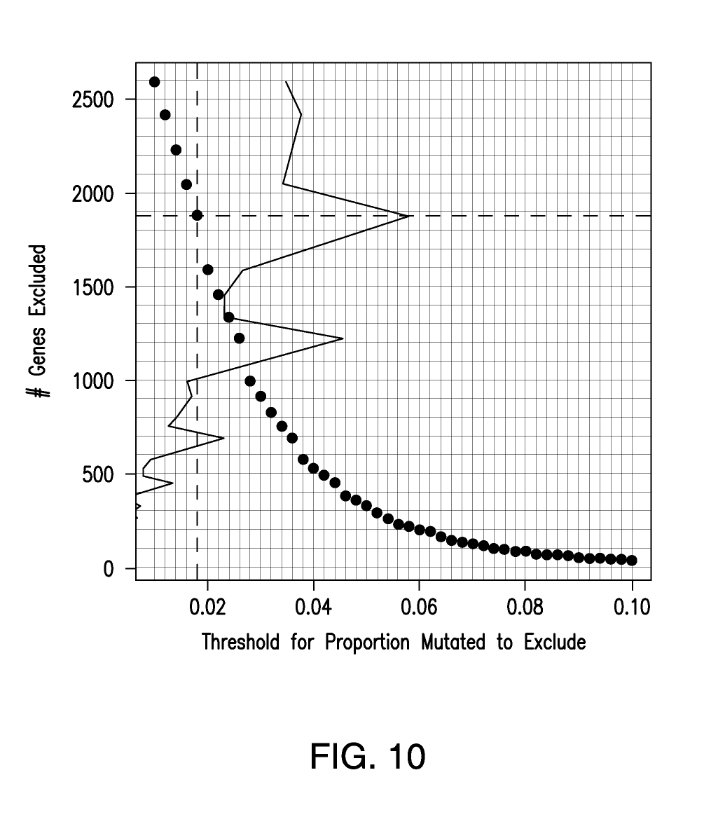

After filtering, 6,573 variants (average of 167.1 SNVs and 20.7 InDels per sample) were observed at 5,222 loci. Most variants were non-synonymous substitutions (FIG. 30). The most frequent substitutions were G-to-A and C-to-T transitions (FIG. 31). The average transition/transversion ratio was 2.35. Most InDels resulted in a net gain/loss of less than seven nucleotides; however, a few were larger (FIG. 10).

To assess the validity of the variant calls, they were compared against PCR-based, commercial genetic test results for BRCA1 and BRCA2. We observed only one false-negative call and two false-positive calls. The false-negative variant (rs80358061) resides in a BRCA1 intronic region outside the splice-site junction points. This variant was identified by the pipeline; however, it was filtered out due to its intronic location. The false positives (rs28897683 and rs80356935) are non-synonymous SNVs that lie outside the BRCT and RING domains of the BRCA1 protein and thus are likely not to be pathogenic despite evolutionary conservation; neither individual from the study who carried a false-positive variant developed cancer, and one had a known pathogenic variant in BRCA2. Additionally, the exome-sequencing analysis identified one individual who carried a pathogenic BRCA2 variant that had not been observed in commercial genetic testing. Subsequent clinical follow-up confirmed the presence of this variant.

To enhance biological interpretation, variants were aggregated at the gene and pathway levels. If a given sample carried any suspected pathogenic variant in a given gene or pathway, that gene or pathway was considered to be “mutated” (typically, a gene had no more than a single variant in a given sample). Under the assumption that frequently mutated genes are unlikely to drive susceptibility due to selective pressure and thus constitute noise at the pathway level, genes that are mutated relatively frequently were excluded. For this step, the 611 germline samples from TCGA were used. Based on gene-mutation frequencies in the TCGA data, genes from the data set that were mutated in more than 1.8% of TCGA germline samples (except BRCA1/2 because the samples were intentionally enriched for mutations in these genes) were excluded. This threshold was selected based on the maximal difference in number of excluded genes for thresholds that fell between 0.2% and 10%. After excluding these genes, the number of “mutated” pathways in the data dropped considerably from 63.0 to 30.0 per patient (FIG. 13). By processing these samples on the same pipeline that was used to process the other samples, systematic biases that can arise due to differences in variant-calling pipelines were avoided. Consequently, variants/genes were identified that were mutated frequently but that had not been identified in the other databases that were queried. Comparisons between the number of mutated and non-mutated samples were performed using a one-sided Fisher's exact test.

### Biomarker Development

Biomarkers were derived in two successive steps: 1) the most discriminatory genes were identified using the SVM-RFE algorithm, then 2) the SVM algorithm was used to generate a probability that each patient belonged to the HBC group. Genes that ranked in the top 25, 50, 75, 100, 125 . . . 300 (when that many were available) of the SVM-RFE iterations were used in the SVM models, and the number of genes included in the SVM models was optimized via internal cross validation.

The quality of the models was assessed via ten-fold cross validation for the Utah cohort and via a training/testing design for the Ontario cohorts. (In training/testing, gene selection and optimization were performed via nested cross validation on the Utah data only.) HBC probabilities were compared against known classes (HBC or not), and an AUC value was calculated. In this context, the AUC quantifies the model's ability to discriminate the groups at various HBC probability thresholds; it can be interpreted as the frequency with which the model would assign two randomly selected patients to the correct group. To assess the robustness of the models, cross validation was repeated 1,000 times using different random seeds. Empirical p-values were also generated by repeating the analysis 1,000 times with permuted class labels.

### Pathway-Level Gene-Expression Analysis

Gene-pathway relationships were obtained from two literature-curated sources. KEGG pathways were downloaded directly from the KEGG FTP site. The remaining gene lists were obtained from the Molecular Signatures Database (v3.0), which aggregates pathways from various sources, including REACTOME (reactome.org) and Biocarta (biocarta.com).

The microarray data and exome-sequencing data was evaluated at the pathway level to identify pathways most likely to influence HBC development when deregulated. For the microarray data, SVM models were derived to predict HBC status using the biomarker development methodology described in the previous subsection, except that the models were derived only from genes that belonged to a given pathway. SVM-RFE was also used to identify the most relevant genes within each pathway. A p-value was then derived for a given pathway by comparing the AUC observed for that pathway against AUCs observed after randomly shuffling the class labels (1,000 permutations); the p-value was calculated as the fraction of permuted AUCs higher than the non-permuted AUCs.

The Lim, et al. data were analyzed in a similar way. Which patients would eventually develop breast cancer was not known and therefore expression patterns between those who carried a family history of breast cancer and those who did not were compared.

### Correlation Between Sequencing Variants and Gene Expression

For the samples that were profiled using both technologies, the relationship between variants in a given gene and expression of that gene was estimated using Spearman's rank correlation coefficient. A threshold was determined using a local False Discovery Rate approach. Variants with absolute correlation coefficients more extreme than this threshold were considered to be associated with expression of that gene.

### Drug Response Assays

Normal mammary epithelial cells isolated from either high-risk women undergoing prophylactic mastectomy or control women with no family history of breast cancer undergoing breast-reduction surgery for non-cancer related health issues were isolated, and single-cell suspensions were generated. 1500 cells/well for each patient samples were plated in 40 microliters of MEBM basal media supplemented with a MEGM Bulletkit [Lonza] in half-well 96-well plates [Nunc] in triplicate for each drug dose. After 24 hours, TRAIL, Afatinib, Gefitinib, and EGF were added. The indicated doses were chosen within the average linear range of drug effect: TRAIL (0.1 ng/mL-5.0 ng/ml), Afatinib (10 nM-1 mM), Gefitinib (0.001 mM-0.5 mM), EGF (5 ng/mL-500 ng/mL) [Millipore (TRAIL) and Selleckchem]. A BIOMEK 3000 (Beckman Coulter, Brea, Calif., USA) robot was used to seed the cells and dispense the drugs. After 96 hours, cell viability was quantified using the CellTiter-Glo Luminescent Cell Viability Assay [Promega]. The proportion of viable cells was calculated for each dosage by comparing against cell counts for non-treated cells. A summary value was calculated for each cell line as the slope of a linear-regression line fitted to the cell-count proportions.

### Cell Adhesion Assays

For the cell adhesion assay, 96-well plates were pre-coated overnight with 1 ug/ml human laminin [Millipore], which were washed in PBS prior to cell plating. 3000 cells from single-cell suspensions for high-risk affected women or breast reduction controls were plated per well, and the average number of viable cells adhering to the plate after three hours across the replicates was compared against total cell counts of viable cells after 14 hours using CellTiter-Glo Luminescent Cell Viability Assay [Promega]. All p-values were calculated using a two-sided t-test.

### Microscopy

Cells were seeded into multi-well slide chambers [Lab-Tek II CC2 Slide, 8 chambers] and grown in MEBM basal media supplemented with a MEGM BulletKit [Lonza] for 3-5 days. Cells were fixed (3.7% formaldehyde, 15 mins), permeabilized (0.5% TritonX-100, 5 mins), and stained for F-actin (Alexa-Fluor568-phalloidin 1:150 [Molecular Probes]), focal adhesions (Vinculin mouse antibody V-9131 1:1000 [Sigma]), with secondary antibodies AlexaFluor488-anti-mouse 1:200 and nuclei (Dapi 0.3 uM). Coverslips were mounted in Mowiol [Sigma]. Cell images were captured with a Zeiss AxioCamMRm camera on a Zeiss Axioskop2 mot plus microscope (40× dry objective, 0.75 NA) using Zeiss AxioVision 4.8.1 software. Normal primary cells from each of the 24 patient cell cultures were captured and analyzed in a blinded manner.

### Software

All software scripts that were used for this study have been aggregated into a software pipeline that can be executed on UNIX-based systems. This pipeline can be accessed at github.com/srp33/BCSP.

SVM-RFE was executed using the SVMAttributeEval module within the Weka software package. It was configured to remove 10% of genes in each iteration. When less than 1% of genes remained, a single gene was removed per iteration. Otherwise, default configuration settings were used. The e1071 R package (cran.r-project.org/package=e1071) was used for SVM predictions. In deriving the models, the radial-basis-function kernel was selected and the C parameter was tuned via internal cross validation. Additionally, the ML-Flex software package enabled the analyses to be executed in parallel on a high-performance computing cluster.

The fdrtool package was used to calculate False Discovery Rates. The Python programming language (python.org) was used for parsing and summarizing data files. A pre-release version of the SCAN software for exon microarray normalization was used; this version is included in the software pipeline.

Microscope images were processed using Adobe Photoshop v.8 and Illustrator CS v.11. Original image files are available from io.genetics.utah.edu/files/BCSP_Microscope_Images/.

### REFERENCES

[1] Amir E, Freedman O C. Assessing women at high risk of breast cancer: A review of risk assessment models. J Natl Cancer Inst. 2010; 102(10):680-91.
[2] Powles T J. Breast cancer prevention. Oncologist. 2002; 7(1):60-4.
[3] Gail M H, Costantino J P, Bryant J, et al. Weighing the risks and benefits of tamoxifen treatment for preventing breast cancer. J Natl Cancer Inst. 1999; 91(21):1829-46.
[4] Vogelstein B, Kinzler K W. Cancer genes and the pathways they control. Nat Med. 2004; 10(8):789-799.
[5] Tyrer J, Duffy S W, Cuzick J. A breast cancer prediction model incorporating familial and personal risk factors. Stat Med. 2004; 23(7):1111-30.
[6] Wacholder S, Hartge P, Prentice R, et al. Performance of common genetic variants in breast-cancer risk models. N Engl J Med. 2010; 362(11):986-93.
[7] Rockhill B, Spiegelman D, Byrne C, Hunter D J, Colditz Ga. Validation of the gail et al. model of breast cancer risk prediction and implications for chemoprevention. J Natl Cancer Inst. 2001; 93(5):358-66.
[8] Amir E, Evans D G, Shenton a, et al. Evaluation of breast cancer risk assessment packages in the family history evaluation and screening programme. J Med Genet. 2003; 40(11): 807-14.
[9] Boughey J C, Hartmann L C, Anderson S S, et al. Evaluation of the tyrer-cuzick (international breast cancer intervention study) model for breast cancer risk prediction in women with atypical hyperplasia. J Clin Oncol. 2010; 28(22):3591-6.
[10] Miki Y, Swensen J, Shattuck-Eidens D, et al. A strong candidate for the breast and ovarian cancer susceptibility gene BRCA1. Science. 1994; 266(5182):66-71.
[11] Easton D F, Steele L, Fields P, et al. Cancer risks in two large breast cancer families linked to BRCA2 on chromosome 13q1213. The American Journal of Human Genetics. 1997; 61(1):120-128.
[12] Sidransky D, Tokino T, Helzlsouer K, et al. Inherited p53 gene mutations in breast cancer. CancerRes. 1992; 52(10):2984-6.
[13] Liaw D, Marsh D J, Li J, et al. Germline mutations of the PTEN gene in cowden disease, an inherited breast and thyroid cancer syndrome. Nat Genet. 1997; 16(1):64-7.
[14] Swift M, Reitnauer P J, Morrell D, Chase C L. Breast and other cancers in families with ataxia-telangiectasia. N Engl J Med. 1987; 316(21): 1289-94.
[15] Stratton M R, Rahman N. The emerging landscape of breast cancer susceptibility. Nat Genet. 2008; 40(1):17-22.
[16] Mavaddat N, Antoniou A C, Easton D F, Garcia-Closas M. Genetic susceptibility to breast cancer. Mol Oncol. 2010; 4(3):174-91.
[17] Frazer Ka, Murray S S, Schork N J, Topol E J. Human genetic variation and its contribution to complex traits. Nat Rev Genet. 2009; 10(4):241-51.
[18] Schadt E E, Monks S A, Drake T A, et al. Genetics of gene expression surveyed in maize, mouse and man. Nature. 2003; 422(6929):297-302.
[19] Morley M, Molony C M, Weber™, et al. Genetic analysis of genome-wide variation in human gene expression. Nature. 2004; 430(7001):743-7.
[20] Cookson W, Liang L, Abecasis G, Moffatt M, Lathrop M. Mapping complex disease traits with global gene expression. Nat Rev Genet. 2009; 10(3):184-94.
[21] Stranger B E, Forrest M S, Ingle C E, et al. Relative impact of nucleotide and copy number variation on gene expression phenotypes. Science. 2007; 315(5813):848-853.
[22] Hedenfalk I, Duggan D, Chen Y, et al. Gene-expression profiles in hereditary breast cancer. N Engl J Med. 2001; 344(8):539-548.
[23] Waddell N, Ten Haaf A, Marsh A, et al. BRCA1 and BRCA2 missense variants of high and low clinical significance influence lymphoblastoid cell line post-irradiation gene expression. PLoS Genet. 2008; 4(5):e1000080.
[24] Johnson W E, Li W, Meyer Ca, et al. Model-based analysis of tiling-arrays for ChIP-chip. Proc Natl Acad Sci USA. 2006; 103(33):12457-62.
[25] Johnson W E, Li C, Rabinovic A. Adjusting batch effects in microarray expression data using empirical bayes methods. Biostatistics. 2007; 8(1):118-27.
[26] Guyon I, Weston J, Barnhill S, Vapnik V. Gene selection for cancer classification using support vector machines. Mach Learn. 2002; 46(1):389-422.
[27] Vapnik V N. Statistical learning theory. New York: Wiley; 1998.
[28] Hall M, Frank E, Holmes G, Pfahringer B, Reutemann P, Witten I H. The WEKA data mining software. ACM SIGKDD Explorations Newsletter. 2009; 11(1):10.
[29] E1071: Misc functions of the department of statistics (e1071), TU wien [computer program]. Dimitriadou E, Hornik K, Leisch F, Meyer D, Weingessel A. 2011. Available from http://cran.r-project.org/package=e1071.
[30] R: A language and environment for statistical computing [computer program]. R Development Core Team. 2011. Available from http://www.r-project.org/.
[31] Chang C, Lin C. LIBSVM: A library for support vector machines. ACM Transactions on IntelligentSystems and Technology. 2011; 2(3):27:1-27:27.
[32] ROCR: Visualizing the performance of scoring classifiers [computer program]. Sing T, Sander O, Beerenwinkel N, Lengauer T. 2009. Available from http://cran.r-project.org/package=ROCR.
[33] Pharoah P D P, Tyrer J, Dunning A M, Easton D F, Ponder BaJ. Association between common variation in 120 candidate genes and breast cancer risk. PLoS Genet. 2007; 3(3):e42.
[34] Venkitaraman A R. Cancer susceptibility and the functions of BRCA1 and BRCA2. Cell. 2002; 108(2):171-82.
[35] Kanehisa M, Goto S, Hattori M, et al. From genomics to chemical genomics: New developments in KEGG. Nucleic Acids Res. 2006; 34(Database issue):D354-7.
[36] Richardson M, Domingos P. Markov logic networks. Mach Learn. 2006; 62(1-2):107-136.
[37] Lakhani S R, Reis-Filho J S, Fulford L, et al. Prediction of BRCA1 status in patients with breast cancer using estrogen receptor and basal phenotype. Clin Cancer Res. 2005; 11(14):5175-80.
[38] Mani Sa, Guo W, Liao M, et al. The epithelial-mesenchymal transition generates cells with properties of stem cells. Cell. 2008; 133(4):704-15.
[39] Statnikov A, Aliferis C F, Tsamardinos I, Hardin D, Levy S. A comprehensive evaluation of multicategory classification methods for microarray gene expression cancer diagnosis. Bioinformatics. 2005; 21(5):631-43.
1. Perou C M, Sørlie T, Eisen M B, et al. Molecular portraits of human breast tumours. Nature. 2000; 406(6797):747-52.
2. Golub T R, Slonim D K, Tamayo P, et al. Molecular classification of cancer: Class discovery and class prediction by gene expression monitoring. Science. 1999; 286(5439):531-537.
3. van't Veer L J, Dai H, van de Vijver M J, et al. Gene expression profiling predicts clinical outcome of breast cancer. Nature. 2002; 415(6871):530-6.
4. Berns K, Horlings H M, Hennessy B T, et al. A functional genetic approach identifies the PI3K pathway as a major determinant of trastuzumab resistance in breast cancer. Cancer Cell. 2007; 12(4):395-402.
5. Cohen A L, Soldi R, Zhang H, et al. A pharmacogenomic method for individualized prediction of drug sensitivity. Mol Syst Biol. 2011; 7:513.
6. Morley M, Molony C M, Weber™, et al. Genetic analysis of genome-wide variation in human gene expression. Nature. 2004; 430(7001):743-7.
7. Jaenisch R, Bird A. Epigenetic regulation of gene expression: How the genome integrates intrinsic and environmental signals. Nat Genet. 2003; 33 Suppl(march):245-54.
8. Bild A H, Chang J T, Joshi M B, et al. Oncogenic pathway signatures in human cancers as a guide to targeted therapies. Nature. 2006; 439(7074):353-357.
9. Koboldt D C, Fulton R S, McLellan M D, et al. Comprehensive molecular portraits of human breast tumours. Nature. 2012; 490(7418):61-70.
10. Waddell N, Ten Haaf A, Marsh A, et al. BRCA1 and BRCA2 missense variants of high and low clinical significance influence lymphoblastoid cell line post-irradiation gene expression. PLoS Genet. 2008; 4(5):e1000080.
11. Cookson W, Liang L, Abecasis G, Moffatt M, Lathrop M. Mapping complex disease traits with global gene expression. Nat Rev Genet. 2009; 10(3):184-94.
12. Stratton M R, Rahman N. The emerging landscape of breast cancer susceptibility. Nat Genet. 2008; 40(1):17-22.
13. Wood L D, Parsons D W, Jones S, et al. The genomic landscapes of human breast and colorectal cancers. Science. 2007; 318(5853):1108-13.
14. Subramanian A, Tamayo P, Mootha V K, et al. Gene set enrichment analysis: A knowledge-based approach for interpreting genome-wide expression profiles. Proc Natl Acad Sci USA. 2005; 102(43): 15545-15550.
15. Steinmetz L M, Sinha H, Richards D R, et al. Dissecting the architecture of a quantitative trait locus in yeast. Nature. 2002; 416(6878):326-30.
16. Schadt E E, Molony C, Chudin E, et al. Mapping the genetic architecture of gene expression in human liver. PLoS Biol. 2008; 6(5):e107.
17. Liu Z, Wang M, Alvarez J V, et al. Singular value decomposition-based regression identifies activation of endogenous signaling pathways in vivo. Genome Biol. 2008; 9(12):R180.
18. Barbie D A, Tamayo P, Boehm J S, et al. Systematic RNA interference reveals that oncogenic KRAS-driven cancers require TBK1. Nature. 2009; 462(7269):108-12.
19. Singh A, Greninger P, Rhodes D, et al. A gene expression signature associated with “kras addiction” reveals regulators of EMT and tumor cell survival. Cancer Cell. 2009; 15(6):489-500.
20. Huang F, Reeves K, Han X, et al. Identification of candidate molecular markers predicting sensitivity in solid tumors to dasatinib: Rationale for patient selection. Cancer Res. 2007; 67(5):2226-2238.
21. Zhang X H, Wang Q, Gerald W, et al. Latent bone metastasis in breast cancer tied to src-dependent survival signals. Cancer Cell. 2009; 16(1):67-78.
22. Hedenfalk I, Duggan D, Chen Y, et al. Gene-expression profiles in hereditary breast cancer. N Engl J Med. 2001; 344(8):539-48.
23. Sawyer S, Mitchell G, McKinley J, et al. A role for common genomic variants in the assessment of familial breast cancer. J Clin Oncol. 2012; 30(35):4330-6.
24. Wacholder S, Hartge P, Prentice R, et al. Performance of common genetic variants in breast-cancer risk models. N Engl J Med. 2010; 362(11):986-93.
25. Xu Z, Bolick S C E, Deroo L A, Weinberg C R, Sandler D P, Taylor J A. Epigenome-wide association study of breast cancer using prospectively collected sister study samples. J Natl Cancer Inst. 2013; 105(10): 694-700.
26. Fisher R A. Statistical methods for research workers. London: Oliver & Boyd; 1932.
27. Chang J T, Nevins J R. GATHER: A systems approach to interpreting genomic signatures. Bioinformatics. 2006; 22(23):2926-33.
28. Hanahan D, Weinberg R A. Hallmarks of cancer: The next generation. Cell. 2011; 144(5):646-74.
29. Somasekharan S P, Koc M, Morizot A, et al. TRAIL promotes membrane blebbing, detachment and migration of cells displaying a dysfunctional intrinsic pathway of apoptosis. Apoptosis. 2013; 18(3):324-36.
30. Lane D, Goncharenko-Khaider N, Rancourt C, Piché A. Ovarian cancer ascites protects from TRAIL-induced cell death through alphavbeta5 integrin-mediated focal adhesion kinase and akt activation. Oncogene. 2010; 29(24):3519-31.
31. Samara R N, Laguinge L M, Jessup J M. Carcinoembryonic antigen inhibits anoikis in colorectal carcinoma cells by interfering with TRAIL-R2 (DR5) signaling. Cancer Res. 2007; 67(10):4774-82.
32. Jiang Y, Chen K, Tang Z, et al. TRAIL gene reorganizes the cytoskeleton and decreases the motility of human leukemic jurkat cells. Cell Motil Cytoskeleton. 2006; 63(8):471-82.
33. Rytomaa M, Martins L M, Downward J. Involvement of FADD and caspase-8 signalling in detachment-induced apoptosis. Curr Biol. 1999; 9(18):1043-6.
34. Peto J, Mack T M. High constant incidence in twins and other relatives of women with breast cancer. Nat Genet. 2000; 26(4):411-4.
35. Guyon I, Weston J, Barnhill S, Vapnik V. Gene selection for cancer classification using support vector machines. Mach Learn. 2002; 46(1):389-422.
36. Vapnik V N. Statistical learning theory. New York: Wiley; 1998.
37. Michailidou K, Hall P, Gonzalez-Neira A, et al. Large-scale genotyping identifies 41 new loci associated with breast cancer risk. Nat Genet. 2013; 45(4):353-361.
38. Nguyen-Ngoc K, Cheung K J, Brenot A, et al. ECM microenvironment regulates collective migration and local dissemination in normal and malignant mammary epithelium. Proc Natl Acad Sci USA. 2012; 109(39):E2595-604.
39. Lim E, Vaillant F, Wu D, et al. Aberrant luminal progenitors as the candidate target population for basal tumor development in BRCA1 mutation carriers. Nat Med. 2009; 15(8):907-13.
40. Piccolo S R, Sun Y, Campbell J D, Lenburg M E, Bild A H, Johnson W E. A single-sample microarray normalization method to facilitate personalized-medicine workflows. Genomics. 2012; 100(6):337-44.
41. Nurtdinov R N, Vasiliev M O, Ershova A S, Lossev I S, Karyagina A S. PLANdbAffy: Probe-level annotation database for affymetrix expression microarrays. Nucleic Acids Res. 2010; 38(Database issue):D726-30.
42. Johnson W E, Li C, Rabinovic A. Adjusting batch effects in microarray expression data using empirical bayes methods. Biostatistics. 2007; 8(1):118-27.
43. Li H, Durbin R. Fast and accurate short read alignment with burrows-wheeler transform. Bioinformatics. 2009; 25(14):1754-60.
44. Li H, Handsaker B, Wysoker A, et al. The sequence Alignment/Map format and SAMtools. Bioinformatics. 2009; 25(16):2078-9.
45. Abecasis G R, Auton A, Brooks L D, et al. An integrated map of genetic variation from 1,092 human genomes. Nature. 2012; 491(7422):56-65.
46. Cingolani P, Platts A, Wang L L, et al. A program for annotating and predicting the effects of single nucleotide polymorphisms, SnpEff: SNPs in the genome of drosophila melanogaster strain w1118; iso-2; iso-3. Fly (Austin). 2012; 6(2):80-92.
47. González-Peŕez A, López-Bigas N. Improving the assessment of the outcome of nonsynonymous SNVs with a consensus deleteriousness score, condel. Am J Hum Genet. 2011; 88(4):440-9.
48. Strimmer K. Fdrtool: A versatile R package for estimating local and tail area-based false discovery rates. Bioinformatics. 2008; 24(12):1461-2.
49. Hall M, Frank E, Holmes G, Pfahringer B, Reutemann P, Witten I H. The WEKA data mining software. ACM SIGKDD Explorations Newsletter. 2009; 11(1):10.
50. Piccolo S R, Frey L J. ML-Flex: A flexible toolbox for performing classification analyses in parallel. Journal of Machine Learning Research. 2012; 13:555-559.

TABLE 1
Utah1 FC Summary Down

| Entrez | | | Fold | |
|---|---|---|---|---|
| Gene ID | Gene Symbol | Gene Name | Change | Description |
| 87688 | RPL7AP50 | ribosomal protein L7a pseudogene 50 | 0.87836 | Down in those who develop hereditary breast cancer |
| 391106 | VDAC1P9 | voltage-dependent anion channel 1 pseudogene 9 | 0.96278 | Down in those who develop hereditary breast cancer |
| 646272 | LOC646272 | cytochrome b-c1 complex subunit 8-like | 0.94686 | Down in those who develop hereditary breast cancer |
| 286495 | TTC3P1 | tetratricopeptide repeat domain 3 pseudogene 1 | 0.95788 | Down in those who develop hereditary breast cancer |
| 541468 | C1orf190 | chromosome 1 open reading frame 190 | 0.95272 | Down in those who develop hereditary breast cancer |
| 100132626 | LOC100132626 | protein FAM103A1-like | 0.95852 | Down in those who develop hereditary breast cancer |
| 440278 | CATSPER2P1 | cation channel, sperm associated 2 pseudogene 1 | 0.93409 | Down in those who develop hereditary breast cancer |
| 390155 | OR5T1 | olfactory receptor, family 5, subfamily T, member 1 | 0.97737 | Down in those who develop hereditary breast cancer |
| 641518 | LOC641518 | hypothetical LOC641518 | 0.93179 | Down in those who develop hereditary breast cancer |
| 28869 | IGKV6D-41 | immunoglobulin kappa variable 6D-41 (non- | 0.98400 | Down in those who develop hereditary breast cancer |
| | | functional) | | |
| 55604 | LRRC16A | leucine rich repeat containing 16A | 0.97138 | Down in those who develop hereditary breast cancer |
| 3809 | KIR2DS4 | killer cell immunoglobulin-like receptor, two | 0.90156 | Down in those who develop hereditary breast cancer |
| | | domains, short cytoplasmic tail, 4 | | |
| 51266 | CLEC1B | C-type lectin domain family 1, member B | 0.85298 | Down in those who develop hereditary breast cancer |
| 100129822 | [No Symbol] | [No Name] | 0.96743 | Down in those who develop hereditary breast cancer |
| 55270 | NUDT15 | nudix (nucleoside diphosphate linked moiety | 0.94819 | Down in those who develop hereditary breast cancer |
| | | X)-type motif 15 | | |
| 339778 | C2orf70 | chromosome 2 open reading frame 70 | 0.98356 | Down in those who develop hereditary breast cancer |
| 348825 | TPRXL | tetra-peptide repeat homeobox-like | 0.98578 | Down in those who develop hereditary breast cancer |
| 221016 | CCDC7 | coiled-coil domain containing 7 | 0.91474 | Down in those who develop hereditary breast cancer |
| 647034 | RPS14P10 | ribosomal protein S14 pseudogene 10 | 0.92096 | Down in those who develop hereditary breast cancer |
| 3805 | KIR2DL4 | killer cell immunoglobulin-like receptor, | 0.98442 | Down in those who develop hereditary breast cancer |
| | | two domains, long cytoplasmic tail, 4 | | |
| 7380 | UPK3A | uroplakin 3A | 0.96288 | Down in those who develop hereditary breast cancer |
| 642677 | LOC642677 | family with sequence similarity 154, member | 0.94190 | Down in those who develop hereditary breast cancer |
| | | B pseudogene | | |
| 340547 | VSIG1 | V-set and immunoglobulin domain containing 1 | 0.89445 | Down in those who develop hereditary breast cancer |
| 51499 | TRIAP1 | TP53 regulated inhibitor of apoptosis 1 | 0.91981 | Down in those who develop hereditary breast cancer |
| 728707 | [No Symbol] | [No Name] | 0.95041 | Down in those who develop hereditary breast cancer |
| 130813 | C2orf50 | chromosome 2 open reading frame 50 | 0.96590 | Down in those who develop hereditary breast cancer |
| 100132310 | LOC100132310 | FCF1 small subunit (SSU) processome component | 0.96241 | Down in those who develop hereditary breast cancer |
| | | homolog (S. cerevisiae) pseudogene | | |
| 390282 | LOC390282 | eukaryotic translation initiation factor 3, | 0.92785 | Down in those who develop hereditary breast cancer |
| | | subunit F pseudogene | | |
| 100128646 | RPL10AP7 | ribosomal protein L10a pseudogene 7 | 0.97663 | Down in those who develop hereditary breast cancer |
| 54035 | PSMD4P1 | proteasome (prosome, macropain) 26S subunit, | 0.94461 | Down in those who develop hereditary breast cancer |
| | | non-ATPase, 4 pseudogene 1 | | |
| 8228 | PNPLA4 | patatin-like phospholipase domain containing 4 | 0.94592 | Down in those who develop hereditary breast cancer |
| 9363 | RAB33A | RAB33A, member RAS oncogene family | 0.97583 | Down in those who develop hereditary breast cancer |
| 344887 | LOC344887 | NmrA-like family domain containing 1 pseudogene | 0.98782 | Down in those who develop hereditary breast cancer |
| 339736 | AK2P2 | adenylate kinase 2 pseudogene 2 | 0.95816 | Down in those who develop hereditary breast cancer |
| 90499 | LOC90499 | hypothetical protein LOC90499 | 0.94649 | Down in those who develop hereditary breast cancer |
| 79625 | C4orf31 | chromosome 4 open reading frame 31 | 0.98147 | Down in those who develop hereditary breast cancer |
| 326617 | PSMA3P | proteasome (prosome, macropain) subunit, | 0.96648 | Down in those who develop hereditary breast cancer |
| | | alpha type, 3 pseudogene | | |
| 3812 | KIR3DL2 | killer cell immunoglobulin-like receptor, | 0.97100 | Down in those who develop hereditary breast cancer |
| | | three domains, long cytoplasmic tail, 2 | | |
| 100129958 | KRT8P44 | keratin 8 pseudogene 44 | 0.97648 | Down in those who develop hereditary breast cancer |
| 5054 | SERPINE1 | serpin peptidase inhibitor, clade E (nexin, | 0.95359 | Down in those who develop hereditary breast cancer |
| | | plasminogen activator inhibitor type 1), member 1 | | |
| 100037267 | LOC100037267 | developmental pluripotency associated 4 pseudogene | 0.96417 | Down in those who develop hereditary breast cancer |
| 219623 | TMEM26 | transmembrane protein 26 | 0.98584 | Down in those who develop hereditary breast cancer |
| 9241 | NOG | noggin | 0.82436 | Down in those who develop hereditary breast cancer |
| 100128050 | LOC100128050 | WD repeat domain 77 pseudogene | 0.96080 | Down in those who develop hereditary breast cancer |
| 100127889 | C10orf131 | chromosome 10 open reading frame 131 | 0.96310 | Down in those who develop hereditary breast cancer |
| 729451 | LOC729451 | hypothetical protein LOC729451 | 0.88014 | Down in those who develop hereditary breast cancer |
| 201798 | TIGD4 | tigger transposable element derived 4 | 0.97953 | Down in those who develop hereditary breast cancer |
| 64410 | KLHL25 | kelch-like 25 (Drosophila) | 0.97533 | Down in those who develop hereditary breast cancer |
| 442524 | DPY19L2P3 | dpy-19-like 2 pseudogene 3 (C. elegans) | 0.96594 | Down in those who develop hereditary breast cancer |
| 259286 | TAS2R40 | taste receptor, type 2, member 40 | 0.96512 | Down in those who develop hereditary breast cancer |
| 731039 | [No Symbol] | [No Name] | 0.97693 | Down in those who develop hereditary breast cancer |
| 401703 | LOC401703 | splicing factor U2AF 35 kDa subunit-like | 0.96591 | Down in those who develop hereditary breast cancer |
| 100130859 | [No Symbol] | [No Name] | 0.95802 | Down in those who develop hereditary breast cancer |
| 26 | ABP1 | amilonde binding protein 1 (amine oxidase | 0.98467 | Down in those who develop hereditary breast cancer |
| | | (copper-containing)) | | |
| 347051 | SLC10A5 | solute carrier family 10 (sodium/bile acid | 0.96335 | Down in those who develop hereditary breast cancer |
| | | cotransporter family), member 5 | | |
| 144715 | RAD9B | RAD9 homolog B (S. pombe) | 0.95746 | Down in those who develop hereditary breast cancer |
| 387856 | C12orf68 | chromosome 12 open reading frame 68 | 0.96793 | Down in those who develop hereditary breast cancer |
| 84833 | USMG5 | up-regulated during skeletal muscle growth 5 | 0.92975 | Down in those who develop hereditary breast cancer |
| | | homolog (mouse) | | |
| 148645 | C1orf211 | chromosome 1 open reading frame 211 | 0.98681 | Down in those who develop hereditary breast cancer |
| 400576 | FLJ45831 | hypothetical FLJ45831 | 0.98243 | Down in those who develop hereditary breast cancer |
| 84332 | DYDC2 | DPY30 domain containing 2 | 0.97996 | Down in those who develop hereditary breast cancer |
| 56891 | LGALS14 | lectin, galactoside-binding, soluble, 14 | 0.97194 | Down in those who develop hereditary breast cancer |
| 100128403 | [No Symbol] | [No Name] | 0.98170 | Down in those who develop hereditary breast cancer |
| 730109 | LOC730109 | hypothetical protein LOC730109 | 0.98596 | Down in those who develop hereditary breast cancer |
| 100131568 | LOC100131568 | RNA binding motif protein 43 pseudogene | 0.96482 | Down in those who develop hereditary breast cancer |
| 100132805 | [No Symbol] | [No Name] | 0.98826 | Down in those who develop hereditary breast cancer |
| 7012 | TERC | telomerase RNA component | 0.89933 | Down in those who develop hereditary breast cancer |
| 729950 | LOC729950 | hypothetical LOC729950 | 0.97018 | Down in those who develop hereditary breast cancer |
| 79022 | TMEM106C | transmembrane protein 106C | 0.90028 | Down in those who develop hereditary breast cancer |
| 144608 | C12orf60 | chromosome 12 open reading frame 60 | 0.97242 | Down in those who develop hereditary breast cancer |
| 91582 | RPS19BP1 | ribosomal protein S19 binding protein 1 | 0.92038 | Down in those who develop hereditary breast cancer |
| 388507 | ZNF788 | zinc finger family member 788 | 0.96818 | Down in those who develop hereditary breast cancer |
| 4112 | MAGEB1 | melanoma antigen family B, 1 | 0.98272 | Down in those who develop hereditary breast cancer |
| 2841 | GPR18 | G protein-coupled receptor 18 | 0.90221 | Down in those who develop hereditary breast cancer |
| 646895 | [No Symbol] | [No Name] | 0.91585 | Down in those who develop hereditary breast cancer |
| 400165 | C13orf35 | chromosome 13 open reading frame 35 | 0.96640 | Down in those who develop hereditary breast cancer |
| 284348 | LYPD5 | LY6/PLAUR domain containing 5 | 0.98710 | Down in those who develop hereditary breast cancer |
| 6555 | SLC10A2 | solute carrier family 10 (sodium/bile acid | 0.98761 | Down in those who develop hereditary breast cancer |
| | | cotransporter family), member 2 | | |
| 151258 | SLC38A11 | solute carrier family 38, member 11 | 0.96230 | Down in those who develop hereditary breast cancer |
| 646316 | TERF1P3 | telomeric repeat binding factor (NIMA- | 0.94636 | Down in those who develop hereditary breast cancer |
| | | interacting) 1 pseudogene 3 | | |
| 388649 | C1orf146 | chromosome 1 open reading frame 146 | 0.97890 | Down in those who develop hereditary breast cancer |
| 100128417 | [No Symbol] | [No Name] | 0.96758 | Down in those who develop hereditary breast cancer |
| 54033 | RBM11 | RNA binding motif protein 11 | 0.94485 | Down in those who develop hereditary breast cancer |
| 26256 | CABYR | calcium binding tyrosine-(Y)-phosphorylation | 0.96896 | Down in those who develop hereditary breast cancer |
| | | regulated | | |
| 100129077 | [No Symbol] | [No Name] | 0.98416 | Down in those who develop hereditary breast cancer |
| 222962 | SLC29A4 | solute carrier family 29 (nucleoside | 0.95925 | Down in those who develop hereditary breast cancer |
| | | transporters), member 4 | | |
| 644588 | DNAJA1P3 | DnaJ (Hsp40) homolog, subfamily A, member 1 | 0.95825 | Down in those who develop hereditary breast cancer |
| | | pseudogene 3 | | |
| 153893 | LOC153893 | DCN1, defective in cullin neddylation 1, | 0.97614 | Down in those who develop hereditary breast cancer |
| | | domain containing 1 (S. cerevisiae) pseudogene | | |
| 51616 | TAF9B | TAF9B RNA polymerase II, TATA box binding | 0.90443 | Down in those who develop hereditary breast cancer |
| | | protein (TBP)-associated factor, 31 kDa | | |
| 645086 | LOC645086 | chromosome 11 open reading frame 58 pseudogene | 0.95211 | Down in those who develop hereditary breast cancer |
| 100130748 | LOC100130748 | 3-hydroxyisobutyryl-Coenzyme A hydrolase | 0.98703 | Down in those who develop hereditary breast cancer |
| | | pseudogene | | |
| 147657 | ZNF480 | zinc finger protein 480 | 0.91736 | Down in those who develop hereditary breast cancer |
| 390860 | LOC390860 | ribosomal protein, large, P0 pseudogene | 0.96564 | Down in those who develop hereditary breast cancer |
| 28466 | IGHV1-45 | immunoglobulin heavy variable 1-45 | 0.97065 | Down in those who develop hereditary breast cancer |
| 1823 | DSC1 | desmocollin 1 | 0.89531 | Down in those who develop hereditary breast cancer |
| 119 | ADD2 | adducin 2 (beta) | 0.98577 | Down in those who develop hereditary breast cancer |
| 55906 | ZC4H2 | zinc finger, C4H2 domain containing | 0.94259 | Down in those who develop hereditary breast cancer |
| 123862 | LOC123862 | interferon induced transmembrane protein pseudogene | 0.95430 | Down in those who develop hereditary breast cancer |
| 1266 | CNN3 | calponin 3, acidic | 0.97602 | Down in those who develop hereditary breast cancer |
| 441502 | RPS26P11 | ribosomal protein S26 pseudogene 11 | 0.89168 | Down in those who develop hereditary breast cancer |
| 57570 | TRMT5 | TRM5 tRNA methyltransferase 5 homolog | 0.92717 | Down in those who develop hereditary breast cancer |
| | | (S. cerevisiae) | | |
| 388182 | FLJ42289 | hypothetical LOC388182 | 0.96063 | Down in those who develop hereditary breast cancer |
| 64838 | FNDC4 | fibronectin type III domain containing 4 | 0.99459 | Down in those who develop hereditary breast cancer |
| 100126481 | TRNAI-AAU | transfer RNA isoleucine (anticodon AAU) | 0.98650 | Down in those who develop hereditary breast cancer |
| 55702 | CCDC94 | coiled-coil domain containing 94 | 0.93784 | Down in those who develop hereditary breast cancer |
| 730058 | LOC730058 | UPF0621 protein C-like | 0.96916 | Down in those who develop hereditary breast cancer |
| 728545 | [No Symbol] | [No Name] | 0.92791 | Down in those who develop hereditary breast cancer |
| 51186 | WBP5 | WW domain binding protein 5 | 0.96834 | Down in those who develop hereditary breast cancer |
| 85352 | KIAA1644 | KIAA1644 | 0.97770 | Down in those who develop hereditary breast cancer |
| 7643 | ZNF90 | zinc finger protein 90 | 0.97669 | Down in those who develop hereditary breast cancer |
| 100128122 | LOC100128122 | Cas-Br-M (murine) ecotropic retroviral | 0.97337 | Down in those who develop hereditary breast cancer |
| | | transforming sequence-like 1 pseudogene | | |
| 100129444 | [No Symbol] | [No Name] | 0.95460 | Down in those who develop hereditary breast cancer |
| 1424 | CRYGGP | crystallin, gamma G, pseudogene | 0.96815 | Down in those who develop hereditary breast cancer |
| 100129945 | LOC100129945 | lupus La protein-like | 0.97758 | Down in those who develop hereditary breast cancer |
| 653492 | PSG10P | pregnancy specific beta-1-glycoprotein 10, pseudogene | 0.97264 | Down in those who develop hereditary breast cancer |
| 119391 | GSTO2 | glutathione S-transferase omega 2 | 0.97955 | Down in those who develop hereditary breast cancer |
| 100130623 | [No Symbol] | [No Name] | 0.99692 | Down in those who develop hereditary breast cancer |
| 100129837 | [No Symbol] | [No Name] | 0.99866 | Down in those who develop hereditary breast cancer |
| 441964 | [No Symbol] | [No Name] | 0.98827 | Down in those who develop hereditary breast cancer |
| 729868 | [No Symbol] | [No Name] | 0.97418 | Down in those who develop hereditary breast cancer |
| 3053 | SERPIND1 | serpin peptidase inhibitor, clade D (heparin | 0.98200 | Down in those who develop hereditary breast cancer |
| | | cofactor), member 1 | | |
| 83844 | USP26 | ubiquitin specific peptidase 26 | 0.96043 | Down in those who develop hereditary breast cancer |
| 9397 | NMT2 | N-myristoyltransferase 2 | 0.92727 | Down in those who develop hereditary breast cancer |
| 197370 | NSMCE1 | non-SMC element 1 homolog (S. cerevisiae) | 0.95993 | Down in those who develop hereditary breast cancer |
| 1068 | CETN1 | centrin, EF-hand protein, 1 | 0.95410 | Down in those who develop hereditary breast cancer |
| 166752 | FREM3 | FRAS1 related extracellular matrix 3 | 0.99312 | Down in those who develop hereditary breast cancer |
| 3625 | INHBB | inhibin, beta B | 0.95366 | Down in those who develop hereditary breast cancer |
| 80086 | TUBA4B | tubulin, alpha 4b (pseudogene) | 0.98422 | Down in those who develop hereditary breast cancer |
| 100131056 | [No Symbol] | [No Name] | 0.98376 | Down in those who develop hereditary breast cancer |
| 26590 | OR8B7P | olfactory receptor, family 8, subfamily B, | 0.97829 | Down in those who develop hereditary breast cancer |
| | | member 7 pseudogene | | |
| 767579 | SNORD114-3 | small nucleolar RNA, C/D box 114-3 | 0.95780 | Down in those who develop hereditary breast cancer |
| 100129046 | LOC100129046 | hypothetical LOC100129046 | 0.97470 | Down in those who develop hereditary breast cancer |
| 10127 | ZNF263 | zinc finger protein 263 | 0.93963 | Down in those who develop hereditary breast cancer |
| 84996 | C21orf119 | chromosome 21 open reading frame 119 | 0.95791 | Down in those who develop hereditary breast cancer |
| 5988 | RFPL1 | ret finger protein-like 1 | 0.97369 | Down in those who develop hereditary breast cancer |
| 81050 | OR5AC2 | olfactory receptor, family 5, subfamily AC, member 2 | 0.97482 | Down in those who develop hereditary breast cancer |

TABLE 2
Utah1 FC Summary Up

| Entrez | | | Fold | |
|---|---|---|---|---|
| Gene ID | Gene Symbol | Gene Name | Change | Description |
| 100128709 | [No Symbol] | [No Name] | 1.05850 | Up in those who develop hereditary breast cancer |
| 100128700 | [No Symbol] | [No Name] | 1.03000 | Up in those who develop hereditary breast cancer |
| 392282 | RPS5P6 | ribosomal protein S5 pseudogene 6 | 1.02827 | Up in those who develop hereditary breast cancer |
| 83604 | TMEM47 | transmembrane protein 47 | 1.02085 | Up in those who develop hereditary breast cancer |
| 400954 | EML6 | echinoderm microtubule associated protein like 6 | 1.02327 | Up in those who develop hereditary breast cancer |
| 647298 | HSPD1P8 | heat shock 60 kDa protein 1 (chaperonin) pseudogene 8 | 1.01504 | Up in those who develop hereditary breast cancer |
| 100128493 | LOC100128493 | ubiquitin-conjugating enzyme E2 variant 2 pseudogene | 1.03824 | Up in those who develop hereditary breast cancer |
| 128774 | MRPS11P1 | mitochondrial ribosomal protein S11 pseudogene 1 | 1.03087 | Up in those who develop hereditary breast cancer |
| 149157 | [No Symbol] | [No Name] | 1.02750 | Up in those who develop hereditary breast cancer |
| 7473 | WNT3 | wingless-type MMTV integration site family, member 3 | 1.01553 | Up in those who develop hereditary breast cancer |
| 54436 | SH3TC1 | SH3 domain and tetratricopeptide repeats 1 | 1.03083 | Up in those who develop hereditary breast cancer |
| 5999 | RGS4 | regulator of G-protein signaling 4 | 1.02586 | Up in those who develop hereditary breast cancer |
| 449518 | LOC449518 | purinergic receptor P2Y, G-protein coupled, 10 | 1.04589 | Up in those who develop hereditary breast cancer |
| | | pseudogene | | |
| 79825 | CCDC48 | coiled-coil domain containing 48 | 1.01918 | Up in those who develop hereditary breast cancer |
| 442673 | TUBG1P | tubulin, gamma 1 pseudogene | 1.04787 | Up in those who develop hereditary breast cancer |
| 100131609 | HNRNPA1P2 | heterogeneous nuclear ribonucleoprotein A1 | 1.05136 | Up in those who develop hereditary breast cancer |
| | | pseudogene 2 | | |
| 389428 | RPL5P18 | ribosomal protein L5 pseudogene 18 | 1.05885 | Up in those who develop hereditary breast cancer |
| 100128386 | LOC100128386 | hypothetical LOC100128386 | 1.02361 | Up in those who develop hereditary breast cancer |
| 649489 | LOC649489 | protein phosphatase 1, regulatory (inhibitor) | 1.02506 | Up in those who develop hereditary breast cancer |
| | | subunit 2 pseudogene | | |
| 643586 | LOC643586 | pyruvate kinase, muscle pseudogene | 1.03424 | Up in those who develop hereditary breast cancer |
| 283314 | MATL2963 | hypothetical LOC283314 | 1.03595 | Up in those who develop hereditary breast cancer |
| 408029 | C2orf27B | chromosome 2 open reading frame 27B | 1.02973 | Up in those who develop hereditary breast cancer |
| 10461 | MERTK | c-mer proto-oncogene tyrosine kinase | 1.03881 | Up in those who develop hereditary breast cancer |
| 10877 | CFHR4 | complement factor H-related 4 | 1.03294 | Up in those who develop hereditary breast cancer |
| 283571 | PROX2 | prospero homeobox 2 | 1.01521 | Up in those who develop hereditary breast cancer |
| 100129915 | [No Symbol] | [No Name] | 1.01662 | Up in those who develop hereditary breast cancer |
| 3426 | CFI | complement factor I | 1.01805 | Up in those who develop hereditary breast cancer |
| 780813 | PAICSP4 | phosphoribosylaminoimidazole carboxylase, | 1.01634 | Up in those who develop hereditary breast cancer |
| | | phosphoribosylaminoimidazole succinocarboxamide | | |
| | | synthetase pseudogene 4 | | |
| 729486 | IL9RP3 | interleukin 9 receptor pseudogene 3 | 1.04554 | Up in those who develop hereditary breast cancer |
| 401433 | LOC401433 | hypothetical LOC401433 | 1.03820 | Up in those who develop hereditary breast cancer |
| 286122 | C8orf31 | chromosome 8 open reading frame 31 | 1.02932 | Up in those who develop hereditary breast cancer |
| 219902 | TMEM136 | transmembrane protein 136 | 1.01357 | Up in those who develop hereditary breast cancer |
| 199713 | NLRP7 | NLR family, pyrin domain containing 7 | 1.03336 | Up in those who develop hereditary breast cancer |
| 1769 | DNAH8 | dynein, axonemal, heavy chain 8 | 1.01179 | Up in those who develop hereditary breast cancer |
| 100130249 | PP2672 | hypothetical LOC100130249 | 1.04144 | Up in those who develop hereditary breast cancer |
| 163778 | SPRR4 | small proline-rich protein 4 | 1.05947 | Up in those who develop hereditary breast cancer |
| 148641 | SLC35F3 | solute carrier family 35, member F3 | 1.01311 | Up in those who develop hereditary breast cancer |
| 400347 | LOC400347 | REX4, RNA exonuclease 4 homolog (S. cerevisiae) | 1.03336 | Up in those who develop hereditary breast cancer |
| | | pseudogene | | |
| 347333 | KRT8P14 | keratin 8 pseudogene 14 | 1.00982 | Up in those who develop hereditary breast cancer |
| 121270 | OR11M1P | olfactory receptor, family 11, subfamily M, member 1 | 1.06053 | Up in those who develop hereditary breast cancer |
| | | pseudogene | | |
| 6461 | SHB | Src homology 2 domain containing adaptor protein B | 1.01215 | Up in those who develop hereditary breast cancer |
| 4744 | NEFH | neuro filament, heavy polypeptide | 1.01566 | Up in those who develop hereditary breast cancer |
| 729041 | LOC729041 | fatty-acid amide hydrolase 1-like | 1.04385 | Up in those who develop hereditary breast cancer |
| 2335 | FN1 | fibronectin 1 | 1.02499 | Up in those who develop hereditary breast cancer |
| 644915 | METTL15P2 | methyltransferase like 15 pseudogene 2 | 1.01990 | Up in those who develop hereditary breast cancer |
| 100132086 | LOC100132086 | adenylate kinase isoenzyme 6-like | 1.02578 | Up in those who develop hereditary breast cancer |
| 2596 | GAP43 | growth associated protein 43 | 1.01796 | Up in those who develop hereditary breast cancer |
| 646576 | LOC646576 | hypothetical LOC646576 | 1.00778 | Up in those who develop hereditary breast cancer |
| 644662 | LOC644662 | hypothetical protein LOC644662 | 1.01804 | Up in those who develop hereditary breast cancer |
| 339983 | NAT8L | N-acetyltransferase 8-like (GCN5-related, putative) | 1.02554 | Up in those who develop hereditary breast cancer |
| 130013 | ACMSD | aminocarboxymuconate semialdehyde decarboxylase | 1.02182 | Up in those who develop hereditary breast cancer |
| 440603 | BCL2L15 | BCL2-like 15 | 1.02400 | Up in those who develop hereditary breast cancer |
| 152078 | C3orf55 | chromosome 3 open reading frame 55 | 1.02245 | Up in those who develop hereditary breast cancer |
| 201895 | C4orf34 | chromosome 4 open reading frame 34 | 1.02999 | Up in those who develop hereditary breast cancer |
| 221711 | SYCP2L | synaptonemal complex protein 2-like | 1.03038 | Up in those who develop hereditary breast cancer |
| 8829 | NRP1 | neuropilin 1 | 1.01069 | Up in those who develop hereditary breast cancer |
| 1472 | CST4 | cystatin S | 1.05145 | Up in those who develop hereditary breast cancer |
| 100128389 | [No Symbol] | [No Name] | 1.04353 | Up in those who develop hereditary breast cancer |
| 118663 | BTBD16 | BTB (POZ) domain containing 16 | 1.01455 | Up in those who develop hereditary breast cancer |
| 6887 | TAL2 | T-cell acute lymphocytic leukemia 2 | 1.03352 | Up in those who develop hereditary breast cancer |
| 50835 | TAS2R9 | taste receptor, type 2, member 9 | 1.02523 | Up in those who develop hereditary breast cancer |
| 4157 | MC1R | melanocortin 1 receptor (alpha melanocyte | 1.03616 | Up in those who develop hereditary breast cancer |
| | | stimulating hormone receptor) | | |
| 785 | CACNB4 | calcium channel, voltage-dependent, beta 4 subunit | 1.01724 | Up in those who develop hereditary breast cancer |
| 100130268 | LOC100130268 | similar to hCG1648866 | 1.03898 | Up in those who develop hereditary breast cancer |
| 100128457 | LOC100128457 | similar to hCG2026341 | 1.04522 | Up in those who develop hereditary breast cancer |
| 100132214 | [No Symbol] | [No Name] | 1.04735 | Up in those who develop hereditary breast cancer |
| 51676 | ASB2 | ankyrin repeat and SOCS box containing 2 | 1.01372 | Up in those who develop hereditary breast cancer |
| 27145 | FILIP1 | filamin A interacting protein 1 | 1.02313 | Up in those who develop hereditary breast cancer |
| 100131819 | [No Symbol] | [No Name] | 1.02809 | Up in those who develop hereditary breast cancer |
| 728780 | ANKDD1B | ankyrin repeat and death domain containing 1B | 1.01894 | Up in those who develop hereditary breast cancer |
| 85481 | PSKH2 | protein serine kinase H2 | 1.01887 | Up in those who develop hereditary breast cancer |
| 100129106 | [No Symbol] | [No Name] | 1.01457 | Up in those who develop hereditary breast cancer |
| 78998 | C8orf51 | chromosome 8 open reading frame 51 | 1.03279 | Up in those who develop hereditary breast cancer |
| 84099 | ID2B | inhibitor of DNA binding 2B, dominant negative | 1.02669 | Up in those who develop hereditary breast cancer |
| | | helix-loop-helix protein (pseudogene) | | |
| 644862 | RPS28P3 | ribosomal protein S28 pseudogene 3 | 1.03776 | Up in those who develop hereditary breast cancer |
| 163183 | C19orf46 | chromosome 19 open reading frame 46 | 1.02242 | Up in those who develop hereditary breast cancer |
| 326288 | RPL17P4 | ribosomal protein L17 pseudogene 4 | 1.04015 | Up in those who develop hereditary breast cancer |
| 100131042 | [No Symbol] | [No Name] | 1.04994 | Up in those who develop hereditary breast cancer |
| 139411 | PTCHD1 | patched domain containing 1 | 1.01407 | Up in those who develop hereditary breast cancer |
| 4085 | MAD2L1 | MAD2 mitotic arrest deficient-like 1 (yeast) | 1.01352 | Up in those who develop hereditary breast cancer |
| 730126 | [No Symbol] | [No Name] | 1.03615 | Up in those who develop hereditary breast cancer |
| 606495 | CYB5RL | cytochrome b5 reductase-like | 1.00749 | Up in those who develop hereditary breast cancer |
| 653194 | LOC653194 | KH homology domain-containing protein 1-like | 1.03238 | Up in those who develop hereditary breast cancer |
| 26647 | OR7E25P | olfactory receptor, family 7, subfamily E, | 1.03869 | Up in those who develop hereditary breast cancer |
| | | member 25 pseudogene | | |
| 63974 | NEUROD6 | neurogenic differentiation 6 | 1.02163 | Up in those who develop hereditary breast cancer |
| 25789 | TMEM59L | transmembrane protein 59-like | 1.00625 | Up in those who develop hereditary breast cancer |
| 353194 | LOC353194 | keratin pseudogene | 1.03225 | Up in those who develop hereditary breast cancer |
| 390084 | OR56A5 | olfactory receptor, family 56, subfamily A, member 5 | 1.03358 | Up in those who develop hereditary breast cancer |
| 285555 | C4orf37 | chromosome 4 open reading frame 37 | 1.01344 | Up in those who develop hereditary breast cancer |
| 23439 | ATP1B4 | ATPase, Na+/K+ transporting, beta 4 polypeptide | 1.01288 | Up in those who develop hereditary breast cancer |
| 644941 | [No Symbol] | [No Name] | 1.06134 | Up in those who develop hereditary breast cancer |
| 643669 | LOC643669 | hypothetical protein LOC643669 | 1.01596 | Up in those who develop hereditary breast cancer |
| 130500 | CISD1P1 | CDGSH iron sulfur domain 1 pseudogene 1 | 1.03521 | Up in those who develop hereditary breast cancer |
| 414318 | C9orf106 | chromosome 9 open reading frame 106 | 1.03024 | Up in those who develop hereditary breast cancer |
| 10316 | NMUR1 | neuromedin U receptor 1 | 1.05132 | Up in those who develop hereditary breast cancer |
| 55057 | AIM1L | absent in melanoma 1-like | 1.01575 | Up in those who develop hereditary breast cancer |
| 100132053 | RPL30P8 | ribosomal protein L30 pseudogene 8 | 1.01456 | Up in those who develop hereditary breast cancer |
| 441505 | LOC441505 | stress-induced-phosphoprotein 1 pseudogene | 1.00827 | Up in those who develop hereditary breast cancer |
| 100128217 | LOC100128217 | general transcription factor IIIA pseudogene | 1.00874 | Up in those who develop hereditary breast cancer |
| 4342 | MOS | v-mos Moloney murine sarcoma viral oncogene homolog | 1.01892 | Up in those who develop hereditary breast cancer |
| 644489 | [No Symbol] | [No Name] | 1.03232 | Up in those who develop hereditary breast cancer |
| 4071 | TM4SF1 | transmembrane 4 L six family member 1 | 1.03137 | Up in those who develop hereditary breast cancer |
| 25925 | ZNF521 | zinc finger protein 521 | 1.01135 | Up in those who develop hereditary breast cancer |
| 100131070 | LOC100131070 | mpv17-like protein 2-like | 1.03546 | Up in those who develop hereditary breast cancer |
| 100128979 | LOC100128979 | hypothetical LOC100128979 | 1.03901 | Up in those who develop hereditary breast cancer |
| 728050 | [No Symbol] | [No Name] | 1.08112 | Up in those who develop hereditary breast cancer |
| 55540 | IL17RB | interleukin 17 receptor B | 1.01748 | Up in those who develop hereditary breast cancer |
| 11341 | SCRG1 | stimulator of chondrogenesis 1 | 1.01927 | Up in those who develop hereditary breast cancer |
| 7070 | THY1 | Thy-1 cell surface antigen | 1.01532 | Up in those who develop hereditary breast cancer |
| 130162 | C2orf63 | chromosome 2 open reading frame 63 | 1.01620 | Up in those who develop hereditary breast cancer |
| 3394 | IRF8 | interferon regulatory factor 8 | 1.03235 | Up in those who develop hereditary breast cancer |
| 3226 | HOXC10 | homeobox C10 | 1.01038 | Up in those who develop hereditary breast cancer |
| 649288 | AK4P6 | adenylate kinase 4 pseudogene 6 | 1.03149 | Up in those who develop hereditary breast cancer |
| 140691 | TRIM69 | tripartite motif containing 69 | 1.02117 | Up in those who develop hereditary breast cancer |
| 100132501 | LOC100132501 | hypothetical LOC100132501 | 1.01055 | Up in those who develop hereditary breast cancer |
| 81849 | ST6GALNAC5 | ST6 (alpha-N-acetyl-neuraminyl-2,3-beta-galactosyl- | 1.01527 | Up in those who develop hereditary breast cancer |
| | | 1,3)-N-acetylgalactosaminide alpha-2,6- | | |
| | | sialyltransferase 5 | | |
| 2151 | F2RL2 | coagulation factor II (thrombin) receptor-like 2 | 1.02926 | Up in those who develop hereditary breast cancer |
| 574447 | MIR146B | microRNA 146b | 1.03471 | Up in those who develop hereditary breast cancer |
| 219429 | OR4C11 | olfactory receptor, family 4, subfamily C, member 11 | 1.02121 | Up in those who develop hereditary breast cancer |
| 100132073 | LOC100132073 | cyclin B2 pseudogene | 1.03497 | Up in those who develop hereditary breast cancer |
| 145259 | RPSAP4 | ribosomal protein SA pseudogene 4 | 1.01434 | Up in those who develop hereditary breast cancer |
| 392288 | LOC392288 | micro tubule-associated proteins 1A/1B light | 1.02050 | Up in those who develop hereditary breast cancer |
| | | chain 3B-like | | |
| 285423 | LOC285423 | hypothetical LOC285423 | 1.01226 | Up in those who develop hereditary breast cancer |
| 128820 | CST9LP1 | cystatin 9-like pseudogene 1 | 1.03984 | Up in those who develop hereditary breast cancer |
| 149837 | LOC149837 | hypothetical LOC149837 | 1.01494 | Up in those who develop hereditary breast cancer |
| 255403 | ZNF718 | zinc finger protein 718 | 1.03097 | Up in those who develop hereditary breast cancer |
| 55808 | ST6GALNAC1 | ST6 (alpha-N-acetyl-neuraminyl-2,3-beta-galactosyl- | 1.00581 | Up in those who develop hereditary breast cancer |
| | | 1,3)-N-acetylgalactosaminide alpha-2,6- | | |
| | | sialyltransferase 1 | | |
| 3626 | INHBC | inhibin, beta C | 1.02582 | Up in those who develop hereditary breast cancer |
| 390844 | ARIH2P1 | ariadne homolog 2 pseudogene 1 | 1.01615 | Up in those who develop hereditary breast cancer |
| 7621 | ZNF70 | zinc finger protein 70 | 1.00639 | Up in those who develop hereditary breast cancer |
| 100132623 | [No Symbol] | [No Name] | 1.03856 | Up in those who develop hereditary breast cancer |
| 338017 | KRT8P1 | keratin 8 pseudogene 1 | 1.01192 | Up in those who develop hereditary breast cancer |
| 143630 | UBQLNL | ubiquilin-like | 1.02513 | Up in those who develop hereditary breast cancer |
| 80097 | MZT2B | mitotic spindle organizing protein 2B | 1.03085 | Up in those who develop hereditary breast cancer |
| 806 | CALM2P2 | calmodulin 2 pseudogene 2 | 1.03895 | Up in those who develop hereditary breast cancer |
| 51659 | GINS2 | GINS complex subunit 2 (Psf2 homolog) | 1.01107 | Up in those who develop hereditary breast cancer |
| 644464 | RPSAP61 | ribosomal protein SA pseudogene 61 | 1.04385 | Up in those who develop hereditary breast cancer |
| 442192 | DDX6P1 | DEAD (Asp-Glu-Ala-Asp) box polypeptide 6 | 1.00649 | Up in those who develop hereditary breast cancer |
| | | pseudogene 1 | | |
| 338094 | FAM151A | family with sequence similarity 151, member A | 1.02257 | Up in those who develop hereditary breast cancer |
| 285987 | DLX6-AS1 | DLX6 antisense RNA 1 (non-protein coding) | 1.02260 | Up in those who develop hereditary breast cancer |
| 100129427 | LOC100129427 | hypothetical LOC100129427 | 1.04063 | Up in those who develop hereditary breast cancer |
| 442709 | EEF1A1P28 | eukaryotic translation elongation factor 1 | 1.02390 | Up in those who develop hereditary breast cancer |
| | | alpha 1 pseudogene28 | | |
| 100129381 | LOC100129381 | ribosomal protein S7 pseudogene | 1.01754 | Up in those who develop hereditary breast cancer |
| 392387 | LOC392387 | adenosylhomocysteinase pseudogene | 1.00870 | Up in those who develop hereditary breast cancer |
| 730338 | LOC730338 | hypothetical LOC730338 | 1.02145 | Up in those who develop hereditary breast cancer |
| 5222 | PGA5 | pepsinogen 5, group I (pepsinogen A) | 1.02036 | Up in those who develop hereditary breast cancer |
| 390435 | OR4T1P | olfactory receptor, family 4, subfamily T, | 1.02804 | Up in those who develop hereditary breast cancer |
| | | member 1 pseudogene | | |
| 8626 | TP63 | tumor protein p63 | 1.01222 | Up in those who develop hereditary breast cancer |
| 391160 | ARPC3P2 | actin related protein 2/3 complex, subunit 3 | 1.03806 | Up in those who develop hereditary breast cancer |
| | | pseudogene 2 | | |
| 157773 | C8orf48 | chromosome 8 open reading frame 48 | 1.02024 | Up in those who develop hereditary breast cancer |
| 2847 | MCHR1 | melanin-concentrating hormone receptor 1 | 1.03315 | Up in those who develop hereditary breast cancer |
| 3229 | HOXC13 | homeobox C13 | 1.01601 | Up in those who develop hereditary breast cancer |
| 100131087 | RPLP1P11 | ribosomal protein, large, P1 pseudogene 11 | 1.03187 | Up in those who develop hereditary breast cancer |
| 647532 | LOC647532 | phenylalanine-tRNA synthetase-like, beta | 1.02208 | Up in those who develop hereditary breast cancer |
| | | subunit pseudogene | | |
| 3381 | IBSP | integrin-binding sialoprotein | 1.02260 | Up in those who develop hereditary breast cancer |
| 219981 | OR5A2 | olfactory receptor, family 5, subfamily A, member 2 | 1.05672 | Up in those who develop hereditary breast cancer |
| 286555 | TBL1YP1 | transducin (beta)-like 1, Y-linked pseudogene 1 | 1.01866 | Up in those who develop hereditary breast cancer |
| 9037 | SEMA5A | sema domain, seven thrombospondin repeats (type | 1.01422 | Up in those who develop hereditary breast cancer |
| | | 1 and type 1-like), transmembrane domain (TM) | | |
| | | and short cytoplasmic domain, (semaphorin) 5A | | |
| 6276 | S100A5 | S100 calcium binding protein A5 | 1.02479 | Up in those who develop hereditary breast cancer |
| 100128956 | [No Symbol] | [No Name] | 1.01327 | Up in those who develop hereditary breast cancer |
| 9145 | SYNGR1 | synaptogyrin 1 | 1.00260 | Up in those who develop hereditary breast cancer |
| 100129062 | [No Symbol] | [No Name] | 1.01261 | Up in those who develop hereditary breast cancer |
| 2444 | FRK | fyn-related kinase | 1.01239 | Up in those who develop hereditary breast cancer |
| 100133286 | LOC100133286 | hypothetical LOC100133286 | 1.01395 | Up in those who develop hereditary breast cancer |

TABLE 3
Utah2 FC Summary Down

| Entrez | | | Fold | |
|---|---|---|---|---|
| Gene ID | Gene Symbol | Gene Name | Change | Description |
| 541468 | C1orf190 | chromosome 1 open reading frame 190 | 0.95272 | Down in those who develop hereditary breast cancer |
| 221016 | CCDC7 | coiled-coil domain containing 7 | 0.91474 | Down in those who develop hereditary breast cancer |
| 7012 | TERC | telomerase RNA component | 0.89933 | Down in those who develop hereditary breast cancer |
| 55270 | NUDT15 | nudix (nucleoside diphosphate linked moiety X)-type | 0.94819 | Down in those who develop hereditary breast cancer |
| | | motif 15 | | |
| 728707 | [No Symbol] | [No Name] | 0.95041 | Down in those who develop hereditary breast cancer |
| 730255 | RPL17P8 | ribosomal protein L17 pseudogene 8 | 0.97608 | Down in those who develop hereditary breast cancer |
| 4112 | MAGEB1 | melanoma antigen family B, 1 | 0.98272 | Down in those who develop hereditary breast cancer |
| 100129822 | [No Symbol] | [No Name] | 0.96743 | Down in those who develop hereditary breast cancer |
| 390282 | LOC390282 | eukaryotic translation initiation factor | 0.92785 | Down in those who develop hereditary breast cancer |
| | | 3, subunit F pseudogene | | |
| 197370 | NSMCE1 | non-SMC element 1 homolog (S. cerevisiae) | 0.95993 | Down in those who develop hereditary breast cancer |
| 728509 | RPS19P7 | ribosomal protein S19 pseudogene 7 | 0.96104 | Down in those who develop hereditary breast cancer |
| 442524 | DPY19L2P3 | dpy-19-like 2 pseudogene 3 (C. elegans) | 0.96594 | Down in those who develop hereditary breast cancer |
| 647034 | RPS14P10 | ribosomal protein S14 pseudogene 10 | 0.92096 | Down in those who develop hereditary breast cancer |
| 339778 | C2orf70 | chromosome 2 open reading frame 70 | 0.98356 | Down in those who develop hereditary breast cancer |
| 642677 | LOC642677 | family with sequence similarity 154, member B | 0.94190 | Down in those who develop hereditary breast cancer |
| | | pseudogene | | |
| 100128050 | LOC100128050 | WD repeat domain 77 pseudogene | 0.96080 | Down in those who develop hereditary breast cancer |
| 729451 | LOC729451 | hypothetical protein LOC729451 | 0.88014 | Down in those who develop hereditary breast cancer |
| 51266 | CLEC1B | C-type lectin domain family 1, member B | 0.85298 | Down in those who develop hereditary breast cancer |
| 100128646 | RPL10AP7 | ribosomal protein L10a pseudogene 7 | 0.97663 | Down in those who develop hereditary breast cancer |
| 148645 | C1orf211 | chromosome 1 open reading frame 211 | 0.98681 | Down in those who develop hereditary breast cancer |
| 440278 | CATSPER2P1 | cation channel, sperm associated 2 pseudogene 1 | 0.93409 | Down in those who develop hereditary breast cancer |
| 653492 | PSG10P | pregnancy specific beta-1-glycoprotein 10, pseudogene | 0.97264 | Down in those who develop hereditary breast cancer |
| 391106 | VDAC1P9 | voltage-dependent anion channel 1 pseudogene 9 | 0.96278 | Down in those who develop hereditary breast cancer |
| 441502 | RPS26P11 | ribosomal protein S26 pseudogene 11 | 0.89168 | Down in those who develop hereditary breast cancer |
| 79022 | TMEM106C | transmembrane protein 106C | 0.90028 | Down in those who develop hereditary breast cancer |
| 326617 | PSMA3P | proteasome (prosome, macropain) subunit, | 0.96648 | Down in those who develop hereditary breast cancer |
| | | alpha type, 3 pseudogene | | |
| 344887 | LOC344887 | NmrA-like family domain containing 1 pseudogene | 0.98782 | Down in those who develop hereditary breast cancer |
| 645086 | LOC645086 | chromosome 11 open reading frame 58 pseudogene | 0.95211 | Down in those who develop hereditary breast cancer |
| 3872 | KRT17 | keratin 17 | 0.97206 | Down in those who develop hereditary breast cancer |
| 1823 | DSC1 | desmocollin 1 | 0.89531 | Down in those who develop hereditary breast cancer |
| 91392 | ZNF502 | zinc finger protein 502 | 0.95017 | Down in those who develop hereditary breast cancer |
| 130813 | C2orf50 | chromosome 2 open reading frame 50 | 0.96590 | Down in those who develop hereditary breast cancer |
| 219623 | TMEM26 | transmembrane protein 26 | 0.98584 | Down in those who develop hereditary breast cancer |
| 87688 | RPL7AP50 | ribosomal protein L7a pseudogene 50 | 0.87836 | Down in those who develop hereditary breast cancer |
| 7380 | UPK3A | uroplakin 3A | 0.96288 | Down in those who develop hereditary breast cancer |
| 84332 | DYDC2 | DPY30 domain containing 2 | 0.97996 | Down in those who develop hereditary breast cancer |
| 100132805 | [No Symbol] | [No Name] | 0.98826 | Down in those who develop hereditary breast cancer |
| 342666 | FLJ43826 | FLJ43826 protein | 0.98080 | Down in those who develop hereditary breast cancer |
| 348825 | TPRXL | tetra-peptide repeat homeobox-like | 0.98578 | Down in those who develop hereditary breast cancer |
| 388182 | FLJ42289 | hypothetical LOC388182 | 0.96063 | Down in those who develop hereditary breast cancer |
| 9241 | NOG | noggin | 0.82436 | Down in those who develop hereditary breast cancer |
| 26 | ABP1 | amiloride binding protein 1 (amine | 0.98467 | Down in those who develop hereditary breast cancer |
| | | oxidase (copper-containing)) | | |
| 26256 | CABYR | calcium binding tyrosine-(Y)- | 0.96896 | Down in those who develop hereditary breast cancer |
| | | phosphorylation regulated | | |
| 100127889 | C10orf131 | chromosome 10 open reading frame 131 | 0.96310 | Down in those who develop hereditary breast cancer |
| 100128417 | [No Symbol] | [No Name] | 0.96758 | Down in those who develop hereditary breast cancer |
| 79625 | C4orf31 | chromosome 4 open reading frame 31 | 0.98147 | Down in those who develop hereditary breast cancer |
| 83844 | USP26 | ubiquitin specific peptidase 26 | 0.96043 | Down in those who develop hereditary breast cancer |
| 8228 | PNPLA4 | patatin-like phospholipase domain containing 4 | 0.94592 | Down in those who develop hereditary breast cancer |
| 26590 | OR8B7P | olfactory receptor, family 8, subfamily | 0.97829 | Down in those who develop hereditary breast cancer |
| | | B, member 7 pseudogene | | |
| 100130859 | [No Symbol] | [No Name] | 0.95802 | Down in those who develop hereditary breast cancer |
| 400165 | C13orf35 | chromosome 13 open reading frame 35 | 0.96640 | Down in those who develop hereditary breast cancer |
| 3809 | KIR2DS4 | killer cell immunoglobulin-like receptor, | 0.90156 | Down in those who develop hereditary breast cancer |
| | | two domains, short cytoplasmic tail, 4 | | |
| 9288 | TAAR3 | trace amine associated receptor 3 (gene/pseudogene) | 0.98837 | Down in those who develop hereditary breast cancer |
| 100129958 | KRT8P44 | keratin 8 pseudogene 44 | 0.97648 | Down in those who develop hereditary breast cancer |
| 100130321 | LOC100130321 | DNA fragmentation factor, 45 kDa, alpha | 0.96556 | Down in those who develop hereditary breast cancer |
| | | polypeptide pseudogene | | |
| 100132626 | LOC100132626 | protein FAM103A1-like | 0.95852 | Down in those who develop hereditary breast cancer |
| 7644 | ZNF91 | zinc finger protein 91 | 0.91174 | Down in those who develop hereditary breast cancer |
| 144715 | RAD9B | RAD9 homolog B (S. pombe) | 0.95746 | Down in those who develop hereditary breast cancer |
| 728545 | [No Symbol] | [No Name] | 0.92791 | Down in those who develop hereditary breast cancer |
| 151825 | KRT18P43 | keratin 18 pseudogene 43 | 0.98744 | Down in those who develop hereditary breast cancer |
| 100128403 | [No Symbol] | [No Name] | 0.98170 | Down in those who develop hereditary breast cancer |
| 347051 | SLC10A5 | solute carrier family 10 (sodium/bile | 0.96335 | Down in those who develop hereditary breast cancer |
| | | acid cotransporter family), member 5 | | |
| 28869 | IGKV6D-41 | immunoglobulin kappa variable 6D-41 (non- | 0.98400 | Down in those who develop hereditary breast cancer |
| | | functional) | | |
| 387856 | C12orf68 | chromosome 12 open reading frame 68 | 0.96793 | Down in those who develop hereditary breast cancer |
| 644588 | DNAJA1P3 | DnaJ (Hsp40) homolog, subfamily A, member 1 | 0.95825 | Down in those who develop hereditary breast cancer |
| | | pseudogene 3 | | |
| 100130184 | [No Symbol] | [No Name] | 0.97631 | Down in those who develop hereditary breast cancer |
| 286495 | TTC3P1 | tetratricopeptide repeat domain 3 pseudogene 1 | 0.95788 | Down in those who develop hereditary breast cancer |
| 387924 | OGFOD1P1 | 2-oxoglutarate and iron-dependent oxygenase | 0.97365 | Down in those who develop hereditary breast cancer |
| | | domain containing 1 pseudogene 1 | | |
| 55702 | CCDC94 | coiled-coil domain containing 94 | 0.93784 | Down in those who develop hereditary breast cancer |
| 5054 | SERPINE1 | serpin peptidase inhibitor, clade E (nexin, | 0.95359 | Down in those who develop hereditary breast cancer |
| | | plasminogen activator inhibitor type 1), member 1 | | |
| 641518 | LOC641518 | hypothetical LOC641518 | 0.93179 | Down in those who develop hereditary breast cancer |
| 388507 | ZNF788 | zinc finger family member 788 | 0.96818 | Down in those who develop hereditary breast cancer |
| 7164 | TPD52L1 | tumor protein D52-like 1 | 0.99643 | Down in those who develop hereditary breast cancer |
| 57570 | TRMT5 | TRM5 tRNA methyltransferase 5 homolog | 0.92717 | Down in those who develop hereditary breast cancer |
| | | (S. cerevisiae) | | |
| 100131602 | [No Symbol] | [No Name] | 0.98327 | Down in those who develop hereditary breast cancer |
| 3812 | KIR3DL2 | killer cell immunoglobulin-like receptor, | 0.97100 | Down in those who develop hereditary breast cancer |
| | | three domains, long cytoplasmic tail, 2 | | |
| 3625 | INHBB | inhibin, beta B | 0.95366 | Down in those who develop hereditary breast cancer |
| 56891 | LGALS14 | lectin, galactoside-binding, soluble, 14 | 0.97194 | Down in those who develop hereditary breast cancer |
| 2841 | GPR18 | G protein-coupled receptor 18 | 0.90221 | Down in those who develop hereditary breast cancer |
| 645233 | LOC645233 | thymine-DNA glycosylase pseudogene | 0.89278 | Down in those who develop hereditary breast cancer |
| 166752 | FREM3 | FRAS1 related extracellular matrix 3 | 0.99312 | Down in those who develop hereditary breast cancer |
| 54035 | PSMD4P1 | proteasome (prosome, macropain) 26S | 0.94461 | Down in those who develop hereditary breast cancer |
| | | subunit, non-ATPase, 4 pseudogene 1 | | |
| 646272 | LOC646272 | cytochrome b-c1 complex subunit 8-like | 0.94686 | Down in those who develop hereditary breast cancer |
| 100133337 | [No Symbol] | [No Name] | 0.97327 | Down in those who develop hereditary breast cancer |
| 100130623 | [No Symbol] | [No Name] | 0.99692 | Down in those who develop hereditary breast cancer |
| 100132310 | LOC100132310 | FCF1 small subunit (SSU) processome | 0.96241 | Down in those who develop hereditary breast cancer |
| | | component homolog ((S. cerevisiae) pseudogene | | |
| 5988 | RFPL1 | ret finger protein-like 1 | 0.97369 | Down in those who develop hereditary breast cancer |
| 148823 | C1orf150 | chromosome 1 open reading frame 150 | 0.93649 | Down in those who develop hereditary breast cancer |
| 23754 | RPL32P5 | ribosomal protein L32 pseudogene 5 | 0.91445 | Down in those who develop hereditary breast cancer |
| 401703 | LOC401703 | splicing factor U2AF 35 kDa subunit-like | 0.96591 | Down in those who develop hereditary breast cancer |
| 1266 | CNN3 | calponin 3, acidic | 0.97602 | Down in those who develop hereditary breast cancer |
| 81889 | FAHD1 | fumarylacetoacetate hydrolase domain containing 1 | 0.93160 | Down in those who develop hereditary breast cancer |
| 339736 | AK2P2 | adenylate kinase 2 pseudogene 2 | 0.95816 | Down in those who develop hereditary breast cancer |
| 64410 | KLHL25 | kelch-like 25 (Drosophila) | 0.97533 | Down in those who develop hereditary breast cancer |
| 28466 | IGHV1-45 | immunoglobulin heavy variable 1-45 | 0.97065 | Down in those who develop hereditary breast cancer |
| 259286 | TAS2R40 | taste receptor, type 2, member 40 | 0.96512 | Down in those who develop hereditary breast cancer |
| 9363 | RAB33A | RAB33A, member RAS oncogene family | 0.97583 | Down in those who develop hereditary breast cancer |
| 729950 | LOC729950 | hypothetical LOC729950 | 0.97018 | Down in those who develop hereditary breast cancer |
| 653712 | LOC653712 | intraflagellar transport 122 homolog | 0.97822 | Down in those who develop hereditary breast cancer |
| | | (Chlamydomonas) pseudogene | | |
| 81050 | OR5AC2 | olfactory receptor, family 5, subfamily | 0.97482 | Down in those who develop hereditary breast cancer |
| | | AC, member 2 | | |
| 126259 | TMIGD2 | transmembrane and immunoglobulin domain | 0.92855 | Down in those who develop hereditary breast cancer |
| | | containing 2 | | |
| 51499 | TRIAP1 | TP53 regulated inhibitor of apoptosis 1 | 0.91981 | Down in those who develop hereditary breast cancer |
| 100129837 | [No Symbol] | [No Name] | 0.99866 | Down in those who develop hereditary breast cancer |
| 441722 | LOC441722 | U2 small nuclear RNA auxiliary factor 1 pseudogene | 0.93089 | Down in those who develop hereditary breast cancer |
| 100129046 | LOC100129046 | hypothetical LOC100129046 | 0.97470 | Down in those who develop hereditary breast cancer |
| 85352 | KIAA1644 | KIAA1644 | 0.97770 | Down in those who develop hereditary breast cancer |
| 731039 | [No Symbol] | [No Name] | 0.97693 | Down in those who develop hereditary breast cancer |
| 401588 | LOC401588 | hypothetical LOC401588 | 0.94851 | Down in those who develop hereditary breast cancer |
| 390155 | OR5T1 | olfactory receptor, family 5, subfamily T, member 1 | 0.97737 | Down in those who develop hereditary breast cancer |
| 81309 | OR4C15 | olfactory receptor, family 4, subfamily C, member 15 | 0.96608 | Down in those who develop hereditary breast cancer |
| 171472 | SRSF10P2 | serine/arginine-rich splicing factor 10 pseudogene 2 | 0.97861 | Down in those who develop hereditary breast cancer |
| 729687 | HMGN2P25 | high mobility group nucleosomal binding | 0.95508 | Down in those who develop hereditary breast cancer |
| | | domain 2 pseudogene 25 | | |
| 646895 | [No Symbol] | [No Name] | 0.91585 | Down in those who develop hereditary breast cancer |
| 51616 | TAF9B | TAF9B RNA polymerase II, TATA box binding | 0.90443 | Down in those who develop hereditary breast cancer |
| | | protein (TBP)- associated factor, 31 kDa | | |
| 113246 | C12orf57 | chromosome 12 open reading frame 57 | 0.86256 | Down in those who develop hereditary breast cancer |
| 100131568 | LOC100131568 | RNA binding motif protein 43 pseudogene | 0.96482 | Down in those who develop hereditary breast cancer |
| 730109 | LOC730109 | hypothetical protein LOC730109 | 0.98596 | Down in those who develop hereditary breast cancer |
| 55604 | LRRC16A | leucine rich repeat containing 16A | 0.97138 | Down in those who develop hereditary breast cancer |
| 119391 | GSTO2 | glutathione S-transferase omega 2 | 0.97955 | Down in those who develop hereditary breast cancer |
| 54033 | RBM11 | RNA binding motif protein 11 | 0.94485 | Down in those who develop hereditary breast cancer |
| 1068 | CETN1 | centrin, EF-hand protein, 1 | 0.95410 | Down in those who develop hereditary breast cancer |
| 9397 | NMT2 | N-myristoyltransferase 2 | 0.92727 | Down in those who develop hereditary breast cancer |
| 729868 | [No Symbol] | [No Name] | 0.97418 | Down in those who develop hereditary breast cancer |
| 153893 | LOC153893 | DCN1, defective in cullin neddylation 1, domain | 0.97614 | Down in those who develop hereditary breast cancer |
| | | containing 1 (S. cerevisiae) pseudogene | | |
| 80032 | ZNF556 | zinc finger protein 556 | 0.97780 | Down in those who develop hereditary breast cancer |
| 400360 | C15orf54 | chromosome 15 open reading frame 54 | 0.84923 | Down in those who develop hereditary breast cancer |
| 7643 | ZNF90 | zinc finger protein 90 | 0.97669 | Down in those who develop hereditary breast cancer |
| 64838 | FNDC4 | fibronectin type III domain containing 4 | 0.99459 | Down in those who develop hereditary breast cancer |
| 285484 | LOC285484 | hypothetical LOC285484 | 0.97920 | Down in those who develop hereditary breast cancer |
| 90499 | LOC90499 | hypothetical protein LOC90499 | 0.94649 | Down in those who develop hereditary breast cancer |
| 2945 | GSTM5P1 | glutathione S-transferase mu 5 pseudogene 1 | 0.97316 | Down in those who develop hereditary breast cancer |
| 1424 | CRYGGP | crystallin, gamma G, pseudogene | 0.96815 | Down in those who develop hereditary breast cancer |
| 3346 | HTN1 | histatin 1 | 0.98200 | Down in those who develop hereditary breast cancer |
| 10127 | ZNF263 | zinc finger protein 263 | 0.93963 | Down in those who develop hereditary breast cancer |
| 131572 | MESTP4 | mesoderm specific transcript homolog | 0.97671 | Down in those who develop hereditary breast cancer |
| | | (mouse) pseudogene 4 | | |
| 25927 | CNRIP1 | cannabinoid receptor interacting protein 1 | 0.98837 | Down in those who develop hereditary breast cancer |
| 4925 | NUCB2 | nucleobindin 2 | 0.91190 | Down in those who develop hereditary breast cancer |
| 236 | AKR1B1P2 | aldo-keto reductase family 1, member | 0.96975 | Down in those who develop hereditary breast cancer |
| | | B1 pseudogene 2 | | |
| 147657 | ZNF480 | zinc finger protein 480 | 0.91736 | Down in those who develop hereditary breast cancer |
| 100128122 | LOC100128122 | Cas-Br-M (murine) ecotropic retroviral | 0.97337 | Down in those who develop hereditary breast cancer |
| | | transforming sequence-like 1 pseudogene | | |
| 123862 | LOC123862 | interferon induced transmembrane protein | 0.95430 | Down in those who develop hereditary breast cancer |
| | | pseudogene | | |
| 6555 | SLC10A2 | solute carrier family 10 (sodium/bile | 0.98761 | Down in those who develop hereditary breast cancer |
| | | acid cotransporter family), member 2 | | |

TABLE 4
Utah2 FC Summary Up

| Entrez | | | Fold | |
|---|---|---|---|---|
| Gene ID | Gene Symbol | Gene Name | Change | Description |
| 55057 | AIM1L | absent in melanoma 1-like | 1.01575 | Up in those who develop hereditary breast cancer |
| 148641 | SLC35F3 | solute carrier family 35, member F3 | 1.01311 | Up in those who develop hereditary breast cancer |
| 647298 | HSPD1P8 | heat shock 60 kDa protein 1 (chaperonin) pseudogene 8 | 1.01504 | Up in those who develop hereditary breast cancer |
| 392282 | RPS5P6 | ribosomal protein S5 pseudogene 6 | 1.02827 | Up in those who develop hereditary breast cancer |
| 389428 | RPL5P18 | ribosomal protein L5 pseudogene 18 | 1.05885 | Up in those who develop hereditary breast cancer |
| 100130249 | PP2672 | hypothetical LOC100130249 | 1.04144 | Up in those who develop hereditary breast cancer |
| 163778 | SPRR4 | small proline-rich protein 4 | 1.05947 | Up in those who develop hereditary breast cancer |
| 157927 | C9orf62 | chromosome 9 open reading frame 62 | 1.02656 | Up in those who develop hereditary breast cancer |
| 83604 | TMEM47 | transmembrane protein 47 | 1.02085 | Up in those who develop hereditary breast cancer |
| 729486 | IL9RP3 | interleukin 9 receptor pseudogene 3 | 1.04554 | Up in those who develop hereditary breast cancer |
| 286122 | C8orf31 | chromosome 8 open reading frame 31 | 1.02932 | Up in those who develop hereditary breast cancer |
| 400954 | EML6 | echinoderm microtubule associated protein like 6 | 1.02327 | Up in those who develop hereditary breast cancer |
| 100128700 | [No Symbol] | [No Name] | 1.03000 | Up in those who develop hereditary breast cancer |
| 730126 | [No Symbol] | [No Name] | 1.03615 | Up in those who develop hereditary breast cancer |
| 644662 | LOC644662 | hypothetical protein LOC644662 | 1.01804 | Up in those who develop hereditary breast cancer |
| 785 | CACNB4 | calcium channel, voltage-dependent, beta 4 subunit | 1.01724 | Up in those who develop hereditary breast cancer |
| 149157 | [No Symbol] | [No Name] | 1.02750 | Up in those who develop hereditary breast cancer |
| 347333 | KRT8P14 | keratin 8 pseudogene 14 | 1.00982 | Up in those who develop hereditary breast cancer |
| 100128386 | LOC100128386 | hypothetical LOC100128386 | 1.02361 | Up in those who develop hereditary breast cancer |
| 100132086 | LOC100132086 | adenylate kinase isoenzyme 6-like | 1.02578 | Up in those who develop hereditary breast cancer |
| 121270 | OR11M1P | olfactory receptor, family 11, subfamily | 1.06053 | Up in those who develop hereditary breast cancer |
| | | M, member 1 pseudogene | | |
| 408029 | C2orf27B | chromosome 2 open reading frame 27B | 1.02973 | Up in those who develop hereditary breast cancer |
| 6461 | SHB | Src homology 2 domain containing adaptor protein B | 1.01215 | Up in those who develop hereditary breast cancer |
| 27145 | FILIP1 | filamin A interacting protein 1 | 1.02313 | Up in those who develop hereditary breast cancer |
| 401433 | LOC401433 | hypothetical LOC401433 | 1.03820 | Up in those who develop hereditary breast cancer |
| 100131042 | [No Symbol] | [No Name] | 1.04994 | Up in those who develop hereditary breast cancer |
| 646576 | LOC646576 | hypothetical LOC646576 | 1.00778 | Up in those who develop hereditary breast cancer |
| 10877 | CFHR4 | complement factor H-related 4 | 1.03294 | Up in those who develop hereditary breast cancer |
| 3394 | IRF8 | interferon regulatory factor 8 | 1.03235 | Up in those who develop hereditary breast cancer |
| 100128493 | LOC100128493 | ubiquitin-conjugating enzyme E2 variant 2 pseudogene | 1.03824 | Up in those who develop hereditary breast cancer |
| 440603 | BCL2L15 | BCL2-like 15 | 1.02400 | Up in those who develop hereditary breast cancer |
| 728050 | [No Symbol] | [No Name] | 1.08112 | Up in those who develop hereditary breast cancer |
| 649288 | AK4P6 | adenylate kinase 4 pseudogene 6 | 1.03149 | Up in those who develop hereditary breast cancer |
| 100128979 | LOC100128979 | hypothetical LOC100128979 | 1.03901 | Up in those who develop hereditary breast cancer |
| 100128457 | LOC100128457 | similar to hCG2026341 | 1.04522 | Up in those who develop hereditary breast cancer |
| 283553 | LOC283553 | hypothetical LOC283553 | 1.01435 | Up in those who develop hereditary breast cancer |
| 199713 | NLRP7 | NLR family, pyrin domain containing 7 | 1.03336 | Up in those who develop hereditary breast cancer |
| 2596 | GAP43 | growth associated protein 43 | 1.01796 | Up in those who develop hereditary breast cancer |
| 441505 | LOC441505 | stress-induced-phosphoprotein 1 pseudogene | 1.00827 | Up in those who develop hereditary breast cancer |
| 643586 | LOC643586 | pyruvate kinase, muscle pseudogene | 1.03424 | Up in those who develop hereditary breast cancer |
| 449518 | LOC449518 | purinergic receptor P2Y, G-protein coupled, | 1.04589 | Up in those who develop hereditary breast cancer |
| | | 10 pseudogene | | |
| 3426 | CFI | complement factor I | 1.01805 | Up in those who develop hereditary breast cancer |
| 128774 | MRPS11P1 | mitochondrial ribosomal protein S11 pseudogene 1 | 1.03087 | Up in those who develop hereditary breast cancer |
| 100129915 | [No Symbol] | [No Name] | 1.01662 | Up in those who develop hereditary breast cancer |
| 6862 | T | T, brachyury homolog (mouse) | 1.01862 | Up in those who develop hereditary breast cancer |
| 1472 | CST4 | cystatin S | 1.05145 | Up in those who develop hereditary breast cancer |
| 4342 | MOS | v-mos Moloney murine sarcoma viral oncogene homolog | 1.01892 | Up in those who develop hereditary breast cancer |
| 78998 | C8orf51 | chromosome 8 open reading frame 51 | 1.03279 | Up in those who develop hereditary breast cancer |
| 780813 | PAICSP4 | phosphoribosylaminoimidazole carboxylase, | 1.01634 | Up in those who develop hereditary breast cancer |
| | | phosphoribosylaminoimidazole succinocarboxamide | | |
| | | synthetase pseudogene 4 | | |
| 55540 | IL17RB | interleukin 17 receptor B | 1.01748 | Up in those who develop hereditary breast cancer |
| 100128709 | [No Symbol] | [No Name] | 1.05850 | Up in those who develop hereditary breast cancer |
| 100130268 | LOC100130268 | similar to hCG1648866 | 1.03898 | Up in those who develop hereditary breast cancer |
| 100128389 | [No Symbol] | [No Name] | 1.04353 | Up in those who develop hereditary breast cancer |
| 353194 | LOC353194 | keratin pseudogene | 1.03225 | Up in those who develop hereditary breast cancer |
| 100131609 | HNRNPA1P2 | heterogeneous nuclear ribonucleoprotein | 1.05136 | Up in those who develop hereditary breast cancer |
| | | A1 pseudogene 2 | | |
| 130500 | CISD1P1 | CDGSH iron sulfur domain 1 pseudogene 1 | 1.03521 | Up in those who develop hereditary breast cancer |
| 128820 | CST9LP1 | cystatin 9-like pseudogene 1 | 1.03984 | Up in those who develop hereditary breast cancer |
| 100132214 | [No Symbol] | [No Name] | 1.04735 | Up in those who develop hereditary breast cancer |
| 26647 | OR7E25P | olfactory receptor, family 7, subfamily | 1.03869 | Up in those who develop hereditary breast cancer |
| | | E, member 25 pseudogene | | |
| 729041 | LOC729041 | fatty-acid amide hydrolase 1-like | 1.04385 | Up in those who develop hereditary breast cancer |
| 221711 | SYCP2L | synaptonemal complex protein 2-like | 1.03038 | Up in those who develop hereditary breast cancer |
| 4157 | MC1R | melanocortin 1 receptor (alpha melanocyte | 1.03616 | Up in those who develop hereditary breast cancer |
| | | stimulating hormone receptor) | | |
| 10461 | MERTK | c-mer proto-oncogene tyrosine kinase | 1.03881 | Up in those who develop hereditary breast cancer |
| 729011 | [No Symbol] | [No Name] | 1.03000 | Up in those who develop hereditary breast cancer |
| 649489 | LOC649489 | protein phosphatase 1, regulatory | 1.02506 | Up in those who develop hereditary breast cancer |
| | | (inhibitor) subunit 2 pseudogene | | |
| 4744 | NEFH | neurofilament, heavy polypeptide | 1.01566 | Up in those who develop hereditary breast cancer |
| 7070 | THY1 | Thy-1 cell surface antigen | 1.01532 | Up in those who develop hereditary breast cancer |
| 653194 | LOC653194 | KH homology domain-containing protein 1-like | 1.03238 | Up in those who develop hereditary breast cancer |
| 7473 | WNT3 | wingless-type MMTV integration site family, member 3 | 1.01553 | Up in those who develop hereditary breast cancer |
| 283314 | MATL2963 | hypothetical LOC283314 | 1.03595 | Up in those who develop hereditary breast cancer |
| 647532 | LOC647532 | phenylalanine-tRNA synthetase-like, beta | 1.02208 | Up in those who develop hereditary breast cancer |
| | | subunit pseudogene | | |
| 644862 | RPS28P3 | ribosomal protein S28 pseudogene 3 | 1.03776 | Up in those who develop hereditary breast cancer |
| 3626 | INHBC | inhibin, beta C | 1.02582 | Up in those who develop hereditary breast cancer |
| 283571 | PROX2 | prospero homeobox 2 | 1.01521 | Up in those who develop hereditary breast cancer |
| 100132510 | GLRXP3 | glutaredoxin (thioltransferase) pseudogene 3 | 1.03741 | Up in those who develop hereditary breast cancer |
| 25925 | ZNF521 | zinc finger protein 521 | 1.01135 | Up in those who develop hereditary breast cancer |
| 606495 | CYB5RL | cytochrome b5 reductase-like | 1.00749 | Up in those who develop hereditary breast cancer |
| 56834 | GPR137 | G protein-coupled receptor 137 | 1.00139 | Up in those who develop hereditary breast cancer |
| 574447 | MIR146B | microRNA 146b | 1.03471 | Up in those who develop hereditary breast cancer |
| 8626 | TP63 | tumor protein p63 | 1.01222 | Up in those who develop hereditary breast cancer |
| 390844 | ARIH2P1 | ariadne homolog 2 pseudogene 1 | 1.01615 | Up in those who develop hereditary breast cancer |
| 4085 | MAD2L1 | MAD2 mitotic arrest deficient-like 1 (yeast) | 1.01352 | Up in those who develop hereditary breast cancer |
| 100131070 | LOC100131070 | mpv17-like protein 2-like | 1.03546 | Up in those who develop hereditary breast cancer |
| 338094 | FAM151A | family with sequence similarity 151, member A | 1.02257 | Up in those who develop hereditary breast cancer |
| 339983 | NAT8L | N-acetyltransferase 8-like (GCN5-related, putative) | 1.02554 | Up in those who develop hereditary breast cancer |
| 100132053 | RPL30P8 | ribosomal protein L30 pseudogene 8 | 1.01456 | Up in those who develop hereditary breast cancer |
| 100131750 | [No Symbol] | [No Name] | 1.02281 | Up in those who develop hereditary breast cancer |
| 285231 | FBXW12 | F-box and WD repeat domain containing 12 | 1.02713 | Up in those who develop hereditary breast cancer |
| 400347 | LOC400347 | REX4, RNA exonuclease 4 homolog | 1.03336 | Up in those who develop hereditary breast cancer |
| | | (S. cerevisiae) pseudogene | | |
| 6887 | TAL2 | T-cell acute lymphocytic leukemia 2 | 1.03352 | Up in those who develop hereditary breast cancer |
| 201895 | C4orf34 | chromosome 4 open reading frame 34 | 1.02999 | Up in those who develop hereditary breast cancer |
| 442673 | TUBG1P | tubulin, gamma 1 pseudogene | 1.04787 | Up in those who develop hereditary breast cancer |
| 392301 | SLC25A5P8 | solute carrier family 25 (mitochondrial carrier; | 1.02652 | Up in those who develop hereditary breast cancer |
| | | adenine nucleotide translocator), member 5 pseudogene 8 | | |
| 414318 | C9orf106 | chromosome 9 open reading frame 106 | 1.03024 | Up in those who develop hereditary breast cancer |
| 23439 | ATP1B4 | ATPase, Na+/K+ transporting, beta 4 polypeptide | 1.01288 | Up in those who develop hereditary breast cancer |
| 219902 | TMEM136 | transmembrane protein 136 | 1.01357 | Up in those who develop hereditary breast cancer |
| 79825 | CCDC48 | coiled-coil domain containing 48 | 1.01918 | Up in those who develop hereditary breast cancer |
| 643669 | LOC643669 | hypothetical protein LOC643669 | 1.01596 | Up in those who develop hereditary breast cancer |
| 145259 | RPSAP4 | ribosomal protein SA pseudogene 4 | 1.01434 | Up in those who develop hereditary breast cancer |
| 2335 | FN1 | fibronectin 1 | 1.02499 | Up in those who develop hereditary breast cancer |
| 81849 | ST6GALNAC5 | ST6 (alpha-N-acetyl-neuraminyl-2,3-beta-galactosyl- | 1.01527 | Up in those who develop hereditary breast cancer |
| | | 1,3)-N-acetylgalactosaminide alpha-2,6- | | |
| | | sialyltransferase 5 | | |
| 100132073 | LOC100132073 | cyclin B2 pseudogene | 1.03497 | Up in those who develop hereditary breast cancer |
| 84099 | ID2B | inhibitor of DNA binding 2B, dominant negative helix- | 1.02669 | Up in those who develop hereditary breast cancer |
| | | loop-helix protein (pseudogene) | | |
| 644464 | RPSAP61 | ribosomal protein SA pseudogene 61 | 1.04385 | Up in those who develop hereditary breast cancer |
| 130162 | C2orf63 | chromosome 2 open reading frame 63 | 1.01620 | Up in those who develop hereditary breast cancer |
| 10316 | NMUR1 | neuromedin U receptor 1 | 1.05132 | Up in those who develop hereditary breast cancer |
| 728780 | ANKDD1B | ankyrin repeat and death domain | 1.01894 | Up in those who develop hereditary breast cancer |
| | | containing 1B | | |
| 643182 | LOC643182 | upstream binding transcription factor, | 1.01711 | Up in those who develop hereditary breast cancer |
| | | RNA polymerase I pseudogene | | |
| 344807 | CD200R1L | CD200 receptor 1-like | 1.01802 | Up in those who develop hereditary breast cancer |
| 140691 | TRIM69 | tripartite motif containing 69 | 1.02117 | Up in those who develop hereditary breast cancer |
| 644915 | METTL15P2 | methyltransferase like 15 pseudogene 2 | 1.01990 | Up in those who develop hereditary breast cancer |
| 139411 | PTCHD1 | patched domain containing 1 | 1.01407 | Up in those who develop hereditary breast cancer |
| 118663 | BTBD16 | BTB (POZ) domain containing 16 | 1.01455 | Up in those who develop hereditary breast cancer |
| 285987 | DLX6-AS1 | DLX6 antisense RNA 1 (non-protein coding) | 1.02260 | Up in those who develop hereditary breast cancer |
| 130013 | ACMSD | aminocarboxymuconate semialdehyde decarboxylase | 1.02182 | Up in those who develop hereditary breast cancer |
| 645474 | S100A11P3 | S100 calcium binding protein A11 pseudogene 3 | 1.03471 | Up in those who develop hereditary breast cancer |
| 3226 | HOXC10 | homeobox C10 | 1.01038 | Up in those who develop hereditary breast cancer |
| 392387 | LOC392387 | adenosylhomocysteinase pseudogene | 1.00870 | Up in those who develop hereditary breast cancer |
| 81431 | OR5AC1 | olfactory receptor, family 5, subfamily | 1.00021 | Up in those who develop hereditary breast cancer |
| | | AC, member 1 (gene/pseudogene) | | |
| 79696 | FAM164C | family with sequence similarity 164, member C | 1.01792 | Up in those who develop hereditary breast cancer |
| 100131087 | RPLP1P11 | ribosomal protein, large, P1 pseudogen 11 | 1.03187 | Up in those who develop hereditary breast cancer |
| 644941 | [No Symbol] | [No Name] | 1.06134 | Up in those who develop hereditary breast cancer |
| 100127983 | LOC100127983 | hypothetical protein LOC100127983 | 1.00087 | Up in those who develop hereditary breast cancer |
| 100129316 | LOC100129316 | hypothetical LOC100129316 | 1.02946 | Up in those who develop hereditary breast cancer |
| 806 | CALM2P2 | calmodulin 2 pseudogene 2 | 1.03895 | Up in those who develop hereditary breast cancer |
| 644489 | [No Symbol] | [No Name] | 1.03232 | Up in those who develop hereditary breast cancer |
| 100129568 | [No Symbol] | [No Name] | 1.01209 | Up in those who develop hereditary breast cancer |
| 6276 | S100A5 | S100 calcium binding protein A5 | 1.02479 | Up in those who develop hereditary breast cancer |
| 100129427 | LOC100129427 | hypothetical LOC100129427 | 1.04063 | Up in those who develop hereditary breast cancer |
| 163183 | C19orf46 | chromosome 19 open reading frame 46 | 1.02242 | Up in those who develop hereditary breast cancer |
| 51676 | ASB2 | ankyrin repeat and SOCS box containing 2 | 1.01372 | Up in those who develop hereditary breast cancer |
| 157773 | C8orf48 | chromosome 8 open reading frame 48 | 1.02024 | Up in those who develop hereditary breast cancer |
| 2151 | F2RL2 | coagulation factor II (thrombin) receptor-like 2 | 1.02926 | Up in those who develop hereditary breast cancer |
| 5999 | RGS4 | regulator of G-protein signaling 4 | 1.02586 | Up in those who develop hereditary breast cancer |
| 993 | CDC25A | cell division cycle 25 homolog A (S. pombe) | 1.01648 | Up in those who develop hereditary breast cancer |
| 442192 | DDX6P1 | DEAD (Asp-Glu-Ala-Asp) box polypeptide 6 | 1.00649 | Up in those who develop hereditary breast cancer |
| | | pseudogene 1 | | |
| 255403 | ZNF718 | zinc finger protein 718 | 1.03097 | Up in those who develop hereditary breast cancer |
| 338557 | O3FAR1 | omega-3 fatty acid receptor 1 | 1.00668 | Up in those who develop hereditary breast cancer |
| 9037 | SEMA5A | sema domain, seven thrombospondin repeats (type 1 and | 1.01422 | Up in those who develop hereditary breast cancer |
| | | type 1-like), transmembrane domain (TM) and short | | |
| | | cytoplasmic domain, (semaphorin) 5A | | |
| 442709 | EEF1A1P28 | eukaryotic translation elongation factor 1 alpha 1 | 1.02390 | Up in those who develop hereditary breast cancer |
| | | pseudogene 28 | | |
| 80097 | MZT2B | mitotic spindle organizing protein 2B | 1.03085 | Up in those who develop hereditary breast cancer |
| 57016 | AKR1B10 | aldo-keto reductase family 1, member B10 | 1.03090 | Up in those who develop hereditary breast cancer |
| | | (aldose reductase) | | |
| 50835 | TAS2R9 | taste receptor, type 2, member 9 | 1.02523 | Up in those who develop hereditary breast cancer |
| 442213 | C6orf138 | chromosome 6 open reading frame 138 | 1.01107 | Up in those who develop hereditary breast cancer |
| 100131991 | RPL29P8 | ribosomal protein L29 pseudogene 8 | 1.02907 | Up in those who develop hereditary breast cancer |
| 1553 | CYP2A13 | cytochrome P450, family 2, subfamily A, polypeptide 13 | 1.02619 | Up in those who develop hereditary breast cancer |
| 646808 | LOC646808 | L antigen family member 3-like | 1.00468 | Up in those who develop hereditary breast cancer |
| 100128956 | [No Symbol] | [No Name] | 1.01327 | Up in those who develop hereditary breast cancer |
| 286544 | MXRA5P1 | matrix-remodelling associated 5 pseudogene 1 | 1.01832 | Up in those who develop hereditary breast cancer |
| 392288 | LOC392288 | micro tubule-associated proteins 1A/1B | 1.02050 | Up in those who develop hereditary breast cancer |
| | | light chain 3B-like | | |
| 646784 | RPL5P6 | ribosomal protein L5 pseudogene 6 | 1.02334 | Up in those who develop hereditary breast cancer |
| 653794 | TRIM60P14 | tripartite motif containing 60 pseudogene 14 | 1.01707 | Up in those who develop hereditary breast cancer |
| 728962 | RPL7P56 | ribosomal protein L7 pseudogene 56 | 1.01247 | Up in those who develop hereditary breast cancer |
| 1769 | DNAH8 | dynein, axonemal, heavy chain 8 | 1.01179 | Up in those who develop hereditary breast cancer |
| 25789 | TMEM59L | transmembrane protein 59-like | 1.00625 | Up in those who develop hereditary breast cancer |
| 221833 | SP8 | Sp8 transcription factor | 1.01794 | Up in those who develop hereditary breast cancer |
| 100131819 | [No Symbol] | [No Name] | 1.02809 | Up in those who develop hereditary breast cancer |
| 100133303 | [No Symbol] | [No Name] | 1.00567 | Up in those who develop hereditary breast cancer |

TABLE 5
PI3K Genes Patent

| | | | Utah Relative | Ontario1 Relative | Ontario2 Relative |
|---|---|---|---|---|---|
| Entrez | | | Expression In BC | Expression In BC | Expression In BC |
| Gene ID | Gene Symbol | Gene Name | Individuals | Individuals | Individuals |
| 801 | CALM1 | calmodulin 1 (phosphorylase kinase, delta) | −0.005868907 | −0.034288056 | −0.073209514 |
| 805 | CALM2 | calmodulin 2 (phosphorylase kinase, delta) | 0.029884779 | −0.103366242 | −0.018766879 |
| 808 | CALM3 | calmodulin 3 (phosphorylase kinase, delta) | −0.041808313 | −0.056287265 | 0.014481458 |
| 810 | CALML3 | calmodulin-like 3 | −0.009634626 | 0.004471252 | −0.024717361 |
| 51806 | CALML5 | calmodulin-like 5 | −0.02286605 | 0.021140162 | 0.03916656 |
| 163688 | CALML6 | calmodulin-like 6 | −0.008166721 | −0.015273123 | −0.005577405 |
| 10423 | CDIPT | CDP-diacylglycerol--inositol 3-phosphatidyltransferase | −0.051092172 | −0.072262869 | 0.042163104 |
| 1040 | CDS1 | CDP-diacylglycerol synthase (phosphatidate | −0.003726478 | −0.010667818 | −0.011381363 |
| | | cytidylyltransferase) 1 | | | |
| 8760 | CDS2 | CDP-diacylglycerol synthase (phosphatidate | −0.036125825 | 0.012370354 | −0.059466994 |
| | | cytidylyltransferase) 2 | | | |
| 1606 | DGKA | diacylglycerol kinase, alpha 80 kDa | −0.05841798 | −0.011296983 | −0.099703428 |
| 1607 | DGKB | diacylglycerol kinase, beta 90 kDa | 0.005001732 | −0.000621839 | 0.006532214 |
| 8527 | DGKD | diacylglycerol kinase, delta 130 kDa | −0.001969979 | 0.071182927 | −0.028379999 |
| 8526 | DGKE | diacylglycerol kinase, epsilon 64 kDa | −0.058271652 | −0.046321391 | −0.026491162 |
| 1608 | DGKG | diacylglycerol kinase, gamma 90 kDa | −0.006297576 | −0.015694053 | 0.00316159 |
| 160851 | DGKH | diacylglycerol kinase, eta | −0.030362684 | −0.021075694 | 0.071652238 |
| 9162 | DGKI | diacylglycerol kinase, iota | 0.011890312 | 0.003042886 | 0.029705028 |
| 1609 | DGKQ | diacylglycerol kinase, theta 110 kDa | −0.014207833 | −0.021745181 | 0.003026248 |
| 8525 | DGKZ | diacylglycerol kinase, zeta | −0.009531233 | 0.011038832 | −0.035008789 |
| 3612 | IMPA1 | inositol(myo)-1(or 4)-monophosphatase 1 | −0.0465624 | −0.032847849 | 0.076108587 |
| 3613 | IMPA2 | inositol(myo)-1(or 4)-monophosphatase 2 | −0.018530574 | −0.008767401 | −0.000808967 |
| 3628 | INPP1 | inositol polyphosphate-1-phosphatase | −0.002667501 | −0.015891572 | 0.021452741 |
| 3631 | INPP4A | inositol polyphosphate-4-phosphatase, type I, 107 kDa | 0.008964483 | −0.014970733 | −0.016228805 |
| 8821 | INPP4B | inositol polyphosphate-4-phosphatase, type II, 105 kDa | −0.02765817 | −0.065399565 | −0.047857731 |
| 3632 | INPP5A | inositol polyphosphate-5-phosphatase, 40 kDa | −0.016320182 | 0.017577047 | −0.038410834 |
| 3633 | INPP5B | inositol polyphosphate-5-phosphatase, 75 kDa | −0.010176361 | 0.002324463 | 0.096761886 |
| 3635 | INPP5D | inositol polyphosphate-5-phosphatase, 145 kDa | 0.01611884 | −0.000221938 | −0.014274004 |
| 56623 | INPP5E | inositol polyphosphate-5-phosphatase, 72 kDa | −0.024859523 | −0.013070128 | 0.063305418 |
| 27124 | INPP5J | inositol polyphosphate-5-phosphatase J | −0.012710432 | −0.008897664 | −0.005971657 |
| 51763 | INPP5K | inositol polyphosphate-5-phosphatase K | −0.036774928 | −0.039704729 | −0.077983112 |
| 3636 | INPPL1 | inositol polyphosphate phosphatase-like 1 | −0.031696342 | 0.078589508 | −0.005371941 |
| 64768 | IPPK | inositol 1,3,4,5,6-pentakisphosphate 2-kinase | −0.027186023 | 0.010787434 | 0.039800144 |
| 3705 | ITPK1 | inositol-tetrakisphosphate 1-kinase | −0.00310463 | −0.018788231 | 0.002272978 |
| 3706 | ITPKA | inositol-trisphosphate 3-kinase A | −0.013390543 | 0.003602212 | 0.05420415 |
| 3707 | ITPKB | inositol-trisphosphate 3-kinase B | −0.022382451 | 0.024313804 | −0.103537846 |
| 3708 | ITPR1 | inositol 1,4,5-trisphosphate receptor, type 1 | −0.00738637 | −0.023494011 | 0.012953349 |
| 3709 | ITPR2 | inositol 1,4,5-trisphosphate receptor, type 2 | −0.023597782 | −0.027884427 | 0.023891588 |
| 3710 | ITPR3 | inositol 1,4,5-trisphosphate receptor, type 3 | −0.010921738 | 0.010189935 | 0.02014193 |
| 4952 | OCRL | oculocerebrorenal syndrome of Lowe | −0.021285205 | −0.03436778 | 0.031803097 |
| 55361 | PI4K2A | phosphatidylinositol 4-kinase type 2 alpha | −0.054616219 | −0.044763903 | −0.080477411 |
| 55300 | PI4K2B | phosphatidylinositol 4-kinase type 2 beta | −0.009969065 | 0.028044147 | 0.079730218 |
| 5297 | PI4KA | phosphatidylinositol 4-kinase, catalytic, alpha | −0.019377488 | −0.010899036 | −0.040647267 |
| 5298 | PI4KB | phosphatidylinositol 4-kinase, catalytic, beta | 0.004723306 | −0.021284618 | 0.037429493 |
| 5286 | PIK3C2A | phosphoinositide-3-kinase, class 2, alpha polypeptide | −0.046132703 | 0.027112212 | −0.007622418 |
| 5287 | PIK3C2B | phosphoinositide-3-kinase, class 2, beta polypeptide | −0.027065318 | 0.045285642 | 0.050845173 |
| 5288 | PIK3C2G | phosphoinositide-3-kinase, class 2, gamma polypeptide | 0.002314968 | 0.010986899 | −0.004823366 |
| 5289 | PIK3C3 | phosphoinositide-3-kinase, class 3 | −0.034725126 | −0.012781531 | 0.041160711 |
| 5290 | PIK3CA | phosphoinositide-3-kinase, catalytic, alpha polypeptide | −0.018855838 | −0.032719808 | −0.038543872 |
| 5291 | PIK3CB | phosphoinositide-3-kinase, catalytic, beta polypeptide | −0.017621415 | −0.003059229 | −0.089249522 |
| 5293 | PIK3CD | phosphoinositide-3-kinase, catalytic, delta polypeptide | −0.021024029 | −0.034590864 | −4.50E−05 |
| 5294 | PIK3CG | phosphoinositide-3-kinase, catalytic, gamma polypeptide | 0.022809021 | −0.02426685 | −0.050973145 |
| 5295 | PIK3R1 | phosphoinositide-3-kinase, regulatory subunit 1 (alpha) | 0.015282806 | −0.022171077 | −0.10639986 |
| 5296 | PIK3R2 | phosphoinositide-3-kinase, regulatory subunit 2 (beta) | −0.042329239 | −0.057456372 | 0.025274178 |
| 8503 | PIK3R3 | phosphoinositide-3-kinase, regulatory subunit 3 (gamma) | 0.01033469 | 0.023964076 | 0.099971866 |
| 23533 | PIK3R5 | phosphoinositide-3-kinase, regulatory subunit 5 | −0.030385677 | 0.101135589 | −0.074130345 |
| 200576 | PIKFYVE | phosphoinositide kinase, FYVE finger containing | −0.003144749 | 0.000187523 | −0.053325366 |
| 5305 | PIP4K2A | phosphatidylinositol-5-phosphate 4-kinase, type II, alpha | −0.008173273 | 0.078190797 | 0.026167758 |
| 8396 | PIP4K2B | phosphatidylinositol-5-phosphate 4-kinase, type II, beta | −0.042621947 | −0.037870838 | 0.070678669 |
| 79837 | PIP4K2C | phosphatidylinositol-5-phosphate 4-kinase, type II, gamma | −0.041639739 | −0.049045079 | 0.040141175 |
| 8394 | PIP5K1A | phosphatidylinositol-4-phosphate 5-kinase, type I, alpha | 0.009675317 | 0.029828603 | −0.026438165 |
| 8395 | PIP5K1B | phosphatidylinositol-4-phosphate 5-kinase, type I, beta | −0.008933258 | −0.014580712 | 0.054372873 |
| 23396 | PIP5K1C | phosphatidylinositol-4-phosphate 5-kinase, type I, gamma | −0.036626462 | 0.040091056 | −0.000379232 |
| 23236 | PLCB1 | phospholipase C, beta 1 (phosphoinositide-specific) | −0.022026224 | −0.019811055 | −0.02677464 |
| 5330 | PLCB2 | phospholipase C, beta 2 | 0.009487544 | −0.040789436 | −0.033308453 |
| 5331 | PLCB3 | phospholipase C, beta 3 (phosphatidylinositol-specific) | −0.030709073 | 0.057247717 | −0.034208085 |
| 5332 | PLCB4 | phospholipase C, beta 4 | −0.005747913 | 0.002829279 | −0.001976635 |
| 5333 | PLCD1 | phospholipase C, delta 1 | −0.018069548 | −0.026657873 | −0.045274097 |
| 113026 | PLCD3 | phospholipase C, delta 3 | −0.014037564 | 0.016192575 | 0.030545611 |
| 84812 | PLCD4 | phospholipase C, delta 4 | 0.005752336 | 0.002104489 | −0.001642951 |
| 51196 | PLCE1 | phospholipase C, epsilon 1 | 0.004952386 | −0.00168187 | 0.004303058 |
| 5335 | PLCG1 | phospholipase C, gamma 1 | −0.06331 | 0.007470207 | 0.02426554 |
| 5336 | PLCG2 | phospholipase C, gamma 2 (phosphatidylinositol-specific) | −0.004092373 | 0.058748801 | −0.011447829 |
| 89869 | PLCZ1 | phospholipase C, zeta 1 | 0.008462462 | 0.017562649 | 0.004098731 |
| 5578 | PRKCA | protein kinase C, alpha | −0.040169309 | −0.067892309 | −0.077256745 |
| 5579 | PRKCB | protein kinase C, beta | −0.023480781 | −0.020362346 | −0.023903074 |
| 5582 | PRKCG | protein kinase C, gamma | −0.014498324 | 0.006776302 | 0.00013148 |
| 5728 | PTEN | phosphatase and tensin homolog | −0.010654453 | 0.02782015 | 0.128758281 |
| 8867 | SYNJ1 | synaptojanin 1 | −0.021592507 | 0.001719874 | −0.06096483 |
| 8871 | SYNJ2 | synaptojanin 2 | −0.02094889 | −0.005098898 | −0.037346223 |

TABLE 6
CellAdhesion Genes

| | | | Utah Relative | Ontario1 Relative | Ontario2 Relative |
|---|---|---|---|---|---|
| Entrez | | | Expression In | Expression In | Expression In |
| Gene ID | Gene Symbol | Gene Name | BC Individuals | BC Individuals | BC Individuals |
| 214 | ALCAM | activated leukocyte cell adhesion molecule | −0.006460525 | −0.014497025 | 0.054872502 |
| 23705 | CADM1 | cell adhesion molecule 1 | 0.00110034 | 0.01758329 | 0.043183389 |
| 57863 | CADM3 | cell adhesion molecule 3 | −0.003951303 | −0.015251455 | 0.007634349 |
| 914 | CD2 | CD2 molecule | −0.020702602 | −0.006600216 | −0.113551569 |
| 10666 | CD226 | CD226 molecule | −0.048948745 | −0.037742755 | 0.076472212 |
| 29126 | CD274 | CD274 molecule | −0.016777871 | 0.015365715 | 0.150193174 |
| 80381 | CD276 | CD276 molecule | 0.004194852 | −0.003650389 | 0.035103109 |
| 940 | CD28 | CD28 molecule | −0.056017848 | −0.09032102 | −0.11342893 |
| 947 | CD34 | CD34 molecule | 0.013031581 | 0.018582251 | −0.019624364 |
| 920 | CD4 | CD4 molecule | −0.053083942 | −0.110953872 | −0.041497737 |
| 958 | CD40 | CD40 molecule, TNF receptor superfamily member 5 | −0.030202508 | 0.062231726 | 0.116708266 |
| 959 | CD40LG | CD40 ligand | −0.103900142 | −0.071402632 | −0.11086035 |
| 965 | CD58 | CD58 molecule | 0.033029873 | 0.024811232 | 0.014796625 |
| 923 | CD6 | CD6 molecule | −0.045083264 | 0.090163225 | −0.109411313 |
| 941 | CD80 | CD80 molecule | 0.028558412 | −0.010086333 | 0.083681009 |
| 942 | CD86 | CD86 molecule | 0.027877251 | −0.009043919 | −0.107504395 |
| 925 | CD8A | CD8a molecule | −0.004102427 | 0.158921804 | −0.105556622 |
| 999 | CDH1 | cadherin 1, type 1, E-cadherin (epithelial) | −0.00412346 | 0.008293442 | 0.039342304 |
| 1013 | CDH15 | cadherin 15, type 1, M-cadherin (myotubule) | −0.015350661 | −0.023015097 | 0.053550596 |
| 1000 | CDH2 | cadherin 2, type 1, N-cadherin (neuronal) | −0.008270834 | −0.011348025 | 0.021183801 |
| 1001 | CDH3 | cadherin 3, type 1, P-cadherin (placental) | −0.010967392 | 0.01393509 | 0.014139428 |
| 1002 | CDH4 | cadherin 4, type 1, R-cadherin (retinal) | −0.002617803 | −0.000718771 | −0.003782769 |
| 1003 | CDH5 | cadherin 5, type 2 (vascular endothelium) | −0.005730892 | −0.021068125 | −0.004565345 |
| 9076 | CLDN1 | claudin 1 | −0.008152484 | −0.014740001 | 0.001550538 |
| 9071 | CLDN10 | claudin 10 | −0.005209284 | 0.01775261 | 0.005534196 |
| 5010 | CLDN11 | claudin 11 | 0.01002259 | 0.017410745 | 0.042640934 |
| 23562 | CLDN14 | claudin 14 | −0.012847641 | 0.004394641 | 0.010270242 |
| 24146 | CLDN15 | claudin 15 | −0.002572652 | 0.0077611 | 0.026167644 |
| 10686 | CLDN16 | claudin 16 | 0.021687776 | 0.028260496 | 0.019109033 |
| 26285 | CLDN17 | claudin 17 | 0.005585359 | −0.004759663 | 0.01021718 |
| 51208 | CLDN18 | claudin 18 | 0.002805619 | 0.018271871 | 0.007608307 |
| 149461 | CLDN19 | claudin 19 | 5.76E−05 | −0.017338313 | −0.002152721 |
| 9075 | CLDN2 | claudin 2 | 0.011709797 | −0.007597714 | −0.021416917 |
| 49861 | CLDN20 | claudin 20 | 0.02199445 | 0.027094781 | 0.019291772 |
| 53842 | CLDN22 | claudin 22 | −0.011427867 | −0.01690586 | −0.015652574 |
| 137075 | CLDN23 | claudin 23 | 0.004348942 | −0.012342353 | 0.066157287 |
| 1365 | CLDN3 | claudin 3 | −0.012307635 | 0.000278002 | 0.038585761 |
| 1364 | CLDN4 | claudin 4 | 0.001011178 | 0.003598941 | 0.02725779 |
| 7122 | CLDN5 | claudin 5 | −0.034540541 | −0.05012505 | 0.014706884 |
| 1366 | CLDN7 | claudin 7 | −0.015118723 | 0.024201759 | 0.048516311 |
| 9073 | CLDN8 | claudin 8 | 0.003863959 | 0.005768418 | 0.003077065 |
| 9080 | CLDN9 | claudin 9 | −0.012389372 | 0.000604549 | 0.018418111 |
| 1272 | CNTN1 | contactin 1 | −0.002774516 | 0.012327461 | 0.009027854 |
| 6900 | CNTN2 | contactin 2 (axonal) | −0.003770164 | 0.006561527 | 0.01843188 |
| 8506 | CNTNAP1 | contactin associated protein 1 | −0.007463407 | −0.019416368 | 0.065878724 |
| 26047 | CNTNAP2 | contactin associated protein-like 2 | 0.004317992 | −0.004430622 | 0.011857705 |
| 1493 | CTLA4 | cytotoxic T-lymphocyte-associated protein 4 | −0.022526478 | −0.109248593 | −0.102287725 |
| 90952 | ESAM | endothelial cell adhesion molecule | −0.047859768 | −0.02710148 | 0.08501458 |
| 50848 | F11R | F11 receptor | −0.020594915 | 0.033692933 | 0.023153873 |
| 2734 | GLG1 | golgi glycoprotein 1 | −0.041673544 | −0.014132418 | 0.027839869 |
| 3108 | HLA-DMA | major histocompatibility complex, class II, DM alpha | 0.011823208 | −0.056858359 | 0.016285572 |
| 3109 | HLA-DMB | major histocompatibility complex, class II, DM beta | 0.020497546 | −0.03308747 | −0.010727873 |
| 3111 | HLA-DOA | major histocompatibility complex, class II, DO alpha | −0.033724355 | −0.044856436 | 0.090380732 |
| 3113 | HLA-DPA1 | major histocompatibility complex, class II, DP alpha 1 | 0.041060178 | −0.102217341 | 0.057369701 |
| 3115 | HLA-DPB1 | major histocompatibility complex, class II, DP beta 1 | 0.048373217 | −0.023639141 | −0.072835793 |
| 3118 | HLA-DQA2 | major histocompatibility complex, class II, DQ alpha 2 | 0.024382014 | −0.049076103 | 0.015871978 |
| 3122 | HLA-DRA | major histocompatibility complex, class II, DR alpha | 0.007385599 | −0.077860694 | 0.058450635 |
| 3127 | HLA-DRB5 | major histocompatibility complex, class II, DR beta 5 | −0.028768599 | −0.000340101 | −0.050489613 |
| 3133 | HLA-E | major histocompatibility complex, class I, E | 0.008650351 | −0.062784603 | −0.062680424 |
| 3134 | HLA-F | major histocompatibility complex, class I, F | 0.022832302 | −0.004862356 | −0.086779223 |
| 3135 | HLA-G | major histocompatibility complex, class I, G | −0.007583133 | −0.096215853 | 0.023067679 |
| 3383 | ICAM1 | intercellular adhesion molecule 1 | −0.027306131 | 0.075547628 | −0.051299584 |
| 3384 | ICAM2 | intercellular adhesion molecule 2 | −0.071881163 | −0.046050405 | 0.037323074 |
| 3385 | ICAM3 | intercellular adhesion molecule 3 | −0.035060751 | −0.068339868 | 0.073119338 |
| 29851 | ICOS | inducible T-cell co-stimulator | −0.063808515 | −0.055749769 | −0.080683129 |
| 23308 | ICOSLG | inducible T-cell co-stimulator ligand | −0.017408697 | 0.052532634 | −0.059764022 |
| 3676 | ITGA4 | integrin, alpha 4 (antigen CD49D, alpha 4 subunit of VLA-4 | 0.028385939 | 0.026396434 | 0.049283953 |
| | | receptor) | | | |
| 3655 | ITGA6 | integrin, alpha 6 | −0.061910554 | −0.075116422 | −0.111035036 |
| 8516 | ITGA8 | integrin, alpha 8 | 0.004872254 | 0.00111313 | 0.004645907 |
| 3680 | ITGA9 | integrin, alpha 9 | 0.014515176 | −0.009883794 | −0.011571422 |
| 3683 | ITGAL | integrin, alpha L (antigen CD11A (p180), lymphocyte function- | 0.012078283 | −0.003282078 | −0.011277773 |
| | | associated antigen 1; alpha polypeptide) | | | |
| 3684 | ITGAM | integrin, alpha M (complement component 3 receptor 3 subunit) | −0.030875214 | −0.022709103 | −0.038947313 |
| 3685 | ITGAV | integrin, alpha V (vitronectin receptor, alpha polypeptide, | −0.003828593 | 0.030595683 | 0.021800215 |
| | | antigen CD51) | | | |
| 3688 | ITGB1 | integrin, beta 1 (fibronectin receptor, beta polypeptide, antigen | 0.002101818 | 0.000100204 | −0.065552763 |
| | | CD29 includes MDF2, MSK12) | | | |
| 3689 | ITGB2 | integrin, beta 2 (complement component 3 receptor 3 and 4 | 0.003127965 | −0.046762142 | −0.016742796 |
| | | subunit) | | | |
| 3695 | ITGB7 | integrin, beta 7 | −0.039717254 | −0.03768125 | 0.102189415 |
| 3696 | ITGB8 | integrin, beta 8 | 0.009464352 | 0.035750953 | 0.086576166 |
| 58494 | JAM2 | junctional adhesion molecule 2 | −0.009222826 | 0.00153459 | 0.029580866 |
| 83700 | JAM3 | junctional adhesion molecule 3 | −0.056205136 | −0.001533202 | −0.084711187 |
| 3897 | L1CAM | L1 cell adhesion molecule | −0.005667548 | 0.014851353 | 0.008886221 |
| 8174 | MADCAM1 | mucosal vascular addressin cell adhesion molecule 1 | 0.010692962 | −0.015635147 | 0.054686378 |
| 4099 | MAG | myelin associated glycoprotein | −0.020323958 | 0.020089562 | 0.035543235 |
| 4359 | MPZ | myelin protein zero | 0.000694181 | −0.007089335 | −0.017141443 |
| 9019 | MPZL1 | myelin protein zero-like 1 | −0.011844804 | −0.01758879 | 0.055876168 |
| 4684 | NCAM1 | neural cell adhesion molecule 1 | −0.004317548 | −0.0076833 | −0.008260968 |
| 4685 | NCAM2 | neural cell adhesion molecule 2 | −0.004400026 | 0.007039572 | 0.007005574 |
| 257194 | NEGR1 | neuronal growth regulator 1 | −0.001057963 | −0.002421025 | 0.013205528 |
| 4756 | NEO1 | neogenin 1 | −0.031696845 | −0.020750089 | −0.03615622 |
| 23114 | NFASC | neurofascin | −0.000308788 | −0.006770357 | 0.005253867 |
| 22871 | NLGN1 | neuroligin 1 | 0.008369802 | 0.006189118 | 0.020046505 |
| 57555 | NLGN2 | neuroligin 2 | −0.000753316 | 0.021053176 | −0.002702811 |
| 54413 | NLGN3 | neuroligin 3 | −0.011908051 | 0.036888142 | −0.044683389 |
| 57502 | NLGN4X | neuroligin 4, X-linked | 2.67E−05 | 0.007668168 | 0.013393044 |
| 4897 | NRCAM | neuronal cell adhesion molecule | −0.013128594 | 0.025167969 | 0.008887784 |
| 9378 | NRXN1 | neurexin 1 | 0.004668303 | 0.00306404 | 0.014473627 |
| 9379 | NRXN2 | neurexin 2 | −0.006733002 | 0.024749788 | 0.022484737 |
| 9369 | NRXN3 | neurexin 3 | 0.005539886 | 0.006189821 | 0.025016294 |
| 5133 | PDCD1 | programmed cell death 1 | −0.010628035 | 0.01836237 | 0.022730327 |
| 80380 | PDCD1LG2 | programmed cell death 1 ligand 2 | −0.004544248 | −0.010975565 | 0.107969099 |
| 5175 | PECAM1 | platelet/endothelial cell adhesion molecule | 0.009384994 | 0.031087671 | −0.062141904 |
| 5788 | PTPRC | protein tyrosine phosphatase, receptor type, C | 0.029953541 | 0.052065502 | −0.079110798 |
| 5792 | PTPRF | protein tyrosine phosphatase, receptor type, F | 0.000257974 | 0.002020447 | 0.00988409 |
| 5797 | PTPRM | protein tyrosine phosphatase, receptor type, M | 0.005420785 | 0.015432856 | −0.010124362 |
| 5817 | PVR | poliovirus receptor | −0.014158252 | −0.036058935 | 0.021723267 |
| 5818 | PVRL1 | poliovirus receptor-related 1 (herpesvirus entry mediator C) | −0.011735308 | 0.000260702 | 0.026818657 |
| 5819 | PVRL2 | poliovirus receptor-related 2 (herpesvirus entry mediator B) | −0.011539664 | 0.002351024 | −0.038437858 |
| 25945 | PVRL3 | poliovirus receptor-related 3 | −0.011905823 | 0.005037865 | 0.003288437 |
| 6382 | SDC1 | syndecan 1 | −0.019857829 | 0.021201147 | 0.038020518 |
| 6383 | SDC2 | syndecan 2 | −0.005418212 | 0.014879004 | −0.057896138 |
| 9672 | SDC3 | syndecan 3 | 0.003838367 | −0.028880609 | −0.001916696 |
| 6385 | SDC4 | syndecan 4 | −0.072386169 | −0.012167268 | −0.017701823 |
| 6401 | SELE | selectin E | 0.013781319 | 0.003429621 | 0.013906607 |
| 6402 | SELL | selectin L | −0.018456498 | −0.127088787 | 0.032598897 |
| 6403 | SELP | selectin P (granule membrane protein 140 kDa, antigen CD62) | −0.075085727 | −0.075282584 | −0.103979756 |
| 6404 | SELPLG | selectin P ligand | −0.062770554 | −0.054784741 | 0.024381444 |
| 6614 | SIGLEC1 | sialic acid binding Ig-like lectin 1, sialoadhesin | −0.026514986 | −0.025197147 | −0.001327582 |
| 6693 | SPN | sialophorin | −0.013962579 | −0.034302249 | 0.113322612 |
| 7412 | VCAM1 | vascular cell adhesion molecule 1 | 0.005986912 | −0.000865252 | 0.011003814 |
| 1462 | VCAN | versican | 0.057379648 | −0.013631561 | −0.01449824 |
| 4950 | [No Symbol] | [No Name] | −0.005497578 | 0.010034093 | 0.048821038 |

TABLE 9
Top pathways for which the number of mutations differed significantly between hereditary
breast cancer cases and controls. For a given sample, a pathway was considered to
be affected if any suspected pathogenic mutation had been observed in any gene in
that pathway. The Fisher's exact test was used to compare the number of mutated
samples between those who developed cancer and those who did not.

| Pathway | p-value |
|---|---|
| KEGG CITRATE CYCLE TCA CYCLE | 0.007 |
| BIOCARTA ARAP PATHWAY | 0.013 |
| REACTOME METABOLISM OF PROTEINS | 0.012 |
| REACTOME MEMBRANE TRAFFICKING | 0.010 |
| REACTOME SIGNALING IN IMMUNE SYSTEM | 0.014 |
| KEGG PARKINSONS DISEASE | 0.015 |
| REACTOME GLUCOSE AND OTHER SUGAR SLC TRANSPORTERS | 0.015 |
| REACTOME METAL ION SLC TRANSPORTERS | 0.035 |
| REACTOME ZINC INFLUX INTO CELLS BY THE SLC39 GENES FAMILY | 0.035 |
| REACTOME ZINC TRANSPORTATION | 0.035 |
| KEGG PROTEIN EXPORT | 0.024 |
| KEGG TASTE TRANSDUCTION | 0.035 |
| REACTOME SLC MEDIATED TRANSMEMBRANE TRANSPORT | 0.030 |
| KEGG VITAMIN DIGESTION AND ABSORPTION | 0.037 |
| REACTOME COLLAGEN MEDIATED ACTIVATION CASCADE | 0.037 |
| KEGG FRUCTOSE AND MANNOSE METABOLISM | 0.037 |
| REACTOME FORMATION AND MATURATION OF MRNA TRANSCRIPT | 0.027 |
| REACTOME CITRIC ACID CYCLE | 0.036 |
| REACTOME REGULATED PROTEOLYSIS OF P75NTR | 0.036 |
| ST B CELL ANTIGEN RECEPTOR | 0.036 |
| KEGG TYPE II DIABETES MELLITUS | 0.037 |
| KEGG CHOLINERGIC SYNAPSE | 0.042 |
| REACTOME AMINE LIGAND BINDING RECEPTORS | 0.042 |
| KEGG CELL ADHESION MOLECULES CAMS | 0.058 |
| KEGG PURINE METABOLISM | 0.058 |
| REACTOME INTEGRIN CELL SURFACE INTERACTIONS | 0.058 |
| REACTOME LATE PHASE OF HIV LIFE CYCLE | 0.058 |
| REACTOME CDT1 ASSOCIATION WITH THE CDC6 ORC ORIGIN COMPLEX | 0.086 |
| REACTOME M G1 TRANSITION | 0.086 |
| REACTOME ORC1 REMOVAL FROM CHROMATIN | 0.086 |
| REACTOME COPI MEDIATED TRANSPORT | 0.086 |
| BIOCARTA HSP27 PATHWAY | 0.086 |
| REACTOME INTRINSIC PATHWAY FOR APOPTOSIS | 0.086 |
| REACTOME AUTODEGRADATION OF CDH1 BY CDH1 APC | 0.086 |
| REACTOME BILE SALT AND ORGANIC ANION SLC TRANSPORTERS | 0.086 |
| REACTOME RNA POLYMERASE II TRANSCRIPTION | 0.052 |
| KEGG LONG TERM POTENTIATION | 0.074 |
| REACTOME RNA POLYMERASE I CHAIN ELONGATION | 0.086 |
| REACTOME RNA POLYMERASE I PROMOTER ESCAPE | 0.086 |
| REACTOME RNA POLYMERASE I TRANSCRIPTION TERMINATION | 0.086 |
| KEGG MISMATCH REPAIR | 0.074 |
| KEGG OLFACTORY TRANSDUCTION | 0.086 |
| REACTOME REGULATION OF GENE EXPRESSION IN BETA CELLS | 0.072 |
| KEGG HOMOLOGOUS RECOMBINATION | 0.072 |
| KEGG RIBOFLAVIN METABOLISM | 0.086 |
| BIOCARTA GH PATHWAY | 0.072 |
| REACTOME PYRUVATE METABOLISM | 0.086 |
| SIG CHEMOTAXIS | 0.072 |
| KEGG RETINOL METABOLISM | 0.074 |
| REACTOME A THIRD PROTEOLYTIC CLEAVAGE RELEASES NICD | 0.074 |
| REACTOME NRIF SIGNALS CELL DEATH FROM THE NUCLEUS | 0.074 |
| BIOCARTA RACCYCD PATHWAY | 0.074 |
| KEGG PANCREATIC SECRETION | 0.052 |
| REACTOME COSTIMULATION BY THE CD28 FAMILY | 0.074 |
| ST GAQ PATHWAY | 0.074 |
| KEGG PENTOSE AND GLUCURONATE INTERCONVERSIONS | 0.074 |
| REACTOME ASSOCIATION OF TRIC CCT WITH TARGET PROTEINS DURING . . . | 0.072 |
| REACTOME CHAPERONIN MEDIATED PROTEIN FOLDING | 0.072 |
| SIG PIP3 SIGNALING IN B LYMPHOCYTES | 0.074 |
| ST ADRENERGIC | 0.074 |
| SIG PIP3 SIGNALING IN CARDIAC MYOCTES | 0.074 |
| KEGG INTESTINAL IMMUNE NETWORK FOR IGA PRODUCTION | 0.074 |
| KEGG DNA REPLICATION | 0.104 |
| KEGG FAT DIGESTION AND ABSORPTION | 0.104 |
| REACTOME PLATELET ACTIVATION TRIGGERS | 0.104 |
| KEGG ERBB SIGNALING PATHWAY | 0.104 |
| REACTOME REGULATION OF BETA CELL DEVELOPMENT | 0.104 |
| REACTOME TRANSCRIPTION OF THE HIV GENOME | 0.104 |
| BIOCARTA IL2RB PATHWAY | 0.104 |
| KEGG PANCREATIC CANCER | 0.077 |
| KEGG NEUROTROPHIN SIGNALING PATHWAY | 0.077 |
| KEGG AMYOTROPHIC LATERAL SCLEROSIS ALS | 0.120 |
| KEGG ALANINE ASPARTATE AND GLUTAMATE METABOLISM | 0.120 |
| BIOCARTA CTCF PATHWAY | 0.132 |
| REACTOME CLATHRIN DERIVED VESICLE BUDDING | 0.120 |
| REACTOME GOLGI ASSOCIATED VESICLE BIOGENESIS | 0.120 |
| KEGG SMALL CELL LUNG CANCER | 0.132 |
| REACTOME OLFACTORY SIGNALING PATHWAY | 0.120 |
| BIOCARTA TGFB PATHWAY | 0.132 |
| REACTOME FORMATION OF FIBRIN CLOT CLOTTING CASCADE | 0.132 |
| KEGG TGF BETA SIGNALING PATHWAY | 0.120 |
| REACTOME AMINE COMPOUND SLC TRANSPORTERS | 0.120 |
| REACTOME TRAFFICKING OF AMPA RECEPTORS | 0.131 |
| REACTOME CELL SURFACE INTERACTIONS AT THE VASCULAR WALL | 0.094 |
| REACTOME FANCONI ANEMIA PATHWAY | 0.202 |
| REACTOME FORMATION OF A POOL OF FREE 40S SUBUNITS | 0.131 |
| REACTOME GTP HYDROLYSIS AND JOINING OF THE 60S RIBOSOMAL SUB . . . | 0.131 |
| REACTOME TRANSLATION | 0.131 |
| KEGG VIRAL MYOCARDITIS | 0.137 |
| KEGG REGULATION OF AUTOPHAGY | 0.202 |
| KEGG PHOSPHATIDYLINOSITOL SIGNALING SYSTEM | 0.094 |
| BIOCARTA ATM PATHWAY | 0.131 |
| REACTOME SIGNALLING TO ERKS | 0.202 |
| REACTOME SIGNALLING TO RAS | 0.202 |
| KEGG PRIMARY BILE ACID BIOSYNTHESIS | 0.202 |
| REACTOME ENDOGENOUS STEROLS | 0.202 |
| REACTOME METABOLISM OF BILE ACIDS AND BILE SALTS | 0.202 |
| REACTOME SYNTHESIS OF BILE ACIDS AND BILE SALTS | 0.202 |
| REACTOME SYNTHESIS OF BILE ACIDS AND BILE SALTS VIA 7ALPHA H . . . | 0.202 |
| REACTOME IMMUNOREGULATORY INTERACTIONS BETWEEN A LYMPHOID AN . . . | 0.131 |
| BIOCARTA P53HYPOXIA PATHWAY | 0.131 |
| BIOCARTA PAR1 PATHWAY | 0.131 |
| REACTOME SYNTHESIS OF GLYCOSYLPHOSPHATIDYLINOSITOL | 0.131 |
| BIOCARTA NKCELLS PATHWAY | 0.131 |
| KEGG PROTEASOME | 0.202 |
| REACTOME REGULATION OF ORNITHINE DECARBOXYLASE | 0.202 |
| REACTOME SCF BETA TRCP MEDIATED DEGRADATION OF EMI1 | 0.202 |
| REACTOME SIGNALING BY WNT | 0.202 |
| REACTOME VIF MEDIATED DEGRADATION OF APOBEC3G | 0.202 |
| SIG BCR SIGNALING PATHWAY | 0.131 |
| BIOCARTA ACH PATHWAY | 0.131 |
| BIOCARTA AGR PATHWAY | 0.131 |
| REACTOME LYSOSOME VESICLE BIOGENESIS | 0.202 |
| KEGG NON HOMOLOGOUS END JOINING | 0.202 |
| REACTOME APOPTOTIC CLEAVAGE OF CELL ADHESION PROTEINS | 0.202 |
| REACTOME CASPASE MEDIATED CLEAVAGE OF CYTOSKELETAL PROTEINS | 0.202 |
| SA FAS SIGNALING | 0.202 |
| ST GRANULE CELL SURVIVAL PATHWAY | 0.202 |
| BIOCARTA PROTEASOME PATHWAY | 0.202 |
| REACTOME NA CL DEPENDENT NEUROTRANSMITTER TRANSPORTERS | 0.202 |
| REACTOME G PROTEIN ACTIVATION | 0.202 |
| BIOCARTA NUCLEARRS PATHWAY | 0.202 |
| BIOCARTA CHREBP2 PATHWAY | 0.202 |
| KEGG FATTY ACID BIOSYNTHESIS | 0.202 |
| REACTOME TAT MEDIATED HIV1 ELONGATION ARREST AND RECOVERY | 0.148 |
| BIOCARTA CELL2CELL PATHWAY | 0.202 |
| BIOCARTA HIF PATHWAY | 0.202 |
| REACTOME UNFOLDED PROTEIN RESPONSE | 0.202 |
| BIOCARTA LEPTIN PATHWAY | 0.202 |
| REACTOME CHOLESTEROL BIOSYNTHESIS | 0.148 |
| REACTOME TRANSMEMBRANE TRANSPORT OF SMALL MOLECULES | 0.176 |
| KEGG AMINOACYL TRNA BIOSYNTHESIS | 0.176 |
| REACTOME MITOCHONDRIAL TRNA AMINOACYLATION | 0.176 |
| REACTOME TRNA AMINOACYLATION | 0.176 |
| ST MYOCYTE AD PATHWAY | 0.148 |
| KEGG ENDOMETRIAL CANCER | 0.176 |
| BIOCARTA EDG1 PATHWAY | 0.148 |
| BIOCARTA EIF4 PATHWAY | 0.148 |
| BIOCARTA MTOR PATHWAY | 0.148 |
| REACTOME CD28 CO STIMULATION | 0.148 |
| REACTOME INFLUENZA LIFE CYCLE | 0.176 |
| KEGG OOCYTE MEIOSIS | 0.176 |
| KEGG ASCORBATE AND ALDARATE METABOLISM | 0.148 |
| SIG INSULIN RECEPTOR PATHWAY IN CARDIAC MYOCYTES | 0.148 |
| KEGG LONG TERM DEPRESSION | 0.176 |
| REACTOME DOWNSTREAM TCR SIGNALING | 0.148 |
| REACTOME G2 M TRANSITION | 0.148 |
| REACTOME TCR SIGNALING | 0.148 |
| KEGG B CELL RECEPTOR SIGNALING PATHWAY | 0.176 |
| REACTOME CELL DEATH SIGNALLING VIA NRAGE NRIF AND NADE | 0.176 |
| KEGG MATURITY ONSET DIABETES OF THE YOUNG | 0.148 |
| KEGG ACUTE MYELOID LEUKEMIA | 0.176 |
| BIOCARTA FCER1 PATHWAY | 0.148 |
| REACTOME PLATELET ADHESION TO EXPOSED COLLAGEN | 0.176 |
| BIOCARTA RAC1 PATHWAY | 0.176 |
| REACTOME MITOTIC M M G1 PHASES | 0.141 |
| REACTOME TRANSMISSION ACROSS CHEMICAL SYNAPSES | 0.156 |
| ST INTEGRIN SIGNALING PATHWAY | 0.212 |
| BIOCARTA TOB1 PATHWAY | 0.212 |
| REACTOME APOPTOSIS INDUCED DNA FRAGMENTATION | 0.238 |
| KEGG PROSTATE CANCER | 0.176 |
| REACTOME ELONGATION AND PROCESSING OF CAPPED TRANSCRIPTS | 0.156 |
| BIOCARTA TNFR1 PATHWAY | 0.212 |
| KEGG HEPATITIS C | 0.176 |
| KEGG PROPANOATE METABOLISM | 0.156 |
| REACTOME HIV LIFE CYCLE | 0.156 |
| KEGG CALCIUM SIGNALING PATHWAY | 0.223 |
| BIOCARTA MITOCHONDRIA PATHWAY | 0.238 |
| KEGG RNA POLYMERASE | 0.238 |
| BIOCARTA IL10 PATHWAY | 0.238 |
| BIOCARTA IL22BP PATHWAY | 0.238 |
| KEGG HUNTINGTONS DISEASE | 0.223 |
| KEGG OSTEOCLAST DIFFERENTIATION | 0.223 |
| KEGG PYRIMIDINE METABOLISM | 0.194 |
| BIOCARTA SET PATHWAY | 0.227 |
| REACTOME INSULIN SYNTHESIS AND SECRETION | 0.234 |
| KEGG NOD LIKE RECEPTOR SIGNALING PATHWAY | 0.234 |
| KEGG PEROXISOME | 0.223 |
| KEGG RIBOSOME | 0.227 |
| REACTOME INFLUENZA VIRAL RNA TRANSCRIPTION AND REPLICATION | 0.227 |
| REACTOME PEPTIDE CHAIN ELONGATION | 0.227 |
| REACTOME VIRAL MRNA TRANSLATION | 0.227 |
| KEGG ARRHYTHMOGENIC RIGHT VENTRICULAR CARDIOMYOPATHY ARVC | 0.234 |
| REACTOME GAP JUNCTION TRAFFICKING | 0.238 |
| REACTOME BIOLOGICAL OXIDATIONS | 0.223 |
| REACTOME RNA POLYMERASE I TRANSCRIPTION INITIATION | 0.238 |
| BIOCARTA 41BB PATHWAY | 0.227 |
| BIOCARTA AMI PATHWAY | 0.227 |
| BIOCARTA CERAMIDE PATHWAY | 0.227 |
| BIOCARTA EXTRINSIC PATHWAY | 0.227 |
| BIOCARTA INTRINSIC PATHWAY | 0.227 |
| BIOCARTA STRESS PATHWAY | 0.227 |
| BIOCARTA TALL1 PATHWAY | 0.227 |
| BIOCARTA TNFR2 PATHWAY | 0.227 |
| REACTOME COMMON PATHWAY | 0.227 |
| REACTOME G ALPHA 12 13 SIGNALLING EVENTS | 0.227 |
| REACTOME GLYCOLYSIS | 0.227 |
| KEGG ALLOGRAFT REJECTION | 0.227 |
| BIOCARTA CARDIACEGF PATHWAY | 0.227 |
| BIOCARTA G2 PATHWAY | 0.227 |
| BIOCARTA NKT PATHWAY | 0.227 |
| KEGG ARGININE AND PROLINE METABOLISM | 0.227 |
| KEGG BASAL CELL CARCINOMA | 0.227 |
| BIOCARTA ECM PATHWAY | 0.227 |
| BIOCARTA SODD PATHWAY | 0.227 |
| BIOCARTA P53 PATHWAY | 0.227 |
| REACTOME SIGNALING BY EGFR | 0.227 |
| BIOCARTA CXCR4 PATHWAY | 0.227 |
| BIOCARTA GCR PATHWAY | 0.227 |
| KEGG LINOLEIC ACID METABOLISM | 0.227 |
| BIOCARTA BIOPEPTIDES PATHWAY | 0.248 |
| BIOCARTA EGF PATHWAY | 0.248 |
| BIOCARTA PDGF PATHWAY | 0.248 |
| REACTOME NEURORANSMITTER RECEPTOR BINDING AND DOWNSTREAM | 0.280 |
| TRA . . . | |
| REACTOME RNA POLYMERASE I PROMOTER CLEARANCE | 0.248 |
| REACTOME HEMOSTASIS | 0.248 |
| REACTOME DOWN STREAM SIGNAL TRANSDUCTION | 0.248 |
| REACTOME PI3K AKT SIGNALLING | 0.280 |
| BIOCARTA CHEMICAL PATHWAY | 0.248 |
| KEGG T CELL RECEPTOR SIGNALING PATHWAY | 0.280 |
| KEGG NUCLEOTIDE EXCISION REPAIR | 0.280 |
| REACTOME MRNA 3 END PROCESSING | 0.280 |
| REACTOME METABOLISM OF VITAMINS AND COFACTORS | 0.280 |
| KEGG BASE EXCISION REPAIR | 0.248 |
| KEGG PATHWAYS IN CANCER | 0.248 |
| REACTOME PHASE 1 FUNCTIONALIZATION OF COMPOUNDS | 0.248 |
| REACTOME HORMONE BIOSYNTHESIS | 0.280 |
| REACTOME RNA POLYMERASE I III AND MITOCHONDRIAL TRANSCRIPTIO . . . | 0.248 |
| BIOCARTA NTHI PATHWAY | 0.280 |
| KEGG AMINO SUGAR AND NUCLEOTIDE SUGAR METABOLISM | 0.280 |
| BIOCARTA IL7 PATHWAY | 0.280 |
| BIOCARTA INTEGRIN PATHWAY | 0.248 |
| KEGG BLADDER CANCER | 0.280 |
| REACTOME PLATELET AGGREGATION PLUG FORMATION | 0.280 |
| REACTOME SEMAPHORIN INTERACTIONS | 0.319 |
| REACTOME SYNTHESIS OF BILE ACIDS AND BILE SALTS VIA 24 HYDRO . . . | 0.457 |
| KEGG STEROID HORMONE BIOSYNTHESIS | 0.280 |
| KEGG GLYCOLYSIS GLUCONEOGENESIS | 0.319 |
| KEGG GLYCEROLIPID METABOLISM | 0.319 |
| KEGG BUTANOATE METABOLISM | 0.319 |
| BIOCARTA AKAP13 PATHWAY | 0.457 |
| KEGG CARDIAC MUSCLE CONTRACTION | 0.248 |
| REACTOME P75NTR RECRUITS SIGNALLING COMPLEXES | 0.457 |
| BIOCARTA PARKIN PATHWAY | 0.457 |
| KEGG THIAMINE METABOLISM | 0.457 |
| REACTOME REGULATION OF INSULIN LIKE GROWTH FACTOR | 0.457 |
| ACTIVITY B . . . | |
| REACTOME SIGNALING BY PDGF | 0.243 |
| SIG CD40PATHWAYMAP | 0.319 |
| BIOCARTA AKAPCENTROSOME PATHWAY | 0.457 |
| BIOCARTA CALCINEURIN PATHWAY | 0.457 |
| KEGG GLYCOSPHINGOLIPID BIOSYNTHESIS GLOBO SERIES | 0.457 |
| KEGG MINERAL ABSORPTION | 0.319 |
| REACTOME INHIBITION OF INSULIN SECRETION BY ADRENALINE | 0.319 |
| NORAD . . . | |
| REACTOME NEF MEDIATED DOWNREGULATION OF MHC CLASS I COMPLEX . . . | 0.457 |
| KEGG METABOLISM OF XENOBIOTICS BY CYTOCHROME P450 | 0.319 |
| REACTOME GENE EXPRESSION | 0.243 |
| BIOCARTA IL17 PATHWAY | 0.457 |
| BIOCARTA RANKL PATHWAY | 0.287 |
| REACTOME AMINO ACID SYNTHESIS AND INTERCONVERSION | 0.457 |
| REACTOME TRKA SIGNALLING FROM THE PLASMA MEMBRANE | 0.242 |
| REACTOME INNATE IMMUNITY SIGNALING | 0.319 |
| REACTOME G ALPHA S SIGNALLING EVENTS | 0.319 |
| REACTOME SIGNALLING BY NGF | 0.330 |
| KEGG ADHERENS JUNCTION | 0.319 |
| KEGG BIOTIN METABOLISM | 0.457 |
| REACTOME CYTOSOLIC SULFONATION OF SMALL MOLECULES | 0.457 |
| REACTOME RETROGRADE NEUROTROPHIN SIGNALLING | 0.457 |
| REACTOME RNA POLYMERASE III CHAIN ELONGATION | 0.457 |
| REACTOME RNA POLYMERASE III TRANSCRIPTION INITIATION FROM TY . . . | 0.457 |
| REACTOME RNA POLYMERASE III TRANSCRIPTION INITIATION FROM TY . . . | 0.457 |
| REACTOME RNA POLYMERASE III TRANSCRIPTION TERMINATION | 0.457 |
| SA PROGRAMMED CELL DEATH | 0.457 |
| BIOCARTA ETS PATHWAY | 0.457 |
| KEGG CYANOAMINO ACID METABOLISM | 0.457 |
| KEGG TAURINE AND HYPOTAURINE METABOLISM | 0.457 |
| REACTOME GLYCOGEN BREAKDOWN GLYCOGENOLYSIS | 0.457 |
| KEGG TRYPTOPHAN METABOLISM | 0.319 |
| KEGG VASOPRESSIN REGULATED WATER REABSORPTION | 0.287 |
| KEGG VALINE LEUCINE AND ISOLEUCINE DEGRADATION | 0.242 |
| REACTOME ADP SIGNALLING THROUGH P2Y PURINOCEPTOR 12 | 0.457 |
| REACTOME SIGNAL AMPLIFICATION | 0.457 |
| KEGG MELANOMA | 0.242 |
| ST G ALPHA I PATHWAY | 0.287 |
| ST WNT CA2 CYCLIC GMP PATHWAY | 0.287 |
| KEGG GLIOMA | 0.242 |
| BIOCARTA PLCE PATHWAY | 0.457 |
| BIOCARTA SARS PATHWAY | 0.457 |
| KEGG AFRICAN TRYPANOSOMIASIS | 0.457 |
| KEGG ENDOCRINE AND OTHER FACTOR REGULATED CALCIUM REABSORPTI . . . | 0.287 |
| KEGG TOLL LIKE RECEPTOR SIGNALING PATHWAY | 0.319 |
| REACTOME CELL CYCLE MITOTIC | 0.330 |
| REACTOME EICOSANOID LIGAND BINDING RECEPTORS | 0.457 |
| REACTOME MRNA SPLICING | 0.319 |
| REACTOME PURINE RIBONUCLEOSIDE MONOPHOSPHATE BIOSYNTHESIS | 0.457 |
| ST GA12 PATHWAY | 0.287 |
| BIOCARTA HIVNEF PATHWAY | 0.267 |
| BIOCARTA STATHMIN PATHWAY | 0.457 |
| KEGG TERPENOID BACKBONE BIOSYNTHESIS | 0.287 |
| REACTOME CDC6 ASSOCIATION WITH THE ORC: ORIGIN COMPLEX | 0.457 |
| REACTOME P38MAPK EVENTS | 0.457 |
| REACTOME POST CHAPERONIN TUBULIN FOLDING PATHWAY | 0.457 |
| KEGG INSULIN SIGNALING PATHWAY | 0.242 |
| BIOCARTA CELLCYCLE PATHWAY | 0.287 |
| KEGG ARACHIDONIC ACID METABOLISM | 0.319 |
| REACTOME E2F TRANSCRIPTIONAL TARGETS AT G1 S | 0.287 |
| REACTOME NRAGE SIGNALS DEATH THROUGH JNK | 0.287 |
| BIOCARTA PTDINS PATHWAY | 0.287 |
| REACTOME REMOVAL OF THE FLAP INTERMEDIATE FROM THE C STRAND | 0.287 |
| REACTOME VITAMIN B5 (PANTOTHENATE) METABOLISM | 0.287 |
| KEGG STEROID BIOSYNTHESIS | 0.287 |
| BIOCARTA CLASSIC PATHWAY | 0.287 |
| BIOCARTA COMP PATHWAY | 0.287 |
| KEGG VALINE LEUCINE AND ISOLEUCINE BIOSYNTHESIS | 0.287 |
| REACTOME CLASSICAL ANTIBODY MEDIATED COMPLEMENT ACTIVATION | 0.287 |
| REACTOME COMPLEMENT CASCADE | 0.287 |
| REACTOME INITIAL TRIGGERING OF COMPLEMENT | 0.287 |
| BIOCARTA AKAP95 PATHWAY | 0.457 |
| BIOCARTA LAIR PATHWAY | 0.287 |
| BIOCARTA LYM PATHWAY | 0.287 |
| BIOCARTA MONOCYTE PATHWAY | 0.287 |
| REACTOME ACTIVATED AMPK STIMULATES FATTY ACID OXIDATION IN M . . . | 0.457 |
| REACTOME APCDC20 MEDIATED DEGRADATION OF CYCLIN B | 0.457 |
| REACTOME CONVERSION FROM APC CDC20 TO APC CDH1 IN LATE ANAPH . . . | 0.457 |
| REACTOME GAP JUNCTION DEGRADATION | 0.457 |
| REACTOME INORGANIC CATION ANION SLC TRANSPORTERS | 0.330 |
| REACTOME PHOSPHORYLATION OF THE APC | 0.457 |
| REACTOME REGULATION OF PYRUVATE DEHYDROGENASE COMPLEX | 0.457 |
| REACTOME REGULATION OF RHEB GTPASE ACTIVITY BY AMPK | 0.457 |
| REACTOME SIGNALING BY TGF BETA | 0.352 |
| REACTOME TRAFFICKING OF GLUR2 CONTAINING AMPA RECEPTORS | 0.287 |
| BIOCARTA IL4 PATHWAY | 0.457 |
| BIOCARTA MEF2D PATHWAY | 0.457 |
| REACTOME DUAL INCISION REACTION IN TC NER | 0.457 |
| REACTOME ERKS ARE INACTIVATED | 0.457 |
| REACTOME ERK MAPK TARGETS | 0.457 |
| REACTOME MAPK TARGETS NUCLEAR EVENTS MEDIATED BY MAP KINASES | 0.457 |
| REACTOME MAP KINASES ACTIVATION IN TLR CASCADE | 0.457 |
| REACTOME MRNA PROCESSING | 0.457 |
| REACTOME NUCLEAR EVENTS KINASE AND TRANSCRIPTION FACTOR ACTI . . . | 0.457 |
| REACTOME RNA POL II CTD PHOSPHORYLATION AND INTERACTION WITH . . . | 0.457 |
| KEGG ECM RECEPTOR INTERACTION | 0.267 |
| REACTOME HIV INFECTION | 0.290 |
| REACTOME PD1 SIGNALING | 0.287 |
| REACTOME VIRAL DSRNA TLR3 TRIF COMPLEX ACTIVATES RIP1 | 0.287 |
| BIOCARTA SPPA PATHWAY | 0.287 |
| KEGG FC EPSILON RI SIGNALING PATHWAY | 0.352 |
| KEGG WNT SIGNALING PATHWAY | 0.352 |
| KEGG LYSINE DEGRADATION | 0.347 |
| REACTOME CLASS C3 METABOTROPIC GLUTAMATE PHEROMONE RECEPTORS | 0.287 |
| REACTOME PLATELET ACTIVATION | 0.330 |
| BIOCARTA ALK PATHWAY | 0.352 |
| KEGG PRION DISEASES | 0.287 |
| REACTOME REGULATION OF INSULIN SECRETION | 0.311 |
| BIOCARTA BCR PATHWAY | 0.287 |
| SA B CELL RECEPTOR COMPLEXES | 0.287 |
| KEGG NATURAL KILLER CELL MEDIATED CYTOTOXICITY | 0.290 |
| KEGG RIG I LIKE RECEPTOR SIGNALING PATHWAY | 0.352 |
| BIOCARTA BAD PATHWAY | 0.287 |
| BIOCARTA CDC42RAC PATHWAY | 0.287 |
| BIOCARTA CREB PATHWAY | 0.287 |
| BIOCARTA CTLA4 PATHWAY | 0.287 |
| BIOCARTA FIBRINOLYSIS PATHWAY | 0.287 |
| BIOCARTA IGF1MTOR PATHWAY | 0.287 |
| BIOCARTA IGF1R PATHWAY | 0.287 |
| BIOCARTA LONGEVITY PATHWAY | 0.287 |
| BIOCARTA NFAT PATHWAY | 0.287 |
| BIOCARTA NGF PATHWAY | 0.287 |
| BIOCARTA PTEN PATHWAY | 0.287 |
| BIOCARTA TRKA PATHWAY | 0.287 |
| KEGG PORPHYRIN AND CHLOROPHYLL METABOLISM | 0.287 |
| REACTOME CD28 DEPENDENT PI3K AKT SIGNALING | 0.287 |
| REACTOME GAB1 SIGNALOSOME | 0.287 |
| REACTOME GLUCURONIDATION | 0.287 |
| REACTOME HDL MEDIATED LIPID TRANSPORT | 0.287 |
| REACTOME LIPOPROTEIN METABOLISM | 0.287 |
| REACTOME TIE2 SIGNALING | 0.287 |
| SA TRKA RECEPTOR | 0.287 |
| ST DIFFERENTIATION PATHWAY IN PC 12 CELLS | 0.287 |
| ST PHOSPHOINOSITIDE 3 KINASE PATHWAY | 0.287 |
| KEGG CHRONIC MYELOID LEUKEMIA | 0.311 |
| REACTOME POST TRANSLATIONAL PROTEIN MODIFICATION | 0.352 |
| REACTOME FORMATION OF PLATELET PLUG | 0.347 |
| KEGG APOPTOSIS | 0.267 |
| KEGG COLORECTAL CANCER | 0.242 |
| REACTOME P75 NTR RECEPTOR MEDIATED SIGNALLING | 0.352 |
| KEGG NOTCH SIGNALING PATHWAY | 0.352 |
| REACTOME AXON GUIDANCE | 0.290 |
| REACTOME METABOLISM OF LIPIDS AND LIPOPROTEINS | 0.347 |
| REACTOME TRANSCRIPTION | 0.330 |
| KEGG ASTHMA | 0.287 |
| REACTOME CENTROSOME MATURATION | 0.287 |
| REACTOME LOSS OF NLP FROM MITOTIC CENTROSOMES | 0.287 |
| KEGG REGULATION OF ACTIN CYTOSKELETON | 0.347 |
| KEGG AXON GUIDANCE | 0.290 |
| KEGG INOSITOL PHOSPHATE METABOLISM | 0.352 |
| KEGG VASCULAR SMOOTH MUSCLE CONTRACTION | 0.311 |
| KEGG GLYCOSPHINGOLIPID BIOSYNTHESIS GANGLIO SERIES | 0.287 |
| KEGG COLLECTING DUCT ACID SECRETION | 0.434 |
| REACTOME HIV1 TRANSCRIPTION ELONGATION | 0.370 |
| BIOCARTA ETC PATHWAY | 0.434 |
| REACTOME TRANSPORT OF MATURE MRNA DERIVED FROM AN INTRON | 0.434 |
| CON . . . | |
| REACTOME TRANSPORT OF THE SLBP INDEPENDENT MATURE MRNA | 0.434 |
| REACTOME P53 INDEPENDENT DNA DAMAGE RESPONSE | 0.434 |
| BIOCARTA CYTOKINE PATHWAY | 0.434 |
| REACTOME EARLY PHASE OF HIV LIFE CYCLE | 0.434 |
| REACTOME GENES INVOLVED IN APOPTOTIC CLEAVAGE OF CELLULAR PR . . . | 0.434 |
| REACTOME DOPAMINE NEUROTRANSMITTER RELEASE CYCLE | 0.434 |
| REACTOME SEROTONIN NEUROTRANSMITTER RELEASE CYCLE | 0.434 |
| KEGG MUCIN TYPE O GLYCAN BIOSYNTHESIS | 0.434 |
| REACTOME GLUCONEOGENESIS | 0.370 |
| KEGG NITROGEN METABOLISM | 0.434 |
| REACTOME RNA POLYMERASE III TRANSCRIPTION | 0.434 |
| REACTOME RNA POLYMERASE III TRANSCRIPTION INITIATION | 0.434 |
| BIOCARTA PITX2 PATHWAY | 0.434 |
| REACTOME G ALPHA Z SIGNALLING EVENTS | 0.434 |
| REACTOME DOUBLE STRAND BREAK REPAIR | 0.434 |
| BIOCARTA DNAFRAGMENT PATHWAY | 0.434 |
| REACTOME SCF SKP2 MEDIATED DEGRADATION OF P27 P21 | 0.434 |
| REACTOME TOLL LIKE RECEPTOR 3 CASCADE | 0.370 |
| REACTOME PACKAGING OF TELOMERE ENDS | 0.434 |
| REACTOME RNA POLYMERASE I PROMOTER OPENING | 0.434 |
| REACTOME BRANCHED CHAIN AMINO ACID CATABOLISM | 0.370 |
| REACTOME MYD88 CASCADE | 0.434 |
| REACTOME TOLL LIKE RECEPTOR 9 CASCADE | 0.434 |
| KEGG OTHER GLYCAN DEGRADATION | 0.434 |
| BIOCARTA CTL PATHWAY | 0.370 |
| KEGG GRAFT VERSUS HOST DISEASE | 0.370 |
| KEGG TYPE I DIABETES MELLITUS | 0.370 |
| REACTOME DEATH RECEPTOR SIGNALLING | 0.370 |
| SA G1 AND S PHASES | 0.370 |
| BIOCARTA AKT PATHWAY | 0.370 |
| BIOCARTA ERYTH PATHWAY | 0.434 |
| BIOCARTA RAS PATHWAY | 0.370 |
| REACTOME DOWNSTREAM SIGNALING OF ACTIVATED FGFR | 0.370 |
| REACTOME PI3K CASCADE | 0.370 |
| REACTOME ACTIVATION OF RAC | 0.434 |
| BIOCARTA SPRY PATHWAY | 0.434 |
| KEGG PENTOSE PHOSPHATE PATHWAY | 0.434 |
| BIOCARTA GLEEVEC PATHWAY | 0.370 |
| REACTOME GAP JUNCTION ASSEMBLY | 0.434 |
| REACTOME DNA STRAND ELONGATION | 0.370 |
| REACTOME LAGGING STRAND SYNTHESIS | 0.370 |
| REACTOME REMOVAL OF THE FLAP INTERMEDIATE | 0.370 |
| BIOCARTA G1 PATHWAY | 0.370 |
| BIOCARTA MCALPAIN PATHWAY | 0.370 |
| REACTOME GRB2 SOS PROVIDES LINKAGE TO MAPK SIGNALING FOR INT . . . | 0.370 |
| REACTOME INTEGRIN ALPHAIIBBETA3 SIGNALING | 0.370 |
| REACTOME P130CAS LINKAGE TO MAPK SIGNALING FOR INTEGRINS | 0.370 |
| SIG REGULATION OF THE ACTIN CYTOSKELETON BY RHO GTPASES | 0.370 |
| KEGG ANTIGEN PROCESSING AND PRESENTATION | 0.434 |
| BIOCARTA ATRBRCA PATHWAY | 0.370 |
| BIOCARTA TID PATHWAY | 0.370 |
| BIOCARTA IL12 PATHWAY | 0.370 |
| REACTOME PLC BETA MEDIATED EVENTS | 0.370 |
| REACTOME PLC GAMMA1 SIGNALLING | 0.370 |
| REACTOME APOPTOTIC EXECUTION PHASE | 0.415 |
| BIOCARTA HCMV PATHWAY | 0.370 |
| ST INTERLEUKIN 4 PATHWAY | 0.370 |
| KEGG GLYCOSPHINGOLIPID BIOSYNTHESIS LACTO AND NEOLACTO SERIE . . . | 0.415 |
| BIOCARTA IL1R PATHWAY | 0.415 |
| KEGG GALACTOSE METABOLISM | 0.415 |
| KEGG GLUTATHIONE METABOLISM | 0.415 |
| REACTOME CDC20 PHOSPHO APC MEDIATED DEGRADATION OF CYCLIN A | 0.415 |
| REACTOME REGULATION OF APC ACTIVATORS BETWEEN G1 S AND EARLY . . . | 0.415 |
| KEGG MALARIA | 0.415 |
| KEGG STAPHYLOCOCCUS AUREUS INFECTION | 0.418 |
| KEGG HEDGEHOG SIGNALING PATHWAY | 0.415 |
| REACTOME ACTIVATION OF ATR IN RESPONSE TO REPLICATION STRESS | 0.418 |
| REACTOME G2 M CHECKPOINTS | 0.418 |
| REACTOME HIV1 TRANSCRIPTION INITIATION | 0.418 |
| BIOCARTA GSK3 PATHWAY | 0.418 |
| REACTOME GLUCOSE TRANSPORT | 0.418 |
| REACTOME SIGNALING BY NOTCH | 0.418 |
| REACTOME EXTENSION OF TELOMERES | 0.418 |
| BIOCARTA UCALPAIN PATHWAY | 0.415 |
| KEGG NICOTINATE AND NICOTINAMIDE METABOLISM | 0.418 |
| REACTOME INTRINSIC PATHWAY | 0.452 |
| KEGG SYSTEMIC LUPUS ERYTHEMATOSUS | 0.398 |
| REACTOME CLASS B2 SECRETIN FAMILY RECEPTORS | 0.418 |
| KEGG MELANOGENESIS | 0.418 |
| REACTOME GPCR LIGAND BINDING | 0.398 |
| REACTOME CYTOCHROME P450 ARRANGED BY SUBSTRATE TYPE | 0.398 |
| BIOCARTA MET PATHWAY | 0.398 |
| KEGG CYSTEINE AND METHIONINE METABOLISM | 0.418 |
| REACTOME GLUCOSE METABOLISM | 0.418 |
| KEGG MTOR SIGNALING PATHWAY | 0.418 |
| KEGG PPAR SIGNALING PATHWAY | 0.398 |
| KEGG NON SMALL CELL LUNG CANCER | 0.452 |
| REACTOME MITOCHONDRIAL FATTY ACID BETA OXIDATION | 0.381 |
| KEGG GLYCOSAMINOGLYCAN BIOSYNTHESIS HEPARAN SULFATE | 0.481 |
| KEGG ADIPOCYTOKINE SIGNALING PATHWAY | 0.481 |
| REACTOME IRS RELATED EVENTS | 0.452 |
| REACTOME PYRUVATE METABOLISM AND TCA CYCLE | 0.452 |
| KEGG SPLICEOSOME | 0.381 |
| KEGG FATTYACID METABOLISM | 0.364 |
| KEGG PROTEIN PROCESSING IN ENDOPLASMIC RETICULUM | 0.404 |
| KEGG GLYCEROPHOSPHOLIPID METABOLISM | 0.452 |
| KEGG BASAL TRANSCRIPTION FACTORS | 0.481 |
| REACTOME ACTIVATION OF THE PRE REPLICATIVE COMPLEX | 0.481 |
| KEGG ENDOCYTOSIS | 0.381 |
| BIOCARTA TPO PATHWAY | 0.452 |
| KEGG HEMATOPOIETIC CELL LINEAGE | 0.379 |
| REACTOME DIABETES PATHWAYS | 0.381 |
| BIOCARTA ARF PATHWAY | 0.452 |
| KEGG GLYCOSYLPHOSPHATIDYLINOSITOLGPI ANCHOR BIOSYNTHESIS | 0.364 |
| REACTOME SYNTHESIS OF GPI ANCHORED PROTEINS | 0.452 |
| KEGG BACTERIAL INVASION OF EPITHELIAL CELLS | 0.452 |
| REACTOME AMINO ACID AND OLIGOPEPTIDE SLC TRANSPORTERS | 0.505 |
| ST FAS SIGNALING PATHWAY | 0.452 |
| KEGG TIGHT JUNCTION | 0.381 |
| REACTOME METABOLISM OF AMINO ACIDS | 0.379 |
| REACTOME METABOLISM OF CARBOHYDRATES | 0.505 |
| ST TUMOR NECROSIS FACTOR PATHWAY | 0.452 |
| KEGG ALZHEIMERS DISEASE | 0.427 |
| KEGG RENAL CELL CARCINOMA | 0.481 |
| KEGG LEUKOCYTE TRANSENDOTHELIAL MIGRATION | 0.449 |
| KEGG CHAGAS DISEASE AMERICAN TRYPANOSOMIASIS | 0.379 |
| KEGG N GLYCAN BIOSYNTHESIS | 0.364 |
| REACTOME APOPTOSIS | 0.364 |
| REACTOME PROCESSING OF CAPPED INTRON CONTAINING PRE MRNA | 0.379 |
| KEGG PROTEIN DIGESTION AND ABSORPTION | 0.505 |
| KEGG SALIVARY SECRETION | 0.379 |
| KEGG PHAGOSOME | 0.404 |
| KEGG AMOEBIASIS | 0.505 |
| KEGG GLUTAMATERGIC SYNAPSE | 0.404 |
| REACTOME G1 S TRANSITION | 0.505 |
| KEGG DILATED CARDIOMYOPATHY | 0.528 |
| BIOCARTA P38MAPK PATHWAY | 0.528 |
| BIOCARTA CASPASE PATHWAY | 0.481 |
| KEGG TUBERCULOSIS | 0.528 |
| REACTOME PEPTIDE LIGAND BINDING RECEPTORS | 0.449 |
| REACTOME PLATELET DEGRANULATION | 0.528 |
| KEGG RNA DEGRADATION | 0.505 |
| KEGG CELL CYCLE | 0.528 |
| KEGG LYSOSOME | 0.528 |
| REACTOME G ALPHA Q SIGNALLING EVENTS | 0.551 |
| BIOCARTA ACE2 PATHWAY | 0.566 |
| BIOCARTA ACTINY PATHWAY | 0.543 |
| BIOCARTA AT1R PATHWAY | 0.752 |
| BIOCARTA BCELLSURVIVAL PATHWAY | 0.762 |
| BIOCARTA CACAM PATHWAY | 0.543 |
| BIOCARTA CARM ER PATHWAY | 0.762 |
| BIOCARTA CCR3 PATHWAY | 0.798 |
| BIOCARTA CCR5 PATHWAY | 0.566 |
| BIOCARTA CD40 PATHWAY | 0.543 |
| BIOCARTA CDMAC PATHWAY | 0.543 |
| BIOCARTA CK1 PATHWAY | 0.543 |
| BIOCARTA D4GDI PATHWAY | 0.752 |
| BIOCARTA DC PATHWAY | 0.543 |
| BIOCARTA DEATH PATHWAY | 0.619 |
| BIOCARTA DREAM PATHWAY | 0.798 |
| BIOCARTA EGFR SMRTE PATHWAY | 0.543 |
| BIOCARTA EIF PATHWAY | 0.798 |
| BIOCARTA EPHA4 PATHWAY | 0.543 |
| BIOCARTA EPONFKB PATHWAY | 0.566 |
| BIOCARTA EPO PATHWAY | 0.566 |
| BIOCARTA ERK5 PATHWAY | 0.566 |
| BIOCARTA ERK PATHWAY | 0.543 |
| BIOCARTA FAS PATHWAY | 0.585 |
| BIOCARTA FEEDER PATHWAY | 0.762 |
| BIOCARTA FMLP PATHWAY | 0.585 |
| BIOCARTA FREE PATHWAY | 0.543 |
| BIOCARTA GABA PATHWAY | 0.798 |
| BIOCARTA GATA3 PATHWAY | 0.543 |
| BIOCARTA GLYCOLYSIS PATHWAY | 0.798 |
| BIOCARTA HDAC PATHWAY | 0.762 |
| BIOCARTA HER2 PATHWAY | 0.566 |
| BIOCARTA IGF1 PATHWAY | 0.762 |
| BIOCARTA IL2 PATHWAY | 0.762 |
| BIOCARTA IL3 PATHWAY | 0.566 |
| BIOCARTA IL5 PATHWAY | 0.543 |
| BIOCARTA IL6 PATHWAY | 0.798 |
| BIOCARTA INFLAM PATHWAY | 0.762 |
| BIOCARTA INSULIN PATHWAY | 0.585 |
| BIOCARTA KERATINOCYTE PATHWAY | 0.602 |
| BIOCARTA MAL PATHWAY | 0.543 |
| BIOCARTA MAPK PATHWAY | 0.551 |
| BIOCARTA MCM PATHWAY | 0.798 |
| BIOCARTA MYOSIN PATHWAY | 0.566 |
| BIOCARTA NEUROTRANSMITTERS PATHWAY | 0.566 |
| BIOCARTA NFKB PATHWAY | 0.798 |
| BIOCARTA NO2IL12 PATHWAY | 0.566 |
| BIOCARTA P27 PATHWAY | 0.543 |
| BIOCARTA P35ALZHEIMERS PATHWAY | 0.543 |
| BIOCARTA PGC1A PATHWAY | 0.798 |
| BIOCARTA PML PATHWAY | 0.543 |
| BIOCARTA PPARA PATHWAY | 0.585 |
| BIOCARTA PS1 PATHWAY | 0.566 |
| BIOCARTA PTC1 PATHWAY | 0.543 |
| BIOCARTA PYK2 PATHWAY | 0.798 |
| BIOCARTA RARRXR PATHWAY | 0.798 |
| BIOCARTA RELA PATHWAY | 0.543 |
| BIOCARTA RHO PATHWAY | 0.566 |
| BIOCARTA RNA PATHWAY | 0.543 |
| BIOCARTA SALMONELLA PATHWAY | 0.543 |
| BIOCARTA SKP2E2F PATHWAY | 0.543 |
| BIOCARTA SRCRPTP PATHWAY | 0.543 |
| BIOCARTA STEM PATHWAY | 0.798 |
| BIOCARTA TCAPOPTOSIS PATHWAY | 0.543 |
| BIOCARTA TCR PATHWAY | 0.585 |
| BIOCARTA TEL PATHWAY | 0.543 |
| BIOCARTA TFF PATHWAY | 0.566 |
| BIOCARTA TH1TH2 PATHWAY | 0.566 |
| BIOCARTA TOLL PATHWAY | 0.762 |
| BIOCARTA VDR PATHWAY | 0.543 |
| BIOCARTA VEGF PATHWAY | 0.566 |
| BIOCARTA VIP PATHWAY | 0.543 |
| BIOCARTA VITCB PATHWAY | 0.798 |
| BIOCARTA WNT PATHWAY | 0.798 |
| KEGG ABC TRANSPORTERS | 0.798 |
| KEGG ALDOSTERONE REGULATED SODIUM REABSORPTION | 0.585 |
| KEGG ALPHA LINOLENIC ACID METABOLISM | 0.566 |
| KEGG AUTOIMMUNE THYROID DISEASE | 0.602 |
| KEGG BETA ALANINE METABOLISM | 0.602 |
| KEGG BILE SECRETION | 0.757 |
| KEGG BIOSYNTHESIS OF UNSATURATED FATTY ACIDS | 0.602 |
| KEGG CARBOHYDRATE DIGESTION AND ABSORPTION | 0.752 |
| KEGG CHEMOKINE SIGNALING PATHWAY | 0.670 |
| KEGG CIRCADIAN RHYTHM MAMMAL | 0.543 |
| KEGG COMPLEMENT AND COAGULATION CASCADES | 0.619 |
| KEGG CYTOKINE CYTOKINE RECEPTOR INTERACTION | 0.602 |
| KEGG CYTOSOLIC DNA SENSING PATHWAY | 0.602 |
| KEGG DRUG METABOLISM CYTOCHROME P450 | 0.602 |
| KEGG DRUG METABOLISM OTHER ENZYMES | 0.585 |
| KEGG D ARGININE AND D ORNITHINE METABOLISM | 0.543 |
| KEGG EPITHELIAL CELL SIGNALING IN HELICOBACTER PYLORI INFECT . . . | 0.602 |
| KEGG ETHER LIPID METABOLISM | 0.585 |
| KEGG FATTY ACID ELONGATION IN MITOCHONDRIA | 0.752 |
| KEGG FC GAMMA R MEDIATED PHAGOCYTOSIS | 0.653 |
| KEGG FOCAL ADHESION | 0.670 |
| KEGG FOLATE BIOSYNTHESIS | 0.543 |
| KEGG GAP JUNCTION | 0.619 |
| KEGG GASTRIC ACID SECRETION | 0.602 |
| KEGG GLYCINE SERINE AND THREONINE METABOLISM | 0.602 |
| KEGG GLYCOSAMINOGLYCAN BIOSYNTHESIS CHONDROITIN SULFATE | 0.585 |
| KEGG GLYCOSAMINOGLYCAN BIOSYNTHESIS KERATAN SULFATE | 0.543 |
| KEGG GLYCOSAMINOGLYCAN DEGRADATION | 0.798 |
| KEGG GLYOXYLATE AND DICARBOXYLATE METABOLISM | 0.585 |
| KEGG GNRH SIGNALING PATHWAY | 0.636 |
| KEGG HISTIDINE METABOLISM | 0.798 |
| KEGG HYPERTROPHIC CARDIOMYOPATHY HCM | 0.653 |
| KEGG INFLUENZA A | 0.777 |
| KEGG JAK STAT SIGNALING PATHWAY | 0.766 |
| KEGG LEISHMANIASIS | 0.636 |
| KEGG LIPOIC ACID METABOLISM | 0.543 |
| KEGG MAPK SIGNALING PATHWAY | 0.752 |
| KEGG MEASLES | 0.551 |
| KEGG MRNA SURVEILLANCE PATHWAY | 0.653 |
| KEGG NEUROACTIVE LIGAND RECEPTOR INTERACTION | 0.762 |
| KEGG OTHER TYPES OF O GLYCAN BIOSYNTHESIS | 0.585 |
| KEGG OXIDATIVE PHOSPHORYLATION | 0.752 |
| KEGG P53 SIGNALING PATHWAY | 0.636 |
| KEGG PANTOTHENATE AND COA BIOSYNTHESIS | 0.762 |
| KEGG PATHOGENIC ESCHERICHIA COLI INFECTION | 0.762 |
| KEGG PHOTOTRANSDUCTION | 0.566 |
| KEGG PRIMARY IMMUNODEFICIENCY | 0.543 |
| KEGG PROGESTERONE MEDIATED OOCYTE MATURATION | 0.636 |
| KEGG PYRUVATE METABOLISM | 0.602 |
| KEGG RENIN ANGIOTENSIN SYSTEM | 0.566 |
| KEGG RHEUMATOID ARTHRITIS | 0.602 |
| KEGG RIBOSOME BIOGENESIS IN EUKARYOTES | 0.602 |
| KEGG RNA TRANSPORT | 0.757 |
| KEGG SELENOCOMPOUND METABOLISM | 0.566 |
| KEGG SHIGELLOSIS | 0.602 |
| KEGG SPHINGOLIPID METABOLISM | 0.752 |
| KEGG STARCH AND SUCROSE METABOLISM | 0.762 |
| KEGG SULFUR METABOLISM | 0.798 |
| KEGG SULFUR RELAY SYSTEM | 0.798 |
| KEGG SYNTHESIS AND DEGRADATION OF KETONE BODIES | 0.543 |
| KEGG THYROID CANCER | 0.543 |
| KEGG TOXOPLASMOSIS | 0.670 |
| KEGG TYROSINE METABOLISM | 0.566 |
| KEGG UBIQUITIN MEDIATED PROTEOLYSIS | 0.766 |
| KEGG VEGF SIGNALING PATHWAY | 0.619 |
| KEGG VIBRIO CHOLERAE INFECTION | 0.762 |
| REACTOME ABORTIVE ELONGATION OF HIV1 TRANSCRIPT IN THE ABSEN . . . | 0.543 |
| REACTOME ACETYLCHOLINE NEUROTRANSMITTER RELEASE CYCLE | 0.566 |
| REACTOME ACTIVATED TLR4 SIGNALLING | 0.762 |
| REACTOME ACTIVATION OF KAINATE RECEPTORS UPON GLUTAMATE BIND . . . | 0.798 |
| REACTOME ADENYLATE CYCLASE ACTIVATING PATHWAY | 0.798 |
| REACTOME AKT PHOSPHORYLATES TARGETS IN THE CYTOSOL | 0.585 |
| REACTOME AMINE DERIVED HORMONES | 0.566 |
| REACTOME AMINO ACID TRANSPORT ACROSS THE PLASMA MEMBRANE | 0.602 |
| REACTOME BASE EXCISION REPAIR | 0.602 |
| REACTOME BASE FREE SUGAR PHOSPHATE REMOVAL VIA THE SINGLE NU . . . | 0.602 |
| REACTOME BASIGIN INTERACTIONS | 0.566 |
| REACTOME CAM PATHWAY | 0.798 |
| REACTOME CELLEXTRACELLULAR MATRIX INTERACTIONS | 0.798 |
| REACTOME CELL CELL ADHESION SYSTEMS | 0.566 |
| REACTOME CELL CYCLE CHECKPOINTS | 0.653 |
| REACTOME CELL JUNCTION ORGANIZATION | 0.762 |
| REACTOME CHEMOKINE RECEPTORS BIND CHEMOKINES | 0.566 |
| REACTOME CLASS A1 RHODOPSIN LIKE RECEPTORS | 0.752 |
| REACTOME CRMPS IN SEMA3A SIGNALING | 0.543 |
| REACTOME CTLA4 INHIBITORY SIGNALING | 0.543 |
| REACTOME CYCLIN A1 ASSOCIATED EVENTS DURING G2 M TRANSITION | 0.543 |
| REACTOME CYCLIN E ASSOCIATED EVENTS DURING G1 S TRANSITION | 0.762 |
| REACTOME CYTOSOLIC TRNA AMINOACYLATION | 0.798 |
| REACTOME DEPOLARIZATION OF THE PRESYNAPTIC TERMINAL TRIGGERS . . . | 0.798 |
| REACTOME DNA REPAIR | 0.766 |
| REACTOME DNA REPLICATION PRE INITIATION | 0.636 |
| REACTOME DOWNSTREAM EVENTS IN GPCR SIGNALING | 0.752 |
| REACTOME DUAL INCISION REACTION IN GG NER | 0.798 |
| REACTOME E2F ENABLED INHIBITION OF PRE REPLICATION COMPLEX F . . . | 0.798 |
| REACTOME E2F MEDIATED REGULATION OF DNA REPLICATION | 0.585 |
| REACTOME EFFECTS OF PIP2 HYDROLYSIS | 0.566 |
| REACTOME EGFR DOWNREGULATION | 0.566 |
| REACTOME ELECTRON TRANSPORT CHAIN | 0.762 |
| REACTOME ENERGY DEPENDENT REGULATION OF MTOR BY LKB1 AMPK | 0.762 |
| REACTOME FACILITATIVE NA INDEPENDENT GLUCOSE TRANSPORTERS | 0.585 |
| REACTOME FGFR LIGAND BINDING AND ACTIVATION | 0.798 |
| REACTOME FORMATION OF THE EARLY ELONGATION COMPLEX | 0.798 |
| REACTOME FORMATION OF THE TERNARY COMPLEX AND SUBSEQUENTLY | 0.566 |
| T . . . | |
| REACTOME FORMATION OF TUBULIN FOLDING INTERMEDIATES BY CCT T . . . | 0.798 |
| REACTOME FRS2MEDIATED CASCADE | 0.798 |
| REACTOME FURTHER PLATELET RELEASATE | 0.798 |
| REACTOME GAMMA CARBOXYLATION TRANSPORT AND AMINO TERMINAL | 0.566 |
| CL . . . | |
| REACTOME GENERIC TRANSCRIPTION PATHWAY | 0.798 |
| REACTOME GLOBAL GENOMIC NER | 0.762 |
| REACTOME GLUCAGON SIGNALING IN METABOLIC REGULATION | 0.798 |
| REACTOME GLUCAGON TYPE LIGAND RECEPTORS | 0.543 |
| REACTOME GLUCOSE REGULATION OF INSULIN SECRETION | 0.551 |
| REACTOME GLUTAMATE NEUROTRANSMITTER RELEASE CYCLE | 0.566 |
| REACTOME GLUTATHIONE CONJUGATION | 0.762 |
| REACTOME GS ALPHA MEDIATED EVENTS IN GLUCAGON SIGNALLING | 0.798 |
| REACTOME G ALPHA I SIGNALLING EVENTS | 0.573 |
| REACTOME G BETA GAMMA SIGNALLING THROUGH PI3KGAMMA | 0.798 |
| REACTOME G PROTEIN BETA GAMMA SIGNALLING | 0.798 |
| REACTOME HOMOLOGOUS RECOMBINATION REPAIR | 0.543 |
| REACTOME HOST INTERACTIONS OF HIV FACTORS | 0.752 |
| REACTOME HUMAN TAK1 ACTIVATES NFKB BY PHOSPHORYLATION AND | 0.543 |
| AC . . . | |
| REACTOME INACTIVATION OF APC VIA DIRECT INHIBITION OF THE AP . . . | 0.566 |
| REACTOME INTEGRATION OF ENERGY METABOLISM | 0.689 |
| REACTOME IONOTROPIC ACTIVITY OF KAINATE RECEPTORS | 0.798 |
| REACTOME METABLISM OF NUCLEOTIDES | 0.757 |
| REACTOME METABOLISM OF MRNA | 0.798 |
| REACTOME METABOLISM OF RNA | 0.762 |
| REACTOME MICRORNA BIOGENESIS | 0.543 |
| REACTOME MITOTIC PROMETAPHASE | 0.752 |
| REACTOME MRNA DECAY BY 3 TO 5 EXORIBONUCLEASE | 0.798 |
| REACTOME MTORC1 MEDIATED SIGNALLING | 0.566 |
| REACTOME MTOR SIGNALLING | 0.762 |
| REACTOME MUSCLE CONTRACTION | 0.752 |
| REACTOME MYOGENESSIS | 0.762 |
| REACTOME NCAM1 INTERACTIONS | 0.752 |
| REACTOME NCAM SIGNALING FOR NEURITE OUT GROWTH | 0.752 |
| REACTOME NEF MEDIATES DOWN MODULATION OF CELL SURFACE RECEPT . . . | 0.798 |
| REACTOME NEP NS2 INTERACTS WITH THE CELLULAR EXPORT MACHINER . . . | 0.798 |
| REACTOME NEUROTRANSMITTER RELEASE CYCLE | 0.585 |
| REACTOME NF KB IS ACTIVATED AND SIGNALS SURVIVAL | 0.798 |
| REACTOME NOREPINEPHRINE NEUROTRANSMITTER RELEASE CYCLE | 0.798 |
| REACTOME NOTCH HLH TRANSCRIPTION PATHWAY | 0.543 |
| REACTOME NUCLEAR IMPORT OF REV PROTEIN | 0.798 |
| REACTOME NUCLEAR RECEPTOR TRANSCRIPTION PATHWAY | 0.602 |
| REACTOME NUCLEOTIDE EXCISION REPAIR | 0.762 |
| REACTOME NUCLEOTIDE LIKE PURINERGIC RECEPTORS | 0.543 |
| REACTOME OPIOID SIGNALLING | 0.752 |
| REACTOME OPSINS | 0.543 |
| REACTOME OTHER SEMAPHORIN INTERACTIONS | 0.566 |
| REACTOME P75NTR SIGNALS VIA NFKB | 0.798 |
| REACTOME PECAM1 INTERACTIONS | 0.543 |
| REACTOME PEROXISOMAL LIPID METABOLISM | 0.762 |
| REACTOME PHASE 1 FUNCTIONALIZATION | 0.798 |
| REACTOME PHASE II CONJUGATION | 0.752 |
| REACTOME PHOSPHOLIPASE CMEDIATED CASCADE | 0.798 |
| REACTOME PKA ACTIVATION | 0.798 |
| REACTOME POLYMERASE SWITCHING | 0.798 |
| REACTOME PP2A MEDIATED DEPHOSPHORYLATION OF KEY METABOLIC FA . . . | 0.798 |
| REACTOME PREFOLDIN MEDIATED TRANSFER OF SUBSTRATE TO CCT TRI . . . | 0.798 |
| REACTOME PROSTANOID HORMONES | 0.543 |
| REACTOME PURINE METABOLISM | 0.762 |
| REACTOME PYRIMIDINE CATABOLISM | 0.585 |
| REACTOME PYRIMIDINE METABOLISM | 0.752 |
| REACTOME REGULATION OF AMPK ACTIVITY VIA LKB1 | 0.762 |
| REACTOME REGULATION OF GLUCOKINASE BY GLUCOKINASE | 0.798 |
| REGULATORY . . . | |
| REACTOME REGULATION OF INSULIN SECRETION BY ACETYLCHOLINE | 0.566 |
| REACTOME REGULATION OF INSULIN SECRETION BY FREE FATTY ACIDS | 0.566 |
| REACTOME REGULATION OF INSULIN SECRETION BY GLUCAGON LIKE PE . . . | 0.602 |
| REACTOME REGULATION OF LIPID METABOLISM BY PEROXISOME PROLIF . . . | 0.762 |
| REACTOME REMOVAL OF DNA PATCH CONTAINING ABASIC RESIDUE | 0.602 |
| REACTOME REPAIR SYNTHESIS OF PATCH 27 30 BASES LONG BY DNA P . . . | 0.566 |
| REACTOME RESOLUTION OF AP SITES VIA THE SINGLE NUCLEOTIDE RE . . . | 0.602 |
| REACTOME REV MEDIATED NUCLEAR EXPORT OF HIV1 RNA | 0.798 |
| REACTOME RHO GTPASE CYCLE | 0.752 |
| REACTOME SEMA3A PAK DEPENDENT AXON REPULSION | 0.543 |
| REACTOME SEMA3A PLEXIN REPULSION SIGNALING BY INHIBITING INT . . . | 0.566 |
| REACTOME SEMA4D INDUCED CELL MIGRATION AND GROWTH CONE | 0.752 |
| COLLA . . . | |
| REACTOME SEMA4D IN SEMAPHORIN SIGNALING | 0.752 |
| REACTOME SIGNALING BY BMP | 0.585 |
| REACTOME SIGNALING BY ROBO RECEPTOR | 0.585 |
| REACTOME SMOOTH MUSCLE CONTRACTION | 0.798 |
| REACTOME SNRNP ASSEMBLY | 0.798 |
| REACTOME SPHINGOLIPID METABOLISM | 0.566 |
| REACTOME STABILIZATION OF P53 | 0.752 |
| REACTOME STEROID HORMONES | 0.566 |
| REACTOME STEROID HORMONE BIOSYNTHESIS | 0.798 |
| REACTOME STEROID METABOLISM | 0.602 |
| REACTOME STRIATED MUSCLE CONTRACTION | 0.752 |
| REACTOME SYNTHESIS AND INTERCONVERSION OF NUCLEOTIDE DI AND . . . | 0.798 |
| REACTOME SYNTHESIS OF DNA | 0.752 |
| REACTOME S PHASE | 0.619 |
| REACTOME TELOMERE MAINTENANCE | 0.619 |
| REACTOME THE ROLE OF NEF IN HIV1 REPLICATION AND DISEASE PAT . . . | 0.798 |
| REACTOME THROMBIN SIGNALLING THROUGH PROTEINASE ACTIVATED RE . . . | 0.543 |
| REACTOME TIGHT JUNCTION INTERACTIONS | 0.566 |
| REACTOME TOLL LIKE RECEPTOR 4 CASCADE | 0.762 |
| REACTOME TOLL RECEPTOR CASCADES | 0.602 |
| REACTOME TRAF6 MEDIATED INDUCTION OF THE ANTIVIRAL CYTOKINE . . . | 0.798 |
| REACTOME TRANSCRIPTION COUPLED NER | 0.762 |
| REACTOME TRANSFORMATION OF LANOSTEROL TO CHOLESTEROL | 0.543 |
| REACTOME TRANSLATION INITIATION COMPLEX FORMATION | 0.566 |
| REACTOME TRANSPORT OF RIBONUCLEOPROTEINS INTO THE HOST NUCLE.. | 0.798 |
| REACTOME VPR MEDIATED NUCLEAR IMPORT OF PICS | 0.798 |
| REACTOME XENOBIOTICS | 0.543 |
| SA CASPASE CASCADE | 0.752 |
| SA G2 AND M PHASES | 0.543 |
| SA MMP CYTOKINE CONNECTION | 0.798 |
| SA PTEN PATHWAY | 0.585 |
| SA REG CASCADE OF CYCLIN EXPR | 0.543 |
| SIG IL4RECEPTOR IN B LYPHOCYTES | 0.566 |
| ST ERK1 ERK2 MAPK PATHWAY | 0.543 |
| ST GA13 PATHWAY | 0.566 |
| ST G ALPHA S PATHWAY | 0.543 |
| ST IL 13 PATHWAY | 0.543 |
| ST INTERFERON GAMMA PATHWAY | 0.543 |
| ST INTERLEUKIN 13 PATHWAY | 0.543 |
| ST JAK STAT PATHWAY | 0.543 |
| ST JNK MAPK PATHWAY | 0.762 |
| ST P38 MAPK PATHWAY | 0.762 |
| ST STAT3 PATHWAY | 0.543 |
| ST TYPE I INTERFERON PATHWAY | 0.543 |
| ST T CELL SIGNAL TRANSDUCTION | 0.798 |
| ST WNT BETA CATENIN PATHWAY | 0.543 |
| WNT SIGNALING | 0.602 |

TABLE 11
Top genes selected from the Utah 1 cohort

| Entrez | | |
|---|---|---|
| Gene ID | Gene Symbol | Gene Name |
| 87688 | RPL7AP50 | ribosomal protein L7a pseudogene 50 |
| 391106 | VDAC1P9 | voltage-dependent anion channel 1 pseudogene 9 |
| 100128709 | [No Symbol] | [No Name] |
| 646272 | LOC646272 | cytochrome b-c1 complex subunit 8-like |
| 100128700 | [No Symbol] | [No Name] |
| 286495 | TTC3P1 | tetratricopeptide repeat domain 3 pseudogene 1 |
| 392282 | RPS5P6 | ribosomal protein S5 pseudogene 6 |
| 541468 | C1orf190 | chromosome 1 open reading frame 190 |
| 83604 | TMEM47 | transmembrane protein 47 |
| 400954 | EML6 | echinoderm microtubule associated protein like 6 |
| 647298 | HSPD1P8 | heat shock 60 kDa protein 1 (chaperonin) pseudogene 8 |
| 100132626 | LOC100132626 | protein FAM103A1-like |
| 440278 | CATSPER2P1 | cation channel, sperm associated 2 pseudogene 1 |
| 100128493 | LOC100128493 | ubiquitin-conjugating enzyme E2 variant 2 pseudogene |
| 390155 | OR5T1 | olfactory receptor, family 5, subfamily T, member 1 |
| 128774 | MRPS11P1 | mitochondrial ribosomal protein S11 pseudogene 1 |
| 641518 | LOC641518 | hypothetical LOC641518 |
| 149157 | [No Symbol] | [No Name] |
| 7473 | WNT3 | wingless-type MMTV integration site family, member 3 |
| 54436 | SH3TC1 | SH3 domain and tetratricopeptide repeats 1 |
| 5999 | RGS4 | regulator of G-protein signaling 4 |
| 28869 | IGKV6D-41 | immunoglobulin kappa variable 6D-41 (non-functional) |
| 55604 | LRRC16A | leucine rich repeat containing 16A |
| 449518 | LOC449518 | purinergic receptor P2Y, G-protein coupled, 10 pseudogene |
| 79825 | CCDC48 | coiled-coil domain containing 48 |
| 3809 | KIR2DS4 | killer cell immunoglobulin-like receptor, two domains, short . . . |
| 442673 | TUBG1P | tubulin, gamma 1 pseudogene |
| 51266 | CLEC1B | C-type lectin domain family 1, member B |
| 100129822 | [No Symbol] | [No Name] |
| 100131609 | HNRNPA1P2 | heterogeneous nuclear ribonucleoprotein A1 pseudogene 2 |
| 389428 | RPL5P18 | ribosomal protein L5 pseudogene 18 |
| 100128386 | LOC100128386 | hypothetical LOC100128386 |
| 55270 | NUDT15 | nudix (nucleoside diphosphate linked moiety X)-type motif 15 |
| 649489 | LOC649489 | protein phosphatase 1, regulatory (inhibitor) subunit 2 pseu . . . |
| 643586 | LOC643586 | pyruvate kinase, muscle pseudogene |
| 339778 | C2orf70 | chromosome 2 open reading frame 70 |
| 348825 | TPRXL | tetra-peptide repeat homeobox-like |
| 221016 | CCDC7 | coiled-coil domain containing 7 |
| 647034 | RPS14P10 | ribosomal protein S14 pseudogene 10 |
| 3805 | KIR2DL4 | killer cell immunoglobulin-like receptor, two domains, long . . . |
| 283314 | MATL2963 | hypothetical LOC283314 |
| 7380 | UPK3A | uroplakin 3A |
| 408029 | C2orf27B | chromosome 2 open reading frame 27B |
| 10461 | MERTK | c-mer proto-oncogene tyrosine kinase |
| 642677 | LOC642677 | family with sequence similarity 154, member B pseudogene |
| 10877 | CFHR4 | complement factor H-related 4 |
| 283571 | PROX2 | prospero homeobox 2 |
| 340547 | VSIG1 | V-set and immunoglobulin domain containing 1 |
| 100129915 | [No Symbol] | [No Name] |
| 3426 | CFI | complement factor I |
| 51499 | TRIAP1 | TP53 regulated inhibitor of apoptosis 1 |
| 780813 | PAICSP4 | phosphoribosylaminoimidazole carboxylase, phosphoribosylamin . . . |
| 728707 | [No Symbol] | [No Name] |
| 130813 | C2orf50 | chromosome 2 open reading frame 50 |
| 100132310 | LOC100132310 | FCF1 small subunit (SSU) processome component homolog (S. ce . . . |
| 729486 | IL9RP3 | interleukin 9 receptor pseudogene 3 |
| 401433 | LOC401433 | hypothetical LOC401433 |
| 286122 | C8orf31 | chromosome 8 open reading frame 31 |
| 219902 | TMEM136 | transmembrane protein 136 |
| 199713 | NLRP7 | NLR family, pyrin domain containing 7 |
| 390282 | LOC390282 | eukaryotic translation initiation factor 3, subunit F pseudo . . . |
| 1769 | DNAH8 | dynein, axonemal, heavy chain 8 |
| 100130249 | PP2672 | hypothetical LOC100130249 |
| 163778 | SPRR4 | small proline-rich protein 4 |
| 148641 | SLC35F3 | solute carrier family 35, member F3 |
| 400347 | LOC400347 | REX4, RNA exonuclease 4 homolog (S. cerevisiae) pseudogene |
| 347333 | KRT8P14 | keratin 8 pseudogene 14 |
| 100128646 | RPL10AP7 | ribosomal protein L10a pseudogene 7 |
| 54035 | PSMD4P1 | proteasome (prosome, macropain) 26S subunit, non-ATPase, 4 p . . . |
| 8228 | PNPLA4 | patatin-like phospholipase domain containing 4 |
| 9363 | RAB33A | RAB33A, member RAS oncogene family |
| 344887 | LOC344887 | NmrA-like family domain containing 1 pseudogene |
| 121270 | OR11M1P | olfactory receptor, family 11, subfamily M, member 1 pseudog . . . |
| 339736 | AK2P2 | adenylate kinase 2 pseudogene 2 |
| 6461 | SHB | Src homology 2 domain containing adaptor protein B |
| 4744 | NEFH | neurofilament, heavy polypeptide |
| 90499 | LOC90499 | hypothetical protein LOC90499 |
| 729041 | LOC729041 | fatty-acid amide hydrolase 1-like |
| 2335 | FN1 | fibronectin 1 |
| 79625 | C4orf31 | chromosome 4 open reading frame 31 |
| 644915 | METTL15P2 | methyltransferase like 15 pseudogene 2 |
| 100132086 | LOC100132086 | adenylate kinase isoenzyme 6-like |
| 2596 | GAP43 | growth associated protein 43 |
| 326617 | PSMA3P | proteasome (prosome, macropain) subunit, alpha type, 3 pseud . . . |
| 646576 | LOC646576 | hypothetical LOC646576 |
| 3812 | KIR3DL2 | killer cell immunoglobulin-like receptor, three domains, lon . . . |
| 100129958 | KRT8P44 | keratin 8 pseudogene 44 |
| 644662 | LOC644662 | hypothetical protein LOC644662 |
| 5054 | SERPINE1 | serpin peptidase inhibitor, clade E (nexin, plasminogen acti . . . |
| 339983 | NAT8L | N-acetyltransferase 8-like (GCN5-related, putative) |
| 100037267 | LOC100037267 | developmental pluripotency associated 4 pseudogene |
| 130013 | ACMSD | aminocarboxymuconate semialdehyde decarboxylase |
| 219623 | TMEM26 | transmembrane protein 26 |
| 9241 | NOG | noggin |
| 100128050 | LOC100128050 | WD repeat domain 77 pseudogene |
| 440603 | BCL2L15 | BCL2-like 15 |
| 152078 | C3orf55 | chromosome 3 open reading frame 55 |
| 201895 | C4orf34 | chromosome 4 open reading frame 34 |
| 100127889 | C10orf131 | chromosome 10 open reading frame 131 |
| 221711 | SYCP2L | synaptonemal complex protein 2-like |
| 8829 | NRP1 | neuropilin 1 |
| 1472 | CST4 | cystatin S |
| 729451 | LOC729451 | hypothetical protein LOC729451 |
| 100128389 | [No Symbol] | [No Name] |
| 201798 | TIGD4 | tigger transposable element derived 4 |
| 118663 | BTBD16 | BTB (POZ) domain containing 16 |
| 6887 | TAL2 | T-cell acute lymphocytic leukemia 2 |
| 64410 | KLHL25 | kelch-like 25 (Drosophila) |
| 442524 | DPY19L2P3 | dpy-19-like 2 pseudogene 3 (C. elegans) |
| 259286 | TAS2R40 | taste receptor, type 2, member 40 |
| 731039 | [No Symbol] | [No Name] |
| 50835 | TAS2R9 | taste receptor, type 2, member 9 |
| 4157 | MC1R | melanocortin 1 receptor (alpha melanocyte stimulating hormon . . . |
| 401703 | LOC401703 | splicing factor U2AF 35 kDa subunit-like |
| 785 | CACNB4 | calcium channel, voltage-dependent, beta 4 subunit |
| 100130268 | LOC100130268 | similar to hCG1648866 |
| 100130859 | [No Symbol] | [No Name] |
| 100128457 | LOC100128457 | similar to hCG2026341 |
| 100132214 | [No Symbol] | [No Name] |
| 26 | ABP1 | amiloride binding protein 1 (amine oxidase (copper-containin . . . |
| 51676 | ASB2 | ankyrin repeat and SOCS box containing 2 |
| 27145 | FILIP1 | filamin A interacting protein 1 |
| 347051 | SLC10A5 | solute carrier family 10 (sodium/bile acid cotransporter fam . . . |
| 100131819 | [No Symbol] | [No Name] |
| 728780 | ANKDD1B | ankyrin repeat and death domain containing 1B |

TABLE 12
Top genes selected from the Utah 2 cohort.

| Entrees | | |
|---|---|---|
| Gene ID | Gene Symbol | Gene Name |
| 541468 | C1orf190 | chromosome 1 open reading frame 190 |
| 55057 | AIM1L | absent in melanoma 1-like |
| 221016 | CCDC7 | coiled-coil domain containing 7 |
| 7012 | TERC | telomerase RNA component |
| 148641 | SLC35F3 | solute carrier family 35, member F3 |
| 55270 | NUDT15 | nudix (nucleoside diphosphate linked moiety X)-type motif 15 |
| 647298 | HSPD1P8 | heat shock 60 kDa protein 1 (chaperonin) pseudogene 8 |
| 392282 | RPS5P6 | ribosomal protein S5 pseudogene 6 |
| 728707 | [No Symbol] | [No Name] |
| 389428 | RPL5P18 | ribosomal protein L5 pseudogene 18 |
| 730255 | RPL17P8 | ribosomal protein L17 pseudogene 8 |
| 100130249 | PP2672 | hypothetical LOC100130249 |
| 4112 | MAGEB1 | melanoma antigen family B, 1 |
| 163778 | SPRR4 | small proline-rich protein 4 |
| 157927 | C9orf62 | chromosome 9 open reading frame 62 |
| 100129822 | [No Symbol] | [No Name] |
| 83604 | TMEM47 | transmembrane protein 47 |
| 729486 | IL9RP3 | interleukin 9 receptor pseudogene 3 |
| 390282 | LOC390282 | eukaryotic translation initiation factor 3, subunit F pseudo . . . |
| 197370 | NSMCE1 | non-SMC element 1 homolog (S. cerevisiae) |
| 728509 | RPS19P7 | ribosomal protein S19 pseudogene 7 |
| 286122 | C8orf31 | chromosome 8 open reading frame 31 |
| 442524 | DPY19L2P3 | dpy-19-like 2 pseudogene 3 (C. elegans) |
| 647034 | RPS14P10 | ribosomal protein S14 pseudogene 10 |
| 400954 | EML6 | E\grave{ }1chinoderm microtubule associated protein like 6 |
| 339778 | C2orf70 | chromosome 2 open reading frame 70 |
| 100128700 | [No Symbol] | [No Name] |
| 730126 | [No Symbol] | [No Name] |
| 644662 | LOC644662 | hypothetical protein LOC644662 |
| 642677 | LOC642677 | family with sequence similarity 154, member B pseudogene |
| 785 | CACNB4 | calcium channel, voltage-dependent, beta 4 subunit |
| 149157 | [No Symbol] | [No Name] |
| 100128050 | LOC100128050 | WD repeat domain 77 pseudogene |
| 729451 | LOC729451 | hypothetical protein LOC729451 |
| 51266 | CLEC1B | C-type lectin domain family 1, member B |
| 100128646 | RPL10AP7 | ribosomal protein L10a pseudogene 7 |
| 148645 | C1orf211 | chromosome 1 open reading frame 211 |
| 440278 | CATSPER2P1 | cation channel, sperm associated 2 pseudogene 1 |
| 653492 | PSG10P | pregnancy specific beta-1-glycoprotein 10, pseudogene |
| 347333 | KRT8P14 | keratin 8 pseudogene 14 |
| 391106 | VDAC1P9 | voltage-dependent anion channel 1 pseudogene 9 |
| 100128386 | LOC100128386 | hypothetical LOC100128386 |
| 441502 | RPS26P11 | ribosomal protein S26 pseudogene 11 |
| 100132086 | LOC100132086 | adenylate kinase isoenzyme 6-like |
| 121270 | OR11M1P | olfactory receptor, family 11, subfamily M, member 1 pseudog . . . |
| 79022 | TMEM106C | transmembrane protein 106C |
| 408029 | C2orf27B | chromosome 2 open reading frame 27B |
| 6461 | SHB | Src homology 2 domain containing adaptor protein B |
| 27145 | FILIP1 | filamin A interacting protein 1 |
| 401433 | LOC401433 | hypothetical LOC401433 |
| 100131042 | [No Symbol] | [No Name] |
| 646576 | LOC646576 | hypothetical LOC646576 |
| 10877 | CFHR4 | complement factor H-related 4 |
| 326617 | PSMA3P | proteasome (prosome, macropain) subunit, alpha type, 3 pseud . . . |
| 3394 | IRF8 | interferon regulatory factor 8 |
| 100128493 | LOC100128493 | ubiquitin-conjugating enzyme E2 variant 2 pseudogene |
| 440603 | BCL2L15 | BCL2-like 15 |
| 344887 | LOC344887 | NmrA-like family domain containing 1 pseudogene |
| 728050 | [No Symbol] | [No Name] |
| 645086 | LOC645086 | chromosome 11 open reading frame 58 pseudogene |
| 649288 | AK4P6 | adenylate kinase 4 pseudogene 6 |
| 3872 | KRT17 | keratin 17 |
| 100128979 | LOC100128979 | hypothetical LOC100128979 |
| 100128457 | LOC100128457 | similar to hCG2026341 |
| 283553 | LOC283553 | hypothetical LOC283553 |
| 1823 | DSC1 | desmocollin 1 |
| 199713 | NLRP7 | NLR family, pyrin domain containing 7 |
| 91392 | ZNF502 | zinc finger protein 502 |
| 130813 | C2orf50 | chromosome 2 open reading frame 50 |
| 219623 | TMEM26 | transmembrane protein 26 |
| 2596 | GAP43 | growth associated protein 43 |
| 441505 | LOC441505 | stress-induced-phosphoprotein 1 pseudogene |
| 643586 | LOC643586 | pyruvate kinase, muscle pseudogene |
| 449518 | LOC449518 | purinergic receptor P2Y, G-protein coupled, 10 pseudogene |
| 3426 | CFI | complement factor I |
| 128774 | MRPS11P1 | mitochondrial ribosomal protein S11 pseudogene 1 |
| 87688 | RPL7AP50 | ribosomal protein L7a pseudogene 50 |
| 7380 | UPK3A | uroplakin 3A |
| 84332 | DYDC2 | DPY30 domain containing 2 |
| 100129915 | [No Symbol] | [No Name] |
| 100132805 | [No Symbol] | [No Name] |
| 342666 | FLJ43826 | FLJ43826 protein |
| 6862 | T | T, brachyury homolog (mouse) |
| 348825 | TPRXL | tetra-peptide repeat homeobox-like |
| 1472 | CST4 | cystatin S |
| 388182 | FLJ42289 | hypothetical LOC388182 |
| 4342 | MOS | v-mos Moloney murine sarcoma viral oncogene homolog |
| 78998 | C8orf51 | chromosome 8 open reading frame 51 |
| 9241 | NOG | noggin |
| 780813 | PAICSP4 | phosphoribosylaminoimidazole carboxylase, phosphoribosylamin . . . |
| 26 | ABP1 | amiloride binding protein 1 (amine oxidase (copper-containin . . . |
| 55540 | IL17RB | interleukin 17 receptor B |
| 100128709 | [No Symbol] | [No Name] |
| 100130268 | LOC100130268 | similar to hCG1648866 |
| 100128389 | [No Symbol] | [No Name] |
| 353194 | LOC353194 | keratin pseudogene |
| 100131609 | HNRNPA1P2 | heterogeneous nuclear ribonucleoprotein A1 pseudogene 2 |
| 130500 | CISD1P1 | CDGSH iron sulfur domain 1 pseudogene 1 |
| 26256 | CABYR | calcium binding tyrosine-(Y)-phosphorylation regulated |
| 100127889 | C10orf131 | chromosome 10 open reading frame 131 |
| 100128417 | [No Symbol] | [No Name] |
| 79625 | C4orf31 | chromosome 4 open reading frame 31 |
| 83844 | USP26 | ubiquitin specific peptidase 26 |
| 128820 | CST9LP1 | cystatin 9-like pseudogene 1 |
| 100132214 | [No Symbol] | [No Name] |
| 26647 | OR7E25P | olfactory receptor, family 7, subfamily E, member 25 pseudog . . . |
| 8228 | PNPLA4 | patatin-like phospholipase domain containing 4 |
| 26590 | OR8B7P | olfactory receptor, family 8, subfamily B, member 7 pseudoge . . . |
| 729041 | LOC729041 | fatty-acid amide hydrolase 1-like |
| 100130859 | [No Symbol] | [No Name] |
| 221711 | SYCP2L | synaptonemal complex protein 2-like |
| 400165 | C13orf35 | chromosome 13 open reading frame 35 |
| 4157 | MC1R | melanocortin 1 receptor (alpha melanocyte stimulating hormon . . . |
| 3809 | KIR2DS4 | killer cell immunoglobulin-like receptor, two domains, short . . . |
| 9288 | TAAR3 | trace amine associated receptor 3 (gene/pseudogene) |
| 10461 | MERTK | c-mer proto-oncogene tyrosine kinase |
| 100129958 | KRT8P44 | keratin 8 pseudogene 44 |
| 100130321 | LOC100130321 | DNA fragmentation factor, 45 kDa, alpha polypeptide pseudogen . . . |
| 100132626 | LOC100132626 | protein FAM103A1-like |
| 729011 | [No Symbol] | [No Name] |
| 649489 | LOC649489 | Protein phosphatase 1, regulatory (inhibitor) subunit 2 pseu . . . |
| 4744 | NEFH | neurofilament, heavy polypeptide |
| 7070 | THY1 | Thy-1 cell surface antigen |
| 7644 | ZNF91 | zinc finger protein 91 |
| 144715 | RAD9B | RAD9 homolog B (S. pombe) |
| 653194 | LOC653194 | KH homology domain-containing protein 1-like |
| 7473 | WNT3 | wingless-type MMTV integration site family, member 3 |
| 283314 | MATL2963 | hypothetical LOC283314 |
| 728545 | [No Symbol] | [No Name] |
| 151825 | KRT18P43 | keratin 18 pseudogene 43 |
| 647532 | LOC647532 | phenylalanine-tRNA synthetase-like, beta subunit pseudogene |
| 644862 | RPS28P3 | ribosomal protein S28 pseudogene 3 |
| 100128403 | [No Symbol] | [No Name] |
| 3626 | INHBC | inhibin, beta C |
| 347051 | SLC10A5 | solute carrier family 10 (sodium/bile acid cotransporter fam . . . |
| 283571 | PROX2 | prospero homeobox 2 |
| 100132510 | GLRXP3 | glutaredoxin (thioltransferase) pseudogene 3 |
| 25925 | ZNF521 | zinc finger protein 521 |
| 28869 | IGKV6D-41 | immunoglobulin kappa variable 6D-41 (non-functional) |
| 387856 | C12orf68 | chromosome 12 open reading frame 68 |
| 606495 | CYB5RL | cytochrome b5 reductase-like |
| 644588 | DNAJA1P3 | DnaJ (Hsp40) homolog, subfamily A, member 1 pseudogene 3 |
| 56834 | GPR137 | G protein-coupled receptor 137 |
| 100130184 | [No Symbol] | [No Name] |
| 574447 | MIR146B | microRNA 146b |
| 8626 | TP63 | tumor protein p63 |
| 286495 | TTC3P1 | tetratricopeptide repeat domain 3 pseudogene 1 |
| 387924 | OGFOD1P1 | 2-oxoglutarate and iron-dependent oxygenase domain containin . . . |
| 55702 | CCDC94 | coiled-coil domain containing 94 |
| 390844 | ARIH2P1 | ariadne homolog 2 pseudogene 1 |
| 5054 | SERPINE1 | serpin peptidase inhibitor, clade E (nexin, plasminogen acti . . . |
| 641518 | LOC641518 | hypothetical LOC641518 |
| 4085 | MAD2L1 | MAD2 mitotic arrest deficient-like 1 (yeast) |
| 100131070 | LOC100131070 | mpv17-like protein 2-like |
| 338094 | FAM151A | family with sequence similarity 151, member A |
| 388507 | ZNF788 | zinc finger family member 788 |
| 7164 | TPD52L1 | tumor protein D52-like 1 |
| 339983 | NAT8L | N-acetyltransferase 8-like (GCN5-related, putative) |
| 57570 | TRMT5 | TRM5 tRNA methyltransferase 5 homolog (S. cerevisiae) |
| 100132053 | RPL30P8 | ribosomal protein L30 pseudogene 8 |
| 100131750 | [No Symbol] | [No Name] |
| 285231 | FBXW12 | F-box and WD repeat domain containing 12 |
| 400347 | LOC400347 | REX4, RNA exonuclease 4 homolog (S. cerevisiae) pseudogene |
| 6887 | TAL2 | T-cell acute lymphocytic leukemia 2 |
| 100131602 | [No Symbol] | [No Name] |
| 3812 | KIR3DL2 | killer cell immunoglobulin-like receptor, three domains, lon . . . |
| 3625 | INHBB | inhibin, beta B |
| 201895 | C4orf34 | chromosome 4 open reading frame 34 |
| 56891 | LGALS14 | lectin, galactoside-binding, soluble, 14 |
| 442673 | TUBG1P | tubulin, gamma 1 pseudogene |
| 2841 | GPR18 | G protein-coupled receptor 18 |
| 392301 | SLC25A5P8 | solute carrier family 25 (mitochondrial carrier; adenine nuc . . . |
| 414318 | C9orf106 | chromosome 9 open reading frame 106 |
| 645233 | LOC645233 | thymine-DNAglycosylase pseudogene |
| 23439 | ATP1B4 | ATPase, Na+/K+ transporting, beta 4 polypeptide |
| 219902 | TMEM136 | transmembrane protein 136 |
| 166752 | FREM3 | FRAS1 related extracellular matrix 3 |
| 79825 | CCDC48 | coiled-coil domain containing 48 |
| 54035 | PSMD4P1 | proteasome (prosome, macropain) 26S subunit, non-ATPase, 4 p . . . |
| 646272 | LOC646272 | cytochrome b-c1 complex subunit 8-like |
| 643669 | LOC643669 | hypothetical protein LOC643669 |
| 145259 | RPSAP4 | ribosomal protein SA pseudogene 4 |
| 2335 | FN1 | fibronectin 1 |
| 81849 | ST6GALNAC5 | ST6 (alpha-N-acetyl-neuraminyl-2,3-beta-galactosyl-1,3)-N-ac . . . |
| 100132073 | LOC100132073 | cyclin B2 pseudogene |
| 100133337 | [No Symbol] | [No Name] |
| 100130623 | [No Symbol] | [No Name] |
| 100132310 | LOC100132310 | FCF1 small subunit (SSU) processome component homolog (S. ce . . . |
| 5988 | RFPL1 | ret finger protein-like 1 |
| 84099 | ID2B | inhibitor of DNA binding 2B, dominant negative helix-loop-he . . . |
| 644464 | RPSAP61 | ribosomal protein SA pseudogene 61 |
| 148823 | C1orf150 | chromosome 1 open reading frame 150 |
| 130162 | C2orf63 | chromosome 2 open reading frame 63 |
| 23754 | RPL32P5 | ribosomal protein L32 pseudogene 5 |
| 10316 | NMUR1 | neuromedin U receptor 1 |
| 401703 | LOC401703 | splicing factor U2AF 35 kDa subunit-like |
| 1266 | CNN3 | calponin 3, acidic |
| 728780 | ANKDD1B | ankyrin repeat and death domain containing 1B |
| 643182 | LOC643182 | upstream binding transcription factor, RNA polymerase I pseu . . . |
| 81889 | FAHD1 | fumarylacetoacetate hydrolase domain containing 1 |
| 344807 | CD200R1L | CD200 receptor 1-like |
| 140691 | TRIM69 | tripartite motif containing 69 |
| 644915 | METTL15P2 | methyltransferase like 15 pseudogene 2 |
| 139411 | PTCHD1 | patched domain containing 1 |
| 339736 | AK2P2 | adenylate kinase 2 pseudogene 2 |
| 118663 | BTBD16 | BTB (POZ) domain containing 16 |
| 64410 | KLHL25 | kelch-like 25 (Drosophila) |
| 28466 | IGHV1-45 | immunoglobulin heavy variable 1-45 |
| 285987 | DLX6-AS1 | DLX6 antisense RNA 1 (non-protein coding) |
| 130013 | ACMSD | aminocarboxymuconate semialdehyde decarboxylase |
| 259286 | TAS2R40 | taste receptor, type 2, member 40 |
| 9363 | RAB33A | RAB33A, member RAS oncogene family |
| 729950 | LOC729950 | hypothetical LOC729950 |
| 645474 | S100A11P3 | S100 calcium binding protein A11 pseudogene 3 |
| 653712 | LOC653712 | intraflagellar transport 122 homolog (Chlamydomonas) pseudog . . . |
| 81050 | OR5AC2 | olfactory receptor, family 5, subfamily AC, member 2 |
| 3226 | HOXC10 | homeobox C10 |
| 392387 | LOC392387 | adenosylhomocysteinase pseudogene |
| 126259 | TMIGD2 | transmembrane and immunoglobulin domain containing 2 |
| 81431 | OR5AC1 | olfactory receptor, family 5, subfamily AC, member 1 (gene/p . . . |
| 79696 | FAM164C | family with sequence similarity 164, member C |
| 100131087 | RPLP1P11 | ribosomal protein, large, P1 pseudogene 11 |
| 644941 | [No Symbol] | [No Name] |
| 100127983 | LOC100127983 | hypothetical protein LOC100127983 |
| 51499 | TRIAP1 | TP53 regulated inhibitor of a poptosis 1 |

TABLE 13
Top genes consistently differentially expressed between individuals
who developed hereditary breast cancer and controls.

| Entrez | | |
|---|---|---|
| Gene ID | Gene Symbol | Gene Name |
| 4675 | NAP1L3 | nucleosome assembly protein 1-like 3 |
| 11123 | RCAN3 | RCAN family member 3 |
| 5769 | PTP4A2P2 | protein tyrosine phosphatase type IVA, member 2 pseudogene 2 |
| 11043 | MID2 | midline 2 |
| 6920 | TCEA3 | transcription elongation factor A (SII), 3 |
| 959 | CD40LG | CD40 ligand |
| 8797 | TNFRSF10A | tumor necrosis factor receptor superfamily, member 10a |
| 91624 | NEXN | nexilin (F actin binding protein) |
| 100132341 | KIAA0664L3 | KIAA0664-like3 |
| 4050 | LTB | lymphotoxin beta (TNF superfamily, member 3) |
| 219670 | ENKUR | enkurin, TRPC channel interacting protein |
| 400360 | C15orf54 | chromosome 15 open reading frame 54 |
| 128553 | TSHZ2 | teashirt zinc finger homeobox 2 |
| 158399 | ZNF483 | zinc finger protein 483 |
| 390980 | ZNF805 | zinc finger protein 805 |
| 51301 | GCNT4 | glucosaminyl (N-acetyl) transferase 4, core 2 |
| 940 | CD28 | CD28 molecule |
| 92106 | OXNAD1 | oxidoreductase NAD-binding domain containing 1 |
| 552 | AVPR1A | arginine vasopressin receptor 1A |
| 643162 | DKFZP779L1853 | hypothetical LOC643162 |
| 56985 | C17orf48 | chromosome 17 open reading frame 48 |
| 9840 | KIAA0748 | KIAA0748 |
| 2791 | GNG11 | guanine nucleotide binding protein (G protein), gamma 11 |
| 161291 | TMEM30B | transmembrane protein 30B |
| 80177 | MYCT1 | myc target 1 |
| 28984 | C13orf15 | chromosome 13 open reading frame 15 |
| 100132760 | [No Symbol] | [No Name] |
| 100133168 | GPR183P1 | G protein-coupled receptor 183 pseudogene 1 |
| 2811 | GP1BA | glycoprotein Ib (platelet), alpha polypeptide |
| 120776 | OR2D2 | olfactory receptor, family 2, subfamily D, member 2 |
| 401093 | LOC401093 | hypothetical LOC401093 |
| 687 | KLF9 | Kruppel-like factor 9 |
| 3957 | LGALS2 | lectin, galactoside-binding, soluble, 2 |
| 3655 | ITGA6 | integrin, alpha 6 |
| 64407 | RGS18 | regulator of G-protein signaling 18 |
| 56271 | BEX4 | brain expressed, X-linked 4 |
| 5920 | RARRES3 | retinoic acid receptor responder (tazarotene induced) 3 |
| 9241 | NOG | noggin |
| 643951 | [No Symbol] | [No Name] |
| 54033 | RBM11 | RNA binding motif protein 11 |
| 342618 | SLFN14 | schlafen family member 14 |
| 340554 | ZC3H12B | zinc finger CCCH-type containing 12B |
| 282997 | LOC282997 | hypothetical LOC282997 |
| 322 | APBB1 | amyloid beta (A4) precursor protein-binding, family B, membe . . . |
| 401303 | ZNF815 | zinc finger protein 815 |
| 5140 | PDE3B | phosphodiesterase 3B, cGMP-inhibited |
| 54557 | SGTB | small glutamine-rich tetratricopeptide repeat (TPR)-containi . . . |
| 51266 | CLEC1B | C-type lectin domain family 1, member B |
| 258010 | SVIP | small VCP/p97-interacting protein |
| 135644 | TRIM40 | tripartite motif containing 40 |
| 100131733 | LOC100131733 | hypothetical LOC100131733 |
| 64805 | P2RY12 | purinergic receptor P2Y, G-protein coupled, 12 |
| 3081 | HGD | homogentisate 1,2-dioxygenase |
| 23531 | MMD | monocyte to macrophage differentiation-associated |
| 55837 | EAPP | E2F-associated phosphoprotein |
| 439949 | LOC439949 | hypothetical LOC439949 |
| 25949 | SYF2 | SYF2 homolog, RNA splicing factor (S. cerevisiae) |
| 57191 | VN1R1 | vomeronasal 1 receptor 1 |
| 80024 | SLC24A6 | solute carrier family 24 (sodium/potassium/calcium exchanger . . . |
| 147727 | LOC147727 | hypothetical LOC147727 |
| 288 | ANK3 | ankyrin 3, node of Ranvier (ankyrin G) |
| 5874 | RAB27B | RAB27B, member RAS oncogene family |
| 283897 | C16orf54 | chromosome 16 open reading frame 54 |
| 144363 | LYRM5 | LYR motif containing 5 |
| 50865 | HEBP1 | heme binding protein 1 |
| 130399 | ACVR1C | activin A receptor, type IC |
| 152217 | LOC152217 | hypothetical LOC152217 |
| 32 | ACACB | acetyl-CoA carboxylase beta |
| 1408 | CRY2 | cryptochrome 2 (photolyase-like) |
| 5578 | PRKCA | protein kinase C, alpha |
| 1823 | DSC1 | desmocollin 1 |
| 8328 | GFI1B | growth factor independent 1B transcription repressor |
| 340547 | VSIG1 | V-set and immunoglobulin domain containing 1 |
| 10778 | ZNF271 | zinc finger protein 271 |
| 8436 | SDPR | serum deprivation response |
| 26119 | LDLRAP1 | low density lipoprotein receptor adaptor protein 1 |
| 51176 | LEF1 | lymphoid enhancer-binding factor 1 |
| 10286 | BCAS2 | breast carcinoma amplified sequence 2 |
| 2053 | EPHX2 | epoxide hydrolase 2, cytoplasmic |
| 168537 | GIMAP7 | GTPase, IMAP family member 7 |
| 113791 | PIK3IP1 | phosphoinositide-3-kinase interacting protein 1 |
| 4240 | MFGE8 | milk fat globule-EGF factor 8 protein |
| 5742 | PTGS1 | prostaglandin-endoperoxide synthase 1 (prostaglandin G/H syn . . . |
| 403313 | PPAPDC2 | phosphatidic acid phosphatase type 2 domain containing 2 |
| 3337 | DNAJB1 | DnaJ (Hsp40) homolog, subfamily B, member 1 |
| 10140 | TOB1 | transducer of ERBB2, 1 |
| 442673 | TUBG1P | tubulin, gamma 1 pseudogene |
| 7644 | ZNF91 | zinc finger protein 91 |
| 87 | ACTN1 | actinin, alpha 1 |
| 163259 | DENND2C | DENN/MADD domain containing 2C |
| 55664 | CDC37L1 | cell division cycle 37 homolog (S. cerevisiae)-like 1 |
| 6403 | SELP | selectin P (granule membrane protein 140 kDa, antigen CD62) |
| 3480 | IGF1R | insulin-likegrowth factor 1 receptor |
| 100101405 | LOC100101405 | chromosome 4 open reading frame 43 pseudogene |
| 91782 | CHMP7 | CHMP family, member 7 |
| 120224 | TMEM45B | transmembrane protein 45B |
| 255231 | MCOLN2 | mucolipin 2 |
| 259215 | LY6G6F | lymphocyte antigen 6 complex, locus G6F |
| 94121 | SYTL4 | synaptotagmin-like 4 |
| 22915 | MMRN1 | multimerin 1 |
| 9925 | ZBTB5 | zinc finger and BTB domain containing 5 |
| 222166 | C7orf41 | chromosome 7 open reading frame 41 |
| 7695 | ZNF136 | zinc finger protein 136 |
| 100129866 | LOC100129866 | etoposide induced 2.4 mRNA pseudogene |
| 644693 | [No Symbol] | [No Name] |
| 23484 | LEPROTL1 | leptin receptor overlapping transcript-like 1 |
| 130500 | CISD1P1 | CDGSH iron sulfur domain 1 pseudogene 1 |
| 10857 | PGRMC1 | progesterone receptor membrane component 1 |
| 100128616 | [No Symbol] | [No Name] |
| 55766 | H2AFJ | H2A histone family, member J |
| 143686 | SESN3 | sestrin 3 |
| 7381 | UQCRB | ubiquinol-cytochrome c reductase binding protein |
| 2686 | GGT7 | gamma-glutamyltransferase 7 |
| 7754 | ZNF204P | zinc finger protein 204, pseudogene |
| 9848 | MFAP3L | microfibrillar-associated protein 3-like |
| 100129436 | [No Symbol] | [No Name] |
| 321 | APBA2 | amyloid beta (A4) precursor protein-binding, family A, membe . . . |
| 2815 | GP9 | glycoprotein IX (platelet) |
| 8654 | PDE5A | phosphodiesterase 5A, cGMP-specific |
| 4054 | LTBP3 | latent transforming growth factor beta binding protein 3 |
| 84749 | USP30 | ubiquitin specific peptidase 30 |
| 7294 | TXK | TXK tyrosine kinase |
| 10023 | FRAT1 | frequently rearranged in advanced T-cell lymphomas |
| 391833 | RPS10P11 | ribosomal protein S10 pseudogene 11 |
| 10179 | RBM7 | RNA binding motif protein 7 |

What is claimed is:

1. A gene expression panel or array indicative of the risk of developing breast cancer, said panel or array consisting of primers or probes capable of detecting at least 1 gene selected from: (i) the genes of Table 1; (ii) the genes of Table 2; and (iii) genes of Table 3; or (iv) the genes of Table 4.

2. The gene expression panel or array of claim 1, wherein a range of 1-300 genes or more genes are selected from Table 1.

3. The gene expression panel or array of claim 1, wherein a range of 1-300 genes or more genes are selected from Table 2.

4. The gene expression panel or array of claim 1, wherein a range of 1-300 genes or more genes are selected from Table 3.

5. The gene expression panel or array of claim 1, wherein a range of 1-300 genes or more genes are selected from Table 4.

6. The gene expression panel or array of claim 1, wherein the panel or array consists of primers or probes capable of detecting between 20 and 300 genes.

7. The gene expression panel or array of claim 1, wherein the risk is specific to hereditary breast cancer.

8. The gene expression panel or array of claim 1, wherein the risk is not specific to hereditary breast cancer.

9. A diagnostic kit containing probes or primers for measuring the expression of one or more genes of Table 1, Table 2, Table 3, and Table 4.

10. A method of assessing a subject's susceptibility to develop cancer, wherein said cancer is a breast cancer comprising:
a. obtaining a nucleic acid for variant detection and/or deregulation of genes or gene expression products, wherein said genes or gene expression products are selected from: (i) the genes of Table 1; (ii) the genes of Table 2; and (iii) genes of Table 3; or (iv) the genes of Table 4; and wherein 1-300 genes or gene expression products are selected
b. obtaining a profile of the expression levels of the selected genes or gene expression products in said sample; and
c. assessing a subject's susceptibility to develop breast cancer based upon genetic variants and/or a variance in the obtained profile of expression levels of the said selected genes or gene expression products in said subject's sample from the same selected genes or gene expression products of a control gene expression profile from a similar biological sample of a healthy subject, or assessing a subject's susceptibility to develop breast cancer based upon a similarity in the obtained profile of expression levels of said selected genes or gene expression products in said subject's sample to the same selected genes or gene expression products in a gene expression profile characteristic of a subject with breast cancer or a subject who has a proven susceptibility to develop breast cancer.

11. The method of claim 10, wherein the nucleic acid is obtained by extracting nucleic acid from a biological sample of the subject containing immune cells, peripheral blood, epithelial cells or cancer cells.

12. The method of claim 10, wherein the nucleic acid is RNA and/or DNA.

13. The method of claim 12, wherein cDNA is generated from the RNA

14. The method according to claim 10, wherein said profile is provided in the form of a graph or tree view.

15. The method according to claim 10, wherein said sample is peripheral blood.

16. The method according to claim 10, wherein said immune cells are peripheral blood mononuclear cells.

17. The method of claim 10, wherein 1-300 or more genes are selected from Table 1.

18. The method of claim 10, wherein 1-300 or more genes are selected from Table 2.

19. The method of claim 10, wherein 1-300 or more genes are selected from Table 3.

20. The method of claim 10, wherein 1-300 or more genes are selected from Table 4.

21. The method of claim 10, wherein the panel or array consists of primers or probes capable of detecting between 20 and 300 genes.

22. The method according to claim 10, wherein a gene profile formed by a decreased level of one or more of the genes of Table 1 and Table 3, and/or increased levels of one or more of the genes of Table 2 and Table 4, as compared to said healthy profile is indicative of breast cancer or a subject's susceptibility to develop breast cancer.

23. A method for diagnosing breast cancer in a mammalian subject comprising
a. obtaining a nucleic acid for analysis of genes or gene expression products, wherein said genes or gene expression products are selected from: (i) the genes of Table 1; (ii) the genes of Table 2; and (iii) genes of Table 3; or (iv) the genes of Table 4; and wherein at least 5 genes or gene expression products are selected
b. obtaining a profile of the expression levels of the selected genes or gene expression products in said sample; and
c. diagnosing breast cancer based upon a pattern of obtained expression levels of the said genes or gene expression products that form a gene expression profile characteristic of breast cancer in said subject's sample.

24. The method of claim 23, wherein the nucleic acid is obtained by extracting nucleic acid from a biological sample of the subject containing immune cells or cancer cells.

25. The method of claim 23, wherein the nucleic acid is RNA or DNA.

26. The method of claim 25, wherein cDNA is generated from the RNA

27. The method according to claim 23, wherein said profile is provided in the form of a graph or tree view.

28. The method according to claim 23, wherein said sample is peripheral blood.

29. The method according to claim 23, wherein said immune cells are peripheral blood mononuclear cells.

30. The method of claim 23, wherein 1-300 or more genes are selected from Table 1.

31. The method of claim 23, wherein 1-300 or more genes are selected from Table 2.

32. The method of claim 23, wherein 1-300 or more genes are selected from Table 3.

33. The method of claim 23, wherein 1-300 or more genes are selected from Table 4.

34. The method of claim 23, wherein the panel or array consists of primers or probes capable of detecting between 20 and 300 genes.

35. The method according to claim 23, wherein a gene profile formed by a decreased level of one or more of the genes of Table 1 and Table 3, and/or increased levels of one or more of the genes of Table 2 and Table 4, as compared to said healthy profile is indicative of breast cancer or a subject's susceptibility to develop breast cancer.

36. The method according to claim 23, wherein said pattern of obtained expression levels of the said genes or gene expression products is compared to a control gene expression profile.

37. The method of claim 36, wherein the control gene expression profile is from a similar biological sample of a healthy subject.

38. A method of assessing a subject's susceptibility to develop cancer, wherein said cancer is a breast cancer comprising:
a. obtaining a nucleic acid for analysis of genes or gene expression products, wherein said genes or gene expression products are selected from: (i) the genes of Table 1; (ii) the genes of Table 2; and (iii) genes of Table 3; or (iv) the genes of Table 4; and wherein at least 20 genes or gene expression products are selected
b. obtaining a profile of the expression levels of the selected genes or gene expression products in said sample; and
c. normalizing said expression level to obtain a normalized expression level of the genes of (b);
d. assessing a subject's susceptibility to develop breast cancer based upon a variance in the obtained profile of expression levels of the said selected genes or gene expression products in said subject's sample from the same selected genes or gene expression products of a control gene expression profile from a similar biological sample of a healthy subject, or assessing a subject's susceptibility to develop breast cancer based upon a similarity in the obtained profile of expression levels of said selected genes or gene expression products in said subject's sample to the same selected genes or gene expression products in a gene expression profile characteristic of a subject with breast cancer or a subject who has a proven susceptibility to develop breast cancer.

39. The method of claim 1, wherein one of the following normalization methods are used in step (c): mass, RMA, DWD, or SCAN.

40. A method for diagnosing breast cancer in a mammalian subject comprising:
a. obtaining a nucleic acid for analysis of genes or gene expression products, wherein said genes or gene expression products are selected from: (i) the genes of Table 1; (ii) the genes of Table 2; and (iii) genes of Table 3; or (iv) the genes of Table 4; and wherein at least 20 genes or gene expression products are selected
b. obtaining a profile of the expression levels of the selected genes or gene expression products in said sample; and
c. normalizing said expression level to obtain a normalized expression level of the genes of (a); and
d. diagnosing breast cancer based upon a pattern of obtained expression levels of the said genes or gene expression products that form a gene expression profile characteristic of breast cancer in said subject's sample.

41. A method of preparing a personalized genomics profile for a breast cancer subject, comprising:
a. obtaining a nucleic acid for analysis of genes or gene expression products, wherein said genes or gene expression products are selected from: (i) the genes of Table 1; (ii) the genes of Table 2; and (iii) genes of Table 3; or (iv) the genes of Table 4; and wherein at least 20 genes or gene expression products are selected
b. obtaining the expression levels of said genes or gene expression products in said sample; wherein the expression level is normalized against the expression level of at least one reference gene to obtain normalized data or the expression levels in a breast cancer reference tissue set; and
c. creating a report summarizing the normalized data obtained by said gene expression analysis, wherein said report includes a prediction of a subject's increased likelihood to develop breast cancer.

42. A gene expression panel or array indicative of the risk of developing breast cancer, said panel or array consisting of primers or probes capable of detecting at least 20 genes selected from: (i) the genes of Table 5 or; (ii) the genes of Table 6.

43. The gene expression panel or array of claim 42, wherein 1-79 genes are selected from Table 5.

44. The gene expression panel or array of claim 42, wherein 1-100 or more genes are selected from Table 6.

45. The gene expression panel or array of claim 1, wherein the panel or array consists of primers or probes capable of detecting between 20 and 200 genes

46. The gene expression panel or array of claim 42, wherein the risk is specific to hereditary breast cancer.

47. The gene expression panel or array of claim 42, wherein the risk is not specific to hereditary breast cancer.

48. A diagnostic kit containing probes or primers for measuring the expression of one or more genes of Table 5 and Table 6.

49. A method of assessing a subject's susceptibility to develop cancer, wherein said cancer is a breast cancer comprising:
a. obtaining a nucleic acid for amplification of genes or gene expression products, wherein said genes or gene expression products are selected from: (i) the genes of Table 5; or (ii) the genes of Table 6; and wherein at least 1 gene or gene expression products are selected
b. obtaining a profile of the expression levels of the selected genes or gene expression products in said sample; and
c. assessing a subject's susceptibility to develop breast cancer based upon a variance in the obtained profile of expression levels of the said selected genes or gene expression products in said subject's sample from the same selected genes or gene expression products of a control gene expression profile from a similar biological sample of a healthy subject, or assessing a subject's susceptibility to develop breast cancer based upon a similarity in the obtained profile of expression levels of said selected genes or gene expression products in said subject's sample to the same selected genes or gene expression products in a gene expression profile characteristic of a subject with breast cancer or a subject who has a proven susceptibility to develop breast cancer.

50. The method of claim 49, wherein the nucleic acid is obtained by extracting nucleic acid from a biological sample of the subject containing immune cells or cancer cells.

51. The method of claim 49, wherein 1-79 genes are selected from Table 5.

52. The method of claim 49, wherein 1-100 or more genes are selected from Table 6.

53. The method of claim 49, wherein the panel or array consists of primers or probes capable of detecting between 1-100 genes.
